(19) United States
(12) Patent Application Publication
(10) Pub. No.: US 2025/0387508 A1
(43) Pub. Date: Dec. 25, 2025

(54) METHODS OF PRODUCING HYDROGEL MICROSPHERES

(71) Applicant: Ascendis Pharma A/S, Hellerup (DK)

(72) Inventors: Sebastian Stark, Heidelberg (DE); Benjamin Breitenbach, Heidelberg (DE); Carsten Roessel, Heidelberg (DE); Dennis Sauer, Heidelberg (DE); Thomas Knappe, Heidelberg (DE); Tobias Voigt, Heidelberg (DE); Till Preuss, Heidelberg (DE)

(21) Appl. No.: 19/254,990

(22) Filed: Jun. 30, 2025

Related U.S. Application Data
(63) Continuation of application No. PCT/EP2024/050090, filed on Jan. 3, 2024.

(30) Foreign Application Priority Data
Jan. 5, 2023 (EP) 23150461.4
Sep. 19, 2023 (EP) 23198176.2

(51) Int. Cl. A61K 47/69; A61K 47/61; C08J 3/24

(52) U.S. Cl. CPC A61K 47/6927; A61K 47/61; C08J 3/24

(57) ABSTRACT

The present invention relates to hydrogel hyaluronic acid (HA) microspheres or pharmaceutically acceptable salts thereof that are prepared via suspension polymerization. Said hydrogel HA microspheres prepared according to the methods of the present invention may be used as carriers of various agents, such as carriers of various drug moieties. The present invention also provides for drug conjugates or pharmaceutically acceptable salts thereof that employ said hydrogel HA microspheres as carriers, methods of making said drug conjugates, pharmaceutical compositions comprising said drug conjugates and their use.

### CROSS-REFERENCE TO RELATED APPLICATIONS

This application is a continuation of International Patent Application No. PCT/EP2024/050090 filed Jan. 3, 2024, which claims priority from EP 23198176.2 filed Sep. 19, 2023, and from EP 23150461.4 filed Jan. 5, 2023; the contents of each of which are herein incorporated by reference in their entireties.

The present invention relates to hydrogel hyaluronic acid (HA) microspheres or pharmaceutically acceptable salts thereof that are prepared via suspension polymerization. Said hydrogel HA microspheres prepared according to the methods of the present invention may be used as carriers of various agents, such as carriers of various drug moieties. The present invention also provides for drug conjugates or pharmaceutically acceptable salts thereof that employ said hydrogel HA microspheres as carriers, methods of making said drug conjugates, pharmaceutical compositions comprising said drug conjugates and their use.

To improve physicochemical or pharmacokinetic properties, such as the in vivo circulation half-life of drugs, such drugs can be conjugated to a carrier, such as a hydrogel. Typically, hydrogels in drug delivery are used in a non-covalent complexation between a drug and hydrogel, the drug can be embedded in the hydrogel, or in a covalent fashion via conjugation of the drug to the hydrogel.

The non-covalent approach requires a highly efficient drug encapsulation to prevent uncontrolled, burst-type release of the drug due to disintegration of the drug-hydrogel complex after administration. Restraining the diffusion of an unbound, water-soluble drug molecule requires strong van der Waals interactions, frequently mediated through hydrophobic and charged moieties for electrostatic binding. Many conformationally sensitive drugs, such as proteins or peptides, are rendered dysfunctional during the complexation process and/or during subsequent storage of the non-covalently bound drug.

Alternatively, a drug may be covalently conjugated to a hydrogel via a linker moiety, whereby the linkage between the drug and the linker is stable or via a linker moiety, whereby the linkage between the drug and the linker moiety is reversible.

Conventional hydrogels are three-dimensional, hydrophilic or amphiphilic polymeric networks capable of taking up large quantities of water. These networks may comprise various polymers and are insoluble due to the presence of covalent and/or physical crosslinks, such as ionic, hydrophobic interactions or entanglements.

Various drug delivery systems comprising drugs covalently attached to hydrogels have been developed. For example, WO 2018/175788 A1 teaches hydrogel prodrug compositions comprising crosslinked hyaluronic acid (HA) to which drug-linker moieties are conjugated. HA is a biocompatible hydrophilic polysaccharide consisting of D-glucuronic acid and N-acetyl-D-glucosamine. Said prodrugs may be used in the treatment of ocular conditions, particularly in the field of neovascular eye diseases, which require repeated intravitreal injections of antivascular endothelial growth factors. In particular, for hydrogel injection applications, it is important to improve the biostability of the hydrogel carrier as well as its injectability to pass easily through the needle. WO 2018/193408 A1 describes a drug delivery system consisting of hyaluronic acid hydrogels that are attached to drug moieties via reversible linkers. In this case, the bulk hydrogel may be rendered injectable via mechanical milling.

There continues to be a need for new drug delivery systems suitable for the sustained release of drugs.

This objective is achieved with a method for preparing hydrogel microspheres or pharmaceutically acceptable salts thereof comprising a crosslinked hyaluronic acid (HA), wherein the method comprises the steps of:
(a) mixing a solution A with a solution B to form an emulsion, wherein solution A comprises a first functionalized HA that is modified with one or more -FG₁ and optionally further functional groups and a second functionalized HA that is modified with one or more -FG₂ and optionally further functional groups, wherein each -FG₁ and -FG₂ are functional group moieties that are different from each other, wherein -FG₁ on the first functionalized HA reacts with -FG₂ on the second functionalized HA to form a plurality of crosslinks which results in the formation of hydrogel HA microspheres;
(b) optionally, adding a pH-adjusting agent to the emulsion of step (a); and
(c) collecting the obtained hydrogel HA microspheres of step (a) or (b).

Described herein are methods for making HA hydrogel microspheres or pharmaceutically acceptable salts thereof that can be easily injected. The injectability is enhanced owing to the relatively uniform size and shape of the hydrogel microspheres. Said HA hydrogel microspheres may be used as carriers which are covalently conjugated to drug moieties and can provide a valuable tool for localized drug delivery.

Within the meaning of the present invention the terms are used as follows.

As used herein, the term “about” in combination with a numerical value is used to indicate a range ranging from and including the numerical value plus and minus no more than 20% of said numerical value, in certain embodiments, no more than 15% of said numerical value, in certain embodiments, no more than 10% of said numerical value and in certain embodiments, no more than 5% of said numerical value. For example, the phrase “about 200” is used to mean a range ranging from and including 200+/−20%, i.e. ranging from and including 160 to 240; in certain embodiments, 200+/−15%, i.e. ranging from and including 170 to 230; in certain embodiments, ranging from and including 200+/−10%, i.e. ranging from and including 180 to 220; and in certain embodiments 200+/−5%, i.e. ranging from and including 190 to 210.

As used herein, the term “polysaccharide(s)” also referred to as “glycan(s)” refers to compounds consisting of monosaccharide moieties linked glycosidically. Typically, this term is used for compounds consisting of a large number of monosaccharide moieties linked glycosidically, e.g., such as more than ten monosaccharide moieties.

As used herein, the term “glycosaminoglycan” or “mucopolysaccharide” refers to a linear polysaccharide consisting of disaccharide units, wherein the repeating two-sugar unit consists of a uronic sugar and an amino sugar, except in the case of sulfated glycosaminoglycan keratan, where, in place of the uronic sugar there is a galactose unit. Examples of glycosaminoglycan molecules may include hyaluronic acid, heparin, heparan sulfate, heparosan, chondroitin sulfate, dermatan sulfate or keratan sulfate molecules.

As used herein, the term “functionalized hyaluronic acid” refers to any hyaluronic acid derivative that may result from a chemical or enzymatic functionalization or modification of a native hyaluronic acid. In particular, said term refers to any hyaluronic acid derivative that may result from the chemical modification or functionalization at the carboxylic acid group.

As used herein, the term “microspheres” refers to micron-scale particles which are typically composed of solid or semi-solid materials and which are substantially spherical. Typically, the average diameter of the microspheres of the present invention, as determined by microscopy such as flow microscopy or laser diffraction or any other suitable method, ranges from about 1 μm to about 1000 μm, such as from about 10 μm to about 500 μm or such as from about 50 μm to about 500 μm.

As used herein, the term “dispersed phase” refers to a phase comprising particles or droplets of any size and of any nature which are distributed through or dispersed in a continuous phase. The diameter of the droplets within the dispersed phase may range from about 1 μm to about 5000 μm, such as from about 10 μm to about 1000 μm or such as from about 50 μm to about 500 μm. In the dispersed phase found in the emulsions of the present invention the average diameter of the droplets in the dispersed phase typically ranges from 1 μm to about 1000 μm, such as from 10 μm to about 500 μm or such as from about 50 μm to about 500 μm.

As used herein, the term “continuous phase” or “continuous phase solution” refers to the fluid phase within which solid or fluid particles or droplets are distributed.

As used herein, the term “emulsion” refers to a fluid system in which droplets of one liquid are dispersed in another liquid in which it is not soluble or miscible with. An emulsion is termed as oil/water (o/w) emulsion if the dispersed phase is an organic material and the continuous phase is water or an aqueous solution and is termed water/oil (w/o) if the dispersed phase is water or an aqueous solution and the continuous phase is an organic liquid.

As used herein, the term “suspension polymerization” refers to a process of polymerization in which a polymer, such as a hydrogel, is formed in monomer or monomer-solvent droplets in a continuous phase that is non-solvent for both the monomer and the formed polymer. As the monomer is converted into polymer, the droplets are transformed into sticky, viscous monomer and/or polymer particles that gradually become spherical solid polymer particles or microspheres. It is understood that in the context of the present invention the beforementioned monomer corresponds to the first and second functionalized HA and the formed polymer to the HA hydrogel.

As used herein, the term “drug” refers to a substance used in the treatment, cure, prevention or diagnosis of a disease or used to otherwise enhance physical or mental well-being of a patient. If a drug is conjugated to another moiety, the moiety of the resulting product that originated from the drug is referred to as “drug moiety”.

As used herein, the term “primary or secondary amine-comprising moiety of a drug D-H” refers to a moiety of a drug comprising at least one primary or secondary amine functional group, which drug may optionally have one or more further functional group(s) including one or more additional primary and/or secondary amine functional group(s).

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “-” indicates attachment to another moiety. Accordingly, a drug moiety is released from a reversible linkage as a drug.

It is understood that if a sequence or chemical structure of a group of atoms is provided which group of atoms is attached to two moieties or is interrupting a moiety, said sequence or chemical structure can be attached to the two moieties in either orientation, unless explicitly stated otherwise. For example, a moiety “—C(O)N(R^{x})—” may be attached to two moieties or interrupting a moiety either as “—C(O)N(R^{x})—” or as “—N(R^{x})C(O)”.

As used herein, the term “protecting group moiety” refers to a moiety which is reversibly connected to a functional group to render it incapable of reacting with, for example, another functional group. Suitable alcohol (—OH) protecting groups are, for example, acetyl, benzoyl, benzyl, β-methoxyethoxymethyl ether, dimethoxytrityl, methoxymethyl ether, methoxytrityl, p-methoxybenzyl ether, methylthiomethyl ether, pivaloyl, tetrahydropyranyl, trityl, trimethylsilyl, tert-butyldimethylsilyl, tri-iso-propylsilyloxymethyl, triisopropylsilyl ether, methyl ether, and ethoxyethyl ether. Suitable carbonyl protecting groups are, for example, acetals and ketals, acylals and dithianes. Suitable carboxylic acid protecting groups are, for example, methyl esters, benzyl esters, tert-butyl esters, 2,6-dimethylphenol, 2,6-diisopropylphenol, 2,6.-di-tert-butylphenol, silyl esters, orthoesters, and oxazoline. Suitable phosphate protecting groups are, for example, 2-cyanoethyl and methyl.

As used herein, the term “amine protecting group moiety” refers to a moiety that is used for the reversible protection of an amine functional group during chemical reaction processes to render said amine incapable of reacting with, for example, another functional group.

As used herein, the term “reducing agent” refers to a chemical compound or element that loses or donates an electron to an electron recipient such as an oxidizing agent in a redox chemical reaction.

As used herein, the term “oxidizing agent” refers to a chemical compound that is able to oxidize other chemical compounds.

As used herein, the term “reagent” means a chemical compound, which comprises at least one functional group for reaction with the functional group of another chemical compound or drug. It is understood that a drug comprising a functional group is also a reagent.

It is recognized by one of ordinary skill in the art that the drug conjugates or pharmaceutically acceptable salt thereof of the present invention are prodrugs. As used herein, the term “prodrug” refers to a drug moiety, that is reversibly and covalently conjugated to hyaluronic acid via a -L¹-L²- moiety. A prodrug releases the reversibly and covalently bound drug moiety -D or D⁺ in the form of its corresponding drug D-H or D. In other words, a prodrug is a conjugate comprising a drug moiety, which is covalently and reversibly conjugated to a polymeric moiety via at least one -L¹-L²- moiety. Such prodrugs or conjugates release the formerly conjugated drug moiety in the form of a free or unmodified drug.

As used herein, the term “reversible linkage” or “biodegradable linkage” is a linkage that is cleavable, in the absence of enzymes under physiological conditions, which are aqueous buffer at pH 7.4 and 37° C., with a half-life ranging from one hour to six months, such as from ten hours to four months, such as from one day to three months, from two days to two months or from three days to one month. It is understood, however, that a reversible linkage may also be cleavable at other conditions, such as for example at a different pH or at a different temperature, but that a test for determining reversibility is performed in the above-described physiological conditions (aqueous buffer, pH 7.4, 37° C.). Accordingly, a “stable linkage” is a linkage having a half-life under physiological conditions of more than six months.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined below. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined below. Optionally, a C₁₋₆ alkyl may be interrupted by one or more moieties as defined below.

Accordingly, “C₁₋₁₀ alkyl”, “C₁₋₂₀ alkyl”, “C₈₋₂₄ alkyl” or “C₁₋₅₀ alkyl” means an alkyl chain having 1 to 10, 1 to 20, 8 to 24 or 1 to 50 carbon atoms, respectively, wherein each hydrogen atom of the C₁₋₁₀, C₁₋₂₀, C₈₋₂₄ or C₁₋₅₀ carbon may optionally be replaced by a substituent as defined below. Optionally, a C₁₋₁₀ alkyl, C₁₋₂₀ alkyl, C₈₋₂₄ alkyl or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example of such C₂₋₆ alkenyl is —CH═CH—. Each hydrogen atom of a C₂₋₆ alkenyl moiety may optionally be replaced by a substituent as defined below. Optionally, a C₂₋₆ alkenyl may be interrupted by one or more moieties as defined below.

Accordingly, the terms “C₂₋₁₀ alkenyl”, “C₂₋₂₀ alkenyl” or “C₂₋₅₀ alkenyl” alone or in combination mean a straight-chain or branched hydrocarbon moiety comprising at least one carbon-carbon double bond having 2 to 10, 2 to 20 or 2 to 50 carbon atoms, respectively. Each hydrogen atom of a C₂₋₁₀ alkenyl, C₂₋₂₀ alkenyl or C₂₋₅₀ alkenyl group may optionally be replaced by a substituent as defined below. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C≡CH₃. When two moieties of a molecule are linked by the alkynyl group, then an example is —C≡C—. Each hydrogen atom of a C₂₋₆ alkynyl group may optionally be replaced by a substituent as defined below. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined below. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₈₋₂₄ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which in certain embodiments are selected from the group consisting of

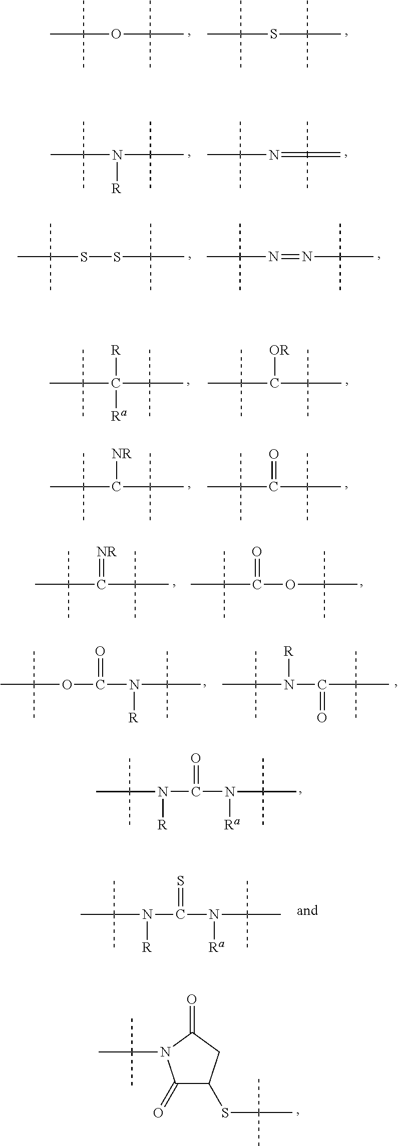

wherein
dashed lines indicate attachment to the remainder of the moiety or reagent;
—R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined below. The term “C₃₋₁₀ cycloalkyl” also includes bridged bicycles like norbornane or norbornene.

As used herein, the term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In certain embodiments, an 8- to 30-membered carbopolycyclyl means a cyclic moiety of two, three, four or five rings. In certain embodiments, an 8- to 30-membered carbopolycyclyl means a cyclic moiety of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including=N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent as defined below.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including=N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

Similarly, the term “8- to 30-membered heteropolycyclyl” or “8- to 30-membered heteropolycycle” means a heterocyclic moiety of more than two rings with 8 to 30 ring atoms, in certain embodiments of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including=N(O)—) and wherein the ring is linked to the rest of a molecule via a carbon or nitrogen atom.

It is understood that the phrase “the pair —R^{x}/—R^{y} is joined together with the atom to which they are attached to form a C₃₋₁₀ cycloalkyl, 3- to 10-membered heterocyclyl or an 8- to 11-membered heterobicyclyl” in relation with a moiety of the structure:

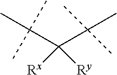

means that —R^{x} and —R^{y} form the following structure:

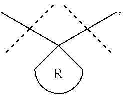

wherein R is C₃₋₁₀ cycloalkyl, 3- to 10-membered heterocyclyl or an 8- to 11-membered heterobicyclyl.

It is also understood that the phrase “the pair —R^{x}/—R^{y} is joined together with the atoms to which they are attached to form a ring -A-” in relation with a moiety of the structure:

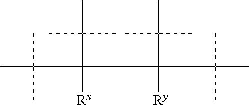

means that —R^{x} and —R^{y} form the following structure:

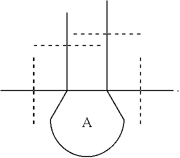

As used herein, the term “a π-electron-pair-donating heteroaromatic N-comprising moiety” refers to the moiety which after cleavage of the linkage between -D and -L¹- results in a drug D-H and wherein the drug moiety -D and analogously the corresponding D-H comprises at least one, such as one, two, three, four, five, six, seven, eight, nine or ten heteroaromatic nitrogen atoms that donate a π-electron pair to the aromatic π-system. Examples of chemical structures comprising such heteroaromatic nitrogen atoms that donate a π-electron pair to the aromatic π-system include, but are not limited to, pyrrole, pyrazole, imidazole, isoindazole, indole, indazole, purine, tetrazole, triazole and carbazole. For example, in the imidazole ring below the heteroaromatic nitrogen which donates a π-electron pair to the aromatic π-system is marked with “#”:

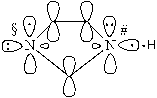

The π-electron-pair-donating heteroaromatic nitrogen atoms do not comprise heteroaromatic nitrogen atoms which only donate one electron (i.e. not a pair of π-electrons) to the aromatic π-system, such as for example the nitrogen that is marked with “§” in the abovementioned imidazole ring structure. The drug D-H may exist in one or more tautomeric forms, such as with one hydrogen atom moving between at least two heteroaromatic nitrogen atoms. In all such cases, the linker moiety is covalently and reversibly attached at a heteroaromatic nitrogen that donates a π-electron pair to the aromatic π-system.

As used herein, the term “excipient” refers to a diluent, adjuvant or vehicle with which the therapeutic, such as a drug conjugate or pharmaceutical composition, is administered.

As used herein, the term “free form” of a drug refers to the drug in its unmodified, pharmacologically active form, e.g. after being released from the conjugate.

As used herein, the term “functional group” means a group of atoms which can react with other groups of atoms. Exemplary functional groups are carboxylic acid, primary amine, secondary amine, tertiary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, disulfide, sulfonamides, sulfuric acid, vinyl sulfone, vinyl ketone, diazoalkane, oxirane and aziridine.

As used herein, the term “halogen” means fluoro, chloro, bromo or iodo. In certain embodiments, halogen is fluoro or chloro.

As used herein, the term “interrupted” means that a moiety is inserted in between two carbon atoms or—if the insertion is at one of the ends of the moiety—between a carbon or heteroatom and a hydrogen atom, in certain embodiments between a carbon and a hydrogen atom.

As used herein, the term “pharmaceutically acceptable” means a substance that does not cause harm when administered to a patient and preferably means approved by a regulatory agency, such as the EMA (Europe) and/or the FDA (US) and/or any other national regulatory agency for use in animals, preferably for use in humans.

As used herein, the term “pharmaceutically acceptable salt(s) thereof” refers to salts that retain the biological effectiveness or properties of the compound and, that typically are not biologically or otherwise undesirable. In certain embodiments, the compound is capable of forming acid/or base salts by virtue of the presence of amino and/or carboxylic functional groups or groups similar thereto. Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllinate, citrate, ethanedisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulfate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, subsalicylate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine, or tromethamine. The pharmaceutically acceptable salts can be synthesized from a parent compound, a basic or acidic moiety, by conventional chemical methods.

As used herein, the term “peptide” as used herein refers to a chain of at least 2 and up to and including 50 amino acid monomer moieties, which may also be referred to as “amino acid residues”, linked by peptide (amide) linkages. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties.

As used herein, the term “protein” refers to a chain of more than 50 amino acid monomer moieties, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein, the term “small molecule drug” refers to drugs that are organic compounds with a molecular weight of less than 1000 Da, such as less than 900 Da or less than 800 Da. It is understood that nucleobase-based drug moieties, such as adenine or guanine analogues, may also be a type of small molecule drugs.

As used herein, the term “medium molecule drug” refers to drugs that are organic compounds which are not peptides and which are not proteins and have a molecular weight ranging from and including 1 kDa to 7.5 kDa.

As used herein, the term “polymer” means a molecule comprising repeating structural units, i.e. the monomers, connected by chemical bonds in a linear, circular, branched, crosslinked or dendritic way or a combination thereof, which may be of synthetic or biological origin or a combination of both. The monomers may be identical, in which case the polymer is a homopolymer, or may be different, in which case the polymer is a heteropolymer. A heteropolymer may also be referred to as a “copolymer” and includes for example alternating copolymers in which monomers of different types alternate; periodic copolymers in which monomers of different types of monomers are arranged in a repeating sequence; statistical copolymers in which monomers of different types are arranged randomly; block copolymers in which blocks of different homopolymers consisting of only one type of monomers are linked by a covalent bond; and gradient copolymers in which the composition of different monomers changes gradually along a polymer chain. It is understood that a polymer may also comprise one or more other moieties, such as, for example, one or more functional groups. Likewise, it is understood that also a peptide or protein is a polymer, even though the side chains of individual amino acid residues may be different. It is understood that for covalently crosslinked polymers, such as hydrogels, no meaningful molecular weight ranges can be provided.

As used herein, the term “hydrogel” means a hydrophilic or amphiphilic polymeric network composed of homopolymers or copolymers, which is insoluble due to the presence of hydrophobic interactions, hydrogen bonds, ionic interactions and/or covalent chemical crosslinks. The crosslinks provide the network structure and physical integrity.

As used herein, the term “weight average molecular weight” or “M_{w}” refers to the statistical average molecular weight of all molecules, taking into account the weight of each molecule in determining its contribution to the molecular weight average, expressed in units g/mol. The higher the molecular weight of a given molecule, the more that molecule will contribute to the M_{w} value. The weight average molecular weight may be calculated by techniques known in the art that are sensitive to molecular size, such as static light scattering, small angle neutron scattering, X-ray scattering or sedimentation velocity.

As used herein, the term “spacer” or “spacer moiety” refers to a moiety suitable for connecting two moieties.

As used herein, the term “substituted” means that one or more —H atom(s) of a molecule or moiety are replaced by a different atom or a group of atoms, which are referred to as “substituent”. As used herein, the term “substituent” refers in certain embodiments to a moiety selected from the group consisting of halogen, —CN, —C(O)OR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1a})(R^{x1b}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a})(R^{x1b}), —OC(O)N(R^{x1})(R^{x1a}), -T⁰, C₁₋₅₀ alkyl, C₂₋₅₀ alkenyl and C₂₋₅₀ alkynyl; wherein -T⁰, C₁₋₅₀ alkyl, C₂₋₅₀ alkenyl and C₂₋₅₀ alkynyl are optionally substituted with one or more —R^{x2}, which are the same or different and wherein C₁₋₅₀ alkyl, C₂₋₅₀ alkenyl and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})— and —OC(O)N(R^{x3})—; —R^{x1}, —R^{x1a}, —R^{x1b} are independently selected from the group consisting of —H, -T⁰, C₁₋₅₀ alkyl, C₂₋₅₀ alkenyl and C₂₋₅₀ alkynyl; wherein -T⁰, C₁₋₅₀ alkyl, C₂₋₅₀ alkenyl and C₂₋₅₀ alkynyl are optionally substituted with one or more —R^{x2}, which are the same or different and wherein C₁₋₅₀ alkyl, C₂₋₅₀ alkenyl and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})— and —OC(O)N(R^{x3})—;
each T⁰ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T⁰ is independently optionally substituted with one or more —R^{x2}, which are the same or different;
each —R^{x2} is independently selected from the group consisting of halogen, —CN, oxo (═O), —C(O)OR^{x4}, —OR^{x4}, —C(O)R^{x4}, —C(O)N(R^{x4})(R^{x4a}), —S(O)₂N(R^{x4})(R^{x4a}), —S(O)N(R^{x4})(R^{x4a}), —S(O)₂R^{x4}, —S(O)R^{x4}, —N(R^{x4})S(O)₂N(R^{x4a})(R^{x4}b), —SR^{x4}, —N(R^{x4})(R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a})(R^{x4b}), —OC(O)N(R^{x4})(R^{x4a}) and C₁₋₆ alkyl; wherein C₁₋₆ alkyl is optionally substituted with one or more halogen, which are the same or different;
each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments, the term “substituent” refers to a moiety selected from the group consisting of halogen, —CN, —C(O)OR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1})(R^{x1a}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1})(R^{x1a}), —OC(O)N(R^{x1})(R^{x1a}), -T⁰, C₁₋₁₀ alkyl, C₂₋₁₀ alkenyl, and C₂₋₁₀ alkynyl; wherein -T⁰, C₁₋₁₀ alkyl, C₂₋₁₀ alkenyl, and C₂₋₁₀ alkynyl are optionally substituted with one or more —R^{x2}, which are the same or different and wherein C₁₋₁₀ alkyl, C₂₋₁₀ alkenyl, and C₂₋₁₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})— and —OC(O)N(R^{x3})—;
each —R^{x1}, —R^{x1a}, —R^{xlb}, —R^{x3}, —R^{x3a} is independently selected from the group consisting of —H, halogen, C₁₋₆ alkyl, C₂₋₆ alkenyl and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different;
each —R^{x2} is independently selected from the group consisting of halogen, —CN, oxo (═O), —C(O)OR^{x4}, —OR^{x4}, —C(O)R⁴, —C(O)N(R^{x4})(R^{x4a}), —S(O)₂N(R^{x4})(R^{x4a}), —S(O)N(R^{x4})(R^{x4a}), —S(O)₂R^{x4}, —S(O)R^{x4}, —N(R^{x4})S(O)₂N(R^{x4a})(R^{x4}b), —SR^{x4}, —N(R^{x4})(R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a})(R^{x4b}), —OC(O)N(R^{x4})(R^{x4a}) and C₁₋₆ alkyl; wherein C₁₋₆ alkyl is optionally substituted with one or more halogen, which are the same or different;
each —R^{x4}, —R^{x4a}, —R^{x4b} is independently selected from the group consisting of —H, halogen, C₁₋₆ alkyl, C₂₋₆ alkenyl, and C₂₋₆ alkynyl.

In certain embodiments, the term “substituent” refers to a moiety selected from the group consisting of halogen, —CN, —C(O)OR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1a})(R^{x1b}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a})(R^{x1b}), —OC(O)N(R^{x1})(R^{x1a}), -T⁰, C₁₋₆ alkyl, C₂₋₆ alkenyl, and C₂₋₆ alkynyl; wherein -T⁰, C₁₋₆ alkyl, C₂₋₆ alkenyl, and C₂₋₆ alkynyl are optionally substituted with one or more —R^{x2}, which are the same or different and wherein C₁₋₆ alkyl, C₂₋₆ alkenyl, and C₂₋₆ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each —R^{x1}, —R^{x1a}, —R^{xlb}, —R^{x2}, —R^{x3}, —R^{x3a} is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2} which are the same or different.

In certain embodiments, a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

As used herein, the term “therapeutically effective amount” means an amount sufficient to cure, alleviate or partially arrest the clinical manifestations of a given disease and its complications. Effective amounts for each purpose will depend on the severity of the disease as well as the weight and general state of the subject.

As used herein, the term “is administered via injection” or “injectability” refers to a combination of factors such as a certain force applied to a plunger of a syringe comprising the drug conjugate described herein that may be swollen in a liquid at a certain concentration (w/v) and at a certain temperature, a needle of a given inner diameter connected to the outlet of such syringe, and the time required to extrude a certain volume of the drug conjugate from the syringe through the needle.

As used herein, the term “subject” refers to an animal. Typically, the animal is a mammal. The term “subject” also refers to for example, primates (e.g. humans), cows, sheep, goats, horses, dogs, cats, rabbits, rats, mice, fish, birds and the like. In certain embodiments, the subject is a primate. In certain embodiments, the subject is a human. In certain embodiments, the subject is a non-human primate.

As used herein, a subject is “in need of” or “in need thereof” a treatment if such subject would benefit biologically, medically, or in quality of life from such treatment.

As used herein, the term “water-insoluble” refers to a compound of which less than 1 g can be dissolved in one liter of water at 20° C. to form a homogeneous solution. Accordingly, the term “water-soluble” refers to a compound of which 1 g or more can be dissolved in one liter of water at 20° C. to form a homogeneous solution.

As used herein, the term “buffer” or “buffering agent” refers to a chemical compound that maintains the Ph of a solution in a desired range.

As used herein, the term “emulsifying agent” or “emulsifier” refers to a chemical compound, such as a surface-active ingredient, which adsorbs at the newly formed interface between the dispersed and continuous phase solutions during emulsion formation, allows the mixing of said solutions and protects the newly formed droplets against immediate recoalescence.

As used herein, the term “pH-adjusting agent” refers to a chemical compound that is used to shift the pH of the droplets within an emulsion, such as within the emulsion of step (a) and to initiate and/or accelerate the crosslinking reaction between the first and second functionalized HA.

As used herein, the term “blocking reagent” refers to a chemical compound that is used to cap unreacted functional groups, such as -FG₁ or -FG₂.

As used herein, the term “flow system” or “continuous flow system” refers to a system where a process, such as precipitation of a polymer, is run in a continuously flowing stream rather than in a batch production.

As used herein, the term “a setup for precipitating and isolating a polymer” refers to an arrangement or equipment comprising a flow system that is connected to a collecting assembly.

As used herein, the term “anti-solvent” refers to a solvent in which a polymer, such as a functionalized HA, is insoluble. The term “insoluble” with reference to a polymer means that less than one gram of said polymer can be dissolved in one liter of said solvent at room temperature (room temperature may range from 17° C. to 30° C., such as from 17° C. to 25° C.) to form a homogenous solution.

As used herein “a screen scroll centrifuge” refers to a filtering centrifuge which separates solids and liquid from a solid-liquid mixture. In a typical screen scroll centrifuge, the basic principle is that the entering feed is separated into liquid and solids as two products.

In general, the term “comprise(s)” or “comprising” also encompasses “consist(s) of” or “consisting of”.

The present invention relates to a method for preparing hydrogel microspheres or pharmaceutically acceptable salts thereof comprising a crosslinked hyaluronic acid (HA), wherein the method comprises the steps of:
(a) mixing a solution A with a solution B to form an emulsion, wherein solution A comprises a first functionalized HA that is modified with one or more -FG₁ and optionally further functional groups and a second functionalized HA that is modified with one or more -FG₂ and optionally further functional groups, wherein -FG₁ and -FG₂ are functional group moieties that are different from each other and wherein -FG₁ on the first functionalized HA reacts with -FG₂ on the second functionalized HA to form a plurality of crosslinks which results in the formation of hydrogel HA microspheres;
(b) optionally, adding a pH-adjusting agent to the emulsion of step (a); and
(c) collecting the obtained hydrogel HA microspheres of step (a) or (b).

It will be recognized by the person skilled in the art that the method of the present invention for preparing hydrogel microspheres comprising a crosslinked HA or pharmaceutically acceptable salt thereof may be applied for the preparation of any hydrogel microspheres comprising any crosslinked glycosaminoglycan. In other words, the method of the present invention may equally be applied for preparing hydrogel microspheres comprising a crosslinked heparin, heparan sulfate, heparosan, chondroitin sulfate, dermatan sulfate or keratan sulfate. In particular, said method may be applied for preparing hydrogel microspheres comprising a crosslinked heparosan.

The polymerization within the method of the present invention occurs via suspension polymerization. In step (a) solutions A and B form an emulsion and after a sufficient mixing time, solution A becomes a dispersed phase, while solution B becomes a continuous phase. The dispersed phase should not be miscible with the continuous phase. Also, in step (a), both the functionalized HA are predominantly or exclusively linear HA strands.

In step (a) the mixing of solutions A and B which form the emulsion may require vigorous agitation, pressure or other forces that may be achieved by stirring, such as by stirring with a pitched blade stirrer in combination with a baffle; shaking, such as by shaking in a vessel, such as in a falcon tube (closed tube); by using a rotor or stator; by using an ultrasound device; by using a static mixer, such as by using a packed bead column or flow plate; by using a membrane with defined pores, such as by using a cross-flow membrane emulsification technique in which the liquid form of the material to be formed into microparticles is pushed through a membrane comprising micro-scale pores into a flowing solution of the dispersed phase or stirred cell membrane emulsification; by using a tubular membrane having a surface made from a hydrophobic plastic, such as the one disclosed in WO 2022/198052 A2 which is herewith incorporated by reference in its entirety; by using a microfluidic droplet generator; or by spray polymerization in the air, such as by using an ultrasonic atomizer. Suitably, the mixing in step (a) is achieved by stirring, such as by stirring with a pitched blade stirrer in combination with a baffle or by using a microfluidic droplet generator.

In certain embodiments, the method of the present invention, comprises the steps of:
(a) mixing a solution A with a solution B to form an emulsion, wherein solution A comprises a first functionalized HA that is modified with one or more -FG₁ and optionally further functional groups and a second functionalized HA that is modified with one or more -FG₂ and optionally further functional groups, wherein -FG₁ and -FG₂ are functional group moieties that are different from each other and wherein -FG₁ on the first functionalized HA reacts with -FG₂ on the second functionalized HA to form a plurality of crosslinks which results in the formation of hydrogel HA microspheres;
(b) adding a pH-adjusting agent to the emulsion of step (a); and
(c) collecting the obtained hydrogel HA microspheres of step (a) or (b).

In certain embodiments, all -FG₁ are the same and all -FG₂ are the same.

Suitably, the first and second functionalized HA of the method of the present invention are not optionally modified with further functional groups.

The method of the present invention optionally further comprises the step of size fractionating the obtained hydrogel microspheres of step (c) to obtain microspheres with a particular particle size distribution.

Also, it will be recognized by the person skilled in the art that throughout the specification there could be optional washing or purification steps in between any of the steps of the method of the present invention.

In certain embodiments, the method of the present invention comprises the steps of.
(a) mixing a solution A with a solution B to form an emulsion, wherein solution A comprises a first functionalized HA that is modified with one or more -FG₁ and optionally further functional groups and a second functionalized HA that is modified with one or more -FG₂ and optionally further functional groups, wherein -FG₁ and -FG₂ are functional group moieties that are different from each other and wherein -FG₁ on the first functionalized HA reacts with -FG₂ on the second functionalized HA to form a plurality of crosslinks which results in the formation of hydrogel HA microspheres;
(b) optionally, adding a pH-adjusting agent to the emulsion of step (a);
(c) collecting the obtained hydrogel HA microspheres of step (a) or (b);
(d) optionally, size fractionating the obtained hydrogel HA microspheres of step (a), (b) or (c) to obtain microspheres with a particular particle size distribution;
(e) optionally, washing the microspheres obtained in step (a), (b), (c) or (d);
(f) optionally, incubating the hydrogel HA microspheres of step (a), (b), (c), (d) or (e) in a buffering agent of a pH ranging from about 7 to about 14, such as from about 8 to about 12 or such as from about 8.5 to about 10;
(g) optionally, incubating the hydrogel HA microspheres of step (a), (b), (c), (d), (e) or (f) with a reducing agent;
(h) optionally, washing the microspheres obtained in step (f) or (g); and
(i) optionally, collecting the hydrogel HA microspheres of step (d), (e), (f), (g) or (h).

It is understood that step (d) may also be performed after steps (e), (f), (g) or (h).

In certain embodiments, the molecular weight of the first and second functionalized HA independently ranges from about 80 kDa to about 250 kDa, such as from about 90 kDa to about 200 kDa or such as from about 100 kDa to about 150 kDa. Suitably, the molecular weight of the first and second functionalized HA ranges from 100 kDa to 150 kDa. It is understood that said HA may be polydisperse and comprise polymer chains of unequal length and so the molecular weight is not a single value, i.e. said HA exists as a distribution of chain lengths and molecular weights. For example, if the molecular weight of the first or second functionalized HA is 125 kDa, said HA will comprise polymeric HA strands ranging from about 30 kDa to about 400 kDa.

In certain embodiments, in step (d) the obtained microspheres have a diameter ranging from about 1 μm to about 1000 μm, such as from about 50 μm to about 900 μm, such as from about 100 μm to about 700 μm, such as from about 200 μm to about 500 μm or such as from about 50 μm to about 500 μm, as determined by flow microscopy or other similar methods known in the art. In certain embodiments, in step (d) the obtained microspheres have a diameter ranging from 1 μm to 1000 μm, such as from 50 μm to 900 μm, such as from 100 μm to 700 μm, such as from 200 μm to 500 μm or such as from 50 μm to 500 μm, as determined by flow microscopy. In certain embodiments, in step (d) the obtained microspheres have a diameter ranging from 50 μm to 500 μm, as determined by flow microscopy. In certain embodiments, in step (d) the obtained microspheres have a diameter ranging from 100 μm to 200 μm, as determined by flow microscopy.

Suitably, in step (d) the obtained microspheres have a d₁₀ value of ≥50 μm and a d₉₀ value of ≤900 μm, such as a d₁₀ value of ≥100 μm and a d₉₀ value of ≤700 μm or a d₁₀ value of ≥200 μm and a d₉₀ value of ≤500 μm, as determined by flow microscopy. In certain embodiments, in step (d) the obtained microspheres have a d₁₀ value of ≥100 μm and a d₉₀ value of ≤200 μm. Preferably, for these measurements said microspheres are in succinate buffer.

As used herein, the parameter d₁₀ value signifies the point in the size distribution, below which 10% of the total volume of material in the sample is contained. Similarly, the d₉₀ value is the size below which 90%; of the volume of the material is contained. It is understood that the swelling of the HA microspheres may be influenced by the buffering agent in which the microspheres are stored during the measurement and/or pH, osmolality and ionic strength, and accordingly this may have an impact on the d₁₀ and values d₉₀.

In certain embodiments, in step (f) the buffering agent has a pH ranging from 8 to 12. In certain embodiments, in step (f) the buffering agent has a pH ranging from 8.5 to 10. In certain embodiments, in step (f) the buffering agent has a pH of about 9. In certain embodiments, in step (f) the buffering agent has a pH of 9.

In certain embodiments, the method of the present invention comprises the steps of.
(a) mixing a solution A with a solution B to form an emulsion, wherein solution A comprises a first functionalized HA that is modified with one or more -FG₁ and optionally further functional groups and a second functionalized HA that is modified with one or more -FG₂ and optionally further functional groups, wherein -FG₁ and -FG₂ are functional group moieties that are different from each other and wherein -FG₁ on the first functionalized HA reacts with -FG₂ on the second functionalized HA to form a plurality of crosslinks which results in the formation of hydrogel HA microspheres;
(b) adding a pH-adjusting agent to the emulsion of step (a);
(c) collecting the obtained hydrogel HA microspheres of step (b);
(d) size fractionating the obtained hydrogel HA microspheres of step (c) to obtain microspheres with a particular particle size distribution;
(e) optionally, washing the microspheres obtained in step (c) or (d);
(f) incubating the hydrogel HA microspheres of step (c), (d) or (e) in a buffering agent of a pH ranging from about 7 to about 14, such as about 8 to about 12, or such as about 8.5 to 10;
(g) incubating the hydrogel HA microspheres of step (c), (d), (e) or (f) with a reducing agent; (h) washing the microspheres obtained in step (f) or (g); and
(i) collecting the hydrogel HA microspheres of step (d), (e), (f), (g) or (h).

The present invention also relates to hydrogel HA microspheres or pharmaceutically acceptable salts thereof obtainable or obtained by any of the methods of the present invention.

It was surprisingly found that the method of the present invention allows for the use of linear functionalized HA strands with low degree of substitution in the synthesis of hydrogel HA microspheres that are effectively crosslinked. It was also observed that owing to the size of the microspheres, the injectability of the HA microspheres and drug conjugates comprising hydrogel HA microspheres was significantly improved over the injectability of a coherent gel, such as of the coherent gel disclosed in WO 2018/175788 A1. Another advantage of using HA hydrogel microspheres as carriers for drug conjugates is that prior to the administration, the microspheres can be washed, and thus soluble by-products can be easily removed. On the other hand, the coherent gel disclosed in WO 2018/175788 A1 cannot undergo washing steps, because said gel is obtained via polymerization in a syringe and thereafter it is directly injected into the eye. Furthermore, the coherent gel disclosed in WO 2018/175788 A1 is prone to hardening while being filled in the injection syringe making the process of filling the syringe more challenging. In contrast, the HA hydrogel microspheres of the present invention can be stored after synthesis and then easily filled or further conjugated to drug moieties in the final container at a later time point.

Suitably, solution A of step (a) comprises the first and second functionalized HA and a solvent in which said functionalized HA can be dissolved.

In certain embodiments, solution A of step (a) comprises the first and second functionalized HA and dimethyl sulfoxide, DMF, DMA or a mixture thereof. In certain embodiments, solution A of step (a) comprises the first and second functionalized HA and dimethyl sulfoxide.

In certain embodiments, solution A of step (a) comprises the first and second functionalized HA, dimethyl sulfoxide and water. In certain embodiments, solution A of step (a) comprises the first and second functionalized HA, dimethyl sulfoxide and a buffering agent.

In certain embodiments, solution A of step (a) is an aqueous solution and comprises the first and second functionalized HA and a buffering agent.

It is clear to the person skilled in the art that in general the phrase “a buffering agent” may refer to one buffering agent or to a mixture of two or more buffering agents.

Exemplary buffering agents may be selected from the group consisting of N-(2-acetamido)-2-aminoethanesulfonic acid (ACES), acetate, 2,2′,2″-nitrilotriacetic acid (ADA), adipate, alanine, ammonium, 2-amino-2-methyl-1-propanol (AMP), 2-amino-2-methyl-1,3-propanediol (AMPD), N-(1,1-dimethyl-2-hydroxyethyl)-3-amino-2-hydroxypropanesulfonic acid (AMPSO), arginine, ascorbate, aspartate, benzoate, N,N-bis(2-hydroxyethyl)-2-aminoethanesulfonic acid (BES), bicarbonate, N,N-bis(2-hydroxyethyl)glycine, bis-(2-hydroxy-ethyl)-amino-tris(hydroxymethyl)-methane, 1,3-bis(tris(hydroxymethyl)methylamino)propane, borate, 4-(cyclohexylamino)butane-1-sulfonic acid (CABS), N-cyclohexyl-3-aminopropanesulfonic acid (CAPS), 3-(cyclohexylamino)-2-hydroxy-1-propanesulfonic acid (CAPSO), carbonate, N-cyclohexyl-2-aminoethanesulfonic acid (CHES), citrate, diethanolamine, 3-(N,N-bis[2-hydroxyethyl]amino)-2-hydroxypropanesulfonic acid (DIPSO), edetate, ethanolamine, ethylenediamine, formate, fumarate, gluconate, glutamate, glycine, glycylglycine, guanidine, N-(2-hydroxyethyl)piperazine-N′-(4-butanesulfonic acid) (HEPBS), 4-(2-hydroxyethyl)-1-piperazineethanesulfonic acid (HEPES), 3-[4-(2-hydroxyethyl)piperazin-1-yl]propane-1-sulfonic acid (HEPPS), N-(2-hydroxyethyl)piperazine-N′-(propanesulphonic acid) (HEPPSO), histidine, hydrazine, imidazole, lactate, lysine, malate, maleate, 2-(N-morpholino)ethanesulfonic acid (MES), metaphosphate, methylamine, 4-(4-morpholinyl)butanesulfonic acid (MOBS), 3-(N-morpholino)propanesulfonic acid (MOPS), 2-hydroxy-3-morpholinopropanesulfonic acid (MOPSO), pentetate, phosphate, piperazine-N,N′-bis(2-ethanesulfonic acid) (PIPES), piperazine, piperidine, piperazine-N,N′-bis(2-hydroxypropanesulfonic acid) (POPSO), propionate, pyridine, pyrophosphate, pyruvate, sorbate, succinate, N-tris(hydroxymethyl)methyl-4-aminobutanesulfonic acid (TABS), ([tris(hydroxymethyl)methylamino]propanesulfonic acid (TAPS), 2-hydroxy-3-[tris(hydroxymethyl)methylamino]-1-propanesulfonic acid (TAPSO), tartrate, taurine, 2-{[1,3-dihydroxy-2-(hydroxymethyl)propan-2-yl]amino}ethane-1-sulfonic acid (TES), tricine, triethanolamine, tromethamine and α-ketoglutarate.

It is clear to the person skilled in the art that the corresponding conjugate acids, bases or salts of the buffering agents and mixtures thereof are also included.

In certain embodiments, the aqueous solution comprises the first and second functionalized HA and a buffering agent, such as a buffering agent selected from the group consisting of citrate and histidine or a mixture thereof. In certain embodiments, the buffering agent comprises a mixture of citrate and histidine. In certain embodiments, the buffering agent consists of a mixture of citrate and histidine.

As defined herein, the term “histidine” is intended to encompass both D-histidine and L-histidine and mixtures thereof. In certain embodiments, the term “histidine” refers to L-histidine. In certain embodiments, the term “histidine” refers to D-histidine. In certain embodiments, the term “histidine” refers to a mixture of L-histidine and D-histidine.

In certain embodiments, the buffering agent is L-histidine.

In certain embodiments, the buffering agent consists of a mixture of citrate, L-histidine and sodium chloride.

In certain embodiments, the aqueous solution comprises the first and second functionalized HA, citrate and sodium chloride.

In certain embodiments, the aqueous solution consists of the first and second functionalized HA, citrate and sodium chloride. In certain embodiments, the aqueous solution comprises the first and second functionalized HA, histidine and sodium chloride.

In certain embodiments, the aqueous solution comprises the first and second functionalized HA and an emulsifying agent, while solution B comprises a solvent.

The buffering agent may be added in general in an amount of about 0.01 mM to about 500 mM.

In certain embodiments, the buffering agent has a concentration ranging from about 0.5 mM to about 350 mM. In certain embodiments, the buffering agent has a concentration ranging from about 1 mM to about 250 mM. In certain embodiments, the buffering agent has a concentration ranging from about 5 mM to 100 mM. In certain embodiments, the buffering agent has a concentration of about 100 mM. In certain embodiments, the buffering agent has a concentration of 100 mM. In certain embodiments, the buffering agent has a concentration of about 5 mM. In certain embodiments, the buffering agent has a concentration of 5 mM.

Suitably, solution B of step (a) comprises an emulsifying agent and a solvent.

It is clear to the person skilled in the art that in general the phrase “a solvent” may refer to one solvent or to a mixture of two or more solvents and that the phrase “an emulsifying agent” may refer to one emulsifying agent or to a mixture of two or more emulsifying agents.

Exemplary emulsifying agents may be selected from the group consisting of sorbitan esters such as sorbitan monolaurate (Span® 20), sorbitan monooleate (Span® 80), sorbitan monopalmitate (Span® 40), sorbitan monostearate (Span® 60), sorbitan sequioleate (Span® 83), sorbitan trioleate (Span® 85) or sorbitan tristearate (Span® 65); PEG-30 dipolyhydroxystearate (Cithrol™ DPHS); polyglyceryl-3 diisostearate; a mixture of sorbitan oleate and copolymeric ester of a hydroxy stearic acid and ethylene glycol (Hypermer™ 1083); polyoxyethylenesorbitan monooleate (Polysorbate 80, Tween® 80 and Tween® 80R); alcohols such as propanol, butanol, pentanol, hexanol, heptanol or octanol; alkyl and aryl amine salts such as primary amine salts, quaternary amine salts, secondary amine salts or tertiary amine salts; alkyl dimethyl betaines; alkyl ethoxylate sulfates; alkyl phenyl polyoxyethylene ethers such as Octoxynol 9, Triton X-100, Igepal™ or Nonidet P40; alkyl phosphates such as monoalkylphosphates or dialkylphosphates; alkyl polyoxyethylene ethers such as Laureth-4, Laureth-9, Laureth-23, Ceteth-2, Ceteth-10, Ceteth-20, Ceteareth-6, Ceteareth-20, Ceteareth-25, Steareth-2, Steareth-10, Steareth-20, Oleth-2, Oleth-10, Oleth-20, Deceth-10 or Trideceth-10; alkyl sulfates such as sodium dodecylsulfate (SDS); alkyl xanthates; bile acid salts such as cholic acid sodium salt or deoxycholic acid sodium salt; cationic lipids such as cetyl trimethylammonium bromide, cetyl trimethylammonium chloride, dioctadecyl dimethyl ammonium bromide, dioctadecyl dimethyl ammonium chloride, 1,2-diacyl-3-trimethylammonium propane, 1,2-diacyl-3-dimethyl ammonium propane, [2,3-bis(oleoyl)propyl]trimethyl ammonium chloride or [N—(N-dimethylaminoethane)-carbamoyl]cholesterol, dioleoyl); dialkyl sulfosuccinate salts such as Aerosol OT; ethylenediamine tetrakis(ethoxylate-block-propoxylate) tetrols such as Tetronic 304, Tetronic 904, Tetronic 90R4 or Tetronic 1304; fatty acids such as palmitic acid, oleic acid, lauric acid, myristic acid, stearic acid, arachidic acid, behenic acid, lignoceric acid, palmitoleic acid, linoleic acid, linolenic acid or arachidonic acid and salts thereof such as sodium or potassium salts; glycosides such as octyl glucoside or dodecyl maltoside; linear and branched alkylbenzene sulfonates; poly(ethylene glycol)-block-poly(propylene glycol)-block-poly(ethylene glycol)s such as Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188 (Pluronic® F68), Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 benzoate or Poloxamer 182 dibenzoate; polyoxyethylenesorbitan esters such as polyethyleneoxy(40)-sorbitol hexaoleate ester, polyoxyethylenesorbitan monolaurate (Polysorbate 20, Tween® 20 and Tween® 21), polyoxyethylenesorbitan monopalmitate (Polysorbate 40, Tween® 40), polyoxyethylenesorbitan monostearate (Polysorbate 60, Tween® 60 and Tween® 61), polyoxyethylenesorbitan trioleate (Polysorbate 85, Tween® 85) or polyoxyethylenesorbitan tristearate (Polysorbate 65, Tween® 65); polyvinyl alcohol; polyvinylpyrrolidone; starch and their derivatives and mixtures thereof.

In certain embodiments, the emulsifying agent is selected from the group consisting of sorbitan monolaurate (Span® 20), sorbitan monooleate (Span® 80), sorbitan monopalmitate (Span® 40), sorbitan monostearate (Span® 60), sorbitan sequioleate (Span® 83), sorbitan trioleate (Span® 85) and sorbitan tristearate (Span® 65).

In certain embodiments, the emulsifying agent is selected from the group consisting of sorbitan monooleate (Span® 80), PEG-30 dipolyhydroxystearate (Cithrol™ DPHS), polyglyceryl-3 diisostearate and a mixture of sorbitan oleate and copolymeric ester of a hydroxy stearic acid and ethylene glycol (Hypermer™ 1083).

In certain embodiments, the emulsifying agent is selected from the group consisting of sorbitan monooleate, PEG-30 dipolyhydroxystearate, polyglyceryl-3 diisostearate and a mixture of sorbitan oleate and copolymeric ester of a hydroxy stearic acid and ethylene glycol.

In certain embodiments, the emulsifying agent is PEG-30 dipolyhydroxystearate. In certain embodiments, the emulsifying agent is polyglyceryl-3 diisostearate. In certain embodiments, the emulsifying agent is a mixture of sorbitan oleate and copolymeric ester of a hydroxy stearic acid and ethylene glycol.

Advantageously, the emulsifying agent is sorbitan monooleate.

The emulsifying agent may be added in an amount of about 0.01% (w/w) to about 15% (w/w). In certain embodiments, the emulsifying agent is added in an amount of about 0.01% (w/w) to about 10% (w/w). In certain embodiments, the emulsifying agent is added in an amount of about 0.1% (w/w) to about 7% (w/w). In certain embodiments, the emulsifying agent is added in an amount of about 1% (w/w) to about 5% (w/w). In certain embodiments, the emulsifying agent is added in an amount of about 1.5% (w/w) to about 3.0% (w/w).

In certain embodiments, the emulsifying agent is added in an amount of about 1.5% (w/w). In certain embodiments, the emulsifying agent is added in an amount of 1.5% (w/w). In certain embodiments, the emulsifying agent is added in an amount of about 0.5% (w/w). In certain embodiments, the emulsifying agent is added in an amount of 0.5% (w/w). In certain embodiments, the emulsifying agent is added in an amount of about 0.25% (w/w). In certain embodiments, the emulsifying agent is added in an amount of 0.25% (w/w).

In certain embodiments, the solvent may be any solvent which is non-miscible with the dispersed phase.

In certain embodiments, the solvent is selected from the group consisting of polar solvents, non-polar solvents, fluorocarbons and ionic liquids.

In certain embodiments, the solvent is selected from the group consisting of hydrocarbons such as 3-carene, benzene, cumene, cycloheptane, cyclohexane, decane, dodecane, ethylbenzene, hemellitene, heptane, hexane, isodurene, limonene, mesitylene, m-xylene, n-butylbenzene, n-propylbenzene, nonane, octane, o-xylene, p-cymene, pentadecane, pentane, pinane, pinene, p-menthane, prehnitene, pseudocumene, p-xylene, styrene, tetradecane, toluene, tridecane or undecane; siloxanes such as cyclomethicones, decamethylcyclopentasiloxane, hexamethyldisiloxane, octamethyltrisiloxane or liquid polysiloxanes (silicon oils) and esters such as acetyltributylcitrate, castor oil, ethyl laurate, glyceryl trioleate, liquid triglycerides, triacetin, tributyrin and triethyl citrate.

In certain embodiments, the solvent is selected from the group consisting of 3-carene, benzene, cumene, cycloheptane, cyclohexane, decane, dodecane, ethylbenzene, hemellitene, heptane, hexane, isodurene, limonene, mesitylene, m-xylene, n-butylbenzene, n-propylbenzene, nonane, octane, o-xylene, p-cymene, pentadecane, pentane, pinane, pinene, p-menthane, prehnitene, pseudocumene, p-xylene, styrene, tetradecane, toluene, tridecane and undecane.

In certain embodiments, the solvent is selected from the group consisting of acetyltributylcitrate, castor oil, ethyl laurate, glyceryl trioleate, liquid triglycerides, triacetin, tributyrin and triethyl citrate.

In certain embodiments, the solvent is heptane or tetradecane. In certain embodiments, the solvent is heptane. In certain embodiments, the solvent is tetradecane.

In certain embodiments, solution B of step (a) comprises sorbitan monooleate and heptane. In certain embodiments, solution B of step (a) consists of sorbitan monooleate and heptane. In certain embodiments, solution B of step (a) comprises PEG-30 dipolyhydroxystearate and heptane.

In certain embodiments, solution B of step (a) comprises sorbitan monooleate and tetradecane. In certain embodiments, solution B of step (a) consists of sorbitan monooleate and tetradecane. In certain embodiments, solution B of step (a) comprises PEG-30 dipolyhydroxystearate and tetradecane.

In certain embodiments, solution B of step (a) comprises Hypermer™ 1083 and heptane. In certain embodiments, solution B of step (a) comprises a mixture of sorbitan oleate and copolymeric ester of a hydroxy stearic acid, ethylene glycol and heptane.

In certain embodiments, solution B of step (a) comprises polyglyceryl-3 diisostearate and heptane.

Step (a) may take place at a temperature ranging from about 0° C. to about 150° C., such as from about 4° C. to about 80° C. for a sufficient time, such as for at least about 10 seconds to at least about 12 hours, such as from at least 30 minutes to at least about 12 hours to allow the functionalized HA to react.

In certain embodiments, the emulsion of step (a) is placed at room temperature for about 5 minutes.

In certain embodiments, the emulsion of step (a) is placed at room temperature for about 12 hours.

It is understood that room temperature may range from 17° C. to 30° C., such as from 17° C. to 25° C.

Suitably, the pH-adjusting agent initiates and/or accelerates the crosslinking reaction between the first and second functionalized HA.

The pH-adjusting agent may be soluble in both solutions A and B of step (a). This provides the advantage of using controlled reaction conditions for the synthesis of the hydrogel HA microspheres of the present invention.

The pH-adjusting agent may be an acid or a base.

In certain embodiments, the base is an aprotic, non-nucleophilic amine that is soluble in both the dispersed and continuous phases.

Exemplary bases may be selected from the group consisting of N,N,N′,N′-tetramethylethylene diamine (TMEDA), 1,4-dimethylpiperazine, 4-methylmorpholine, 4-ethylmorpholine, 1,4-diazabicyclo[2.2.2]octane, 1,1,4,7,10,10-hexamethyltriethylenetetramine, 1,4,7-trimethyl-1,4,7-triazacyclononane, tris[2-(dimethylamino)ethyl]amine, triethylamine, diisopropylethylamine (DIPEA), trimethylamine, N,N-dimethylethylamine, N,N,N′,N′-tetramethyl-1,6-hexanediamine, N,N,N′,N″,N″-pentamethyldiethylenetriamine, 1,8-diazabicyclo[5.4.0]undec-7-ene, 1,5-diazabicyclo[4.3.0]non-5-ene and hexamethylenetetramine.

In certain embodiments, the base is selected from the group consisting of N,N,N′,N′-tetramethylethylene diamine (TMEDA), 1,4-dimethylpiperazine, 4-methylmorpholine, 4-ethylmorpholine, 1,4-diazabicyclo[2.2.2]octane, 1,1,4,7,10,10-hexamethyltriethylenetetramine, 1,4,7-trimethyl-1,4,7-triazacyclononane, tris[2-(dimethylamino)ethyl]amine, 1,8-diazabicyclo[5.4.0]undec-7-ene, 1,5-diazabicyclo[4.3.0]non-5-ene and hexamethylenetetramine.

In certain embodiments, the base is N,N,N′,N′-tetramethylethylene diamine (TMEDA).

In certain embodiments, before step (c) the suspension of step (b) is diluted with a solution comprising a buffering agent, such as a buffering agent having a pH less than or equal to 4. In certain embodiments, said solution comprises succinic acid, sodium sulfate and ethylenediaminetetraacetic acid (EDTA). In certain embodiments, said solution comprises succinic acid, ethylenediaminetetraacetic acid (EDTA) and isopropanol. In certain embodiments, said solution comprises sodium chloride.

In certain embodiments, in step (b) the pH-adjusting agent is added to the emulsion of step (a) and then incubated at temperatures ranging from 4 to 50° C., such as from 10 to 40° C., such as from 20 to 30° C. In certain embodiments, in step (b) said incubation occurs at about 25° C., such as at 25° C.

In certain embodiments, in step (b) the pH-adjusting agent increases the pH of the emulsion to around 4. In certain embodiments, in step (a) the pH of the emulsion, i.e. before the addition of the pH-adjusting agent is at least 1, such as around 2. It is understood that in step (a) the pH of the emulsion is measured in the aqueous phase.

Suitably, in step (d) the size fractionation may occur via sieving, such as via wet sieving, such as by using a vibrating sieving machine; stirred cell filtration; cross-flow filtration; sedimentation; or centrifugation.

Advantageously, the size fractionation occurs via wet sieving.

In certain embodiments, in step (d) the obtained hydrogel HA microspheres are wet sieved in a solvent in which the particles of the dispersed phase are swellable.

In certain embodiments, in step (d) the obtained hydrogel HA microspheres are wet sieved in a solvent, such as a solvent comprising a buffering agent and optionally a water-miscible organic solvent, such as a polar solvent. Exemplary solvents may be selected from the group consisting of ethanol, methanol, isopropanol, acetonitrile, dioxane, dimethylformamide, dimethylsulfoxide, tert-butanol, dimethylacetamide and N-methylpyrrolidone.

In certain embodiments, in steps (e) and (h) the microspheres are washed with a solution comprising a buffering agent, such as succinic acid. In certain embodiments, said solution comprises succinic acid, sodium chloride, ethylenediaminetetraacetic acid and polyoxyethylenesorbitan monolaurate. In certain embodiments, in step (e) the microspheres are washed with a solution comprising succinic acid, sodium chloride, histidine and Poloxamer (Pluronic™ F-68).

Suitably, in step (f) the hydrogel HA microspheres are incubated in a buffering agent of a pH above 8, such as borate. In certain embodiments, the pH of said buffering agent is about 9. In certain embodiments, the pH of said buffering agent is 9. In certain embodiments, in step (f) the hydrogel HA microspheres are incubated in borate and Poloxamer (Pluronic™ F-68).

Said incubation may take place at temperatures ranging from 4 to 50° C., such as from 10 to 40° C., such as from 20 to 30° C. In certain embodiments, in step (f) said incubation occurs at 25° C.

In certain embodiments, the hydrogel HA microspheres of step (f) are treated with a reducing agent.

Exemplary reducing agents may be selected from the group consisting of 1,3-propanedithiol, 2-mercaptoethanol, 3-mercaptopropionic acid, ascorbic acid, dihydrolipoic acid, dithioerythritol, dithiothreitol, sodium borohydride, sodium cyanoborohydride, tris(2-carboxyethyl)phosphine hydrochloride (TCEP) and thioglycolic acid.

In certain embodiments, the reducing agent is selected from the group consisting of dithiothreitol and tris(2-carboxyethyl)phosphine hydrochloride (TCEP).

Suitably, the reducing agent is dithiothreitol.

Chemical modifications of HA with functional groups impart functionality to the HA. The person skilled in the art will recognize that one functionalized HA can have more than one reactive functional group, such as -FG₁ or -FG₂.

In certain embodiments, -FG₁, -FG₂ and -FG₃ are independently selected from the group consisting of:

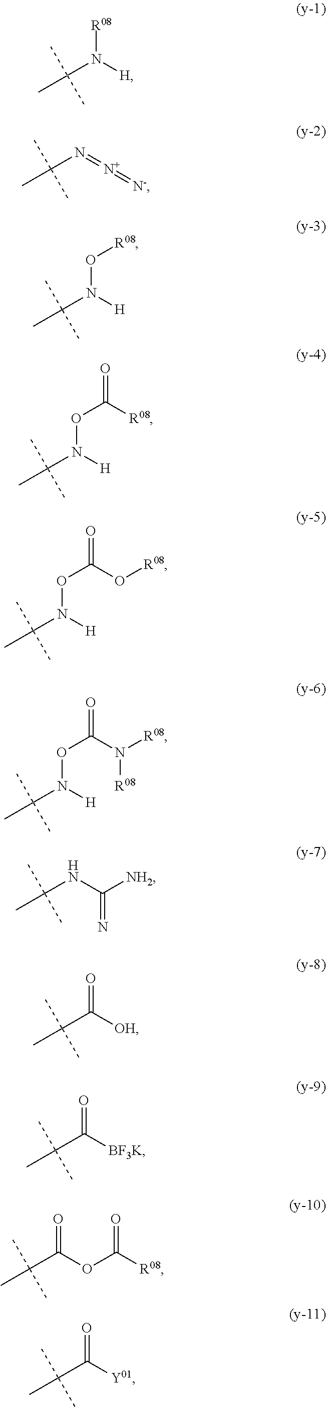

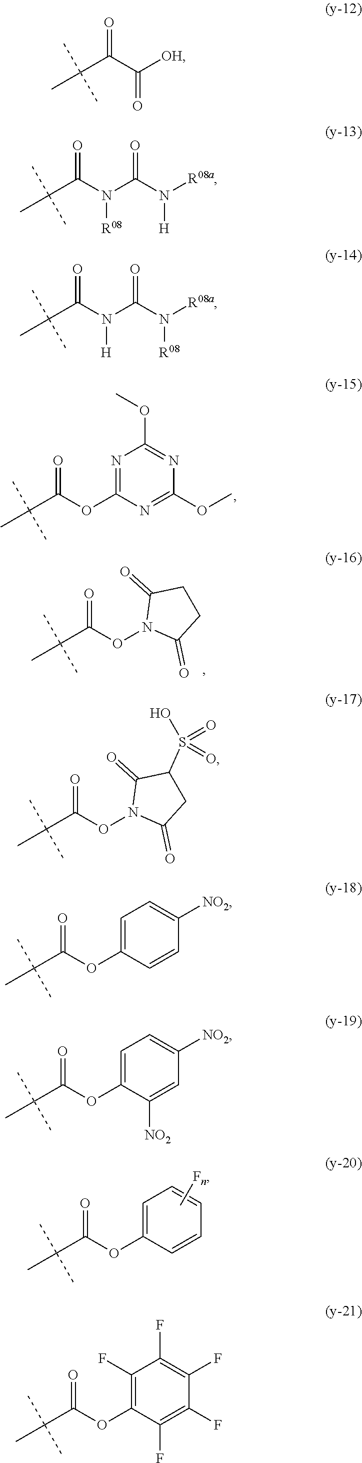

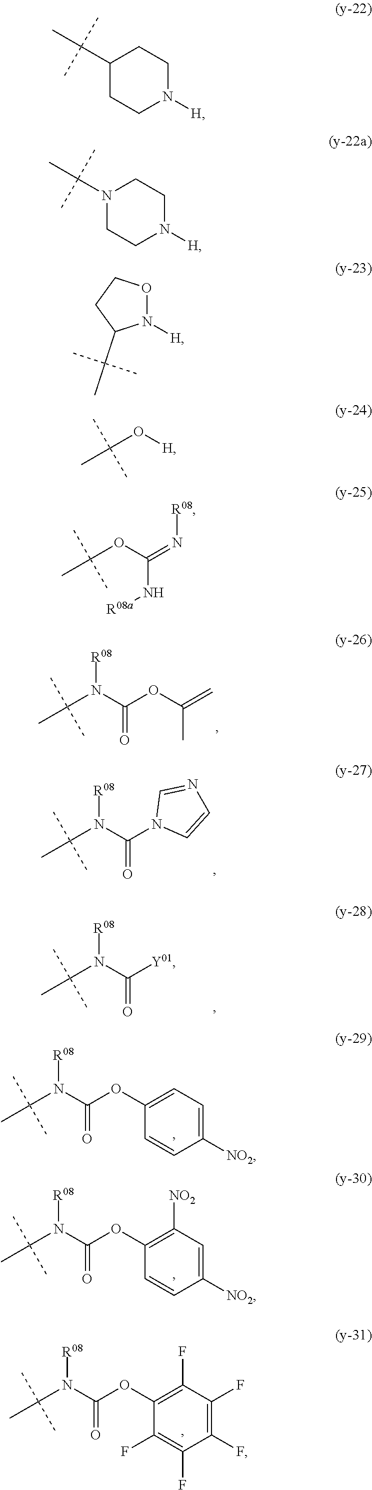

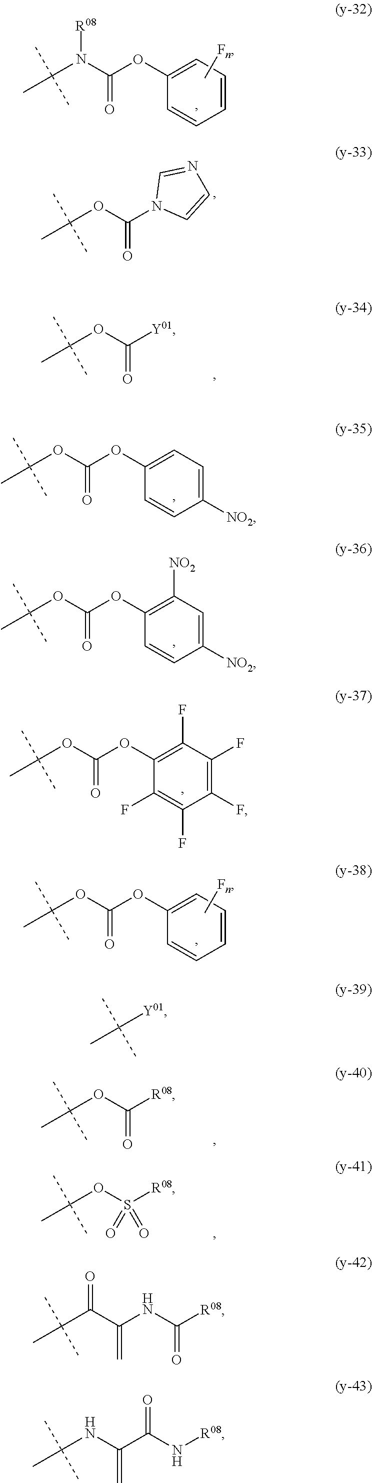

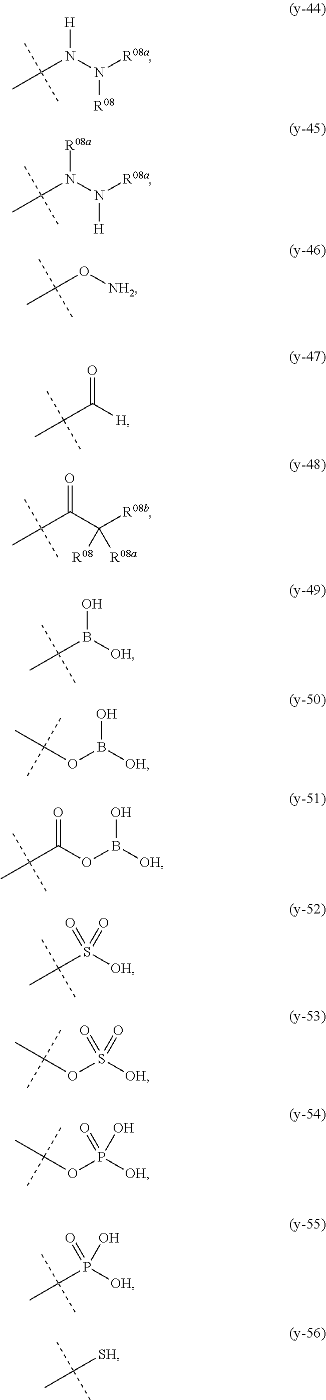

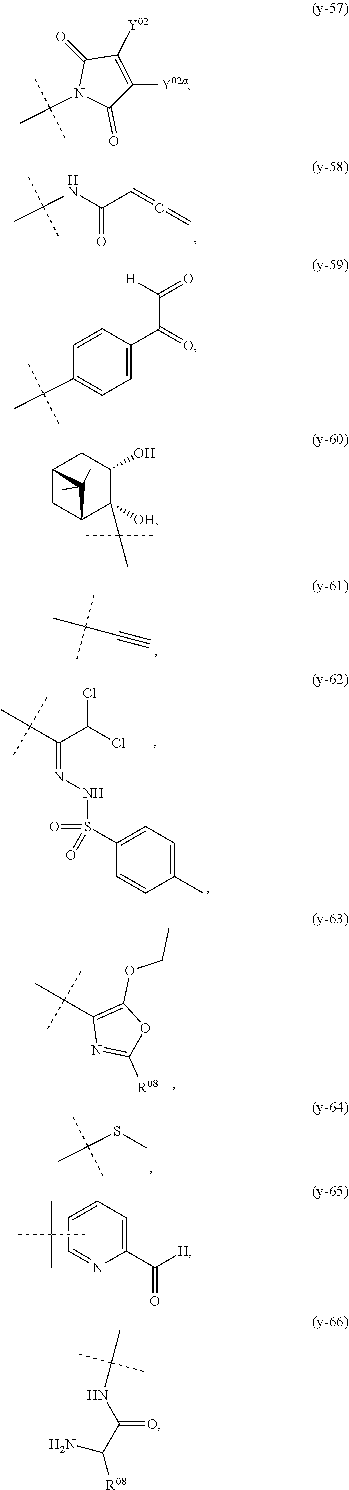

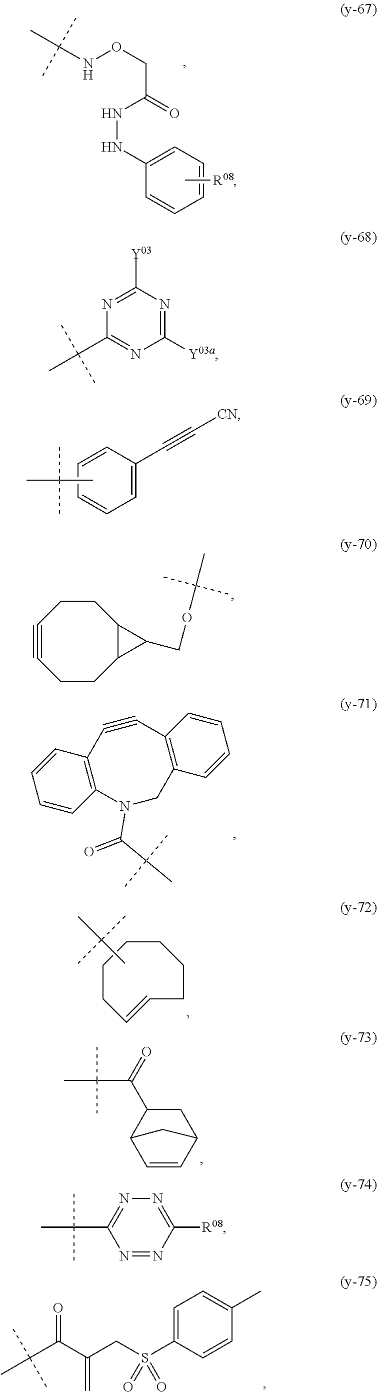

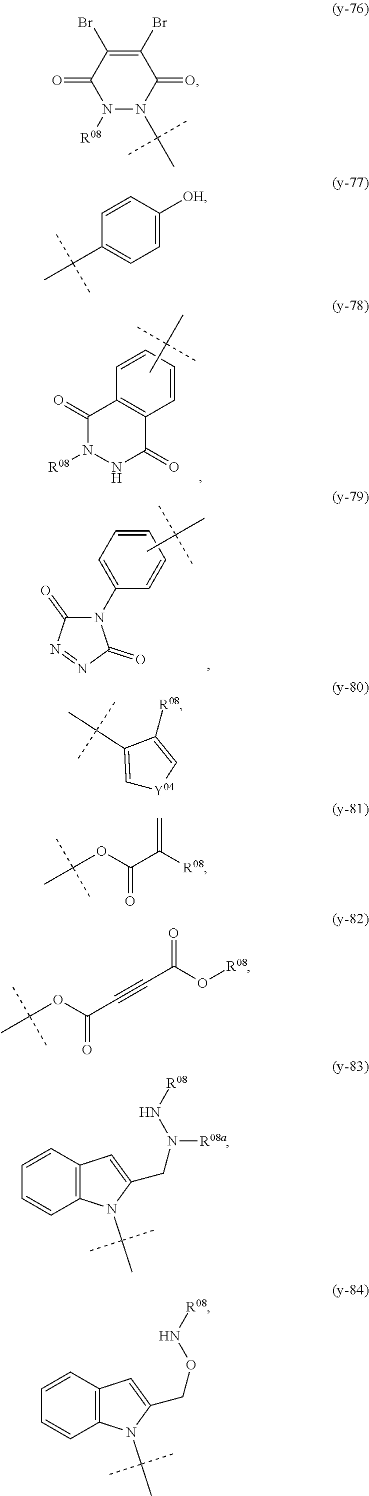

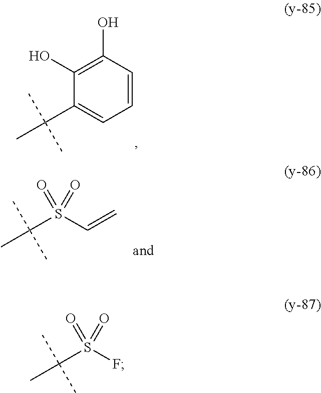

wherein the dashed line indicates the attachment to the first or second functionalized HA, such as to variable —X′— or —Y′—;
each —R⁰⁸, —R^{08a} and —R^{01b} is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰⁹, which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰¹⁰)—, —S(O)₂N(R⁰¹⁰)—, —S(O)N(R⁰¹⁰)—, —S(O)₂—, —S(O)—, —N(R⁰¹⁰)S(O)₂N(R^{010a})—, —S—, —N(R⁰¹⁰)—, —OC(OR⁰¹⁰)(R^{010a})—, —N(R⁰¹⁰)C(O)N(R^{010a})— and —OC(O)N(R⁰¹⁰)—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R⁰⁹, which are the same or different;
each —R⁰⁹, —R⁰¹⁰ and —R^{010a} is independently selected from the group consisting of —H and C₁₋₆ alkyl; wherein C₁₋₆ alkyl is optionally substituted with one or more halogen, which are the same or different;
each —Y⁰¹ is independently selected from the group consisting of —F, —Cl, —Br and —I; each n is independently 1, 2, 3 or 4;
each —Y⁰² and —Y^{02a} is independently selected from the group consisting of —H and —Br; each —Y⁰³ and —Y^{03a} is independently selected from the group consisting of —F, —Cl, —Br, —I, —OR, —NR⁰¹¹R^{011a} and —SR⁰¹¹;
each —Y⁰⁴— is independently selected from —O—, —S—, —NR⁰¹¹—, —CR⁰¹¹R^{011a}—; and
each —R⁰¹¹ and —R^{011a} is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰¹², which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰¹³)—, —S(O)₂N(R⁰¹³)—, —S(O)N(R⁰¹³)—, —S(O)₂—, —S(O)—, —N(R⁰¹³)S(O)₂N(R^{013a})—, —S—, —N(R⁰¹³)—, —OC(OR⁰¹³)(R^{013a})—, —N(R⁰¹³)C(O)N(R^{013a})— and —OC(O)N(R⁰¹³)—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R⁰¹², which are the same or different; and
each —R¹², —R⁰¹³ and —R^{013a} is independently selected from the group consisting of —H and C₁₋₆ alkyl; wherein C₁₋₆ alkyl is optionally substituted with one or more halogen, which are the same or different.

In certain embodiments, -FG₁, -FG₂ and -FG₃ are independently selected from the group consisting of:

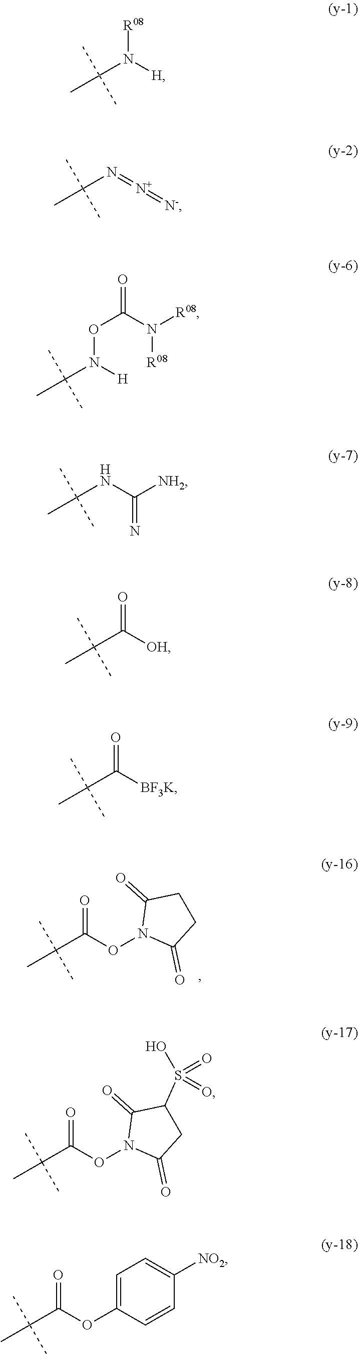

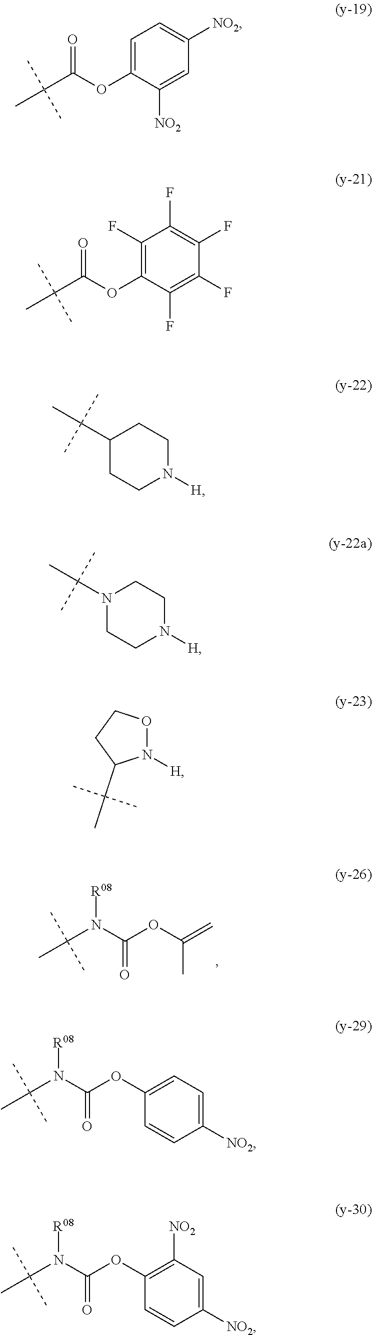

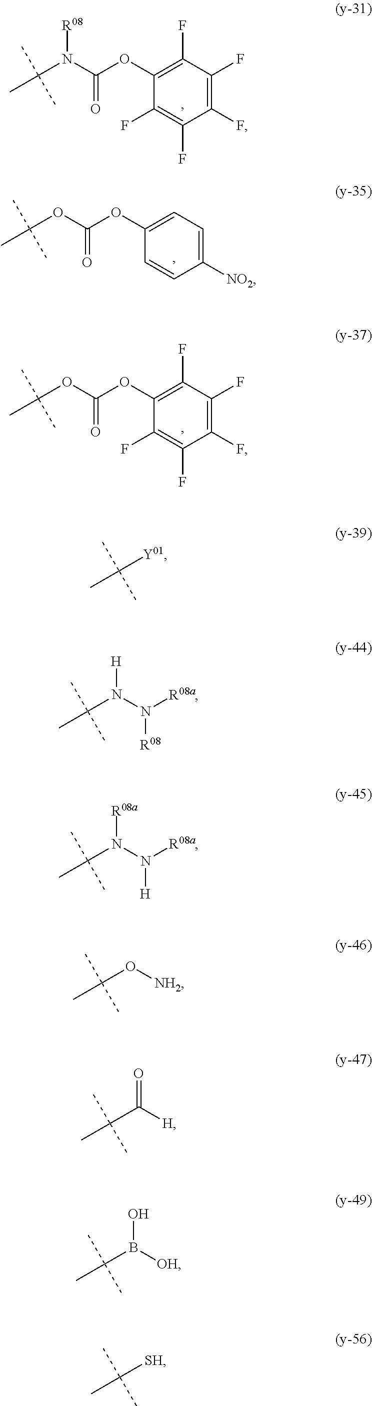

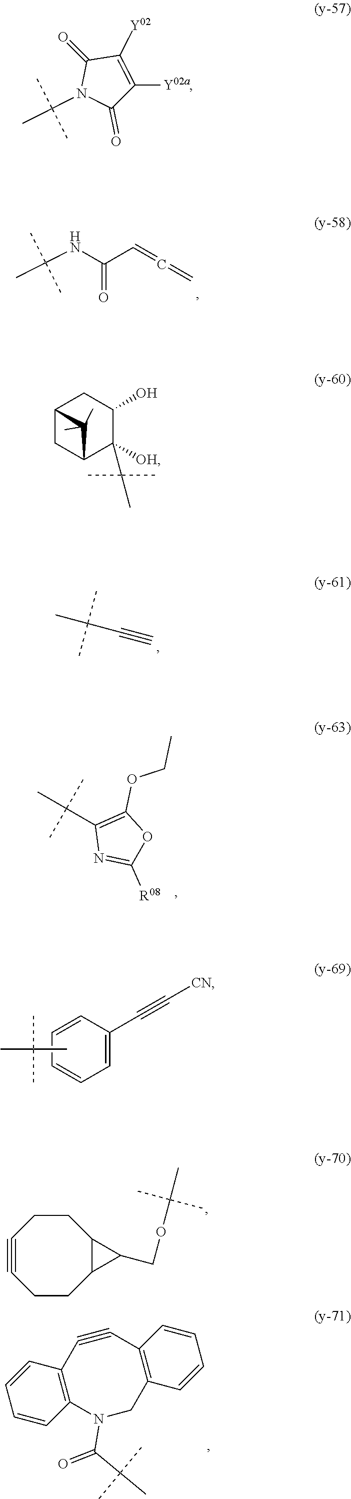

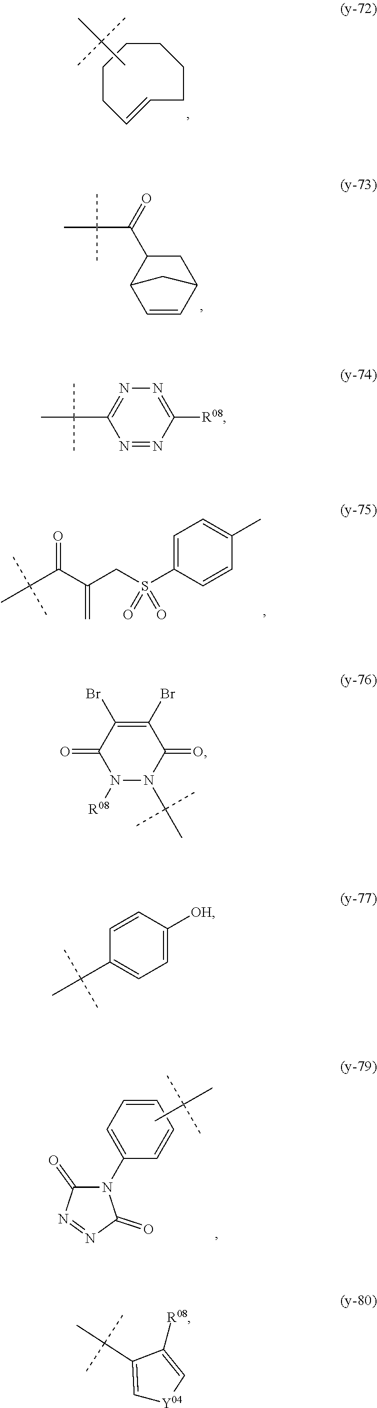

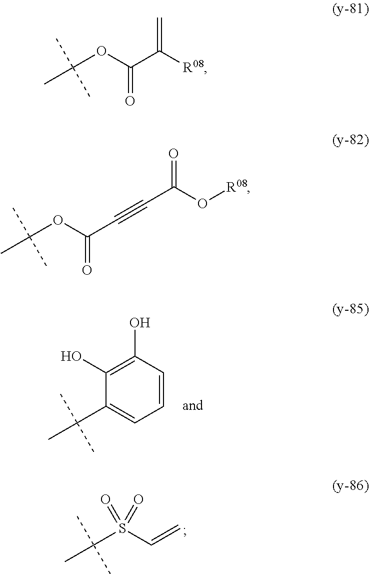

wherein the dashed line indicates the attachment to the first or second functionalized HA, such as to variable —X′— or —Y′—;
each —R⁰⁸, —R^{08a} and —R^{08b} is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰⁹, which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰¹⁰)—, —S(O)₂N(R⁰¹⁰)—, —S(O)N(R⁰¹⁰)—, —S(O)₂—, —S(O)—, —N(R⁰¹⁰)S(O)₂N(R^{010a})—, —S—, —N(R⁰¹⁰)—, —OC(OR⁰¹⁰)(R^{010a})—, —N(R⁰¹⁰)C(O)N(R^{010a})— and —OC(O)N(R⁰¹⁰)—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R⁰⁹, which are the same or different;
each —R⁰⁹, —R⁰¹⁰ and —R^{010a} is independently selected from the group consisting of —H and C₁₋₆ alkyl; wherein C₁₋₆ alkyl is optionally substituted with one or more halogen, which are the same or different;
each —Y⁰¹ is independently selected from the group consisting of —F, —Cl, —Br and —I; each n is independently 1, 2, 3 or 4;
each —Y⁰² and —Y^{02a} is independently selected from the group consisting of —H and —Br;
each —Y⁰³ and —Y^{03a} is independently selected from the group consisting of —F, —Cl, —Br, —I, —OR, —NR⁰¹¹R^{011a} and —SR⁰¹¹;
each —Y⁰⁴— is independently selected from —O—, —S—, —NR⁰¹¹—, —CR⁰¹¹R^{011a}—; each —R⁰¹¹ and —R^{011a} is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰¹², which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰¹³)—, —S(O)₂N(R⁰¹³)—, —S(O)N(R³)—, —S(O)₂—, —S(O)—, —N(R⁰¹³)S(O)₂N(R^{013a})—, —S—, —N(R⁰¹³)—, —OC(OR⁰¹³)(R^{013a})—, —N(R⁰¹³)C(O)N(R^{013a})— and —OC(O)N(R⁰¹³)—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R⁰¹², which are the same or different; and
each —R¹², —R⁰¹³ and —R^{013a} is independently selected from the group consisting of —H and C₁₋₆ alkyl; wherein C₁₋₆ alkyl is optionally substituted with one or more halogen, which are the same or different.

In certain embodiments, the pH-adjusting agent increases the pH of the emulsion of step (a) and -FG₁ and -FG₂ are independently selected from the group consisting of:

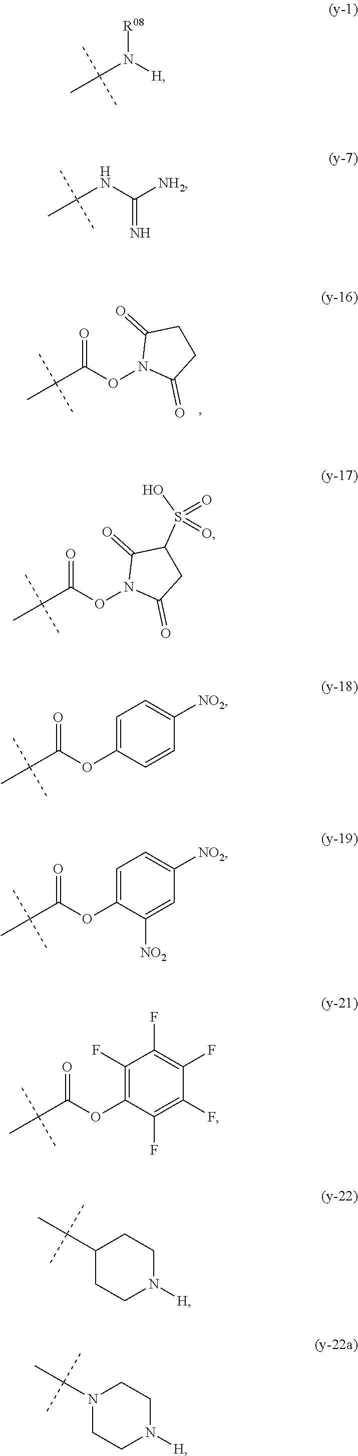

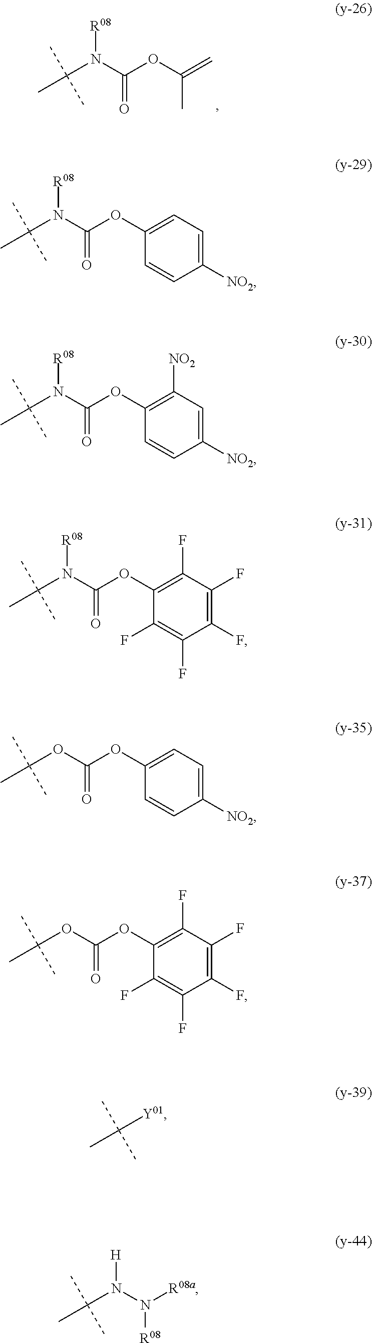

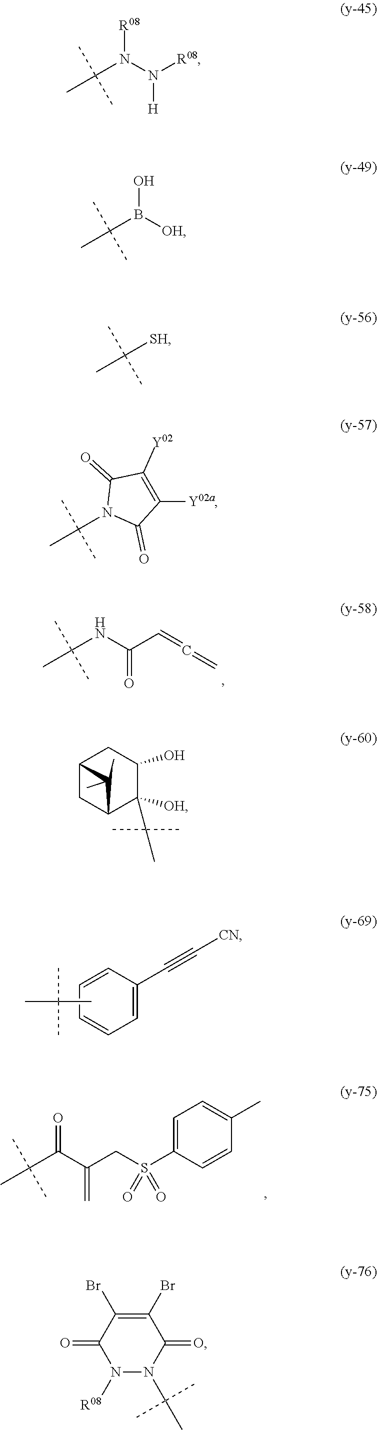

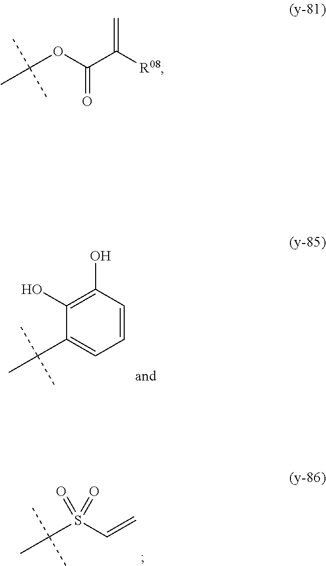

wherein the dashed line indicates the attachment to the first or second functionalized HA, such as to variable —X′— or —Y′—;
each —R⁰⁸ and —R^{08a} is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰⁹, which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰¹⁰)—, —S(O)₂N(R⁰¹⁰)—, —S(O)N(R⁰¹⁰)—, —S(O)₂—, —S(O)—, —N(R⁰¹⁰)S(O)₂N(R^{010a})—, —S—, —N(R⁰¹⁰)—, —OC(OR⁰¹⁰)(R^{010a})—, —N(R⁰¹⁰)C(O)N(R^{010a})— and —OC(O)N(R⁰¹⁰)—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R⁰⁹, which are the same or different;
each —R⁰⁹, —R⁰¹⁰ and —R^{010a} is independently selected from the group consisting of —H and C₁₋₆ alkyl; wherein C₁₋₆ alkyl is optionally substituted with one or more halogen, which are the same or different;
each —Y⁰¹ is independently selected from the group consisting of —F, —Cl, —Br and —I; each n is independently 1, 2, 3 or 4;
each —Y⁰² and —Y^{02a} is independently selected from the group consisting of —H and —Br; each —Y⁰³ and —Y^{03a} is independently selected from the group consisting of —F, —Cl, —Br, —I, —OR, —NR⁰¹¹R^{011a} and —SR⁰¹¹;
each —R⁰¹¹ and —R^{011a} is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰¹², which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰¹³)—, —S(O)₂N(R⁰¹³)—, —S(O)N(R⁰¹³)—, —S(O)₂—, —S(O)—, —N(R⁰¹³)S(O)₂N(R^{013a})—, —S—, —N(R⁰¹³)—, —OC(OR⁰¹³)(R^{013a})—, —N(R⁰¹³)C(O)N(R^{013a})— and —OC(O)N(R⁰¹³)—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R⁰¹², which are the same or different;
each —R⁰¹², —R⁰¹³ and —R^{013a} is independently selected from the group consisting of —H and C₁₋₆ alkyl; wherein C₁₋₆ alkyl is optionally substituted with one or more halogen, which are the same or different.

In certain embodiments, the pH-adjusting agent decreases the pH of the emulsion of step (a) and -FG₁ and -FG₂ are independently selected from the group consisting of:

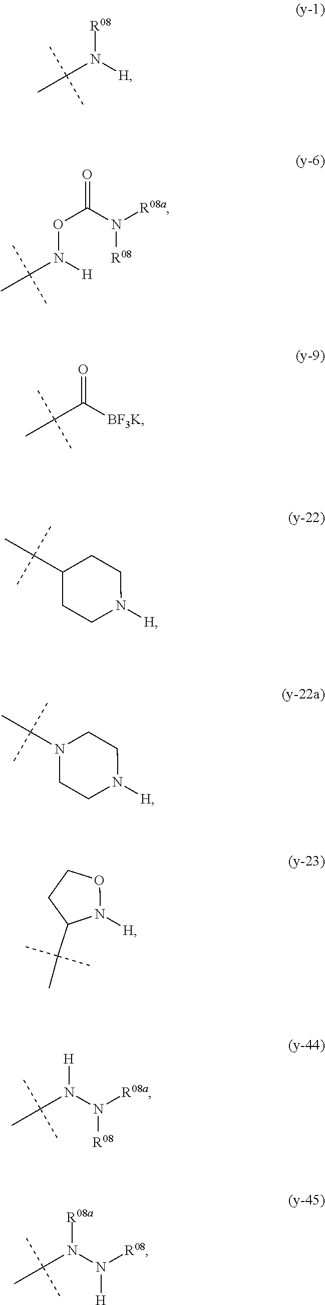

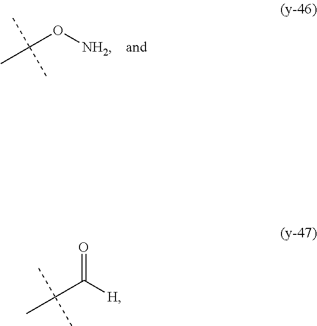

wherein the dashed line indicates the attachment to the first or second functionalized HA, such as to variable —X′— or —Y′—;
each —R⁰⁸ and —R^{08a} is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰⁹, which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰¹⁰)—, —S(O)₂N(R⁰¹⁰)—, —S(O)N(R⁰¹⁰)—, —S(O)₂—, —S(O)—, —N(R⁰¹⁰)S(O)₂N(R^{010a})—, —S—, —N(R⁰¹⁰)—, —OC(OR⁰¹⁰)(R^{010a})—, —N(R⁰¹⁰)C(O)N(R^{010a})— and —OC(O)N(R⁰¹⁰)—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R⁰⁹, which are the same or different; and
each —R⁰⁹, —R⁰¹⁰ and —R^{010a} is independently selected from the group consisting of —H and C₁₋₆ alkyl; wherein C₁₋₆ alkyl is optionally substituted with one or more halogen, which are the same or different.

In certain embodiments, -FG₁ is independently selected from the group consisting of:

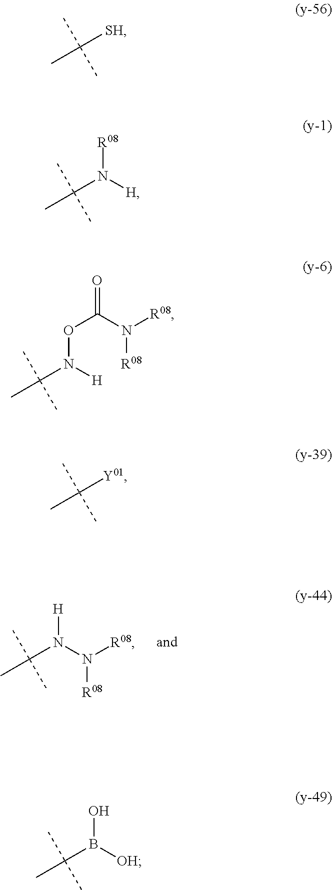

wherein the dashed line indicates the attachment to the first functionalized HA, such as to variable —X′—;
—Y⁰¹ is independently selected from the group consisting of —F, —Cl, —Br and —I;
each —R⁰⁸ and —R^{08a} is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰⁹, which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰¹⁰)—, —S(O)₂N(R⁰¹⁰)—, —S(O)N(R⁰¹⁰)—, —S(O)₂—, —S(O)—, —N(R⁰¹⁰)S(O)₂N(R^{010a})—, —S—, —N(R⁰¹⁰)—, —OC(OR⁰¹⁰)(R^{010a})—, —N(R⁰¹⁰)C(O)N(R^{010a})— and —OC(O)N(R⁰¹⁰)—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R⁰⁹, which are the same or different; and
each —R⁰⁹, —R⁰¹⁰ and —R^{010a} is independently selected from the group consisting of —H and C₁₋₆ alkyl; wherein C₁₋₆ alkyl is optionally substituted with one or more halogen, which are the same or different; and
-FG₂ is independently selected from the group consisting of

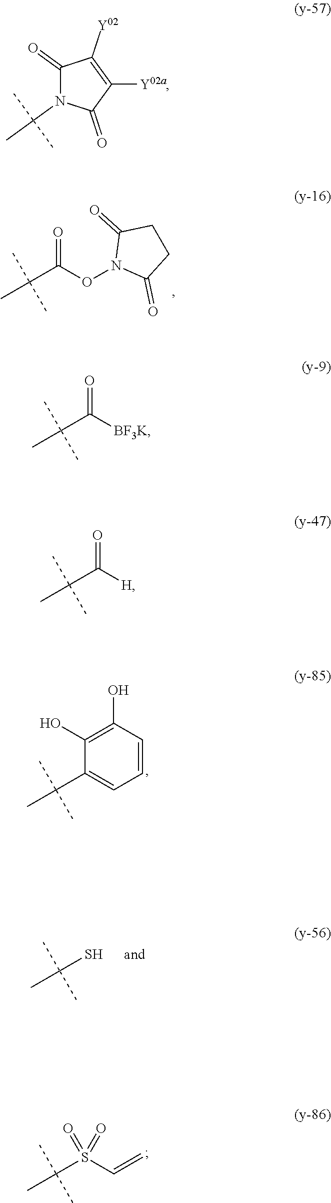

wherein the dashed line indicates the attachment to the second functionalized HA, such as to variable —Y′—;
each —Y⁰² and —Y^{02a} is independently selected from the group consisting of —H and —Br; provided that -FG₁ is of formula (y-56) then -FG₂ is of formula (y-57) or (y-86); if -FG₁ is of formula (y-1) then -FG₂ is of formula (y-16) or (y-47); if -FG₁ is of formula (y-44) then -FG₂ is of formula (y-16) or (y-47); if -FG₁ is of formula (y-6) then -FG₂ is of formula (y-9); if -FG₁ is of formula (y-49) then -FG₂ is of formula (y-85); if -FG₁ is of formula (y-44) then -FG₂ is of formula (y-47); or if -FG₁ is of formula (y-39) then -FG₂ is of formula (y-56).

In certain embodiments, the pH-adjusting agent increases the pH of the emulsion of step (a) and -FG₁ is independently selected from the group consisting of

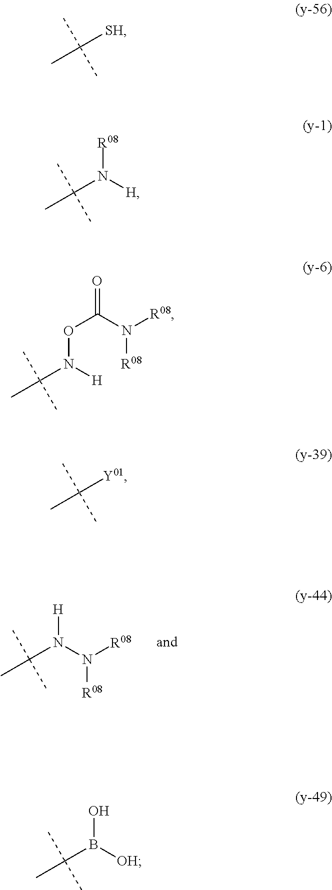

wherein the dashed line indicates the attachment to the first functionalized HA, such as to variable —X′—;
—Y⁰¹ is independently selected from the group consisting of —F, —Cl, —Br and —I;
each —R⁰⁸ and —R^{08a} is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰⁹, which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰¹⁰)—, —S(O)₂N(R⁰¹⁰)—, —S(O)N(R⁰¹⁰)—, —S(O)₂—, —S(O)—, —N(R⁰¹⁰)S(O)₂N(R^{010a})—, —S—, —N(R⁰¹⁰)—, —OC(OR⁰¹⁰)(R^{010a})—, —N(R⁰¹⁰)C(O)N(R^{010a})— and —OC(O)N(R⁰¹⁰)—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R⁰⁹, which are the same or different; and
each —R⁰⁹, —R⁰¹⁰ and —R^{010a} is independently selected from the group consisting of —H and C₁₋₆ alkyl; wherein C₁₋₆ alkyl is optionally substituted with one or more halogen, which are the same or different; and
-FG₂ is independently selected from the group consisting of

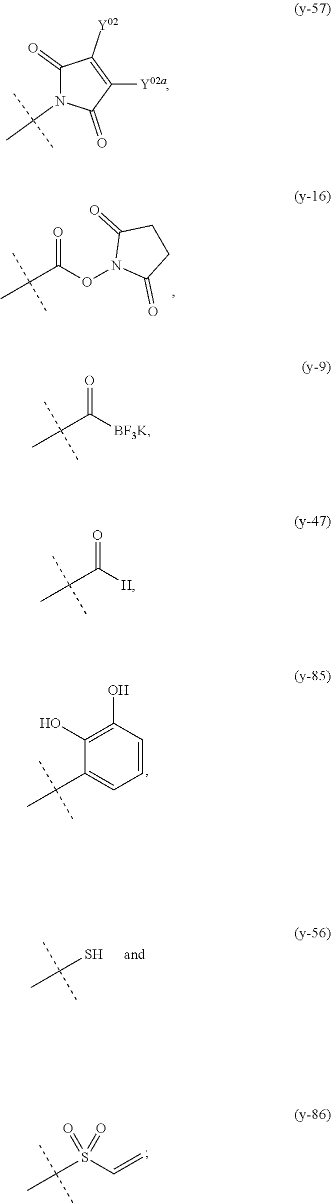

wherein the dashed line indicates the attachment to the second functionalized HA, such as to variable —Y′—;
each —Y⁰² and —Y^{02a} is independently selected from the group consisting of —H and —Br; provided that -FG₁ is of formula (y-56) then -FG₂ is of formula (y-57) or (y-86); if -FG₁ is of formula (y-1) then -FG₂ is of formula (y-16); if -FG₁ is of formula (y-44) then -FG₂ is of formula (y-16); or if -FG₁ is of formula (y-39) then -FG₂ is of formula (y-56).

In certain embodiments, the pH-adjusting agent decreases the pH of the emulsion of step (a) and -FG₁ is independently selected from the group consisting of

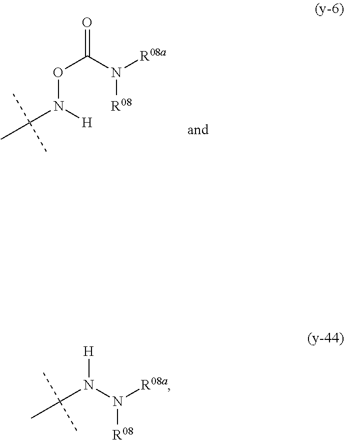

wherein the dashed line indicates the attachment to the first functionalized HA, such as to variable —X′—;
each —R⁰⁸, —R^{08a} is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰⁹, which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰¹⁰)—, —S(O)₂N(R⁰¹⁰)—, —S(O)N(R⁰¹⁰)—, —S(O)₂—, —S(O)—, —N(R⁰¹⁰)S(O)₂N(R^{010a})—, —S—, —N(R⁰¹⁰)—, —OC(OR⁰¹⁰)(R^{010a})—, —N(R⁰¹⁰)C(O)N(R^{010a})— and —OC(O)N(R⁰¹⁰)—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R⁰⁹, which are the same or different; and
each —R⁰⁹, —R⁰¹⁰ and —R^{010a} is independently selected from the group consisting of —H and C₁₋₆ alkyl; wherein C₁₋₆ alkyl is optionally substituted with one or more halogen, which are the same or different; and
-FG₂ is independently selected from the group consisting of

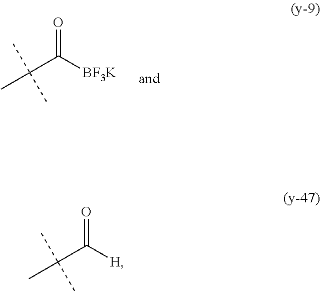

wherein the dashed line indicates the attachment to the second functionalized HA, such as to variable —Y′—;
provided that -FG₁ is of formula (y-6) then -FG₂ is of formula (y-9) and if -FG₁ is of formula (y-44) then -FG₂ is of formula (y-47).

In certain embodiments, -FG₁ is

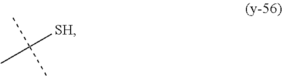

wherein the dashed line indicates the attachment to the first functionalized HA, such as to variable —X′—, -FG₂ is

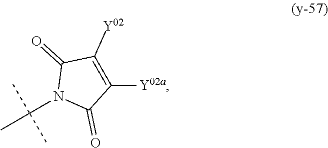

wherein the dashed line indicates the attachment to the second functionalized HA, such as to variable —Y′—, the pH-adjusting agent increases the pH of the emulsion of step (a) from about 1 to about 9, such as from about 1 to about 5.5 or such as from about 2 to about 4 and each —Y⁰² and —Y^{02a} is independently selected from the group consisting of —H and —Br.

In certain embodiments, -FG₁ is

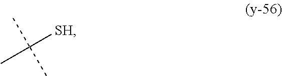

wherein the dashed line indicates the attachment to the first functionalized HA, such as to variable —X′—, -FG₂ is

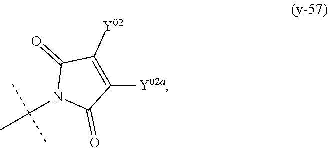

wherein the dashed line indicates the attachment to the second functionalized HA, such as to variable —Y′—, the pH-adjusting agent increases the pH of the emulsion of step (a) from about 1 to about 9, such as from about 1 to about 5.5 or such as from about 2 to about 4 and both —Y⁰² and —Y^{02a} are —H.

In certain embodiments, -FG₁ is

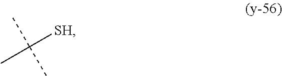

wherein the dashed line indicates the attachment to the first functionalized HA, such as to variable —X′—, -FG₂ is

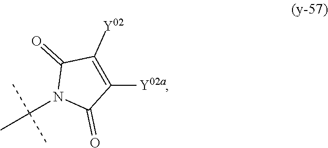

wherein the dashed line indicates the attachment to the second functionalized HA, such as to variable —Y′—, the pH-adjusting agent increases the pH of the emulsion of step (a) from 1 to 5.5 and each —Y⁰² and —Y^{02a} is independently selected from the group consisting of —H and —Br.

In certain embodiments, -FG₁ is

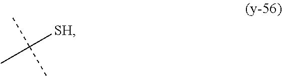

wherein the dashed line indicates the attachment to the first functionalized HA, such as to variable —X′—, -FG₂ is

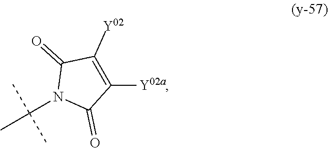

wherein the dashed line indicates the attachment to the second functionalized HA, such as to variable —Y′, the pH-adjusting agent increases the pH of the emulsion of step (a) from 1 to 5.5 and both —Y⁰² and —Y^{02a} are —H.

In certain embodiments, -FG₁ is

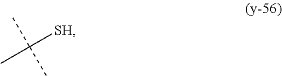

wherein the dashed line indicates the attachment to the first functionalized HA, such as to variable —X′—, -FG₂ is

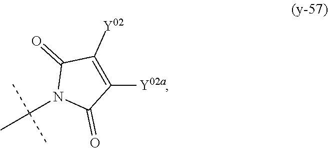

wherein the dashed line indicates the attachment to the second functionalized HA, such as to variable —Y′—, the pH-adjusting agent increases the pH of the emulsion of step (a) from 2 to 4 and each —Y⁰² and —Y^{02a} is independently selected from the group consisting of —H and —Br.

In certain embodiments, -FG₁ is

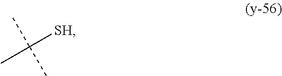

wherein the dashed line indicates the attachment to the first functionalized HA, such as to variable —X′—, -FG₂ is

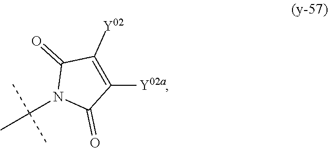

wherein the dashed line indicates the attachment to the second functionalized HA, such as to variable —Y′—, the pH-adjusting agent increases the pH of the emulsion of step (a) from 2 to 4 and both —Y⁰² and —Y^{02a} are —H.

In certain embodiments, the method of the present invention comprises the steps of.
(a) mixing a solution A with a solution B to form an emulsion, wherein solution A comprises a first functionalized HA that is modified with one or more -FG₁ and optionally further functional groups and a second functionalized HA that is modified with one or more -FG₂ and optionally further functional groups, wherein -FG₁ and -FG₂ are functional group moieties that are different from each other and wherein -FG₁ on the first functionalized HA reacts with -FG₂ on the second functionalized HA to form a plurality of crosslinks which results in the formation of hydrogel HA microspheres, wherein
   the first functionalized HA comprises a plurality of each of the following linearly connected Z¹ and Z⁵ units:

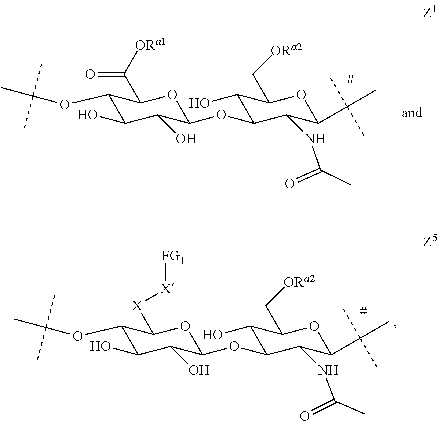

the second functionalized HA comprises a plurality of each of the following linearly connected Z¹ and Z⁶ units:

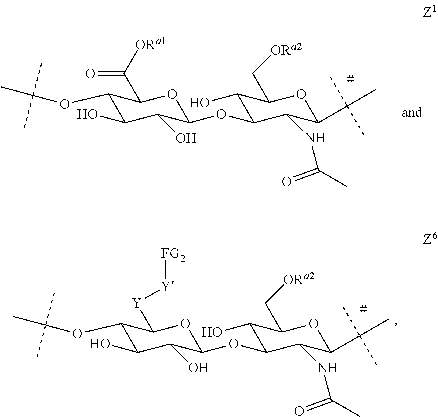

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is independently selected from the group consisting of —H, C₁₋₁₀ alkyl, an ammonium ion, a tetrabutylammonium ion, a cetyl trimethylammonium ion, an alkali metal ion and an alkaline earth metal ion;
each —R^{a2} is independently —H or C₁₋₁₀ alkyl;
each -FG₁, -FG₂ is defined as elsewhere herein;
each —X—, —Y— is independently a carbonyl group or absent;
each —X′—, —Y′— is independently a spacer moiety or absent;
(b) optionally, adding a pH-adjusting agent to the emulsion of step (a);
(c) collecting the obtained hydrogel HA microspheres of step (a) or (b), wherein said hydrogel comprises a plurality of Z³ units:

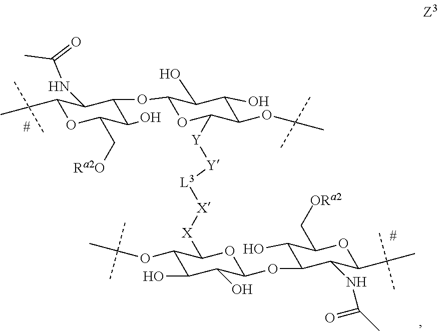

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each —R^{a2}, —X—, —Y—, —X′— and —Y′— is defined as in step (a);
each -L³- is independently a linkage moiety or absent;
(d) optionally, size fractionating the obtained hydrogel HA microspheres of step (a), (b) or (c) to obtain microspheres with a particular particle size distribution;
(e) optionally, washing the hydrogel HA microspheres obtained in step (a), (b), (c) or (d); (f) optionally, incubating the hydrogel HA microspheres of step (a), (b), (c), (d) or (e) in a buffering agent of a pH ranging from about 8 to about 12;
(g) optionally, incubating the hydrogel HA microspheres of step (a), (b), (c), (d), (e) or (f) with a reducing agent;
(h) optionally, washing the microspheres obtained in step (f) or (g); and
(i) optionally, collecting the hydrogel HA microspheres of step (d), (e), (f), (g) or (h).

The present invention also relates to the method described above wherein steps (b), (d) and (e) are not optional, while steps (f), (g) and (h) are not present.

The present invention also relates to the method described above wherein steps (b), (d) and (e) are not optional, while steps (f), (g) and (h) are not present and solution A of step (a) further comprises a buffering agent, solution B of step (a) comprises an emulsifying agent and a solvent, wherein said buffering agent, emulsifying agent and solvent are used as defined elsewhere herein.

It is understood that throughout the specification -L³- is the linkage that results from the reaction of -FG₁ with -FG₂.

Specific embodiments for —X′— and —Y′— are as described elsewhere herein.

The person skilled in the art will recognize that the hydrogel HA microspheres obtained from any one of the steps above may also comprise Z⁵ and/or Z⁶ units that have one or more unreacted -FG₁ or -FG₂ respectively, i.e. the Z⁵ and/or Z⁶ units of step (a) wherein the one or more -FG₁ did not react with the one of more -FG₂. It is also understood that said hydrogel may also comprise Z⁵ and/or Z⁶ units whereby one or more -FG₁ and/or -FG₂ underwent hydrolysis or was rendered inactive.

In certain embodiments, the majority of the -FG₁ or -FG₂ moieties do not self-react. As used herein, the term “self-react” with respect to -FG₁ or -FG₂ means that a moiety -FG₁ does not react with another moiety -FG₁ and that a moiety -FG₂ does not react with another moiety -FG₂.

The hydrogel HA microspheres may be non-degradable or biodegradable based on the chemical structure of the moiety that is part of the Z³ unit shown below:

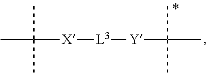

wherein the unmarked dashed line indicates the attachment to —X—, while the dashed line marked with the asterisk indicates the attachment to —Y—.

Suitably, the hydrogel HA microspheres of the present invention are biodegradable under physiological conditions.

In certain embodiments, both —X— and —Y— are carbonyl moieties.

In certain embodiments, the method of the present invention comprises the following steps:
(a) mixing a solution A with a solution B to form an emulsion, wherein solution A comprises a first functionalized HA that is modified with one or more thiol functional groups and a second functionalized HA that is modified with one or more maleimide functional groups, wherein the thiol functional groups on the first functionalized HA react with the maleimide functional groups on the second functionalized HA to form a plurality of crosslinks which results in the formation of hydrogel HA microspheres, wherein
   the first functionalized HA comprises a plurality of each of the following linearly connected Z¹ and Z⁵-i units:

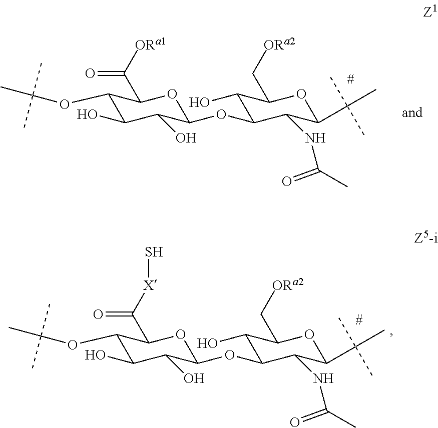

the second functionalized HA comprises a plurality of each of the following linearly connected Z¹ and Z⁶-i units:

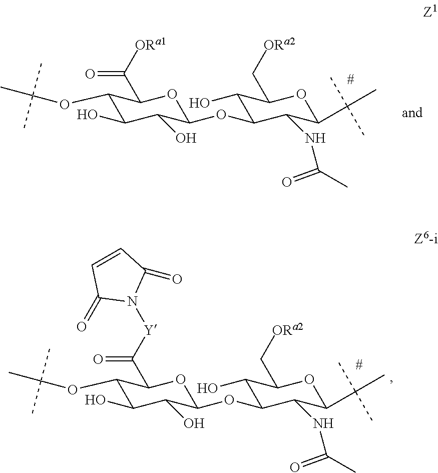

wherein
an unmarked dashed line indicates a point of attachment to an adjacent unit
at a dashed line marked with # or to a hydrogen atom;
a dashed line marked with # indicates a point of attachment to an adjacent unit at an unmarked dashed line or to a hydroxyl group;
each R^{a} is independently selected from the group consisting of —H, C₁₋₁₀ alkyl, an ammonium ion, a tetrabutylammonium ion, a cetyl trimethylammonium ion, an alkali metal ion and an alkaline earth metal ion;
each —R^{a2} is independently —H or C₁₋₁₀ alkyl;
each —X′—, —Y′— is independently a spacer moiety or absent;
(b) optionally, adding a pH-adjusting agent to the emulsion of step (a);
(c) collecting the obtained hydrogel HA microspheres of step (a) or (b), wherein said hydrogel comprises a plurality of Z³-i units:

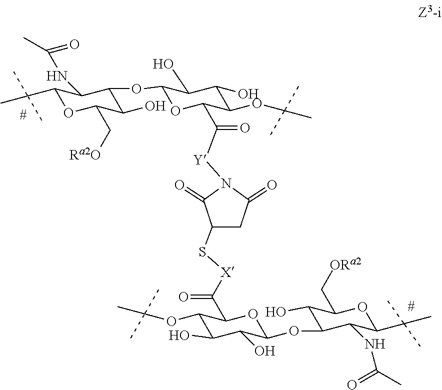

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each —R^{a2}, —X′— and —Y′— are defined as in step (a);
(d) optionally, size fractionating the obtained hydrogel HA microspheres of step (a), (b) or (c) to obtain microspheres with a particular particle size distribution;
(e) optionally, washing the hydrogel HA microspheres obtained in step (a), (b), (c) or (d);
(f) optionally, incubating the hydrogel HA microspheres of step (a), (b), (c), (d) or (e) in a buffering agent of a pH ranging from about 8 to about 12, to provide hydrogel HA microspheres comprising a plurality of Z³-i′ units:

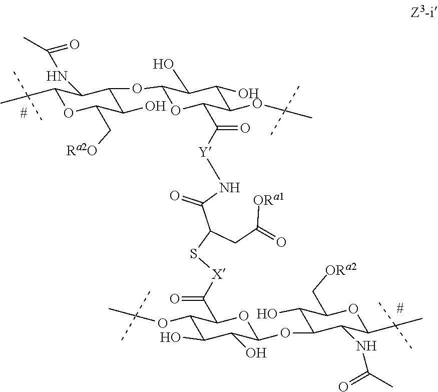

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1}, —R^{a2}, —X′— and —Y′— are defined as in step (a);
(g) optionally, incubating the hydrogel HA microspheres of step (a), (b), (c), (d), (e) or (f) with a reducing agent;
(h) optionally, washing the microspheres obtained in step (f) or (g); and
(i) optionally, collecting the hydrogel HA microspheres of step (d), (e), (f), (g) or (h).

The present invention also relates to the method described above wherein steps (b), (d) and (e) are not optional, while steps (f), (g) and (h) are not present.

The present invention also relates to the method described above wherein steps (b), (d) and (e) are not optional, while steps (f), (g) and (h) are not present and solution A of step (a) further comprises a buffering agent, solution B of step (a) comprises an emulsifying agent and a solvent and wherein said buffering agent, emulsifying agent and solvent are used as defined elsewhere herein.

The present invention also relates to the method described above wherein steps (b), (d) and (e) are not optional, while steps (f), (g), (h) and (i) are optional.

Specific embodiments for —X′— and —Y′— are as described elsewhere herein.

The person skilled in the art will recognize that in step (f) above, the resulted hydrogel HA microspheres or pharmaceutically acceptable salts thereof may comprise a plurality of Z³-i′ units and/or a plurality of Z³-i″ units depending on which of the two carbonyl groups of the thiosuccinimide ring undergoes the ring-opening hydrolysis. The structure of the Z³-i″ unit is shown below:

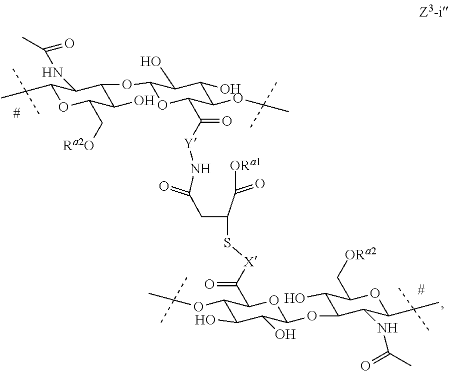

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is independently selected from the group consisting of —H, C₁₋₁₀ alkyl, an ammonium ion, a tetrabutylammonium ion, a cetyl trimethylammonium ion, an alkali metal ion and an alkaline earth metal ion;
each R^{a1}, —R^{a2} are independently —H or C₁₋₁₀ alkyl; and
each —X′—, —Y′— is independently a spacer moiety or chemical bond.

In certain embodiments, the hydrogel HA microspheres of step (f) comprise a plurality of Z³-i′ and Z³-i″ units.

In certain embodiments, the method of the present invention comprises the steps of:
(a) mixing a solution A with a solution B to form an emulsion, wherein solution A comprises a first functionalized HA that is modified with one or more thiol functional groups and a second functionalized HA that is modified with one or more maleimide functional groups, wherein the thiol functional groups on the first functionalized HA react with the maleimide functional groups on the second functionalized HA to form a plurality of crosslinks which results in the formation of hydrogel HA microspheres, wherein solution A further comprises a buffering agent, such as citrate and/or histidine and solution B comprises a solvent, such as heptane or tetradecane and an emulsifying agent, such as sorbitan monooleate; wherein,
   the first functionalized HA comprises a plurality of each of the following linearly connected Z¹ and Z⁵-i units:

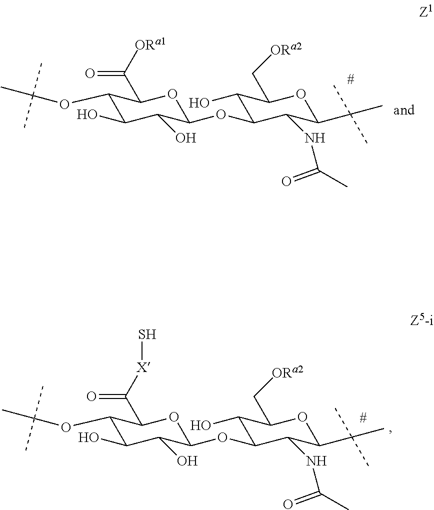

the second functionalized HA comprises a plurality of each of the following linearly connected Z¹ and Z⁶-i units:

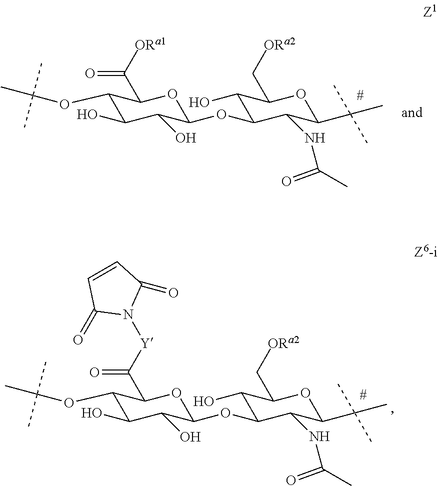

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is H or an alkali metal ion;
each —R^{a2} is —H;
each —X′— is of formula (x0);
each —Y′— is of formula (y0);
(b) adding a pH-adjusting agent to the emulsion of step (a);
(c) collecting the obtained hydrogel HA microspheres of step (b), wherein said hydrogel comprises a plurality of Z³-i units:

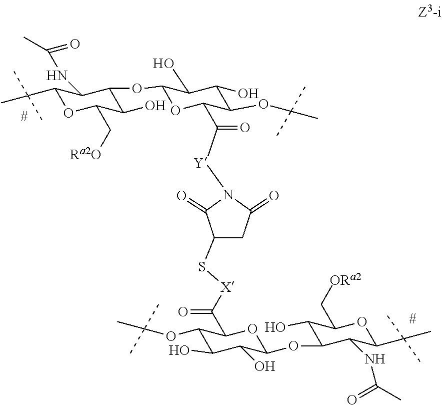

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each —R^{a2}, —X′— and —Y′— are defined as in step (a);
(d) size fractionating the obtained hydrogel HA microspheres of step (c) to obtain microspheres with a particular particle size distribution;
(e) washing the hydrogel HA microspheres obtained in step (d); and
(f) collecting the hydrogel HA microspheres of step (e).

It is understood that Z³-i unit in step (c) above depicts the connection that has formed between the first and second functionalized HA. As recognized by the skilled person, HA hydrogels are complex chemical entities and thus there are multiple ways of describing their chemical structure.

Accordingly, an alternate way of depicting the obtained hydrogel HA microspheres of step (a) or (b) is shown below, i.e. as a hydrogel HA microsphere comprising a plurality of HA strands 1A and a plurality of HA strands 1B, wherein
each 1A comprises a plurality of linearly connected units Z¹ and Z³-i (a):

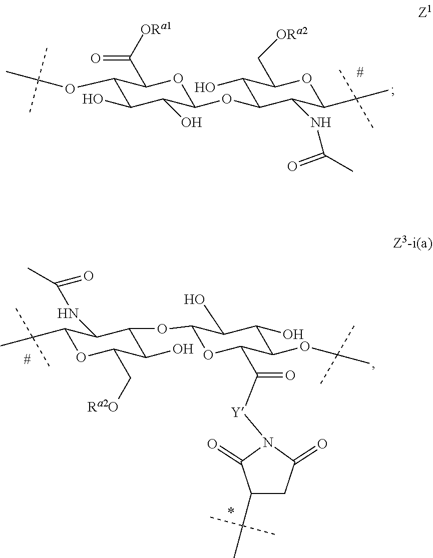

each 1B comprises a plurality of linearly connected units Z¹ and Z³-i (b):

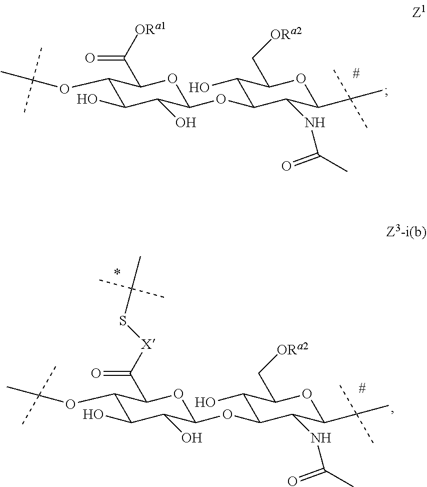

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
a dashed line marked with * indicates a point of crosslinking attachment between a unit Z³-i (a) and Z³-i (b) such that at least one 1A is crosslinked to at least 1B; and
each R^{a1}, —R^{a2}, —X′— and —Y′— are defined as in step (a).

It is understood that each 1A will also comprise unreacted Z⁶-i units

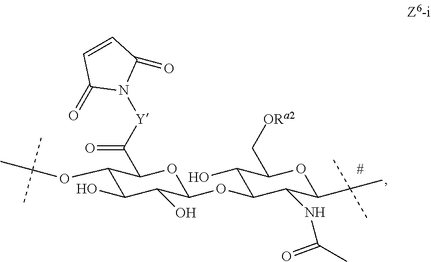

that will react with -FG₃ as defined as elsewhere therein.

In certain embodiments, the method of the present invention comprises the steps of.
(a) mixing a solution A with a solution B to form an emulsion, wherein solution A comprises a first functionalized HA that is modified with one or more thiol functional groups and a second functionalized HA that is modified with one or more maleimide functional groups, wherein the thiol functional groups on the first functionalized HA react with the maleimide functional groups on the second functionalized HA to form a plurality of crosslinks which results in the formation of hydrogel HA microspheres, wherein solution A further comprises citrate and/or histidine and solution B comprises heptane and sorbitan monooleate or tetradecane and sorbitan monooleate; wherein,
   the first functionalized HA comprises a plurality of each of the following linearly connected Z¹ and Z⁵-i units:

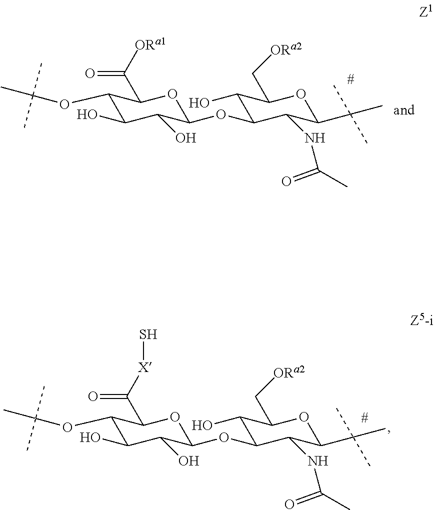

the second functionalized HA comprises a plurality of each of the following linearly connected Z¹ and Z⁶-i units:

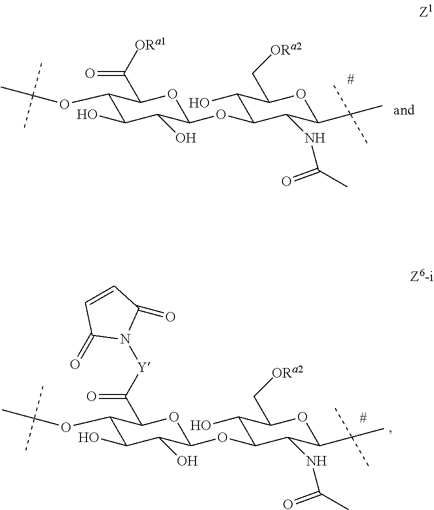

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is H or an alkali metal ion;
each —R^{a2} is —H;
each —X′— is of formula (x4):

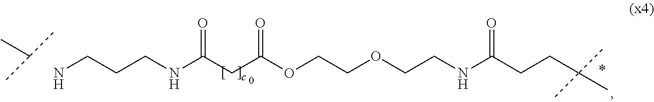

wherein the unmarked dashed line indicates the attachment to the carbonyl group, the dashed line marked with an asterisk indicates the attachment to the sulfur atom and c₀ is 7;
each —Y′— is of formula (y4):

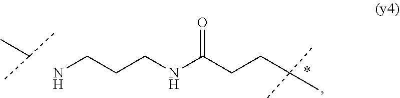

wherein the unmarked dashed line indicates the attachment to the carbonyl group and the dashed line marked with an asterisk indicates the attachment to the nitrogen atom of the maleimide ring;
(b) adding TMEDA to the emulsion of step (a);
(c) collecting the obtained hydrogel HA microspheres of step (b), wherein said hydrogel comprises a plurality of Z³-i units:

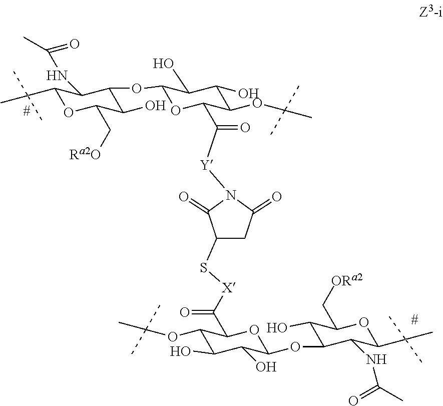

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each —R^{a2}, —X′— and —Y′— are defined as in step (a);
(d) size fractionating the obtained hydrogel HA microspheres of step (c) to obtain microspheres with a particular particle size distribution;
(e) washing the hydrogel HA microspheres obtained in step (d); and
(f) collecting the hydrogel HA microspheres of step (e).

The present invention also relates to the method described above, wherein solution A further comprises a suitable salt, such as NaCl.

In certain embodiments, the method of the present invention comprises the following steps:
(a) mixing a solution A with a solution B to form an emulsion, wherein solution A comprises a first functionalized HA that is modified with one or more thiol functional groups and a second functionalized HA that is modified with one or more maleimide functional groups, wherein the thiol functional groups on the first functionalized HA react with the maleimide functional groups on the second functionalized HA to form a plurality of crosslinks which results in the formation of hydrogel HA microspheres, wherein solution A further comprises a buffering agent such as histidine and solution B comprises a solvent such as heptane or tetradecane and an emulsifying agent such as PEG-30 dipolyhydroxystearate; wherein,
   the first functionalized HA comprises a plurality of each of the following linearly connected Z¹ and Z⁵-i units:

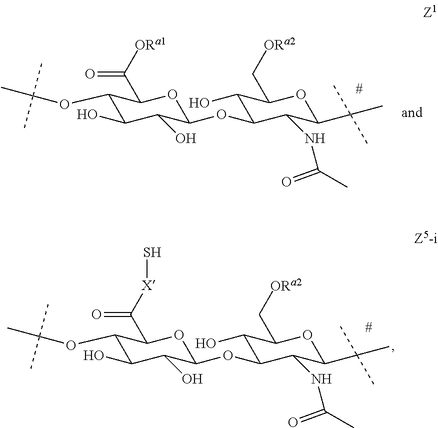

the second functionalized HA comprises a plurality of each of the following linearly connected Z¹ and Z⁶-i units:

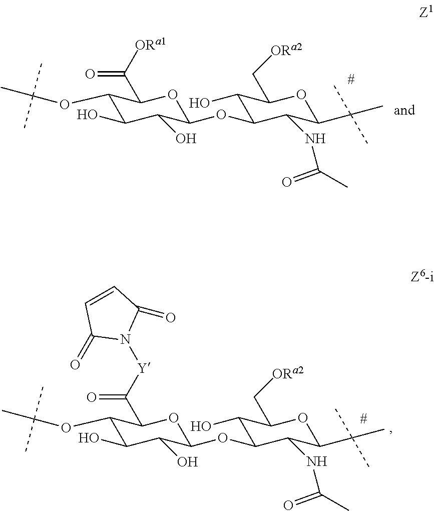

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is H or an alkali metal ion;
each —R^{a2} is —H;
each —X′— and —Y′— is of formula (y0);
(b) adding a pH-adjusting agent to the emulsion of step (a);
(c) collecting the obtained hydrogel HA microspheres of step (b), wherein said hydrogel comprises a plurality of Z³-i units:

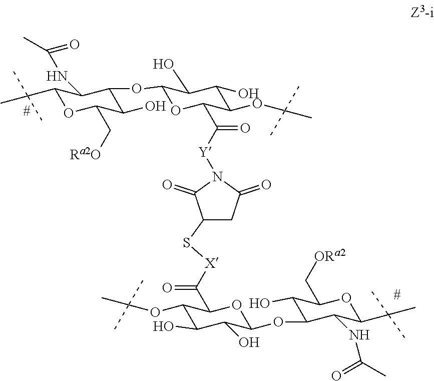

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each —R^{a2}, —X′— and —Y′— are defined as in step (a);
(d) size fractionating the obtained hydrogel HA microspheres of step (c) to obtain microspheres with a particular particle size distribution;
(e) washing the hydrogel HA microspheres obtained in step (d);
(f) incubating the hydrogel HA microspheres of step (e) in borate, to provide hydrogel HA microspheres comprising a plurality of Z³-i′ units:

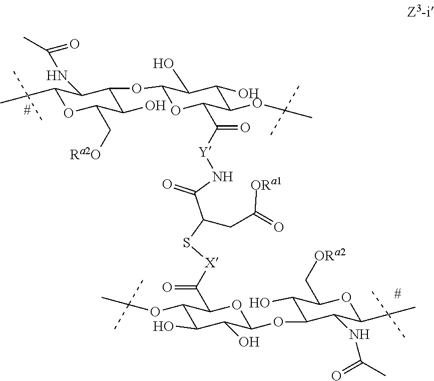

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1}, —R^{a2}, —X′— and —Y′— are defined as in step (a);
(g) incubating the hydrogel HA microspheres of step (f) with DTT;
(h) washing the microspheres obtained in step (g); and
(i) collecting the hydrogel HA microspheres of step (h).

In certain embodiments, the method of the present invention comprises the following steps:
(a) mixing a solution A with a solution B to form an emulsion, wherein solution A comprises a first functionalized HA that is modified with one or more thiol functional groups and a second functionalized HA that is modified with one or more maleimide functional groups, wherein the thiol functional groups on the first functionalized HA react with the maleimide functional groups on the second functionalized HA to form a plurality of crosslinks which results in the formation of hydrogel HA microspheres, wherein solution A further comprises histidine and solution B comprises heptane or tetradecane and PEG-30 dipolyhydroxystearate; wherein,
   the first functionalized HA comprises a plurality of each of the following linearly connected Z¹ and Z⁵-i units:

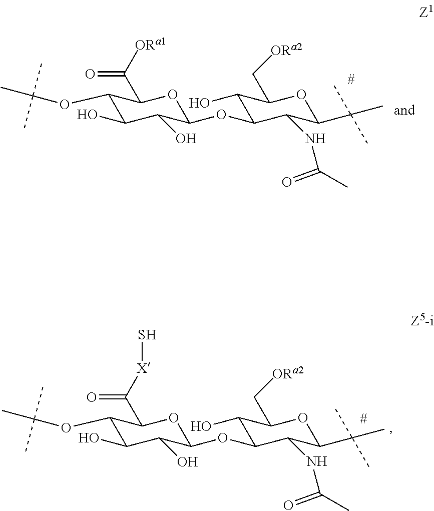

the second functionalized HA comprises a plurality of each of the following linearly connected Z¹ and Z⁶-i units:

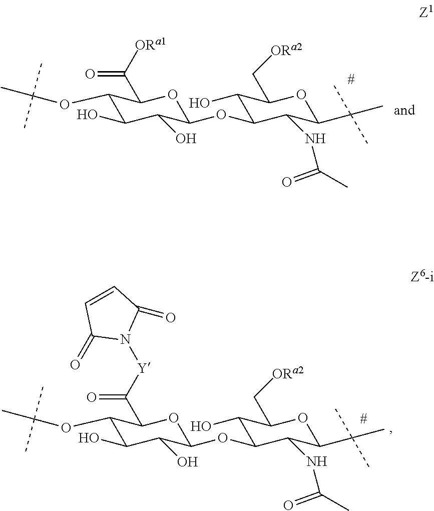

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is H or an alkali metal ion;
each —R^{a2} is —H;
each —X′— and —Y′— is of formula (y4):

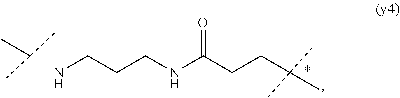

wherein for —X′— the unmarked dashed line indicates the attachment to the carbonyl group and the dashed line marked with an asterisk indicates the attachment to the sulfur atom;
wherein for —Y′— the unmarked dashed line indicates the attachment to the carbonyl group and the dashed line marked with an asterisk indicates the attachment to the nitrogen atom of the maleimide ring;
(b) adding TMEDA to the emulsion of step (a);
(c) collecting the obtained hydrogel HA microspheres of step (b), wherein said hydrogel comprises a plurality of Z³-i units:

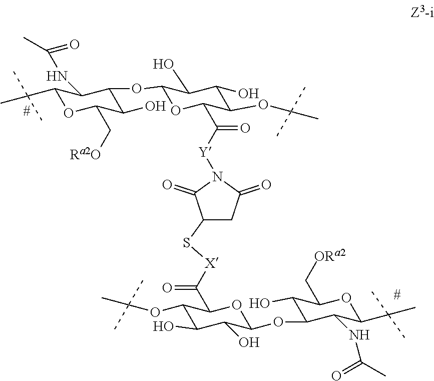

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each —R^{a2}, —X′— and —Y′— are defined as in step (a);
(d) size fractionating the obtained hydrogel HA microspheres of step (c) to obtain microspheres with a particular particle size distribution;
(e) washing the hydrogel HA microspheres obtained in step (d);
(f) incubating the hydrogel HA microspheres of step (e) in borate, to provide hydrogel HA microspheres comprising a plurality of Z³-i′ units:

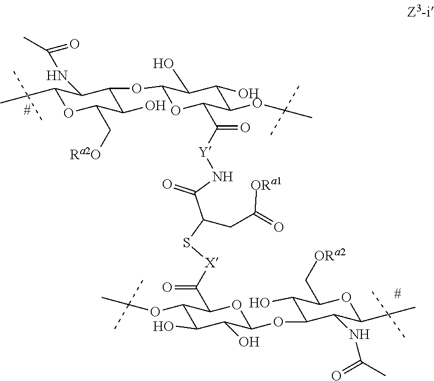

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1}, —R^{a2} —X′— and —Y′— are defined as in step (a);
(g) incubating the hydrogel HA microspheres of step (f) with DTT;
(h) washing the microspheres obtained in step (g); and
(i) collecting the hydrogel HA microspheres of step (h).

The present invention also relates to the method described above, wherein solution A further comprises a salt, such as NaCl.

It is understood by the skilled person that throughout the specification concerning unit Z³ the corresponding adjacent units that are attached to the part of the unit comprising —X′— may either be crosslinked units as defined in any one of the corresponding Z³ units or attached to unreacted Z⁵ units or Z¹ units or to any other units that are also present in the first functionalized HA or which could have been generated during the polymerization. Similarly, it is understood that the corresponding adjacent units that are attached to the part of the unit comprising —Y′— may either be crosslinked units as defined in any one of the corresponding Z³ units or attached to unreacted Z⁶ units or Z¹ units, or to any other units that are also present in the second functionalized HA or which could have been generated during the polymerization. The same rationale applies to the Z³-i, Z³-i′ and Z³-ii″ units and their corresponding Z⁵-i and Z⁶-i units.

The degree of -FG₁ functionalization of the first functionalized HA may range from about 0.001% to 100%, such as from about 0.01% to about 90%, such as from about 0.1% to about 80%, such as from about 1% to about 70%, such as from about 1% to about 60%, such as from about 1% to about 50%, such as from about 1% to about 40%, such as from about 1% to about 30%, such as from about 1% to about 20%, such as from about 1% to about 15%, such as from about 1% to about 10%, such as from about 1% to 7%, such as from about 2% to about 6% or such as from about 3% to about 5%. In certain embodiments, the degree of -FG₁ functionalization of the first functionalized HA is about 5%.

The degree of -FG₂ functionalization of the second functionalized HA may range from about 0.001% to 100%, such as from about 0.01% to about 90%, such as from about 0.1% to about 80%, such as from about 1% to about 70%, such as from about 1% to about 60%, such as from about 1% to about 50%, such as from about 1% to about 40%, such as from about 1% to about 30%, such as from about 1% to about 20%, such as from about 1% to about 15%, such as from about 1% to about 10%, such as from about 5% to about 15%, such as from about 6% to about 14%, such as from about 7% to about 13%, such as from about 8% to about 12%, such as from about 10% to about 12% or such as from about 9% to about 11%. In certain embodiments, the degree of -FG₂ functionalization of the second functionalized HA is about 10%.

In certain embodiments, the degree of -FG₁ functionalization of the first functionalized HA is about 5% and the degree of -FG₂ functionalization of the second functionalized HA is about 10%.

More particularly, thiol functional groups are introduced to provide a degree of functionalization of the HA ranging from about 0.001% to 100%, such as from about 0.01% to about 90%, such as from about 0.1% to about 80%, such as from about 1% to about 70%, such as from about 1% to about 60%, such as from about 1% to about 50%, such as from about 1% to about 40%, such as from about 1% to about 30%, such as from about 1% to about 20%, such as from about 1% to about 15%, such as from about 1% to about 10%, such as from about 1% to 7%, such as from about 2% to about 6% or such as from about 3% to about 5%.

Suitably, thiol functional groups are introduced to provide a degree of functionalization of the HA of about 5%.

More particularly, maleimide functional groups are introduced to provide a degree of functionalization of HA that may range from about 0.001% to 100%, such as from about 0.01% to about 90%, such as from about 0.1% to about 80%, such as from about 1% to about 70%, such as from about 1% to about 60%, such as from about 1% to about 50%, such as from about 1% to about 40%, such as from about 1% to about 30%, such as from about 1% to about 20%, such as from about 1% to about 15%, such as from about 1% to about 10%, such as from about 5% to about 15%, such as from about 6% to about 14%, such as from about 7% to about 13%, such as from about 8% to about 12%, such as from about 10% to about 12% or such as from about 9% to about 11%.

Suitably, maleimide functional groups are introduced to provide a degree of functionalization of the HA of about 10%.

Suitably, thiol functional groups are introduced to provide a degree of functionalization of the HA of about 5% and maleimide functional groups are introduced to provide a degree of functionalization of the HA of about 10%.

As used herein, the term “degree of functionalization (of HA)” refers to the relative ratio between the number of functionalized HA disaccharide units and the total number of all the disaccharide units comprised within a specific HA.

In certain embodiments, each -L³- is of formula (x-101):

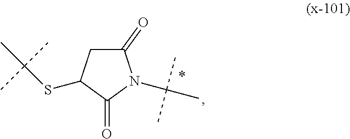

wherein the dashed line marked with the asterisk indicates the attachment to —Y′— and the unmarked dashed line indicates the attachment to —X′—.

In certain embodiments, each —X′— and —Y′— are independently a spacer moiety selected from the group consisting of -T′-, C₁₋₅₀ alkyl, C₂₋₅₀ alkenyl, and C₂₋₅₀ alkynyl; wherein C₁₋₅₀ alkyl, C₂₋₅₀ alkenyl and C₂₋₅₀ alkynyl are optionally substituted with one or more —R^{y1}, which are the same or different and wherein C₁₋₅₀ alkyl, C₂₋₅₀ alkenyl, and C₂₋₅₀ alkynyl are optionally interrupted by one or more groups selected from the group consisting of -T′-, —C(O)O—, —O—, —C(O)—, —C(O)N(R^{y2})—, —S(O)₂N(R^{y2})—, —S(O)N(R^{y2})—, —S(O)₂—, —S(O)—, —N(R^{y2})S(O)₂N(R^{y2a})—, —S—, —N(R^{y2})—, —OC(OR^{y2})(R^{y2a})—, —N(R^{y2})C(O)N(R^{y2a})— and —OC(O)N(R^{y2})—; each -T′- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each -T′- is independently optionally substituted with one or more —R^{y1}, which are the same or different;
each —R^{y1} is independently selected from the group consisting of halogen, —CN, oxo (═O), —COOR^{y3}, —OR^{y3}, —C(O)R^{y3}, —C(O)N(R³R^{y3a}), —S(O)₂N(R³R^{y3a}), —S(O)N(R³R^{y3a}), —S(O)₂R^{y3}, —S(O)R^{y3}, —N(R^{y3})S(O)₂N(R^{y3a}R^{y3}b), —SR^{y3}, —N(R^{y3}R^{y3a}), —NO₂, —OC(O)R^{y3}, —N(R^{y3})C(O)R^{y3a}, —N(R^{y3})S(O)₂R^{y3a}, —N(R^{y3})S(O)R^{y3a}, —N(R^{y3})C(O)OR^{y3a}, —N(R^{y3})C(O)N(R^{y3a}R^{y3b}), —OC(O)N(R³R^{y3a}), and C₁₋₆ alkyl; wherein C₁₋₆ alkyl is optionally substituted with one or more halogen, which are the same or different; and
each —R^{y2}, —R^{y2a}, —R^{y3}, —R^{y3a}, —R^{y3b} is independently selected from the group consisting of —H and C₁₋₆ alkyl; wherein C₁₋₆ alkyl is optionally substituted with one or more halogen, which are the same or different.

In certain embodiments, each —X′— and —Y′— are independently a spacer moiety selected from the group consisting of -T′-, C₁₋₂₅ alkyl, C₂₋₂₅ alkenyl, and C₂₋₂₅ alkynyl; wherein C₁₋₂₅ alkyl, C₂₋₂₅ alkenyl and C₂₋₂₅ alkynyl are optionally substituted with one or more —R^{y1}, which are the same or different and wherein C₁₋₂₅ alkyl, C₂₋₂₅ alkenyl, and C₂₋₂₅ alkynyl are optionally interrupted by one or more groups selected from the group consisting of -T′-, —C(O)O—, —O—, —C(O)—, —C(O)N(R^{y2})—, —S(O)₂N(R^{y2})—, —S(O)N(R^{y2})—, —S(O)₂—, —S(O)—, —N(R^{y2})S(O)₂N(R^{y2a})—, —S—, —N(R^{y2})—, —OC(OR^{y2})(R^{y2a})—, —N(R^{y2})C(O)N(R^{y2a})— and —OC(O)N(R^{y2})—; each -T′- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each -T′- is independently optionally substituted with one or more —R^{y1}, which are the same or different;
each —R^{y1} is independently selected from the group consisting of halogen, —CN, oxo (═O), —COOR^{y3}, —OR^{y3}, —C(O)R^{y3}, —C(O)N(R^{y3}R^{y3a}), —S(O)₂N(R^{y3}R^{y3a}), —S(O)N(R^{y3}R^{y3a}), —S(O)₂R^{y3}, —S(O)R^{y3}, —N(R^{y3})S(O)₂N(R^{y3a}R^{y3}b), —SR^{y3}, —N(R^{y3}R^{y3a}), —NO₂, —OC(O)R^{y3}, —N(R^{y3})C(O)R^{y3a}, —N(R^{y3})S(O)₂R^{y3a}, —N(R^{y3})S(O)R^{y3a}, —N(R^{y3})C(O)OR^{y3a}, —N(R^{y3})C(O)N(R^{y3a}R^{y3b}), —OC(O)N(R³R^{y3a}), and C₁₋₆ alkyl; wherein C₁₋₆ alkyl is optionally substituted with one or more halogen, which are the same or different; and
each —R^{y2}, —R^{y2a}, —R^{y3}, —R^{y3a}, —R^{y3b} is independently selected from the group consisting of —H and C₁₋₆ alkyl; wherein C₁₋₆ alkyl is optionally substituted with one or more halogen, which are the same or different.

In certain embodiments, each —X′— and —Y′— are independently a spacer moiety selected from the group consisting of -T′-, C₁₋₁₈ alkyl, C₂₋₁₈ alkenyl, and C₂₋₁₈ alkynyl; wherein C₁₋₁₈ alkyl, C₂₋₁₈ alkenyl and C₂₋₁₈ alkynyl are optionally substituted with one or more —R^{y1}, which are the same or different and wherein C₁₋₁₈ alkyl, C₂₋₁₈ alkenyl, and C₂₋₁₈ alkynyl are optionally interrupted by one or more groups selected from the group consisting of -T′-, —C(O)O—, —O—, —C(O)—, —C(O)N(R^{y2})—, —S(O)₂N(R^{y2})—, —S(O)N(R^{y2})—, —S(O)₂—, —S(O)—, —N(R^{y2})S(O)₂N(R^{y2a})—, —S—, —N(R^{y2})—, —OC(OR^{y2})(R^{y2a})—, —N(R^{y2})C(O)N(R^{y2a})— and —OC(O)N(R^{y2})—; each -T′- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each -T′- is independently optionally substituted with one or more —R^{y1}, which are the same or different;
each —R^{y1} is independently selected from the group consisting of halogen, —CN, oxo (═O), —COOR^{y3}, —OR^{y3}, —C(O)R^{y3}, —C(O)N(R³R^{y3a}), —S(O)₂N(R³R^{y3a}), —S(O)N(R³R^{y3a}), —S(O)₂R^{y3}, —S(O)R^{y3}, —N(R^{y3})S(O)₂N(R^{y3a}R^{y3}b), —SR^{y3}, —N(R^{y3}R^{y3a}), —NO₂, —OC(O)R^{y3}, —N(R^{y3})C(O)R^{y3a}, —N(R^{y3})S(O)₂R^{y3a}, —N(R^{y3})S(O)R^{y3a}, —N(R^{y3})C(O)OR^{y3a}, —N(R^{y3})C(O)N(R^{y3a}R^{y3b}), —OC(O)N(R³R^{y3a}), and C₁₋₆ alkyl; wherein C₁₋₆ alkyl is optionally substituted with one or more halogen, which are the same or different; and
each —R^{y2}, —R^{y2a}, —R^{y3}, —R^{y3a}, —R^{y3b} is independently selected from the group consisting of —H and C₁₋₆ alkyl; wherein C₁₋₆ alkyl is optionally substituted with one or more halogen, which are the same or different.

In certain embodiments, each —X′— and —Y′— are independently a spacer moiety selected from the group consisting of -T′-, C₁₋₁₀ alkyl, C₂₋₁₀ alkenyl, and C₂₋₁₀ alkynyl; wherein C₁₋₁₀ alkyl, C₂₋₁₀ alkenyl and C₂₋₁₀ alkynyl are optionally substituted with one or more —R^{y1}, which are the same or different and wherein C₁₋₁₀ alkyl, C₂₋₁₀ alkenyl, and C₂₋₁₀ alkynyl are optionally interrupted by one or more groups selected from the group consisting of -T′-, —C(O)O—, —O—, —C(O)—, —C(O)N(R^{y2})—, —S(O)₂N(R^{y2})—, —S(O)N(R^{y2})—, —S(O)₂—, —S(O)—, —N(R^{y2})S(O)₂N(R^{y2a})—, —S—, —N(R^{y2})—, —OC(OR^{y2})(R^{y2a})—, —N(R^{y2})C(O)N(R^{y2a})— and —OC(O)N(R^{y2})—; each -T′- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each -T′- is independently optionally substituted with one or more —R^{y1}, which are the same or different;
each —R^{y1} is independently selected from the group consisting of halogen, —CN, oxo (═O), —COOR^{y3}, —OR^{y3}, —C(O)R^{y3}, —C(O)N(R^{y3}R^{y3a}), —S(O)₂N(R^{y3}R^{y3a}), —S(O)N(R^{y3}R^{y3a}), —S(O)₂R^{y3}, —S(O)R^{y3}, —N(R^{y3})S(O)₂N(R^{y3a}R^{y3}b), —SR^{y3}, —N(R^{y3}R^{y3a}), —NO₂, —OC(O)R^{y3}, —N(R^{y3})C(O)R^{y3a}, —N(R^{y3})S(O)₂R^{y3a}, —N(R^{y3})S(O)R^{y3a}, —N(R^{y3})C(O)OR^{y3a}, —N(R^{y3})C(O)N(R^{y3a}R^{y3b}), —OC(O)N(R³R^{y3a}), and C₁₋₆ alkyl; wherein C₁₋₆ alkyl is optionally substituted with one or more halogen, which are the same or different; and
each —R^{y2}, —R^{y2a}, —R^{y3}, —R^{y3a}, —R^{y3b} is independently selected from the group consisting of —H and C₁₋₆ alkyl; wherein C₁₋₆ alkyl is optionally substituted with one or more halogen, which are the same or different.

In certain embodiments, each —X′— and —Y′— are independently a spacer moiety selected from the group consisting of -T′-, C₁₋₅ alkyl, C₂₋₅ alkenyl, and C₂₋₅ alkynyl; wherein C₁₋₅ alkyl, C₂₋₅ alkenyl and C₂₋₅ alkynyl are optionally substituted with one or more —R^{y1}, which are the same or different and wherein C₁₋₅ alkyl, C₂₋₅ alkenyl, and C₂₋₅ alkynyl are optionally interrupted by one or more groups selected from the group consisting of -T′-, —C(O)O—, —O—, —C(O)—, —C(O)N(R^{y2})—, —S(O)₂N(R^{y2})—, —S(O)N(R^{y2})—, —S(O)₂—, —S(O)—, —N(R^{y2})S(O)₂N(R^{y2a})—, —S—, —N(R^{y2})—, —OC(OR^{y2})(R^{y2a})—, —N(R^{y2})C(O)N(R^{y2a})— and —OC(O)N(R^{y2})—; each -T′- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each -T′- is independently optionally substituted with one or more —R^{y1}, which are the same or different;
each —R^{y1} is independently selected from the group consisting of halogen, —CN, oxo (═O), —COOR^{y3}, —OR^{y3}, —C(O)R^{y3}, —C(O)N(R³R^{y3a}), —S(O)₂N(R³R^{y3a}), —S(O)N(R^{y3}R^{y3a}), —S(O)₂R^{y3}, —S(O)R^{y3}, —N(R^{y3})S(O)₂N(R^{y3a}R^{y3}b), —SR^{y3}, —N(R^{y3}R^{y3a}), —NO₂, —OC(O)R^{y3}, —N(R^{y3})C(O)R^{y3a}, —N(R^{y3})S(O)₂R^{y3a}, —N(R^{y3})S(O)R^{y3a}, —N(R^{y3})C(O)OR^{y3a}, —N(R^{y3})C(O)N(R^{y3a}R^{y3b}), —OC(O)N(R³R^{y3a}), and C₁₋₄ alkyl; wherein C₁₋₄ alkyl is optionally substituted with one or more halogen, which are the same or different; and
each —R^{y2}, —R^{y2a}, —R^{y3}, —R^{y3a}, —R^{y3b} is independently selected from the group consisting of —H and C₁₋₄ alkyl; wherein C₁₋₄ alkyl is optionally substituted with one or more halogen, which are the same or different.

In certain embodiments, —X′— is of formula (x0):

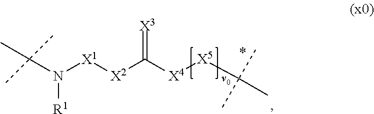

wherein
the unmarked dashed line indicates the attachment to —X— and the dashed line marked with an asterisk indicates the attachment to -FG₁ or -L³-;
v₀ is selected from the group consisting of 0 and 1;
—X¹—, —X⁴— are independently C₁₋₁₀ alkyl, which C₁₋₁₀ alkyl is optionally interrupted by one or more groups independently selected from —O—, -T-, —N(R^{y1})—, —C(O)O— and —C(O)N(R^{y1})—; and which C₁₋₁₀ alkyl chain is optionally substituted with one or more groups independently selected from —OH, -T, —N(R^{y1})— and —C(O)N(R^{y2}R^{y2a});
wherein —R^{y1}, —R^{y2}, —R^{y2a} are independently selected from the group consisting of H and C₁₋₄ alkyl;
—X²— is selected from the group consisting of —N(R¹)—, —O—, —S— and —Se—;
═X³ is selected from the group consisting of ═O, ═N(R¹) and ═S;
—X⁵— is C₁₋₂₀ alkyl, which C₁₋₂₀ alkyl is optionally interrupted by one or more groups independently selected from —O—, —C(O)O—, -T-, —N(R^{y1})— and —N(R^{y1})C(O)—; and which C₁₋₂₀ alkyl chain is optionally substituted with one or more groups independently selected from —OH, -T, —N(R^{y1})— and —C(O)N(R^{y2}R^{y2a});
—R¹ is independently selected from the group consisting of —H, C₁₋₅ alkyl and -T;
wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R², which are the same or different;
   —R² is selected from the group consisting of halogen, —CN, oxo, —C(O)OR³, —OR³, —C(O)R³, —C(O)N(R³)(R^{3a}), —S(O)₂N(R³)(R^{3a}), —S(O)N(R³)(R^{3a}), —S(O)₂R³, —S(O)R³, —N(R³)S(O)₂N(R^{3a})(R^{3b}), —SR³, —N(R³)(R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a})(R^{3b}), —OC(O)N(R³)(R^{3a}) and C₁₋₆ alkyl; wherein C₁₋₆ alkyl is optionally substituted with one or more halogen, which are the same or different; and
   wherein —R³, —R^{3a} and —R^{3b} are independently selected from the group consisting of —H and C₁₋₆ alkyl; wherein C₁₋₆ alkyl is optionally substituted with one or more halogen, which are the same or different.

In certain embodiments, v₀ of formula (x0) is 0. In certain embodiments, v₀ of formula (x0) is 1.

In certain embodiments, —X′— is of formula (x1):

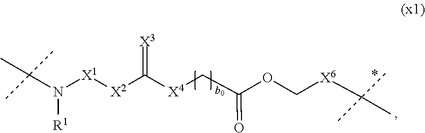

wherein
the unmarked dashed line indicates the attachment to —X— and the dashed line marked with an asterisk indicates the attachment to -FG₁ or -L³-;
b₀ is selected from the group consisting of 0, 1, 2, 3, 4, 5, 6, 7, 8, 9 and 10;
—X¹—, —X⁴— are independently C₁₋₅ alkyl, which C₁₋₅ alkyl is optionally interrupted by one or more groups independently selected from —O—, -T-, —N(R^{y1})— and —C(O)N(R^{y1})—; and which C₁₋₅ alkyl chain is optionally substituted with one or more groups independently selected from —OH, -T, —NH(R^{y1}) and —C(O)N(R^{y2}R^{y2a}); wherein —R^{y1}, —R^{y2}, —R^{y2a} are independently selected from the group consisting of —H and C₁₋₄ alkyl;
—X²— is selected from the group consisting of —N(R¹)—, —O—, —S— and —Se—;
═X³ is selected from the group consisting of ═O, ═N(R¹) and ═S;
—X⁶— is C₁₋₁₀ alkyl, which C₁₋₁₀ alkyl is optionally interrupted by one or more groups independently selected from —O—, —C(O)O—, -T-, —N(R^{y1})— and —N(R^{y1})C(O)—; and which C₁₋₁₀ alkyl chain is optionally substituted with one or more groups independently selected from —OH, -T, —NH(R^{y1}) and —C(O)N(R^{y2}R^{y2a});
—R¹ is independently selected from the group consisting of —H, C₁₋₅ alkyl and -T;
wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R², which are the same or different;
   —R² is selected from the group consisting of halogen, —CN, oxo, —C(O)OR³, —OR³, —C(O)R³, —C(O)N(R³)(R^{3a}), —S(O)₂N(R³)(R^{3a}), —S(O)N(R³)(R^{3a}), —S(O)₂R³, —S(O)R³, —N(R³)S(O)₂N(R^{3a})(R^{3b}), —SR³, —N(R³)(R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a})(R^{3b}), —OC(O)N(R³)(R^{3a}) and C₁₋₆ alkyl; wherein C₁₋₆ alkyl is optionally substituted with one or more halogen, which are the same or different; and
      wherein —R³, —R^{3a} and —R^{3b} are independently selected from the group consisting of —H and C₁₋₆ alkyl; wherein C₁₋₆ alkyl is optionally substituted with one or more halogen, which are the same or different.

In certain embodiments, —X′— is of formula (x2):

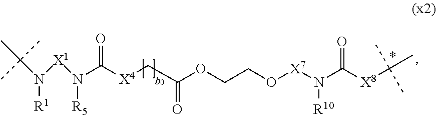

wherein
the unmarked dashed line indicates the attachment to —X— and the dashed line marked with an asterisk indicates the attachment to -FG₁ or -L³-;
b₀ is selected from the group consisting of 0, 1, 2, 3, 4, 5, 6, 7, 8, 9 and 10;
—X¹—, —X⁴—, —X⁷—, —X′— are independently C₁₋₅ alkyl, which C₁₋₅ alkyl is optionally interrupted by one or more groups independently selected from —O—, -T-, —N(R^{y1})— and —C(O)N(R^{y1})—; and which C₁₋₅ alkyl chain is optionally substituted with one or more groups independently selected from —OH, -T, —NH(R^{y1}) and —C(O)N(R^{y2}R^{y2a}); wherein —R^{y1}, —R^{y2}, —R^{y2a} are independently selected from the group consisting of H and C₁₋₄ alkyl;
—R¹, —R⁵, —R¹⁰ are independently selected from the group consisting of —H, C₁₋₅ alkyl and -T;
   wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R⁶, which are the same or different;
      —R⁶ is selected from the group consisting of halogen, —CN, oxo, —C(O)OR⁷, —OR⁷, —C(O)R⁷, —C(O)N(R⁷)(R^{7a}), —S(O)₂N(R⁷)(R^{7a}), —S(O)N(R⁷)(R^{7a}), —S(O)₂R⁷, —S(O)R⁷, —N(R⁷)S(O)₂N(R^{7a})(R^{7b}), —SR⁷, —N(R⁷)(R^{7a}), —NO₂, —OC(O)R⁷, —N(R⁷)C(O)R^{7a}, —N(R⁷)S(O)₂R⁷a, —N(R⁷)S(O)R^{7a}, —N(R⁷)C(O)OR^{7a}, —N(R⁷)C(O)N(R^{7a})(R⁷b), —OC(O)N(R⁷)(R^{7a}) and C₁₋₆ alkyl; wherein C₁₋₆ alkyl is optionally substituted with one or more halogen, which are the same or different; and
         wherein —R⁷, —R^{7a} and —R^{7b} are independently selected from the group consisting of —H and C₁₋₆ alkyl; wherein C₁₋₆ alkyl is optionally substituted with one or more halogen, which are the same or different.

In certain embodiments, —X′— is of formula (x3).

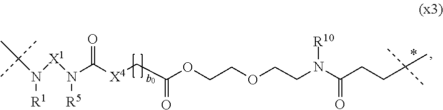

wherein
the unmarked dashed line indicates the attachment to —X— and the dashed line marked with an asterisk indicates the attachment to -FG₁ or -L³-;
b₀ is selected from the group consisting of 0, 1, 2, 3, 4, 5, 6, 7, 8, 9 and 10;
—X¹—, —X⁴— are independently C₁₋₅ alkyl, which C₁₋₅ alkyl is optionally interrupted by one or more groups independently selected from —O—, -T-, —N(R^{y1})— and —C(O)N(R^{y1})—; and which C₁₋₅ alkyl chain is optionally substituted with one or more groups independently selected from —OH, -T, —NH(R^{y1}) and —C(O)N(R^{y2}R^{y2a}); wherein —R^{y1}, —R^{y2}, —R^{y2a} are independently selected from the group consisting of H and C₁₋₄ alkyl;
—R¹, —R⁵, —R¹⁰ are independently selected from the group consisting of —H, C₁₋₅ alkyl and -T;
   wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R⁶, which are the same or different;
   —R⁶ is selected from the group consisting of halogen, —CN, oxo, —C(O)OR⁷, —OR⁷, —C(O)R⁷, —C(O)N(R⁷)(R^{7a}), —S(O)₂N(R⁷)(R^{7a}), —S(O)N(R⁷)(R^{7a}), —S(O)₂R⁷, —S(O)R⁷, —N(R⁷)S(O)₂N(R^{7a})(R^{7b}), —SR⁷, —N(R⁷)(R^{7a}), —NO₂, —OC(O)R⁷, —N(R⁷)C(O)R^{7a}, —N(R⁷)S(O)₂R^{7a}, —N(R⁷)S(O)R^{7a}, —N(R⁷)C(O)OR^{7a}, —N(R⁷)C(O)N(R^{7a})(R^{7b}), —OC(O)N(R⁷)(R^{7a}) and C₁₋₆ alkyl; wherein C₁₋₆ alkyl is optionally substituted with one or more halogen, which are the same or different; and
      wherein —R⁷, —R^{7a} and —R^{7b} are independently selected from the group consisting of —H and C₁₋₆ alkyl; wherein C₁₋₆ alkyl is optionally substituted with one or more halogen, which are the same or different.

In certain embodiments, b₀ of formula (x1), (x2) or (x3) is 1. In certain embodiments, b₀ of formula (x1), (x2) or (x3) is 2. In certain embodiments, b₀ of formula (x1), (x2) or (x3) is 3. In certain embodiments, b₀ of formula (x1), (x2) or (x3) is 4. In certain embodiments, b₀ of formula (x1), (x2) or (x3) is 5. In certain embodiments, b₀ of formula (x1), (x2) or (x3) is 6. In certain embodiments, b₀ of formula (x1), (x2) or (x3) is 7. In certain embodiments, b₀ of formula (x1), (x2) or (x3) is 8. In certain embodiments, b₀ of formula (x1), (x2) or (x3) is 9. In certain embodiments, b₀ of formula (x1), (x2) or (x3) is 10.

In certain embodiments, —X′— is of formula (x4):

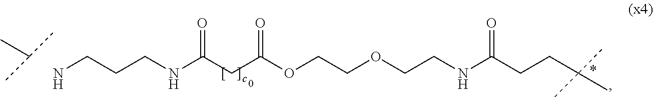

wherein
the unmarked dashed line indicates the attachment to —X— and the dashed line marked with an asterisk indicates the attachment to -FG₁ or -L³-; and
c₀ is selected from the group consisting of 1, 2, 3, 4, 5, 6, 7, 8, 9 and 10.

In certain embodiments, c₀ of formula (x4) is 1. In certain embodiments, c₀ of formula (x4) is 2.

In certain embodiments, c₀ of formula (x4) is 3. In certain embodiments, c₀ of formula (x4) is 4.

In certain embodiments, c₀ of formula (x4) is 5. In certain embodiments, c₀ of formula (x4) is 6.

In certain embodiments, c₀ of formula (x4) is 7. In certain embodiments, c₀ of formula (x4) is 8.

In certain embodiments, c₀ of formula (x4) is 9. In certain embodiments, c₀ of formula (x4) is 10.

In certain embodiments, —Y′— is of formula (y0):

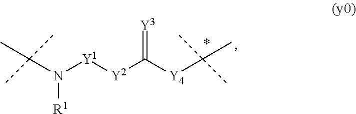

wherein
the unmarked dashed line indicates the attachment to —Y— and the dashed line marked with an asterisk indicates the attachment to -FG₂, -L³-, -L⁴- or -L⁵-;
—Y¹—, —Y⁴— are independently C₁₋₁₀ alkyl, which C₁₋₁₀ alkyl is optionally interrupted by one or more groups independently selected from —O—, -T-, —N(R^{y1})—, —C(O)O— and —C(O)N(R^{y1})—; and which C₁₋₁₀ alkyl chain is optionally substituted with one or more groups independently selected from —OH, -T, —NH(R^{y1}) and —C(O)N(R^{y2}R^{y2a});
wherein —R^{y1}, —R^{y2}, —R^{y2a} are independently selected from the group consisting of H and C₁₋₄ alkyl;
—Y²— is selected from the group consisting of —N(R²)—, —O—, —S— and —Se—;
═Y³ is selected from the group consisting of ═O, ═N(R²) and ═S;
—R¹, —R² are independently selected from the group consisting of —H, C₁₋₅ alkyl and -T; wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R³, which are the same or different;
   —R³ is selected from the group consisting of halogen, —CN, oxo, —C(O)OR⁴, —OR⁴, —C(O)R⁴, —C(O)N(R⁴)(R^{4a}), —S(O)₂N(R⁴)(R^{4a}), —S(O)N(R⁴)(R^{4a}), —S(O)₂R⁴, —S(O)R⁴, —N(R⁴)S(O)₂N(R^{4a})(R^{4b}), —SR⁴, —N(R⁴)(R^{4a}), —NO₂, —OC(O)R⁴, —N(R⁴)C(O)R^{4a}, —N(R⁴)S(O)₂R^{4a}, —N(R⁴)S(O)R^{4a}, —N(R⁴)C(O)OR^{4a}, —N(R⁴)C(O)N(R^{4a})(R^{4b}), —OC(O)N(R⁴)(R^{4a}) and C₁₋₆ alkyl; wherein C₁₋₆ alkyl is optionally substituted with one or more halogen, which are the same or different; and
      wherein —R⁴, —R^{4a} and —R^{4b} are independently selected from the group consisting of —H and C₁₋₆ alkyl; wherein C₁₋₆ alkyl is optionally substituted with one or more halogen, which are the same or different.

In certain embodiments, —X′— is of formula (y0), wherein the unmarked dashed line indicates the attachment to —X— and the dashed line marked with an asterisk indicates the attachment to -FG₁.

In certain embodiments, —Y²— of formula (y0) is —N(R²)—. In certain embodiments, —Y²— of formula (y0) is —O—. In certain embodiments, —Y²— of formula (y0) is —S—. In certain embodiments, —Y²— of formula (y0) is —Se—.

In certain embodiments, =Y³ of formula (y0) is ═O. In certain embodiments, =Y³ of formula (y0) is ═N(R²). In certain embodiments, =Y³ of formula (y0) is ═S.

In certain embodiments, —Y′— is of formula (y1).

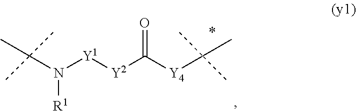

wherein
the unmarked dashed line indicates the attachment to —Y— and the dashed line marked with an asterisk indicates the attachment to -FG₂, -L³-, -L⁴- or -L⁵-;
—Y¹—, —Y⁴— are independently C₁₋₁₀ alkyl, which C₁₋₁₀ alkyl is optionally interrupted by one or more groups independently selected from —O—, -T-, —N(R^{y1})—, —C(O)O— and —C(O)N(R^{y1})—; and which C₁₋₁₀ alkyl chain is optionally substituted with one or more groups independently selected from —OH, -T, —NH(R^{y1}) and —C(O)N(R^{y2}R^{y2a});
wherein —R^{y1}, —R^{y2}, —R^{y2a} are independently selected from the group consisting of H and C₁₋₄ alkyl;
—Y²— is selected from the group consisting of —N(R²)—, —O—, —S— and —Se—;
—R¹, —R² are independently selected from the group consisting of —H, C₁₋₅ alkyl and -T; wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R³, which are the same or different;
   —R³ is selected from the group consisting of halogen, —CN, oxo, —C(O)OR⁴, —OR⁴, —C(O)R⁴, —C(O)N(R⁴)(R^{4a}), —S(O)₂N(R⁴)(R^{4a}), —S(O)N(R⁴)(R^{4a}), —S(O)₂R⁴, —S(O)R⁴, —N(R⁴)S(O)₂N(R^{4a})(R^{4b}), —SR⁴, —N(R⁴)(R^{4a}), —NO₂, —OC(O)R⁴, —N(R⁴)C(O)R^{4a}, —N(R⁴)S(O)₂R^{4a}, —N(R⁴)S(O)R^{4a}, —N(R⁴)C(O)OR^{4a}, —N(R⁴)C(O)N(R^{4a})(R^{4b}), —OC(O)N(R⁴)(R^{4a}) and C₁₋₆ alkyl; wherein C₁₋₆ alkyl is optionally substituted with one or more halogen, which are the same or different; and
      wherein —R⁴, —R^{4a} and —R^{4b} are independently selected from the group consisting of —H and C₁₋₆ alkyl; wherein C₁₋₆ alkyl is optionally substituted with one or more halogen, which are the same or different.

In certain embodiments, —X′— is of formula (y1), wherein the unmarked dashed line indicates the attachment to —X— and the dashed line marked with an asterisk indicates the attachment to -FG₁.

In certain embodiments, —Y′— is of formula (y2):

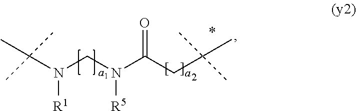

wherein
the unmarked dashed line indicates the attachment to —Y— and the dashed line marked with an asterisk indicates the attachment to -FG₂, -L³-, -L⁴- or -L⁵-;
—R¹, —R⁵ are independently selected from the group consisting of —H, methyl, ethyl, propyl and isopropyl; and
a₁, a₂ are independently selected from the group consisting of 1, 2, 3, 4, 5, 6, 7, 8, 9 and 10, preferably from the group consisting of 1, 2, 3, 4, 5, 6, 7 and 8, more preferably from the group consisting of 1, 2, 3, 4, 5 and 6 or even more preferably from the group consisting of 1,2 and 3.

In certain embodiments, —X′— is of formula (y2), wherein the unmarked dashed line indicates the attachment to —X— and the dashed line marked with an asterisk indicates the attachment to -FG₁.

In certain embodiments, —Y′— is of formula (y3):

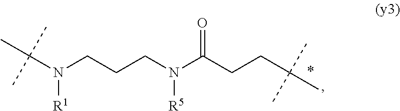

wherein
the unmarked dashed line indicates the attachment to —Y— and the dashed line marked with an asterisk indicates the attachment to -FG₂, -L³-, -L⁴- or -L⁵-; and
—R¹, —R⁵ are independently selected from the group consisting of —H, methyl, ethyl, propyl and isopropyl.

In certain embodiments, —X′— is of formula (y3), wherein the unmarked dashed line indicates the attachment to —X— and the dashed line marked with an asterisk indicates the attachment to -FG₁ or -L³-.

In certain embodiments, —Y′— is of formula (y4):

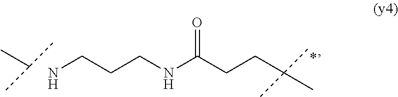

wherein
the unmarked dashed line indicates the attachment to —Y— and the dashed line marked with an asterisk indicates the attachment to -FG₂, -L³-, -L⁴- or -L⁵-.

In certain embodiments, —X′— is of formula (y4), wherein the unmarked dashed line indicates the attachment to —X— and the dashed line marked with an asterisk indicates the attachment to -FG₁ or -L³-.

The hydrogel HA microspheres or pharmaceutically acceptable salt thereof of the present invention may be used advantageously as drug carriers because they extend the half-life of a drug and exhibit physiological tolerability. Alternatively, they may be used for encapsulation of drugs or other agents. Moreover, as hyaluronic acid displays inert properties when exposed to tissues, the hydrogel HA microspheres may have medical utility and may be employed as a dilatant of Schlemm's canal during viscocanalostomy. Other uses include medical aesthetic fillers e.g., dermal fillers (to fill tissue space under epidermis); biodegradable implants; injectable materials for regenerating joint cartilage; constructing artificial structures useful in construction of vascularized tissues; tissue engineering/augmentation or regeneration materials; graft materials or injectable bone graft compositions or as a stem cell microsphere gel complex for subcutaneous injection.

The present invention also relates to a method of preparing a drug conjugate or pharmaceutically acceptable salt thereof, wherein the method comprises the following steps:
(a) providing the hydrogel HA microspheres or pharmaceutically acceptable salts thereof obtained by any of the methods of the present invention, wherein said hydrogel comprises a plurality of Z⁵ and/or Z⁶ units that have one or more unreacted -FG₁ or -FG₂;
(b) providing a monoconjugate reagent D-L¹-L²-FG₃, a bisconjugate reagent FG₃-L²-L¹-D-L¹-L²-FG₃ or a trisconjugate reagent of formula (t):

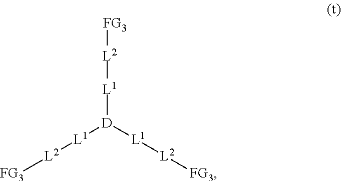

wherein -D is independently a drug moiety that is covalently and reversibly conjugated to -L¹-;
each -L¹- is independently a reversible linker moiety;
each -L²- is independently a spacer moiety or chemical bond;
each -FG₃ is independently a functional group that reacts with -FG₁ on Z⁵ or with -FG₂ on Z⁶;
(c) mixing the hydrogel HA microspheres of step (a) with the monoconjugate, bisconjugate or trisconjugate reagent of step (b);
(d) optionally, mixing the drug conjugate or pharmaceutically salt thereof of step (c) with a blocking reagent; and
(e) collecting the drug conjugate or pharmaceutically acceptable salt thereof of step (c) or (d).

The method of preparing a drug conjugate or pharmaceutically acceptable salt thereof may also comprise optional washing or purification steps of the drug conjugate obtained in step (c) or (d).

The present invention also relates to drug conjugates or pharmaceutically acceptable salts thereof obtainable by the methods of the present invention.

The person skilled in the art will recognize that the drug conjugate or pharmaceutically acceptable salt thereof of the present invention may also comprise one or more unreacted -FG₁ or -FG₂. To avoid undesired reactions between said groups and other functional groups on the drug moieties or compounds that may be found at the locations where the drug conjugates are administered, blocking reagents are used.

Exemplary blocking reagents may be selected from the group consisting of:

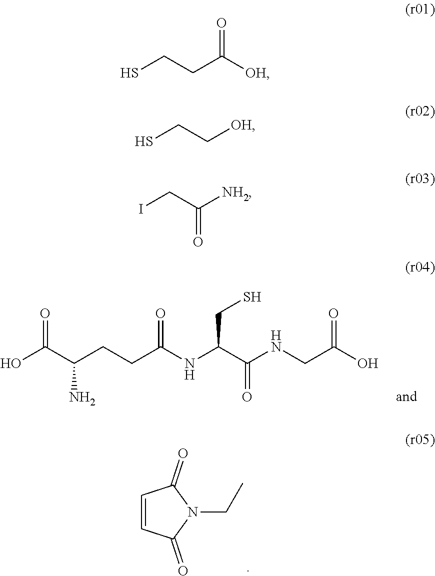

More specifically, to avoid undesired reactions between unreacted maleimides on the HA hydrogel microspheres and nucleophilic compounds such as thiols, in particular glutathione, or functional groups on the drug moieties such as amines, said unreacted maleimides are reacted with a blocking reagent. Advantageously, a blocking reagent of formula (r01) suppresses retro-Michael and exchange reactions of the formed thiosuccinimide in the presence of other thiol-containing compounds at physiological pH and temperature. This is particularly beneficial for drug conjugates or pharmaceutically acceptable salts thereof administered into a tissue or organ, in which glutathione is naturally found, such as in the eye.

In certain embodiments, the reagent of step (b) is a monoconjugate reagent D-L¹-L²-FG₃. In certain embodiments, the reagent of step (b) is a bisconjugate reagent FG₃-L²-L¹-D-L¹-L²-FG₃. In certain embodiments, the reagent of step (b) is a trisconjugate reagent of formula (t).

The monoconjugate reagent D-L¹-L²-FG₃ may be obtained by any methods known in the art. This is followed by purification by tagging the resulted drug-reversible prodrug linker conjugates with a purification tag to form a mixture. The monoconjugate reagent D-L¹-L²-FG₃ is purified from said mixture by chromatographic separation and then the purification tag is removed from the tagged D-L¹-L²-FG₃ to form the purified D-L¹-L²-FG₃.

As used herein, “purification tag” refers to a moiety which, when conjugated to a second moiety, confers a physical and/or chemical property/properties not present in said second moiety without the tag moiety and which different physical and/or chemical property/properties allow for the purification of such a conjugate.

Purification tags within the scope of the present disclosure are described in WO 2015/052155 A1, which is hereby incorporated by reference in its entirety. Accordingly, in certain embodiments the purification tag comprises a moiety of formula (ax):

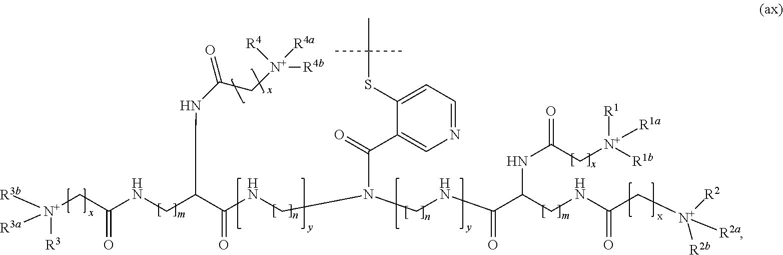

wherein the dashed line indicates attachment to D-L¹-L²-FG₃ via -FG₃;
—R¹, —R^{1a}, —R^{1b}, —R², —R^{2a}, —R^{2b}, —R³, —R^{3a}, —R^{3b}, —R⁴, —R^{4a}, —R^{4b} are independently of each other H or methyl;
each m is independently of each other 1, 2, 3, 4, 5, 6, 7 or 8;
each n is independently of each other 1, 2, 3, 4, 5, 6, 7 or 8;
each x is independently of each other 1, 2, 3, 4, 5, 6, 7 or 8; and
each y is independently of each other 0, 1, 2, 3, 4, 5, 6, 7 or 8.

In certain embodiments, -FG₃ is either of formula (y-56) or (y-57):

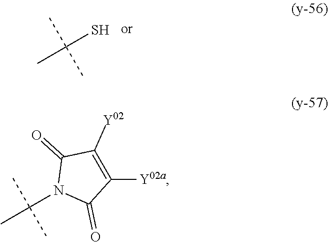

wherein the dashed line indicates the attachment to -L²-; and
—Y⁰² and —Y^{02a} are independently selected from the group consisting of —H and —Br.

In certain embodiments, the reagent of step (b) is a monoconjugate reagent D-L¹-L²-FG₃ of formula (m1) or (m2):

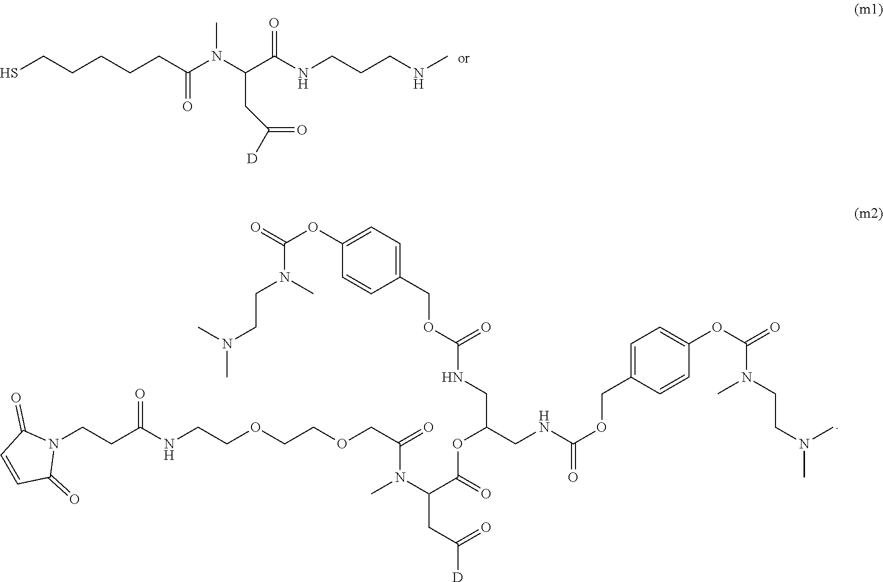 [SMILES: [2H]C(=O)CC(C(=O)NCCCNC)N(C)C(=O)CCCCCS ; [2H]C(=O)CC(C(=O)OC(CNC(=O)OCc1ccc(OC(=O)N(C)CCN(C)C)cc1)CNC(=O)OCc1ccc(OC(=O)N(C)CCN(C)C)cc1)N(C)C(=O)COCCOCCNC(=O)CCN1C(=O)C=CC1=O]

As used herein, throughout the specification, as a matter of convenience most structures do not depict stereochemistry and thus represent all possible stereoisomers.

In certain embodiments, the reagent of step (b) is a monoconjugate reagent D-L¹-L²-FG₃ of formula (m′1) or (m′2):

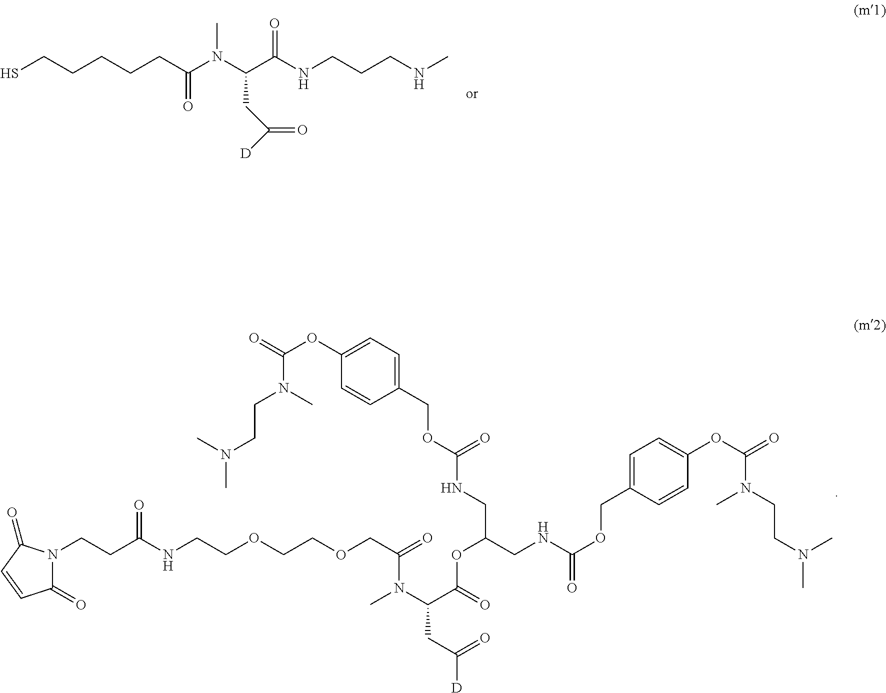 [SMILES: [2H]C(=O)C[C@@H](C(=O)NCCCNC)N(C)C(=O)CCCCCS ; [2H]C(=O)C[C@@H](C(=O)OC(CNC(=O)OCc1ccc(OC(=O)N(C)CCN(C)C)cc1)CNC(=O)OCc1ccc(OC(=O)N(C)CCN(C)C)cc1)N(C)C(=O)COCCOCCNC(=O)CCN1C(=O)C=CC1=O]

The present invention also relates to the use of the hydrogel microspheres or pharmaceutically acceptable salts thereof of the present invention as a carrier in a drug conjugate or pharmaceutically acceptable salt thereof.

Another aspect of the present invention relates to a drug conjugate or pharmaceutically acceptable salt thereof comprising hydrogel HA microspheres obtainable by the methods of the present invention.

The present invention also relates to a drug conjugate or pharmaceutically acceptable salts thereof comprising a HA hydrogel microsphere comprising crosslinked HA chains or pharmaceutically acceptable salt thereof to which a plurality of drug moieties is covalently and reversibly conjugated, said drug conjugate comprising a plurality of the following units:

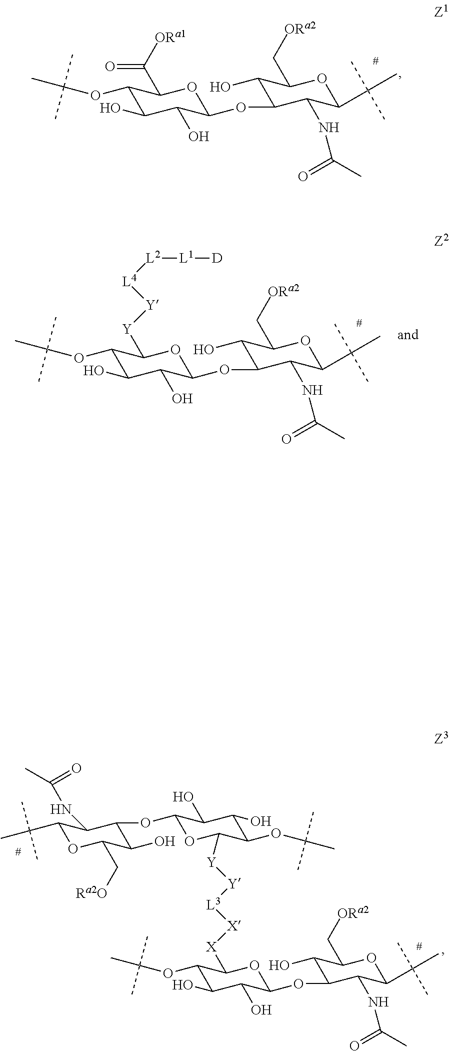

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
wherein each -D is independently a drug moiety that is covalently and reversibly conjugated to -L¹-;
each -L⁴- is independently a linkage moiety; and
each R^{a1}, —R^{a2}, —X—, —Y—, —X′—, —Y′—, -L¹-, -L²- and -L³- are used as defined elsewhere herein.

It is understood by the person skilled in the art that the drug conjugate or pharmaceutically acceptable salt thereof of the present invention may comprise low amounts of other HA units that could have resulted from various intramolecular reactions, such as for example HA units in which -D acts as a crosslinker between two functionalized HA strands. In addition, the drug conjugate or pharmaceutically acceptable salt thereof may comprise low amounts of HA units in which degradation or other modification(s) occurred.

In certain embodiments, the drug conjugate or pharmaceutically acceptable salt thereof comprises a HA hydrogel microsphere comprising crosslinked HA chains or pharmaceutically acceptable salt thereof to which a plurality of drug moieties is covalently and reversibly conjugated, said drug conjugate comprising a plurality of each of the following units:

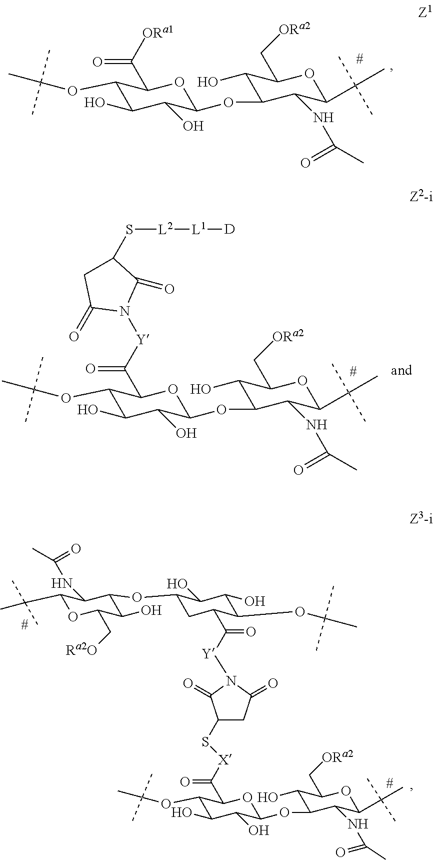

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group; and
each R^{a1}, —R^{a2}, —X′—, —Y′—, -D, -L¹- and -L²- are used as defined elsewhere herein.

In certain embodiments, each -L³-, -L⁴- or -L⁵- is independently selected from the group consisting of:

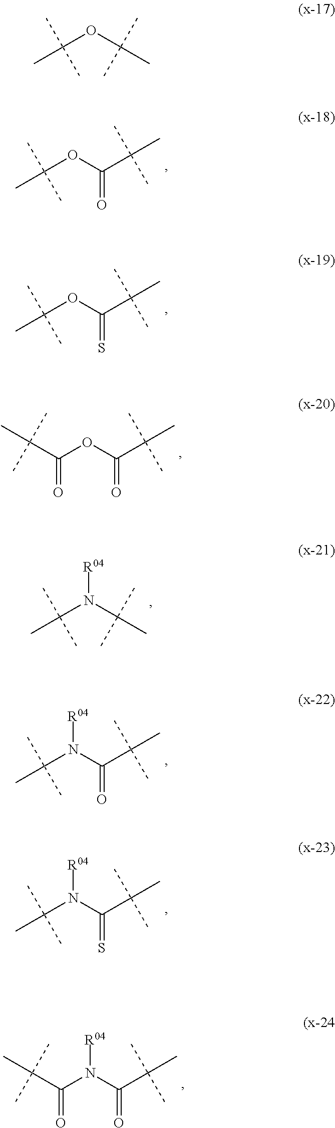

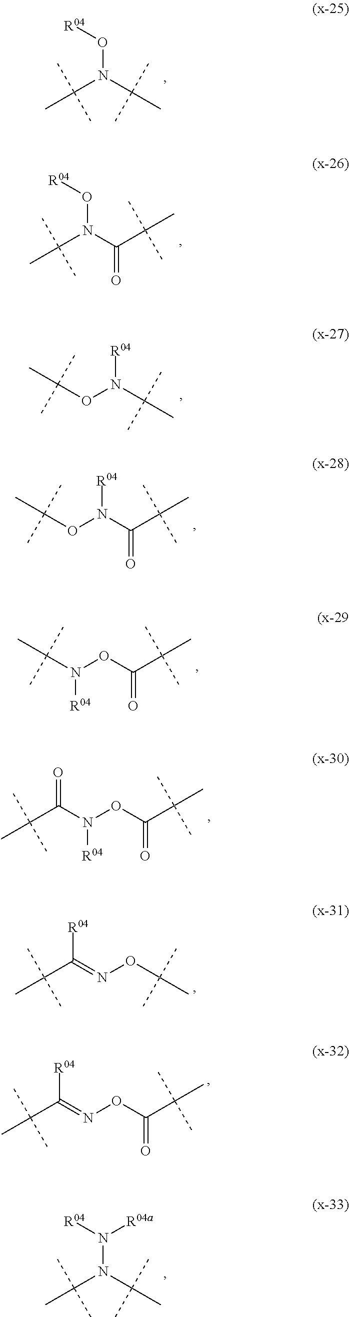

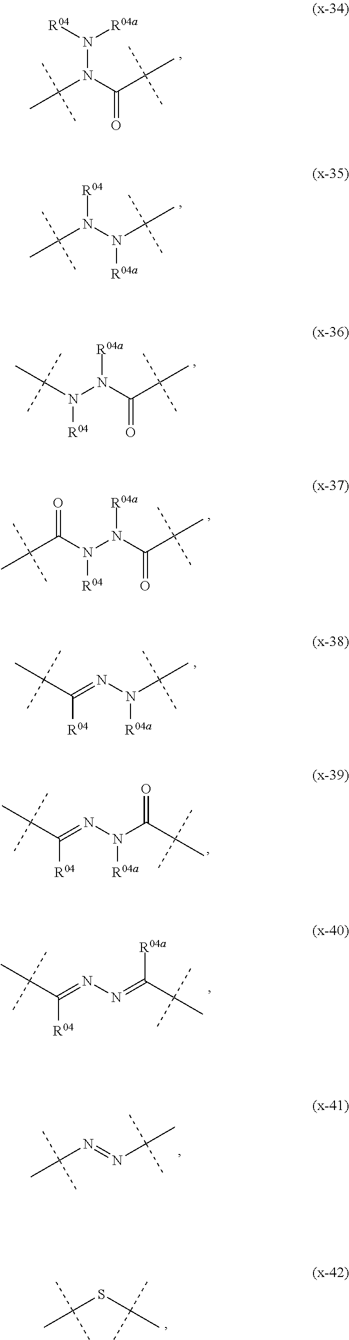

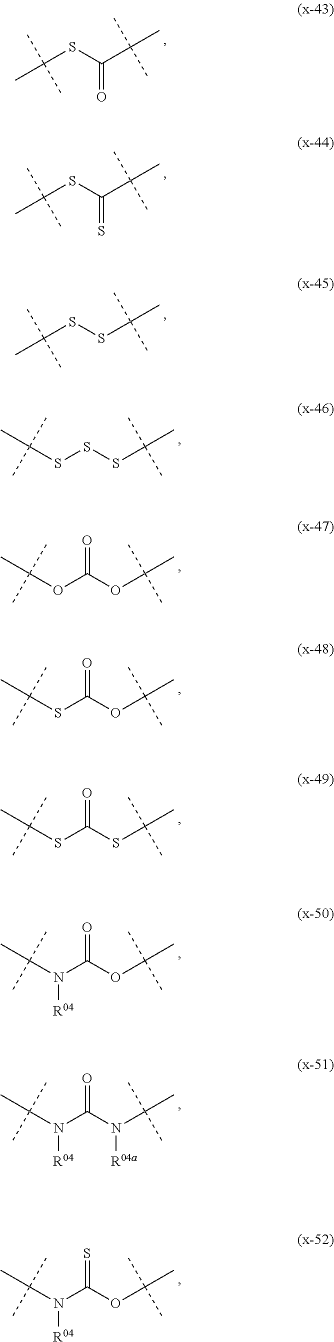

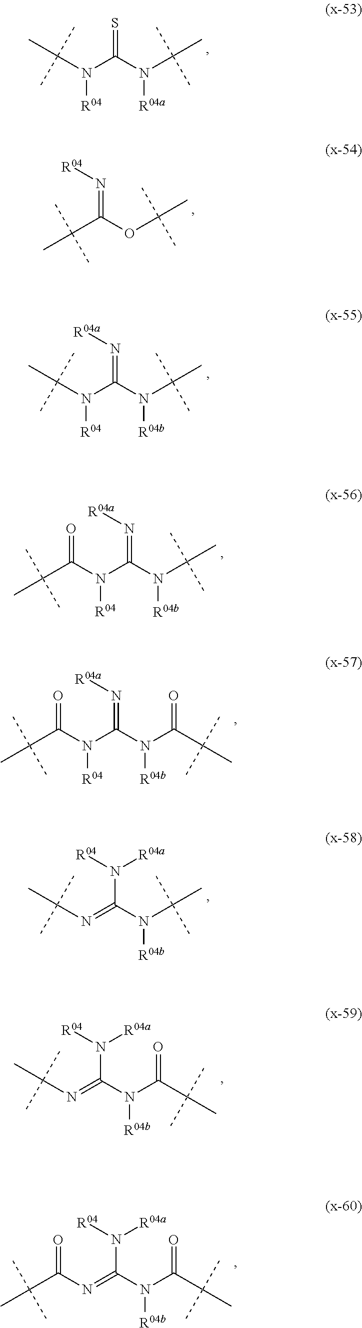

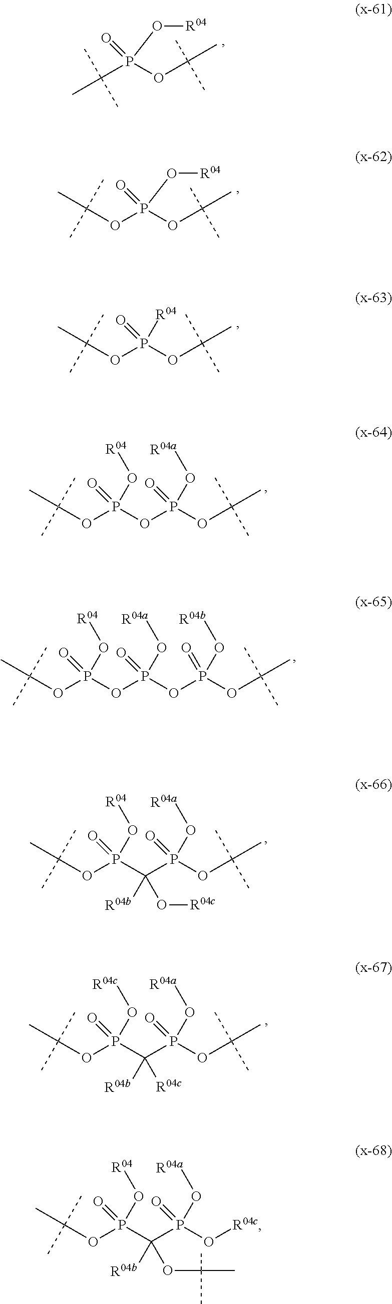

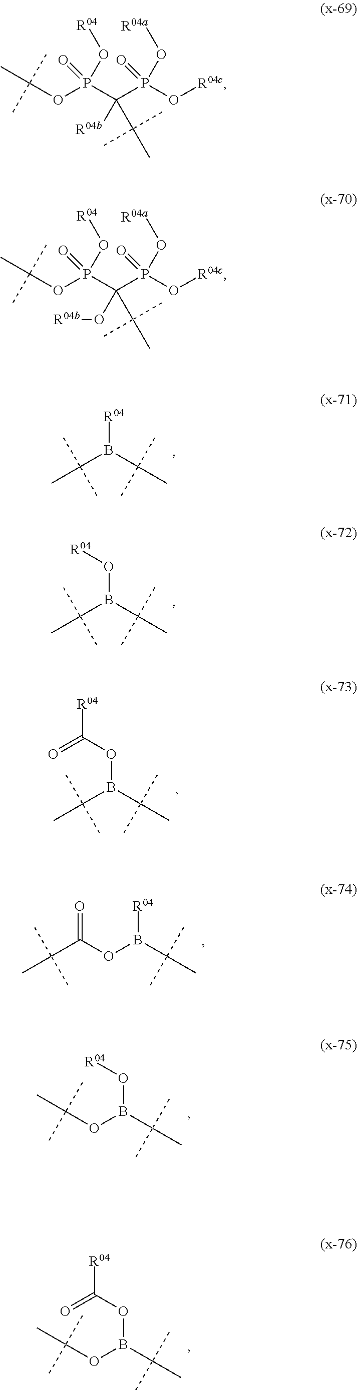

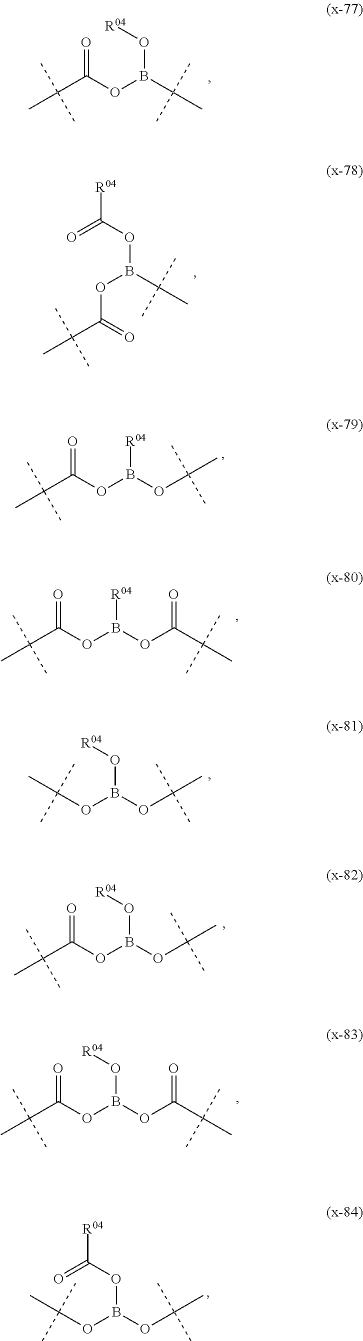

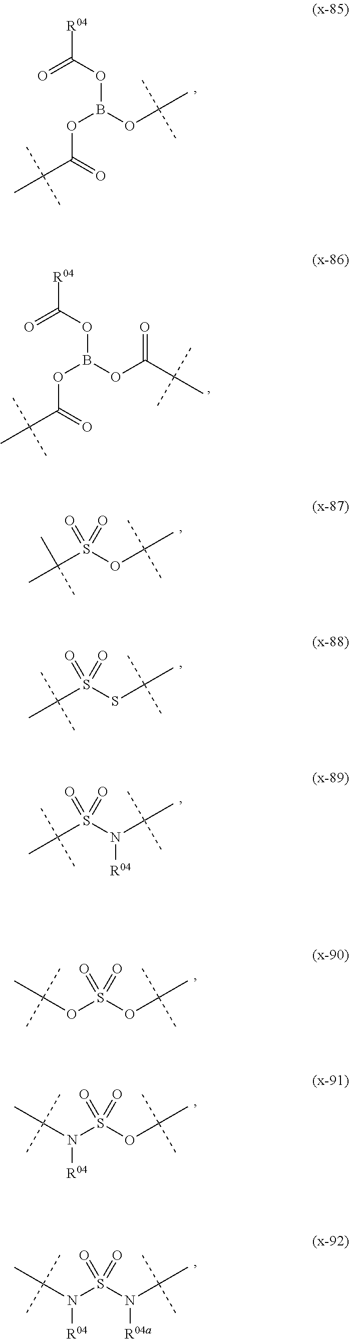

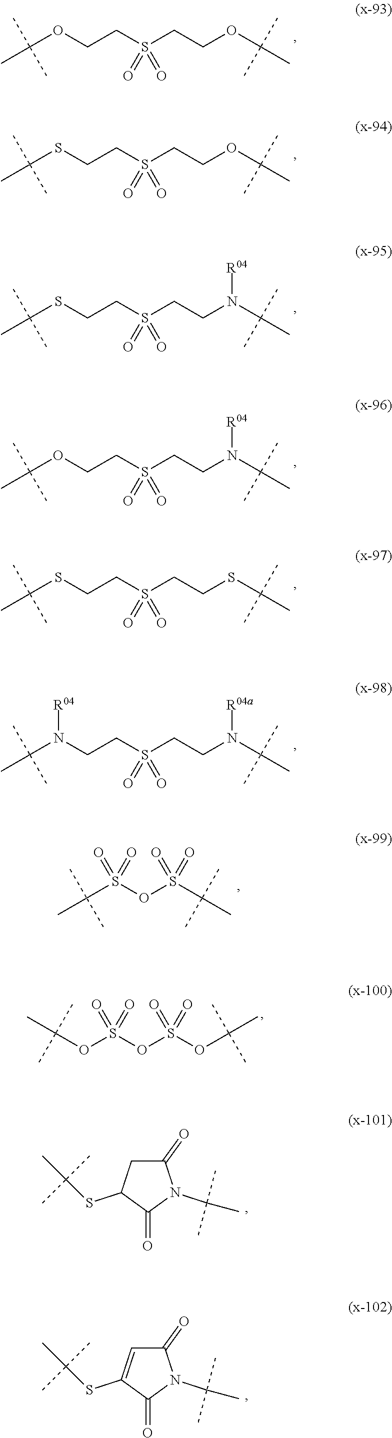

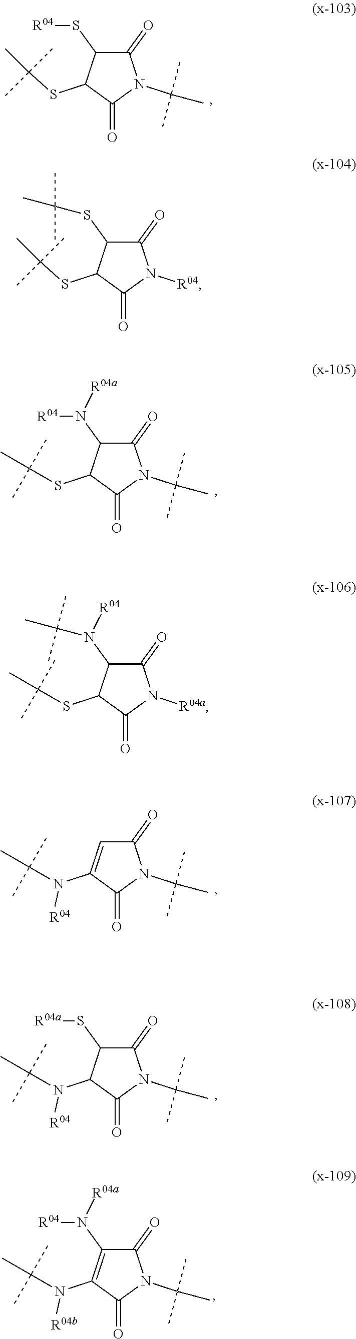

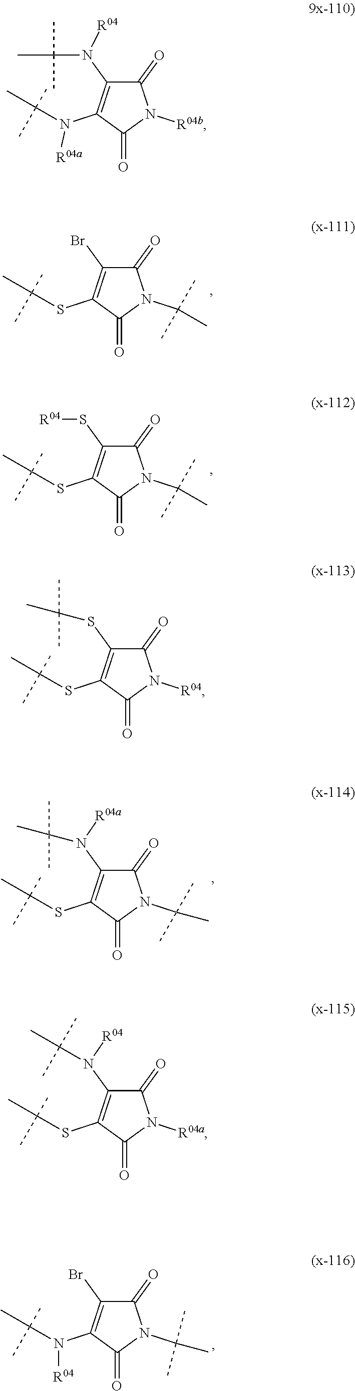

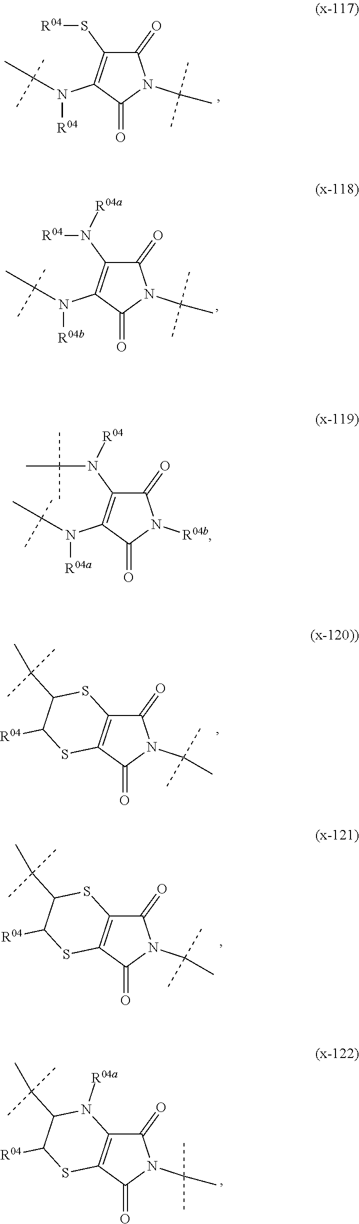

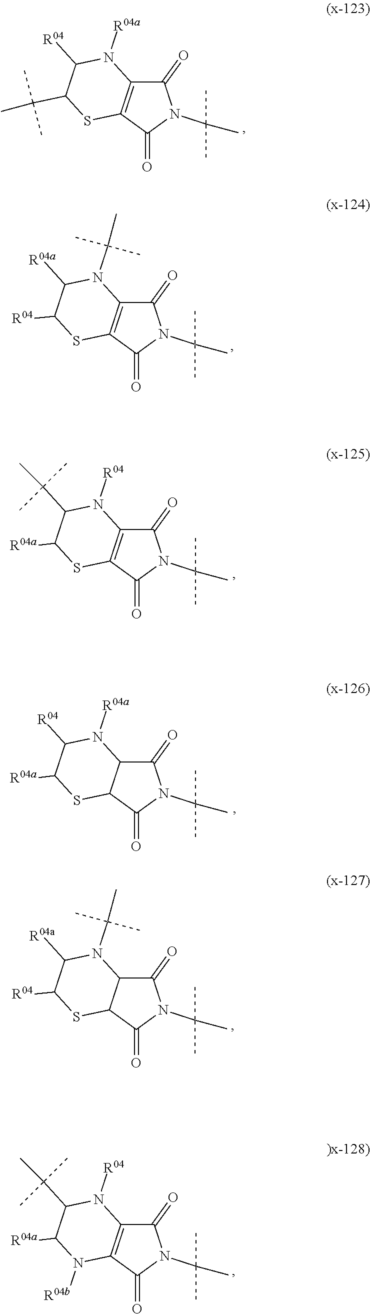

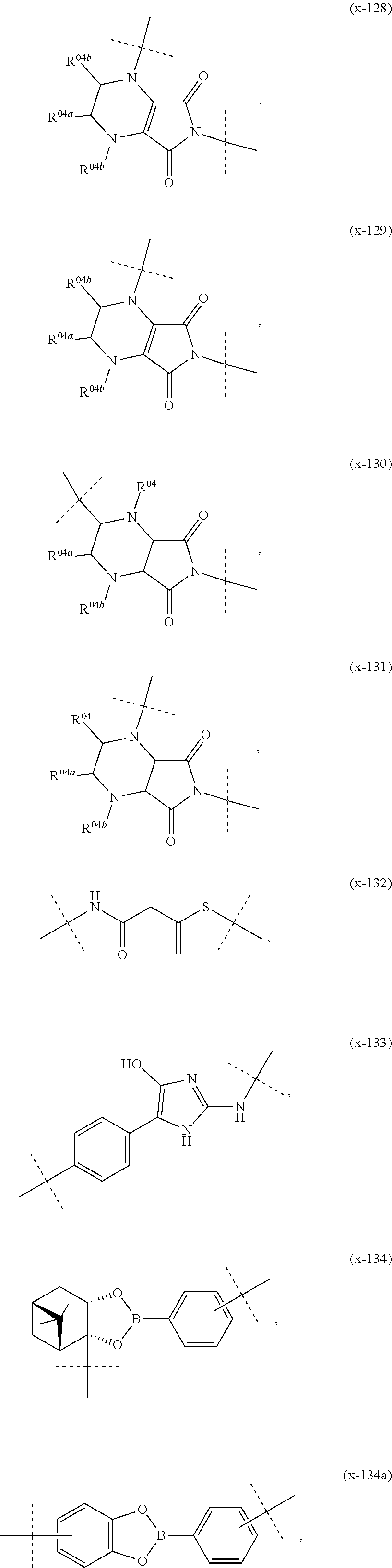

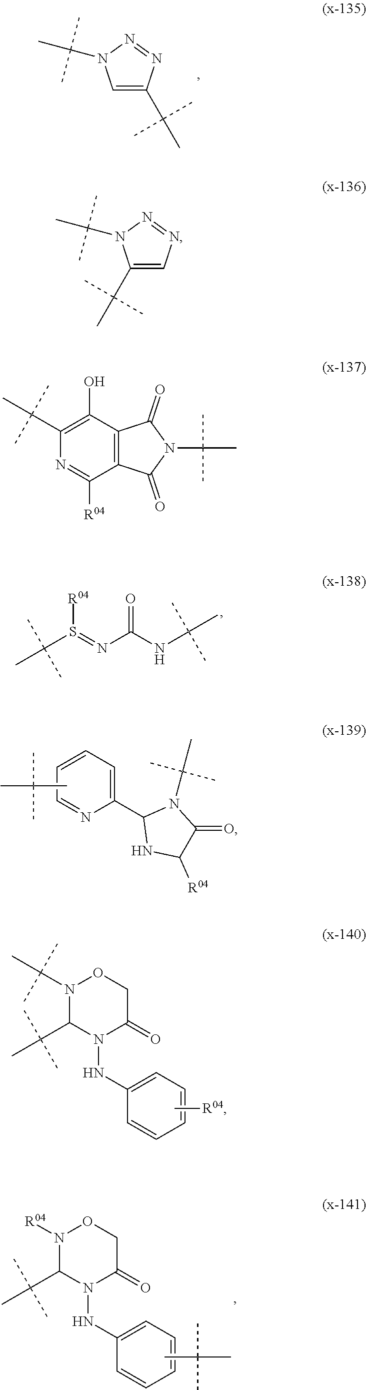

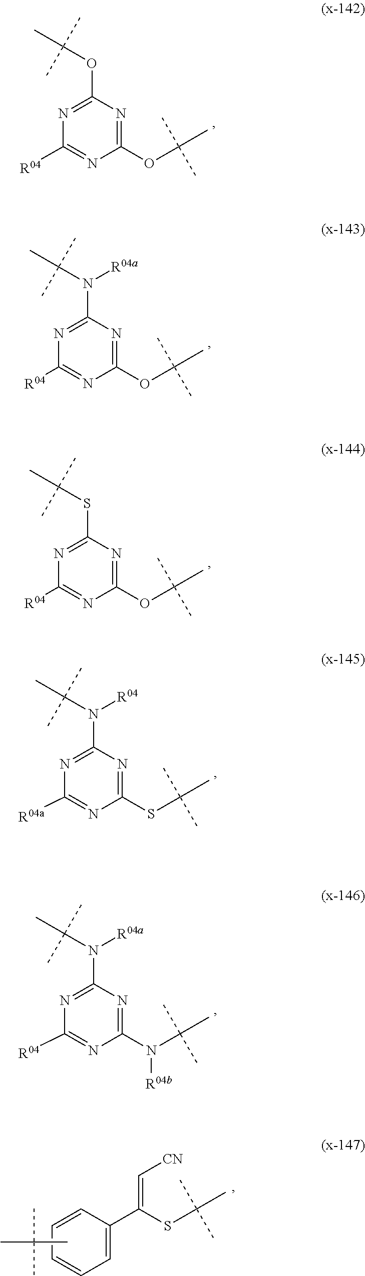

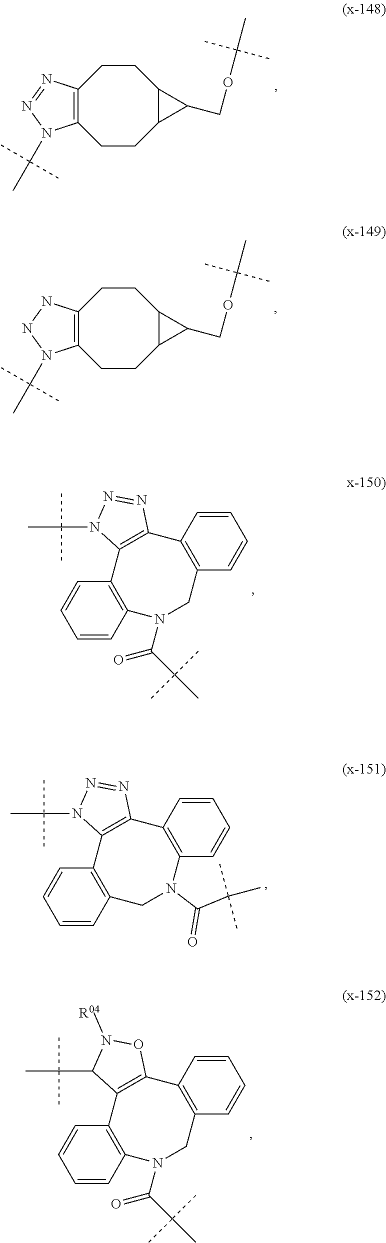

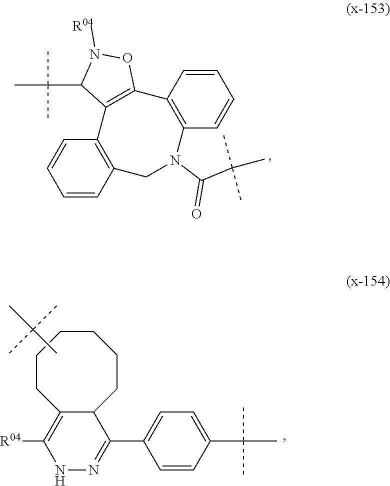

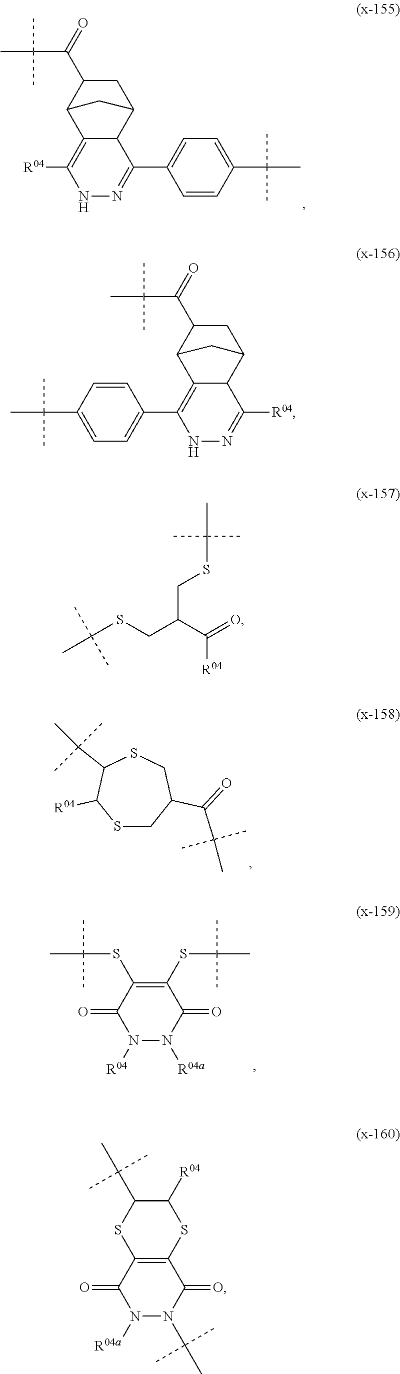

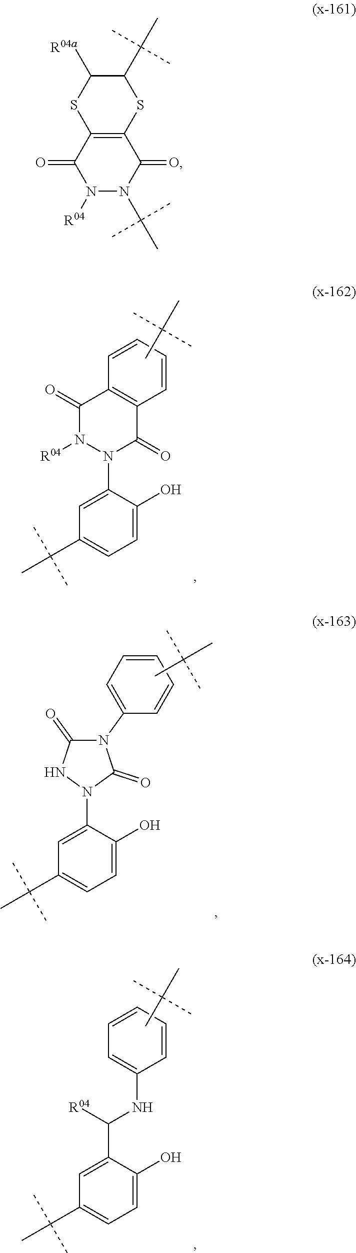

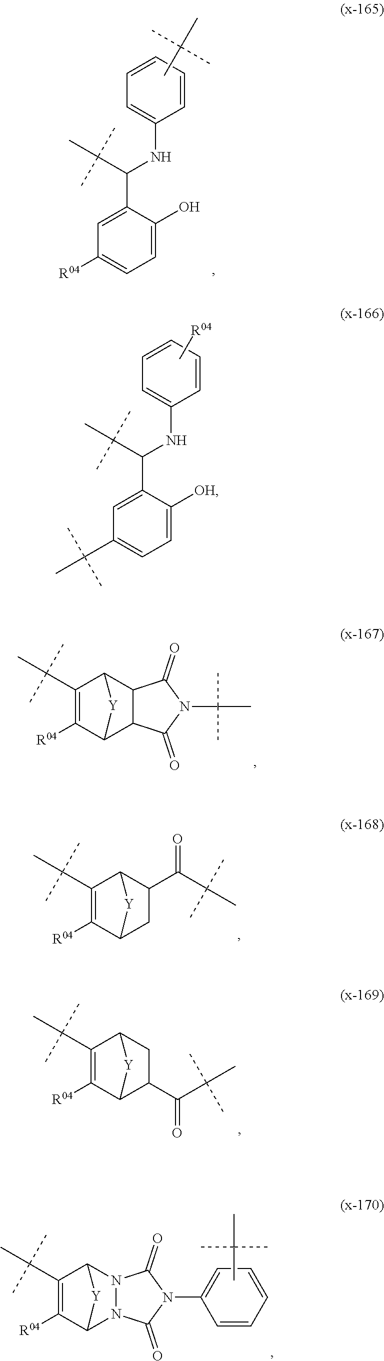

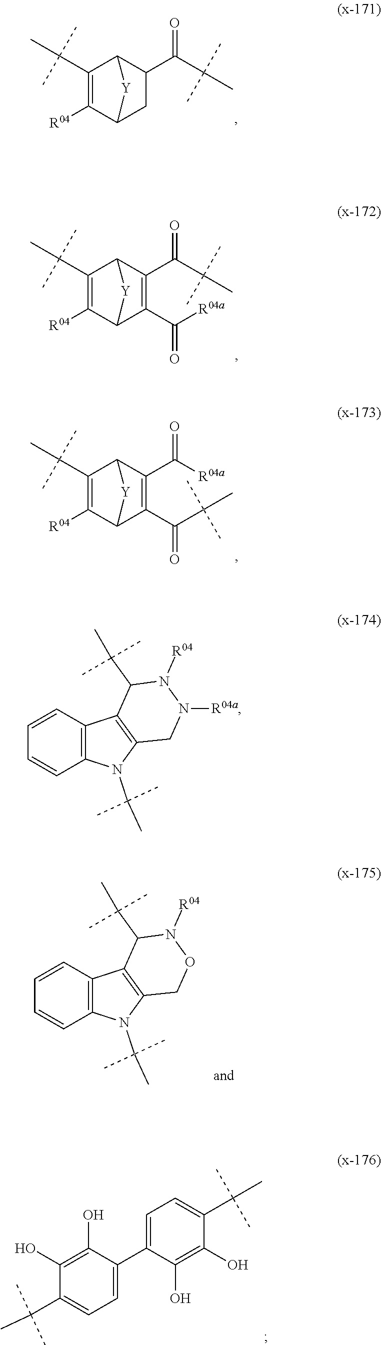

wherein for -L³- one dashed line indicates the attachment to —X′— and the other dashed line to —Y′—, for -L⁴- one dashed line indicates the attachment to -L²- and the other dashed line to —Y′— and for -L⁵- one dashed line indicates the attachment to —Y′— and the other dashed line to -BA;
—Y— is selected from the group consisting of —O—, —S—, —NR⁰⁵— and —CR⁰⁵R^{05a};
each —R⁰⁴, —R^{04a}, —R^{04b}, —R^{04c}, —R⁰⁵ and —R^{05a} is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰⁶ which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰⁷)—, —S(O)₂N(R⁰⁷)—, —S(O)N(R⁰⁷)—, —S(O)₂—, —S(O)—, —N(R⁰⁷)S(O)₂N(R^{07a})—, —S—, —N(R⁰⁷)—, —OC(OR⁷)(R^{07a})—, —N(R⁰⁷)C(O)N(R^{07a})—, and —OC(O)N(R⁰⁷)—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R⁰⁶, which are the same or different; and
each —R⁰⁶, —R⁰⁷ and —R^{07a} is independently selected from the group consisting of —H and C₁₋₆ alkyl; wherein C₁₋₆ alkyl is optionally substituted with one or more halogen, which are the same or different.

In certain embodiments, all moieties -L³- have the same structure. In certain embodiments, all moieties -L⁴- have the same structure. In certain embodiments all moieties -L⁵- have the same structure. In certain embodiments, all moieties -L³- have the same structure, all moieties -L⁴- have the same structure and all moieties -L⁵- have the same structure, which may be the same or different between the different moieties. In certain embodiments, all moieties -L³-, all moieties -L⁴- and all moieties -L⁵- have the same chemical structure.

In certain embodiments, the drug conjugate or pharmaceutically acceptable salts thereof comprises an HA hydrogel microsphere comprising crosslinked HA chains or pharmaceutically acceptable salt thereof to which a plurality of drug moieties is covalently and reversibly conjugated, said drug conjugate comprising a plurality of each of the following units:

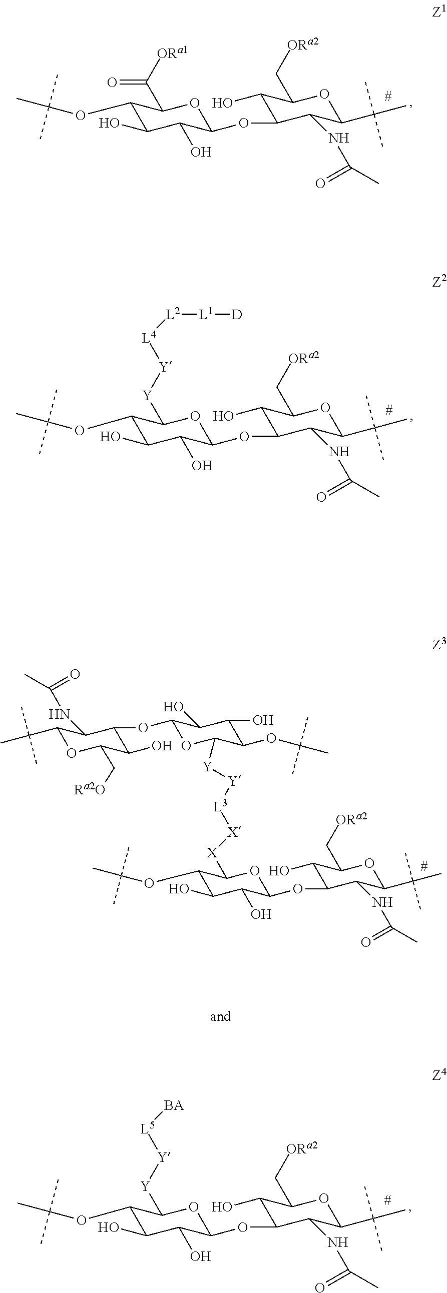

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1}, —R^{a2}, —X—, —Y—, —X′—, —Y′—, -D, -L¹-, -L²-, -L³- and -L⁴- are used as defined elsewhere herein;
each -L⁵- is independently a linkage moiety; and
each -BA is a blocking agent.

The drug conjugate or pharmaceutically acceptable salt thereof of the present invention may comprise Z¹ in a range of about 50% to about 98%, Z² in a range of about 0.1% to about 20%, Z³ in a range of about 0.1% to about 20% and Z⁴ in a range of about 0.1% to about 10%.

In certain embodiments, the drug conjugate or pharmaceutically acceptable salt thereof comprises Z¹ in a range of about 75% to about 98%, Z² in a range of about 0.1% to about 10%, Z³ in a range of about 0.1% to about 10% and Z⁴ in a range of about 0.1% to about 5%.

In certain embodiments, the drug conjugate or pharmaceutically acceptable salt thereof comprises Z¹ in a range of about 78% to about 96%, Z² in a range of about 2% to about 10%, Z³ in a range of about 1% to about 7% and Z⁴ in a range of about 0.5% to about 5%.

In certain embodiments, the drug conjugate or pharmaceutically acceptable salt thereof comprises Z¹ in a range of about 90.5% to about 94.8%, Z² in a range of about 2.7% to about 6.4%, Z³ in a range of about 1.1% to about 2.1% and Z⁴ in a range of about 0.8% to about 1.9%.

Suitably, the drug conjugate or pharmaceutically acceptable salt thereof comprises about 92.9% Z¹, about 4.3% Z², about 1.5% Z³ and about 1.3% Z⁴.

It is understood that the percentages provided above are calculated based on the total number of units present in a drug conjugate.

Exemplary blocking agents may be selected from the group consisting of.

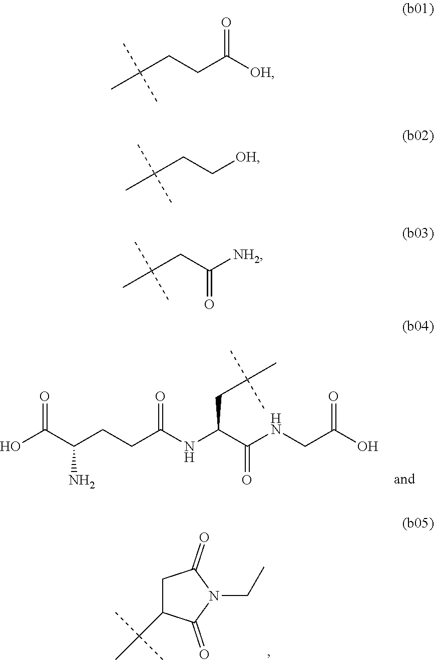

wherein the dashed line indicates the attachment to -L⁵-.

In certain embodiments, the blocking agent is of formula (b01), (b02) or (b04). In certain embodiments, the blocking agent is of formula (b01). In certain embodiments, the blocking agent is of formula (b02). In certain embodiments, the blocking agent is of formula (b03). In certain embodiments, the blocking agent is of formula (b04). In certain embodiments, the blocking agent is of formula (b05).

Suitably, the drug conjugate or pharmaceutically acceptable salt thereof comprises a HA hydrogel microsphere comprising crosslinked HA chains or pharmaceutically acceptable salts thereof to which a plurality of drug moieties is covalently and reversibly conjugated, wherein the drug conjugate comprises a plurality of each of the following units:

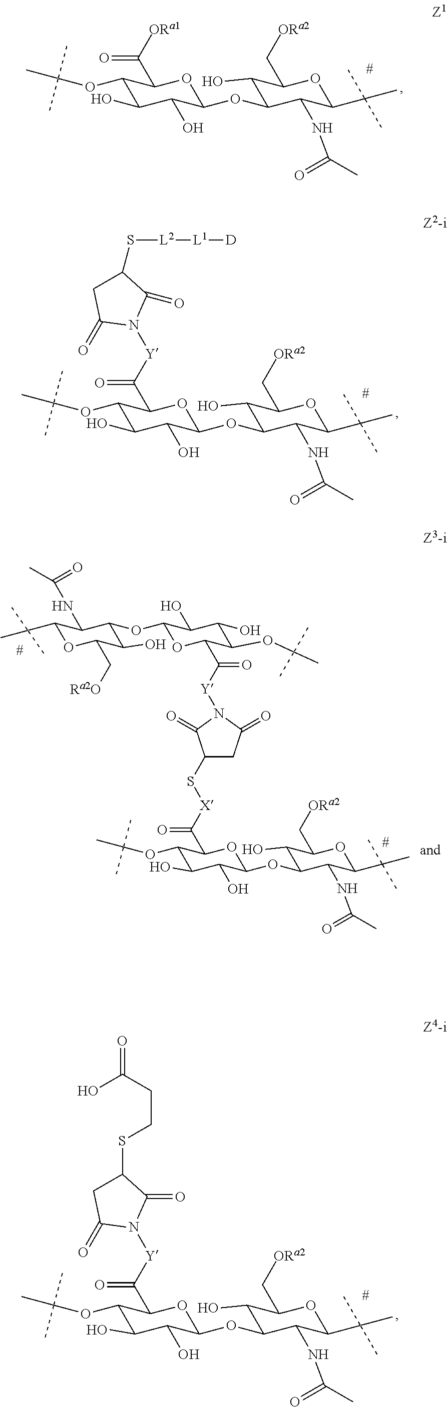

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is independently selected from the group consisting of —H, C₁₋₁₀ alkyl, an ammonium ion, a tetrabutylammonium ion, a cetyl trimethylammonium ion, an alkali metal ion and an alkaline earth metal ion;
each —R^{a2} is independently —H or C₁₋₁₀ alkyl;
each —X′—, —Y′— is independently a spacer moiety or absent;
wherein each -D is independently a drug moiety that is covalently and reversibly conjugated to -L¹-;
each -L¹- is independently a reversible linker moiety;
each -L²- is independently a spacer moiety or absent; and
wherein said drug conjugate comprises Z¹ in a range of about 50% to about 98%, Z²-i in a range of about 0.1% to about 20%, Z³-i in a range of about 0.1% to about 20% and Z⁴-i in a range of about 0.1% to about 10%.

In particular, the drug conjugate described above comprises Z¹ in a range of about 86% to about 96%, Z²-i in a range of about 0.1% to about 12.9%, Z³-i in a range of about 0.34% to about 3.57% and Z⁴-i in a range of about 0.1% to about 9.5%.

Advantageously, the drug conjugate or pharmaceutically acceptable salt thereof comprises a HA hydrogel microsphere comprising crosslinked HA chains or pharmaceutically acceptable salts thereof to which a plurality of drug moieties is covalently and reversibly conjugated, wherein the drug conjugate comprises a plurality of each of the following units:

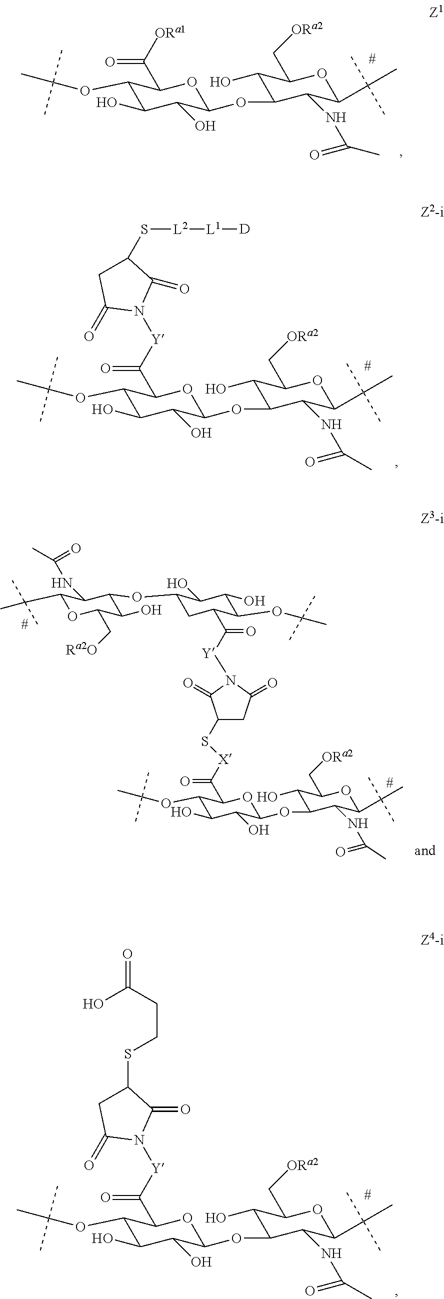

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group; and
each R^{a1} is —H or an alkali metal ion;
each —R^{a2} is —H;
each —X′— is of formula (x0):

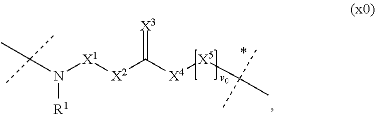

wherein
the unmarked dashed line indicates the attachment to the carbonyl group and the dashed line marked with an asterisk indicates the attachment to the sulfur atom;
v₀ is selected from the group consisting of 0 and 1;
—X¹—, —X⁴— are independently C₁₋₁₀ alkyl, which C₁₋₁₀ alkyl is optionally interrupted by one or more groups independently selected from —O—, -T-, —N(R^{y1})—, —C(O)O— and —C(O)N(R^{y1})—; and which C₁₋₁₀ alkyl chain is optionally substituted with one or more groups independently selected from —OH, -T, —NH(R^{y1}) and —C(O)N(R^{y2}R^{y2a}); wherein —R^{y1}, —R^{y2}, —R^{y2a} are independently selected from the group consisting of —H and C₁₋₄ alkyl;
—X²— is selected from the group consisting of —N(R¹)—, —O—, —S— and —Se—;
═X³ is selected from the group consisting of ═O, ═N(R¹) and ═S;
—X⁵— is C₁₋₂₀ alkyl, which C₁₋₂₀ alkyl is optionally interrupted by one or more groups independently selected from —O—, —C(O)O—, -T-, —N(R^{y1})— and —N(R^{y1})C(O)—; and which C₁₋₂₀ alkyl chain is optionally substituted with one or more groups independently selected from —OH, -T, —NH(R^{y1}) and —C(O)N(R^{y2}R^{y2a});
—R¹ is independently selected from the group consisting of —H, C₁₋₅ alkyl and -T; wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R², which are the same or different;
—R² is selected from the group consisting of halogen, —CN, oxo, —C(O)OR³, —OR³, —C(O)R³, —C(O)N(R³)(R^{3a}), —S(O)₂N(R³)(R^{3a}), —S(O)N(R³)(R^{3a}), —S(O)₂R³, —S(O)R³, —N(R³)S(O)₂N(R^{3a})(R^{3b}), —SR³, —N(R³)(R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a})(R^{3b}), —OC(O)N(R³)(R^{3a}) and C₁₋₆ alkyl; wherein C₁₋₆ alkyl is optionally substituted with one or more halogen, which are the same or different; and
wherein —R³, —R^{3a} and —R^{3b} are independently selected from the group consisting of —H and C₁₋₆ alkyl; wherein C₁₋₆ alkyl is optionally substituted with one or more halogen, which are the same or different;
each —Y′— is of formula (y0):

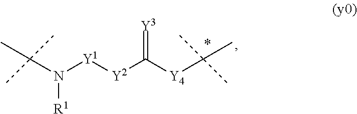

wherein
the unmarked dashed line indicates the attachment to the carbonyl group and the dashed line marked with an asterisk indicates the attachment to the nitrogen atom of the thiosuccinimide ring;
—Y¹—, —Y⁴— are independently C₁₋₁₀ alkyl, which C₁₋₁₀ alkyl is optionally interrupted by one or more groups independently selected from —O—, -T-, —N(R^{y1})—, —C(O)O— and —C(O)N(R^{y1})—; and which C₁₋₁₀ alkyl chain is optionally substituted with one or more groups independently selected from —OH, -T, —NH(R^{y1}) and —C(O)N(R^{y2}R^{y2a}); wherein —R^{y1}, —R^{y2}, —R^{y2a} are independently selected from the group consisting of H and C₁₋₄ alkyl;
—Y²— is selected from the group consisting of —N(R²)—, —O—, —S— and —Se—;
=Y³ is selected from the group consisting of ═O, ═N(R²) and ═S;
—R¹, —R² are independently selected from the group consisting of —H, C₁₋₅ alkyl and -T;
wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R³, which are the same or different;
—R³ is selected from the group consisting of halogen, —CN, oxo, —C(O)OR⁴, —OR⁴, —C(O)R⁴, —C(O)N(R⁴)(R^{4a}), —S(O)₂N(R⁴)(R^{4a}), —S(O)N(R⁴)(R^{4a}), —S(O)₂R⁴, —S(O)R⁴, —N(R⁴)S(O)₂N(R^{4a})(R^{4b}), —SR⁴, —N(R⁴)(R^{4a}), —NO₂, —OC(O)R⁴, —N(R⁴)C(O)R^{4a}, —N(R⁴)S(O)₂R^{4a}, —N(R⁴)S(O)R^{4a}, —N(R⁴)C(O)OR^{4a}, —N(R⁴)C(O)N(R^{4a})(R^{4b}), —OC(O)N(R⁴)(R^{4a}) and C₁₋₆ alkyl; wherein C₁₋₆ alkyl is optionally substituted with one or more halogen, which are the same or different; and
wherein —R⁴, —R^{4a} and —R^{4b} are independently selected from the group consisting of —H and C₁₋₆ alkyl; wherein C₁₋₆ alkyl is optionally substituted with one or more halogen, which are the same or different;
each -D is independently a drug moiety that is covalently and reversibly conjugated to -L¹;
each -L¹- is a linker moiety of formula (I):

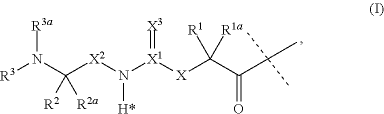

wherein
the dashed line indicates the attachment to a nitrogen of -D by forming an amide bond;
—X— is —C(R⁴R^{4a})—; —N(R⁴)—; —O—; —C(R⁴R^{4a})—C(R⁵R^{5a})—; —C(R⁵R^{5a})—C(R⁴R^{4a})—; —C(R⁴R^{4a})—N(R⁶)—; —N(R⁶)—C(R⁴R^{4a})—; —C(R⁴R^{4a})—O—; —O—C(R⁴R^{4a})—;
or —C(R⁷R^{7a})—;
X¹ is C; or S(O);
—X²— is —C(R⁸R^{8a})—; or —C(R⁸R^{8a})—C(R⁹R^{9a})—;
═X³ is ═O; ═S; or ═N—CN;
—R¹, —R^{1a}, —R², —R^{2a}, —R⁴, —R^{4a}, —R⁵, —R^{5a}, —R⁶, —R⁸, —R^{8a}, —R⁹, —R^{9a} are independently
selected from the group consisting of —H; and C₁₋₆ alkyl;
—R³, —R^{3a} are independently selected from the group consisting of —H; and C₁₋₆ alkyl, provided that in case one of —R³, —R^{3a} or both are other than —H they are connected to N to which they are attached through a sp³-hybridized carbon atom;
—R⁷ is —N(R¹⁰R^{10a}); or —NR¹⁰—(C═O)—R¹¹; —R^{7a}, —R¹⁰, —R^{10a}, —R¹¹ are independently of each other —H; or C₁₋₁₀ alkyl; optionally, one or more of the pairs —R^{1a}/—R^{4a}, —R^{1a}/—R^{5a}, —R^{1a}/—R^{7a}, —R^{4a}/—R^{5a}, —R^{8a}/—R^{9a} form a chemical bond;
optionally, one or more of the pairs —R¹/—R^{1a}, —R²/—R^{2a}, —R⁴/—R^{4a}, —R⁵/—R^{5a}, —R¹/—R^{8a}, —R⁹/—R^{9a} are joined together with the atom to which they are attached to form a C₃₋₁₀ cycloalkyl; or 3- to 10-membered heterocyclyl;
optionally, one or more of the pairs —R¹/—R⁴, —R¹/—R⁵, —R¹/—R⁶, —R¹/—R^{7a}, —R⁴/—R⁵, —R⁴/—R⁶, —R¹/—R⁹, —R²/—R³ are joined together with the atoms to which they are attached to form a ring A;
optionally, —R³/—R^{3a} are joined together with the nitrogen atom to which they are attached to form a 3- to 10-membered heterocycle;
ring A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
each -L¹- is substituted with -L²- provided that the hydrogen marked with the asterisk in formula (I) is not replaced by a substituent; or
each -L¹- is a linker moiety of formula (XI):

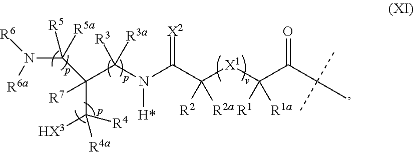

wherein
the dashed line indicates the attachment to the nitrogen of the primary or secondary amine of -D;
v is selected from the group consisting of 0 or 1;
—X¹— is selected from the group consisting of —C(R⁸)(R^{8a})—, —N(R⁹)— and —O—;
═X² is selected from the group consisting of ═O and ═N(R¹⁰);
—X³ is selected from the group consisting of —O, —S and —Se;
each p is independently selected from the group consisting of 0 or 1, provided that at most one p is 0;
—R⁶, —R^{6a}, —R¹⁰ are independently selected from the group consisting of —H, —C(R¹¹)(R^{11a})(R^{11b}) and -T;
—R⁹ is selected from the group consisting of —C(R¹¹)(R^{11a})(R^{11b}) and -T;
—R¹, —R^{1a}, —R², —R^{2a}, —R³, —R^{3a}, —R⁴, —R^{4a}, —R⁵, —R^{5a}, —R⁷, —R⁸ —R^{8a}, —R¹¹, —R^{11a} and —R^{11b} are independently selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
   —R¹², —R^{12a}, —R^{12b} are independently selected from the group consisting of —H, -T, C₁₋₆ alkyl, C₂₋₆ alkenyl and C₂₋₆ alkynyl; wherein -T, C₁₋₆ alkyl, C₂₋₆ alkenyl and C₂₋₆ alkynyl are optionally substituted with one or more —R¹³, which are the same or different and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
   wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R¹³, which are the same or different;
   —R¹³ is selected from the group consisting of halogen, —CN, oxo, —C(O)OR¹⁵, —OR¹⁵, —C(O)R¹⁵, —C(O)N(R¹⁵)(R^{15a}), —S(O)₂N(R¹⁵)(R^{15a}), S(O) N(R¹⁵)(R^{15a}), —S(O)₂R¹⁵, —S(O)R¹⁵, —N(R¹⁵)S(O)₂N(R^{15a})(R^{15b}), —SR¹⁵, —N(R¹⁵)(R^{15a}), —NO₂, —OC(O)R¹⁵, —N(R¹⁵)C(O)R^{15a}, —N(R¹⁵)S(O)₂R^{15a}, —N(R¹⁵)S(O)R^{15a}, —N(R¹⁵)C(O)OR^{15a}, —N(R¹⁵)C(O)N(R^{15a})(R^{15b}), —OC(O)N(R¹⁵)(R^{15a}) and C₁₋₆ alkyl; wherein C₁₋₆ alkyl is optionally substituted with one or more halogen, which are the same or different;
   wherein —R¹⁴, —R^{14a}, —R¹⁵, —R^{a15a} and —R^{15b} are independently selected from the group consisting of —H and C₁₋₆ alkyl; wherein C₁₋₆ alkyl is optionally substituted with one or more halogen, which are the same or different;
   optionally, one or more of the pairs —R¹/—R^{1a}, —R²/—R^{2a}, —R³/—R^{3a}, —R⁴/—R^{4a}, —R⁵/—R^{5a} or —R¹/—R^{8a} are joined together with the atom to which they are attached to form a C₃₋₁₀ cycloalkyl, 3- to 10-membered heterocyclyl or an 8- to 11-membered heterobicyclyl;
   optionally, one or more of the pairs —R¹/—R², —R¹/—R⁸, —R¹/—R⁹, —R²/—R⁹ or —R²/—R¹⁰ are joined together with the atoms to which they are attached to form a ring -A-;
   wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
   optionally, one or more of the pairs —R³/—R⁶, —R⁴/—R⁶, —R⁵/—R⁶, —R⁶/—R^{6a} or —R⁶/—R⁷ form together with the atoms to which they are attached a ring -A′-;
   wherein -A′- is selected from the group consisting of 3- to 10-membered heterocyclyl and 8- to 11-membered heterobicyclyl; and
   -L¹- is substituted with -L²- provided that the hydrogen marked with the asterisk in formula (XI) is not replaced by a substituent;
each -L²- is a spacer moiety; and
wherein said drug conjugate comprises Z¹ in a range of about 50% to about 98%, Z²-i in a range of about 0.1% to about 20%, Z³-i in a range of about 0.1% to about 20% and Z⁴-i in a range of about 0.1% to about 10%.

The unit percentages within the HA microspheres or drug conjugates and pharmaceutically acceptable salt thereof provided throughout the application may be determined by any method known to those skilled in the art and based on the information included in the following paragraphs. A functionalized HA may be obtained by introducing an amine group by coupling a diamine to one or more carboxyl groups of an HA of a M_{w} ranging from about 100 kDa to about 150 kDa. HA may optionally be purified prior to functionalization according to methods known in the art. The degree of substitution of the obtained amine functionalized HA may range from about 3% to about 15%, such as from about 3% to about 6% or such as from about 9% to 15%. Said degree of substitution may be determined by any method known in the art, such as by an o-phthalaldehyde (OPA) assay based on the reaction of OPA with primary amines or via an enzymatic assay. A maleimide functionalized HA was obtained by further converting the end amine groups on the amine functionalized HA into N-substituted amides having a spacer moiety (-L^{2″}-) comprising a maleimide group at its end. The degree of substitution of the obtained maleimide functionalized HA may range from about 9% to about 15%. Said degree of substitution may be determined by any method known in the art, such as by inverse Ellman's assay (i.e. by reaction of the free maleimide groups with a known excess of 2-mercaptoethanol at neutral pH, followed by determining the leftover thiols by Ellman's assay) or via an enzymatic assay. A thiol functionalized HA was obtained by further converting the end amine groups on an amine functionalized HA into N-substituted amides having a spacer moiety (-L^{2″}-) comprising a thiol group at its end. The degree of substitution of the obtained thiol functionalized HA may range from about 3% to about 6%. Said degree of substitution may be determined by any method known in the art, such as by Ellman's assay or via an enzymatic assay. Variation of the degree of maleimide and thiol functionalization allows different cross-link densities and different degrees of loading of drug in the hydrogel conjugate as described throughout the application. Also, methods for preparing thiol and maleimide functionalized HA are described in WO 2018/175788 A1 which is hereby incorporated by reference in its entirety.

The obtained maleimide functionalized HA and thiol functionalized HA are reacting with each other to provide the HA microspheres in a ratio (w/w) that may range from about 10:1 to 0.6:1, such as from about 8:1 to 1.1:1, such as from about 5:1 to 1.2:1 or such as from about 3:1 to 1.5:1, advantageously from about 2.15:1. It is assumed that this reaction goes to completion, i.e. that all thiol functional groups have reacted. Thus, the content of unreacted maleimide on the HA microspheres can be determined, for example, by the inverse Ellman's assay or an enzymatic assay. The determined maleimide content on the obtained hydrogel HA microspheres may range from 80 to 250 μmol/g based on the non-swollen, completely dry (lyophilized) hydrogel, i.e. no water or swelling behaviour is taken into account for the beforementioned calculations. To obtain a drug conjugate or pharmaceutically acceptable salt thereof, the hydrogel HA microspheres were mixed with a monoconjugate D-L¹-L²-FG₃ (wherein -D, -L¹-, -L²- and -FG₃ are used as described elsewhere herein) in a molar ratio that may range from about 0.5 (maleimide functionalities):1 (monoconjugate) to about 3 (maleimide functionalities):1 (monoconjugate), advantageously from 1.2 (maleimide functionalities):1 (monoconjugate). This is followed by the addition of an excess of blocking reagent (as described elsewhere herein). When the drug is a protein, there are several methods to determine the protein content. The protein content was determined by means of quantitative amino acid analysis. The drug conjugate or pharmaceutically acceptable salt thereof was incubated with 6 M HCl/TFA for approximately 30 minutes at 190° C. in a microwave device, which degrades the protein in its individual amino acids. After hydrolysis, the amino acids were derivatized with 6-aminoquinolyl-N-hydroxysuccinimidyl carbamate to yield stable, UV active urea compounds. Subsequently, the derivatives were separated by reversed-phase chromatography and quantified by UV detection. The concentration of the amino acid derivatives can then be converted into the protein concentration. An alternate method of determining the protein loading relies on using enzyme hydrolysis (e.g., hyaluronate lyase or chondroitinase) to undergo the HA hydrolysis or HA crosslinker hydrolysis at increased pH/temperature, followed by protein quantification at 280 nm.

In certain embodiments, an alternate way of depicting the drug conjugate or pharmaceutically acceptable salt thereof the present invention is shown below, i.e. as a drug conjugate comprising a plurality of HA strands 2A and a plurality of HA strands 2B, wherein:
each 2A comprises a plurality of linearly connected units Z¹, Z³(a), Z² and Z⁴:

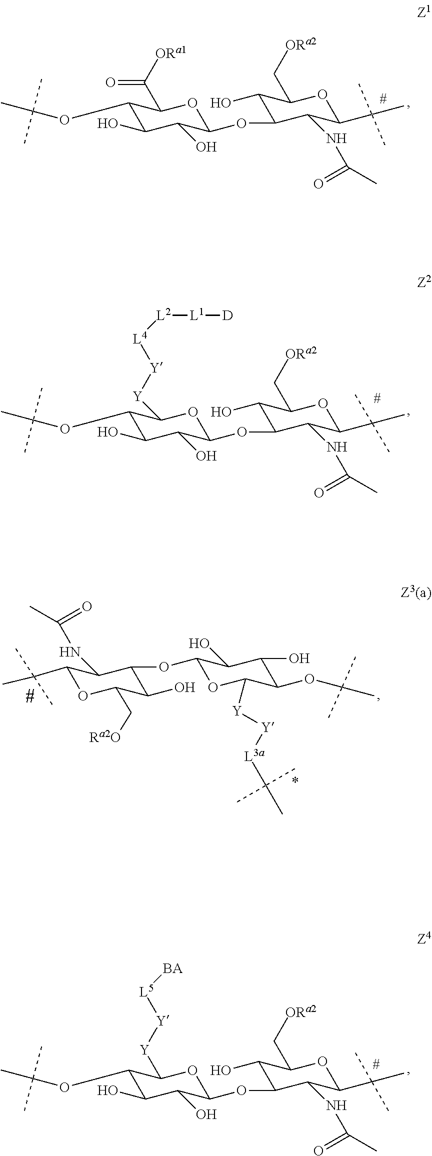

each 2B comprises a plurality of linearly connected units Z¹ and Z³(b):

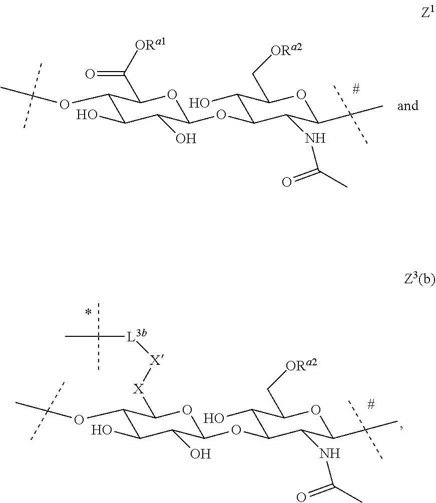

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
a dashed line marked with * indicates a point of crosslinking attachment between a unit Z³(a) and Z³(b) such that at least one 2A is crosslinked to at least 2B;
each -D, R^{a1}, —R^{a2}, —X—, —Y—, —X′—, —Y′—, -L¹-, -L²-, -L⁴-, -L⁵-, -BA are as defined elsewhere therein; and
each -L^{3a}- is

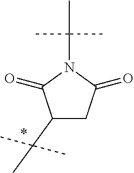

and each -L^{3b}- is

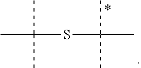

In certain embodiments, the drug conjugate or pharmaceutically acceptable salt thereof the present invention is shown below, i.e. as a drug conjugate comprising a plurality of HA strands 2A and a plurality of HA strands 2B, wherein:
each 2A comprises a plurality of linearly connected units Z¹, Z³-i (a), Z²-i and Z⁴-i:

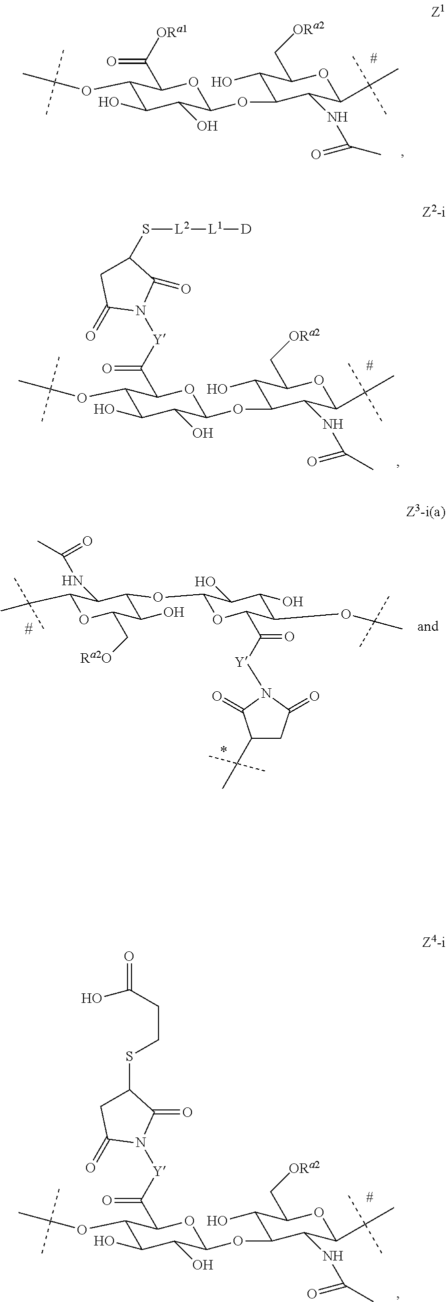

each 2B comprises a plurality of linearly connected units Z¹ and Z³-i (b):

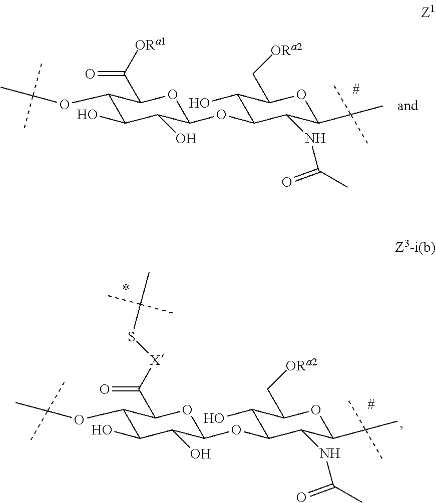

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
a dashed line marked with * indicates a point of crosslinking attachment between a unit Z³-i (a) and Z³-i (b) such that at least one 2A is crosslinked to at least 2B;
each R^{a1} is H or an alkali metal ion;
each —R^{a2} is —H;
each -D is defined as elsewhere therein;
each —X′— is of formula (x4):

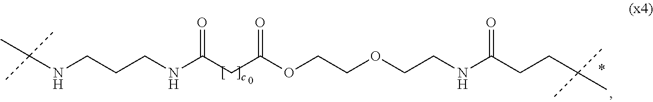

wherein the unmarked dashed line indicates the attachment to the carbonyl group, the dashed line marked with an asterisk indicates the attachment to the sulfur atom and c₀ is 7;
each —Y′— is of formula (y4):

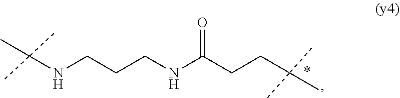

wherein the unmarked dashed line indicates the attachment to the carbonyl group and the dashed line marked with an asterisk indicates the attachment to the nitrogen atom of the thiosuccinimide ring;
each -L²-L¹- is of formula (s1) or (s2):

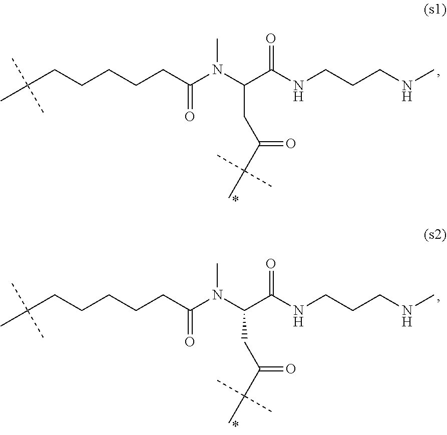

and wherein the dashed line indicates the attachment to the sulfur atom and the dashed line marked with the asterisk indicates the attachment to -D.

It is to be emphasized that in the embodiments described above moiety -D is attached suitably to 2A only, i.e. not to 2B. Similarly, the blocking agent is only attached to strand 2A.

Generally throughout the application it is understood that within the functionalized HA (HA strands) and 1A, 1B, 2A and 2B the disacharide units are connected to each other via the β-1,4 and β-1,3 glycosidic bonds.

The present invention also relates to drug conjugates or pharmaceutically acceptable salts thereof obtainable or obtained by the methods of the present invention.

Another aspect of the present invention is a pharmaceutical composition comprising the drug conjugate or pharmaceutically acceptable salt thereof of the present invention and at least one pharmaceutically acceptable excipient.

The present invention also relates to a drug conjugate or pharmaceutically acceptable salt thereof or pharmaceutical composition of the present invention for use as a medicament.

The present invention also relates to a drug conjugate or pharmaceutically acceptable salt thereof or pharmaceutical composition of the present invention for use in a method of treating a disease, such as an ocular disease, that can be treated with D-H or D or its pharmaceutically acceptable salt thereof.

The present invention also relates to a drug conjugate or pharmaceutically acceptable salt thereof or pharmaceutical composition of the present invention for use in the manufacture of a medicament, such as a medicament for the treatment of an ocular disease.

Exemplary ocular disorders are selected from the group consisting of age-related macular degeneration (AMD), macular degeneration, macular edema, diabetic macular edema (DME), retinopathy, diabetic retinopathy (DR), other ischemia-related retinopathies, retinopathy of prematurity (ROP), retinal vein occlusion (RVO), CNV, corneal neovascularization, a disease associated with corneal neovascularization, retinal neovascularization, uveitic macular edema, branched retinal vein occlusion (BRVO), central retinal vein occlusion (CRVO), submacular hemorrhage, polypoidal choroidal vasculopathy (PCV), retinal microaneurysm, retinal artery occlusion (RAO), branch retinal artery occlusion (BRAO), central retinal artery occlusion (CRAO), subfoveal hemorrhage, subretinal hemorrhage, radiation retinopathy, exudative retinal detachment, Eales disease, neovascular macular telangiectasia, ischemic retinal vasculitis, a disease associated with retinal or choroidal neovascularization, pathologic myopia, von Hippel-Lindau disease, histoplasmosis of the eye, familial exudative vitreoretinopathy (FEVR), Coats' disease, Norrie disease, osteoporosis-pseudoglioma syndrome (OPPG), subconjunctival hemorrhage, rubeosis, ocular neovascular disease, neovascular glaucoma, retinitis pigmentosa (RP), hypertensive retinopathy, retinal angiomatous proliferation, macular telangiectasia, iris neovascularization, intraocular neovascularization, retinal degeneration, cystoid macular edema (CME), vasculitis, papilloedema, retinitis, conjunctivitis, Leber congenital amaurosis, uveitis, choroiditis, ocular histoplasmosis, blepharitis, dry eye, traumatic eye injury and Sjögren's disease.

The ocular disease may also be selected from the group consisting of age-related macular degeneration (AMD), macular degeneration, macular edema, diabetic macular edema (DME), retinopathy, diabetic retinopathy (DR), other ischemia-related retinopathies, retinopathy of prematurity (ROP), retinal vein occlusion (RVO), CNV, corneal neovascularization, a disease associated with corneal neovascularization, retinal neovascularization, a disease associated with retinal/choroidal neovascularization, pathologic myopia, von Hippel-Lindau disease, histoplasmosis of the eye, familial exudative vitreoretinopathy (FEVR), Coats' disease, Norrie disease, osteoporosis-pseudoglioma syndrome (OPPG), subconjunctival hemorrhage, rubeosis, ocular neovascular disease, neovascular glaucoma, retinitis pigmentosa (RP), hypertensive retinopathy, retinal angiomatous proliferation, macular telangiectasia, iris neovascularization, intraocular neovascularization, retinal degeneration, cystoid macular edema (CME), vasculitis, papilloedema, retinitis, conjunctivitis, Leber congenital amaurosis, uveitis, choroiditis, ocular histoplasmosis, blepharitis, dry eye, traumatic eye injury and Sjögren's disease.

Suitably the ocular disease is selected from the group consisting of AMD, DME, DR or RVO. Preferably, the ocular disease is AIMD.

Another aspect of the present invention is a method of preventing or treating a disease, such as an ocular disease, that can be prevented or treated with D-H or D, the method comprising the step of administering to a subject in need thereof a therapeutically effective amount of the drug conjugate or pharmaceutically acceptable salt thereof or the pharmaceutical composition of the present invention.

In certain embodiments, all moieties -D of the drug conjugate or pharmaceutically acceptable salt thereof are identical, i.e. have the same chemical structure. In such case all moieties -D of the drug conjugate derive from the same type of drug molecule.

In certain embodiments, the drug conjugate or pharmaceutically acceptable salt thereof of the present invention comprises different moieties -D, i.e. comprises moieties -D with different chemical structures. These different structures derive from different types of drug molecules. In certain embodiments, the drug conjugate of the present invention comprises two different types of moieties -D. In certain embodiments, the drug conjugate of the present invention comprises three different types of moieties -D. In certain embodiments, the drug conjugate of the present invention comprises four different types of moieties -D. In certain embodiments, the drug conjugate of the present invention comprises five different types of moieties -D.

If the drug conjugates of the present invention comprise more than one type of -D, all moieties -D may be conjugated to the same type of -L¹- or may be conjugated to different types of -L¹-, i.e. a first type of -D may be conjugated to a first type of -L¹-, a second type of -D may be conjugated to a second type of -L¹- and so on. Using different types of -L¹- may, in certain embodiments, allow different release kinetics for different types of -D, such as for example a faster release for a first type of -D, a medium release for a second type of -D and a slow release for a third type of -D. Accordingly, in certain embodiments the drug conjugates of the present invention comprise one type of -L¹-. In certain embodiments, the drug conjugates of the present invention comprise two types of -L¹-. In certain embodiments, the drug conjugates of the present invention comprise three types of -L¹-. In certain embodiments, the drug conjugates of the present invention comprise four types of -L¹-.

In certain embodiments, the drug conjugates of the present invention comprise one type of -D and one type of -L¹-. In certain embodiments, the drug conjugates of the present invention comprise two types of -D and two types of -L¹-. In certain embodiments, the drug conjugates of the present invention comprise three types of -D and three types of -L¹-. In certain embodiments, the drug conjugates of the present invention comprise four types of -D and four types of -L¹-.

In certain embodiments, all moieties -L¹- of the drug conjugate have the same structure. In certain embodiments, the drug conjugate comprises two or more different types of moiety -L¹-, such as for example two, three, four or five different types of moiety -L¹-. Such two or more different types of moiety -L¹- may be conjugated to the same or different type of -D. Using different types of -L¹- allows releasing the same or different type of drug D-H from the conjugate of the present invention with different release half-lives, such as when combining a first group of moieties -L¹- with a short release half-life with a second group of moiety -L¹- with a long half-life.

In certain embodiments, -D is selected from the group consisting of small molecule drug moieties, medium size drug moieties, peptide drug moieties and protein drug moieties.

In certain embodiments, -D is a small molecule drug moiety.

In certain embodiments, -D is a peptide drug moiety. In certain embodiments, -D is a protein drug moiety. In certain embodiments, such protein moiety is a monoclonal or polyclonal antibody or fragment or fusion thereof.

In certain embodiments, -L¹- is attached to a cysteine residue of -D. In certain embodiments, -L¹- is attached to a histidine residue of -D. In certain embodiments, -L¹- is attached to a lysine residue of -D. In certain embodiments, -L¹- is attached to a tryptophan residue of -D. In certain embodiments, -L¹- is attached to a serine residue of -D. In certain embodiments, -L¹- is attached to a threonine residue of -D. In certain embodiments, -L¹- is attached to a tyrosine residue of -D. In certain embodiments, -L¹- is attached to an aspartic acid residue of -D. In certain embodiments, -L¹- is attached to a glutamic acid residue of -D. In certain embodiments, -L¹- is attached to an arginine residue of -D.

In certain embodiments, at least one moiety -L¹- is attached to an amino acid residue of -D and one or more additional moieties -L¹- are attached to a modifying moiety present in -D.

The moiety -L¹- may be connected to -D through any type of linkage, provided that it is reversible.

In certain embodiments, -L¹- is connected to -D through a linkage selected from the group consisting of amide, ester, carbamate, acetal, aminal, imine, oxime, hydrazone, disulfide and acylguanidine. In certain embodiments, -L¹- is connected to -D through a linkage selected from the group consisting of amide, ester, carbamate and acylguanidine. It is understood that these linkages may not be reversible per se, but that reversibility may be an effect of certain groups of atoms or moieties present in -L¹-.

In certain embodiments, -L¹- is connected to -D through an ester linkage. In certain embodiments, -L¹- is connected to -D through a carbamate linkage. In certain embodiments, -L¹- is connected to -D through an acylguanidine. In certain embodiments, -L¹- is connected to -D through an amide linkage.

In certain embodiments, -L¹- is connected to -D via the nitrogen of an amine functional group of a side chain of a lysine residue of -D. Suitably, -L¹- is connected to -D via the nitrogen of an amine functional group, such as that of an amine functional group of a side chain of a lysine residue of -D and the linkage formed between -D and -L¹- is an amide.

In certain embodiments, -L¹- has a structure as disclosed in WO 2009/095479 A2, which is hereby incorporated by reference in its entirety. Accordingly, in certain embodiments the moiety -L¹- is of formula (I):

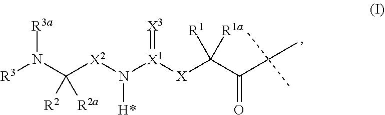

wherein
the dashed line indicates the attachment to a nitrogen of -D by forming an amide bond;
—X— is —C(R⁴R^{4a})—; —N(R⁴)—; —O—; —C(R⁴R^{4a})—C(R⁵R^{5a})—; —C(R⁵R^{5a})—C(R⁴R^{4a})—; —C(R⁴R^{4a})—N(R⁶)—; —N(R⁶)—C(R⁴R^{4a})—; —C(R⁴R^{4a})—O—; —O—C(R⁴R^{4a})—; or —C(R⁷R^{7a})—;
X¹ is C; or S(O);
—X²— is —C(R⁸R^{8a})—; or —C(R⁸R^{8a})—C(R⁹R^{9a})—;
═X³ is ═O; ═S; or ═N—CN;
—R¹, —R^{1a}, —R², —R^{2a}, —R⁴, —R^{4a}, —R⁵, —R^{5a}, —R⁶, —R⁸, —R^{8a}, —R⁹, —R^{9a} are independently selected from the group consisting of —H; and C₁₋₆ alkyl;
—R³, —R^{3a} are independently selected from the group consisting of —H; and C₁₋₆ alkyl, provided that in case one of —R³, —R^{3a} or both are other than —H they are connected to N to which they are attached through a sp³-hybridized carbon atom;
—R⁷ is —N(R¹⁰R^{10a}); or —NR¹⁰—(C═O)—R¹¹;
—R^{7a}, —R¹⁰, —R^{10a}, —R¹¹ are independently of each other —H; or C₁₋₁₀ alkyl;
optionally, one or more of the pairs —R^{1a}/—R^{4a}, —R^{1a}/—R^{5a}, —R^{1a}/—R^{7a}, —R^{4a}/—R^{5a}, —R^{8a}/—R^{9a} form a chemical bond;
optionally, one or more of the pairs —R¹/—R^{1a}, —R²/—R^{2a}, —R⁴/—R^{4a}, —R⁵/—R^{5a}, —R¹/—R^{8a}, —R⁹/—R^{9a} are joined together with the atom to which they are attached to form a C₃₋₁₀ cycloalkyl; or
3- to 10-membered heterocyclyl;
optionally, one or more of the pairs —R¹/—R⁴, —R¹/—R⁵, —R¹/—R⁶, —R¹/—R^{7a}, —R⁴/—R⁵, —R⁴/—R⁶, —R¹/—R⁹, —R²/—R³ are joined together with the atoms to which they are attached to form a ring A;
optionally, —R³/—R^{3a} are joined together with the nitrogen atom to which they are attached to form a 3- to 10-membered heterocycle;
ring A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
each -L¹- is substituted with -L²- and optionally further substituted provided that the hydrogen marked with the asterisk in formula (I) is not replaced by a substituent.

In certain embodiment, -L¹- is of formula (I), wherein the dashed line indicates attachment to a nitrogen of an amine of a lysine side chain of -D. In certain embodiments, -L¹- is of formula (I), wherein the dashed line indicates attachment to the nitrogen of the amine of the N-terminus of -D. In certain embodiments, -L¹- of formula (I) is not further substituted.

It is understood that if —R³/—R^{3a} of formula (I) are joined together with the nitrogen atom to which they are attached to form a 3- to 10-membered heterocycle, only such 3- to 10-membered heterocycles may be formed in which the atoms directly attached to the nitrogen are sp³-hybridized carbon atoms. In other words, such 3- to 10-membered heterocycle formed by —R³/—R^{3a} together with the nitrogen atom to which they are attached has the following structure:

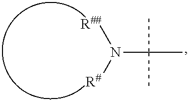

wherein
the dashed line indicates attachment to the rest of -L¹-;
the ring comprises 3 to 10 atoms comprising at least one nitrogen; and
R^{#} and R^{##} represent a sp³-hydridized carbon atom.

It is also understood that the 3- to 10-membered heterocycle may be further substituted.

Exemplary embodiments of suitable 3- to 10-membered heterocycles formed by —R³/—R^{3a} of formula (I together with the nitrogen atom to which they are attached are the following:

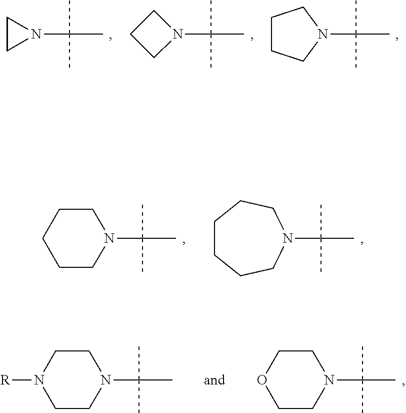

wherein
dashed lines indicate attachment to the rest of the molecule; and
—R is selected from the group consisting of —H and C₁₋₆ alkyl.
L¹- of formula (I) may optionally be further substituted. In general, any substituent may be used as far as the cleavage principle is not affected, i.e. the hydrogen marked with the asterisk in formula (I) is not replaced and the nitrogen of the moiety

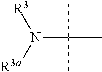

of formula (I) remains part of a primary, secondary or tertiary amine, i.e. —R³ and —R^{3a} are independently of each other —H or are connected to —N<through a sp³-hybridized carbon atom.

In certain embodiments, —R¹ or —R^{1a} of formula (I) is substituted with -L²-. In certain embodiments, —R² or —R^{2a} of formula (I) is substituted with -L²-. In certain embodiments, —R³ or —R^{3a} of formula (I) is substituted with -L²-. In certain embodiments, —R⁴ of formula (I) is substituted with -L²-. In certain embodiments, —R⁵ or —R^{5a} of formula (I) is substituted with -L²-.

In certain embodiments, —R⁶ of formula (I) is substituted with -L²-. In certain embodiments, —R⁷ or —R^{7a} of formula (I) is substituted with -L²-. In certain embodiments, —R⁸ or —R^{8a} of formula (I) is substituted with -L²-. In certain embodiments, —R⁹ or —R^{9a} of formula (I) is substituted with -L²-.

Suitably, —R¹¹ of formula (I) is substituted with -L²-.

In certain embodiments, -L¹- has a structure as disclosed in WO 2018/193408 A1, which is hereby incorporated by reference in its entirety. Accordingly, in certain embodiments the moiety -L¹- is of formula (II):

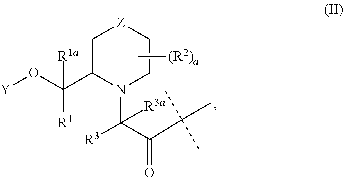

wherein
the dashed line indicates attachment to -D which is a primary amine, secondary amine, or ring nitrogen atom of an azaheteroaryl ring;
—R¹ is —H or C₁-C₄ alkyl;
—R^{1a} is —H or C₁-C₄ alkyl, or —CR¹R^{1a}, taken in combination form a C₃-C₆ cycloalk-1,1-diyl;
—R² is independently selected at each occurrence from C₁-C₄ alkyl or oxo, or two —R² groups taken in combination form a fused C₃-C₆ cycloalkyl or spiro C₃-C₆ cycloalk-1,1-diyl group;
a is 0, 1, 2, 3, or 4;
—R³ is —H or C₁-C₄ alkyl;
—R^{3a} is —H or C₁-C₄ alkyl, or —CR³R^{3a}, taken in combination form a C₃-C₆ cycloalk-1,1-diyl;
—Y is —C(O)R⁴, —C(O)OR⁴, —C(O)NHR⁴, —C(O)NR⁵R⁶, —SiR⁵R⁶R⁷, or —CR¹²R^{12a}OR¹³;
—R¹² is —H or C₁-C₄ alkyl;
—R^{12a} is —H or C₁-C₄ alkyl, or —CR¹²R^{12a}, taken in combination form a C₃-C₆ cycloalk-1,1-diyl;
—R¹³ is C₁-C₄ alkyl; or —CHR¹²OR¹³, taken in combination from a 5-, 6-, or 7-membered cyclic ether;
—R⁴ is C₁-C₈ alkyl or C₃-C₇ cycloalkyl, wherein cycloalkyl is optionally substituted with 0, 1, or 2 independently selected C₁-C₄ alkyl groups and wherein alkyl is optionally substituted by C₁-C₄ alkoxy;
—R⁵ and —R⁶ are each independently selected from C₁-C₄ alkyl and C₃-C₆ cycloalkyl;
—R⁷ is C₁-C₈ alkyl, C₃-C₇ cycloalkyl, C₁-C₈ alkoxy, C₃-C₇ cycloalkyloxy, heterocycloalkyloxy, or —(OCHR³CH₂)_{b}O—C₁-C₄ alkyl, wherein the heterocycloalkyloxy is a 4- to 7-membered saturated heterocyclic ring having one heteroatom selected from N, O, and S and optionally substituted with 0, 1, or 2 independently selected C₁-C₄ alkyl groups;
b is an integer ranging from 1 to 10;
—Z is —CH-L²-, or —N-L²-; and
wherein -L¹- is optionally further substituted.

In certain embodiments, -L¹- is of formula (IIa):

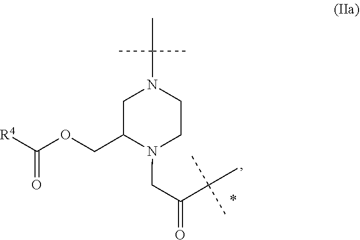

wherein
the dashed line marked with the asterisk indicates attachment to a nitrogen atom of -D by forming an amide bond;
the unmarked dashed line indicates attachment to -L²-; and
—R⁴ is —CH₃, —CH₂—O—CH₃, —CH₂CH₃, or —CH(CH₃)₂.

In certain embodiments, -L¹- is of formula (IIa) and -L²- is of formula (IIa′):

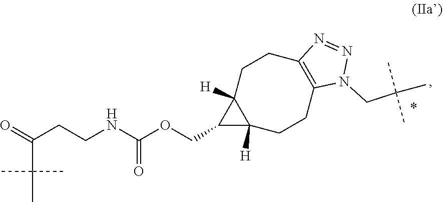

wherein
the unmarked dashed line indicates attachment to -L¹-; and
the dashed line marked with the asterisk indicates attachment to -L⁴-.

In certain embodiments, -L¹- has a structure as disclosed in WO2016/020373A1, which is hereby incorporated by reference in its entirety. Accordingly, in certain embodiments the moiety -L¹- is of formula (III):

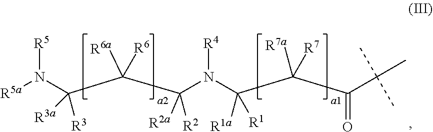

wherein
the dashed line indicates attachment to a primary or secondary amine or hydroxyl of -D by forming an amide or ester linkage, respectively;
—R¹, —R^{1a}, —R², —R^{2a}, —R³ and —R^{3a} are independently of each other selected from the group consisting of —H, —C(R⁸R^{8a}R^{8b}), —C(═O)R′, —C≡N, —C(═NR′)R^{8a}, —CR⁸(═CR^{8a}R^{8b}), —C≡CR⁸ and -T;
—R⁴, —R⁵ and —R^{5a} are independently of each other selected from the group consisting of —H, —C(R⁹R^{9a}R^{9b}) and -T;
a₁ and a₂ are independently of each other 0 or 1;
each —R⁶, —R^{6a}, —R⁷, —R^{7a}, —R⁸, —R^{8a}, —R^{8b}, —R⁹, —R^{9a}, —R^{9b} are independently of each other selected from the group consisting of —H, halogen, —CN, —COOR¹⁰, —OR¹⁰, —C(O)R¹⁰, —C(O)N(R¹⁰R^{10a}), —S(O)₂N(R¹⁰R^{10a}), —S(O)N(R¹⁰R^{10a}), —S(O)₂R¹⁰, —S(O)R¹⁰, —N(R¹⁰)S(O)₂N(R¹⁰R^{10b}), —SR¹⁰, —N(R¹⁰R^{10a}), —NO₂, —OC(O)R¹⁰, —N(R¹⁰)C(O)R^{10a}, —N(R¹⁰)S(O)₂R^{10a}, —N(R¹⁰)S(O)R^{10a}, —N(R¹⁰)C(O)OR^{10a}, —N(R¹⁰)C(O)N(R^{10a}R^{10b}), —OC(O)N(R¹⁰R^{10a}), -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each —R¹⁰, —R^{10a}, —R^{10b} is independently selected from the group consisting of —H, -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each T is independently of each other selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T is independently optionally substituted with one or more —R¹¹, which are the same or different;
each —R¹¹ is independently of each other selected from halogen, —CN, oxo (═O), —COOR¹³, —OR¹³, —C(O)R¹³, —C(O)N(R¹³R^{13a}), —S(O)₂N(R¹³R^{13a}), —S(O)N(R¹³R^{13a}), —S(O)₂R¹³, —S(O)R¹³, —N(R¹³)S(O)₂N(R^{13a}R^{13b}), —SR¹³, —N(R¹³R^{13a}), —NO₂, —OC(O)R¹³, —N(R¹³)C(O)R^{13a}, —N(R¹³)S(O)₂R^{13a}, —N(R¹³)S(O)R^{3a}, —N(R¹³)C(O)OR^{3a}, —N(R¹³)C(O)N(R^{13a}R^{13b}), —OC(O)N(R¹³R^{13a}), and C₁₋₆ alkyl; wherein C₁₋₆ alkyl is optionally substituted with one or more halogen, which are the same or different;
each —R¹², —R^{12a}, —R¹³, —R^{13a}, —R^{13b} is independently selected from the group consisting of —H, and C₁₋₆ alkyl; wherein C₁₋₆ alkyl is optionally substituted with one or more halogen, which are the same or different;
optionally, one or more of the pairs —R¹/—R^{1a}, —R²/—R^{2a} —R³/—R^{3a} —R⁶/—R^{6a}, —R⁷/—R^{7a} arejoined together with the atom to which they are attached to form a C₃₋₁₀ cycloalkyl or a 3- to 10-membered heterocyclyl;
optionally, one or more of the pairs —R¹/—R², —R¹/—R³, —R¹/—R⁴, —R¹/—R⁵, —R¹/—R⁶, —R¹/—R⁷, —R²/—R³, —R²/—R⁴, —R²/—R⁵, —R²/—R⁶, —R²/—R⁷, —R³/—R⁴, —R³/—R⁵, —R³/—R⁶, —R³/—R⁷, —R⁴/—R⁵, —R⁴/—R⁶, —R⁴/—R⁷, —R⁵/—R⁶, —R⁵/—R⁷, —R⁶/—R⁷ are joined together with the atoms to which they are attached to form a ring A;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl;
wherein -L¹- is substituted with -L²- and wherein -L¹- is optionally further substituted.

The optional further substituents of -L¹- of formula (III) are preferably as described above.

In certain embodiments, -L¹- of formula (III) is not further substituted.

In certain embodiments, -L¹- has a structure as disclosed in EP1536334B1, WO2009/009712A1, WO2008/034122A1, WO2009/143412A2, WO2011/082368A2, and U.S. Pat. No. 8,618,124B2, which are herewith incorporated by reference.

In certain embodiments, -L¹- has a structure as disclosed in U.S. Pat. No. 8,946,405B2 and U.S. Pat. No. 8,754,190B2, which are hereby incorporated by reference in their entirety. Accordingly, in certain embodiments -L¹- is of formula (IV):

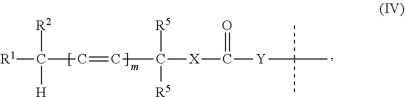

wherein
the dashed line indicates attachment to -D through a functional group of -D selected from the group consisting of —OH, —SH and —NH₂;
m is 0 or 1;
at least one or both of —R¹ and —R² is/are independently of each other selected from the group consisting of —CN, —NO₂, optionally substituted aryl, optionally substituted heteroaryl, optionally substituted alkenyl, optionally substituted alkynyl, —C(O)R³, —S(O)R³, —S(O)₂R³, and —SR⁴,
one and only one of —R¹ and —R² is selected from the group consisting of —H, optionally substituted alkyl, optionally substituted arylalkyl, and optionally substituted heteroarylalkyl;
—R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
—R⁴ is selected from the group consisting of optionally substituted alkyl, optionally substituted aryl, optionally substituted arylalkyl, optionally substituted heteroaryl, and optionally substituted heteroarylalkyl;
each —R⁵ is independently selected from the group consisting of —H, optionally substituted alkyl, optionally substituted alkenylalkyl, optionally substituted alkynylalkyl, optionally substituted aryl, optionally substituted arylalkyl, optionally substituted heteroaryl and optionally substituted heteroarylalkyl;
—R⁹ is selected from the group consisting of —H and optionally substituted alkyl;
—Y— is absent and —X— is —O— or —S—; or
—Y— is —N(Q)CH₂— and —X— is —O—;
Q is selected from the group consisting of optionally substituted alkyl, optionally substituted aryl, optionally substituted arylalkyl, optionally substituted heteroaryl and optionally substituted heteroarylalkyl;
optionally, —R¹ and —R² may be joined to form a 3 to 8-membered ring; and
optionally, both —R⁹ together with the nitrogen to which they are attached form a heterocyclic ring;
wherein -L¹- is substituted with -L²- and wherein -L¹- is optionally further substituted.

Only in the context of formula (IV) the terms used have the following meaning: The term “alkyl” as used herein includes linear, branched or cyclic saturated hydrocarbon groups of 1 to 8 carbons, or in some embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, preferably 6 to 10 carbons, including groups such as phenyl, naphthyl, and anthracenyl. The term “heteroaryl” includes aromatic rings comprising 3 to 15 carbons containing at least one N, O or S atom, preferably 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In some instance, alkenyl, alkynyl, aryl or heteroaryl moieties may be coupled to the remainder of the molecule through an alkylene linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4 to 8 membered aromatic or non-aromatic ring comprising 3 to 7 carbon atoms and at least one N, O, or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl, or an additional ring, each optionally further substituted.

Optional substituents on any group, including the above, include halo, nitro, cyano, —OR, —SR, —NR₂, —OCOR, —NRCOR, —COOR, —CONR₂, —SOR, —SO₂R, —SONR₂, —SO₂NR₂, wherein each R is independently alkyl, alkenyl, alkynyl, aryl or heteroaryl, or two R groups taken together with the atoms to which they are attached form a ring.

In certain embodiments, -L¹- has a structure as disclosed in WO2013/036857A1, which is hereby incorporated by reference in its entirety. Accordingly, in certain embodiments, -L¹- is of formula (V):

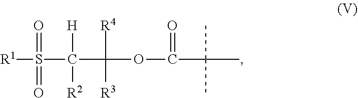

wherein
the dashed line indicates attachment to -D through an amine functional group of -D;
—R¹ is selected from the group consisting of optionally substituted C₁-C₆ linear, branched, or cyclic alkyl; optionally substituted aryl; optionally substituted heteroaryl; alkoxy; and —NR⁵²;
—R² is selected from the group consisting of —H; optionally substituted C₁-C₆ alkyl; optionally substituted aryl; and optionally substituted heteroaryl;
—R³ is selected from the group consisting of —H; optionally substituted C₁-C₆ alkyl; optionally substituted aryl; and optionally substituted heteroaryl;
—R⁴ is selected from the group consisting of —H; optionally substituted C₁-C₆ alkyl; optionally substituted aryl; and optionally substituted heteroaryl;
each —R⁵ is independently of each other selected from the group consisting of —H; optionally substituted C₁-C₆ alkyl; optionally substituted aryl; and optionally substituted heteroaryl; or when taken together two —R⁵ can be cycloalkyl or cycloheteroalkyl;
wherein -L¹- is substituted with -L²- and wherein -L¹- is optionally further substituted.

Only in the context of formula (V) the terms used have the following meaning: “Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 C atoms.

“Aryl” includes aromatic hydrocarbon groups of 6-18 carbons, preferably 6-10 carbons, including groups such as phenyl, naphthyl, and anthracene “Heteroaryl” includes aromatic rings comprising 3-15 carbons containing at least one N, O or S atom, preferably 3-7 carbons containing at least one N, O or S atom, including groups such as pyrrolyl, pyridyl, pyrimidinyl, imidazolyl, oxazolyl, isoxazolyl, thiszolyl, isothiazolyl, quinolyl, indolyl, indenyl, and similar.

The term “substituted” means an alkyl, alkenyl, alkynyl, aryl, or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketone; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

In certain embodiments, -L¹- has a structure as disclosed in U.S. Pat. No. 7,585,837B2, which is hereby incorporated by reference in its entirety. Accordingly, in certain embodiments, -L¹- is of formula (VI):

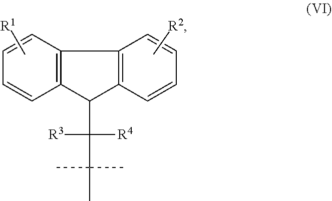

wherein
the dashed line indicates attachment to -D through an amine functional group of -D;
R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl;
wherein -L¹- is substituted with -L²- and wherein -L¹- is optionally further substituted.

Suitable substituents for formulas (VI) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

Only in the context of formula (VI) the terms used have the following meaning:

The terms “alkyl”, “alkoxy”, “alkoxyalkyl”, “aryl”, “alkaryl” and “aralkyl” mean alkyl radicals of 1-8, preferably 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

In certain embodiments, -L¹- has a structure as disclosed in WO2002/089789A1, which is hereby incorporated by reference in its entirety. Accordingly, in certain embodiments, -L¹- is of formula (VII):

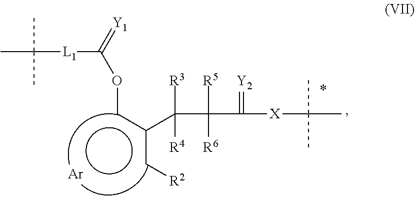

wherein
the dashed line indicates attachment to -D through an amine functional group of -D;
Y₁ and Y₂ are independently O, S or NR⁷;
R², R³, R⁴, R⁵, R⁶ and R⁷ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
Ar is a moiety which when included in formula (VII) forms a multi-substituted aromatic hydrocarbon or a multi-substituted heterocyclic group;
X is a chemical bond or a moiety that is actively transported into a target cell, a hydrophobic moiety, or a combination thereof,
y is 0 or 1;
wherein -L¹- is substituted with -L²- and wherein -L¹- is optionally further substituted.

Only in the context of formula (VII) the terms used have the following meaning:

The term “alkyl” shall be understood to include, e.g. straight, branched, substituted C₁₋₁₂ alkyls, including alkoxy, C₃₋₈ cycloalkyls or substituted cycloalkyls, etc.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substtued cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophone; alkoxy includes moieties such as methoxy; and phenoxy includes moieties such as 3-nitrophenoxy. Halo- shall be understood to include fluoro, chloro, iodo and bromo.

In certain embodiments, -L¹- comprises a substructure of formula (VIII):

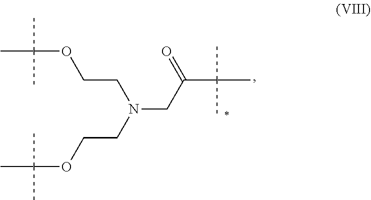

wherein
the dashed line marked with the asterisk indicates attachment to a nitrogen of -D by forming an amide bond;
the unmarked dashed lines indicate attachment to the remainder of -L¹-; and
wherein -L¹- is substituted with -L²- and wherein -L¹- is optionally further substituted.

In certain embodiments, -L¹- is of formula (VIII), wherein the dashed line marked with the asterisk indicates attachment to a nitrogen of an amine of a lysine side chain of -D. In certain embodiments, -L¹- is of formula (VIII), wherein the dashed line marked with the asterisk indicates attachment to the nitrogen of the amine of the N-terminus of -D.

In certain embodiments, -L¹- of formula (VIII) is substituted with one moiety -L²-. In certain embodiments, -L¹- of formula (VIII) is not further substituted.

In certain embodiments, -L¹- comprises a substructure of formula (IX):

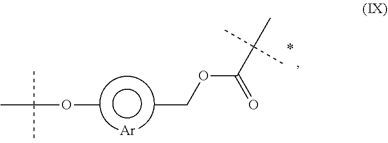

wherein
the dashed line marked with the asterisk indicates attachment to a nitrogen of -D by forming a carbamate bond;
the unmarked dashed lines indicate attachment to the remainder of -L¹-; and
wherein -L¹- is substituted with -L²- and wherein -L¹- is optionally further substituted.

In certain embodiments, -L¹- is of formula (IX), wherein the dashed line marked with the asterisk indicates attachment to a nitrogen of an amine of a lysine side chain of -D.

In certain embodiments, -L¹- is of formula (IX), wherein the dashed line marked with the asterisk indicates attachment to the nitrogen of the amine of the N-terminus of -D.

In certain embodiments, -L¹- of formula (IX) is not further substituted.

In certain embodiments, -L¹- is of formula (IX-a):

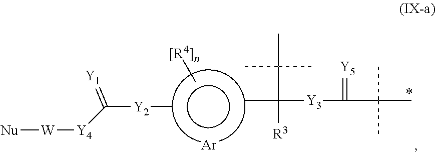

wherein
the dashed line marked with the asterisk indicates attachment to a nitrogen of -D and the unmarked dashed line indicates attachment to -L²-;
n is 0, 1, 2, 3, or 4;
═Y₁ is selected from the group consisting of ═O and ═S;
—Y₂— is selected from the group consisting of —O— and —S—;
—Y₃— is selected from the group consisting of —O— and —S—;
—Y₄— is selected from the group consisting of —O—, —NR⁵— and —C(R⁶R^{6a})—;
═Y₅ is selected from the group consisting of ═O and ═S;
—R³, —R⁵, —R⁶, —R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—W— is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—;
-Nu is a nucleophile selected from the group consisting of —N(R⁷R^{7a}), —N(R⁷OH), —N(R⁷)—N(R^{7a}R^{7b}), —S(R⁷), —COOH,

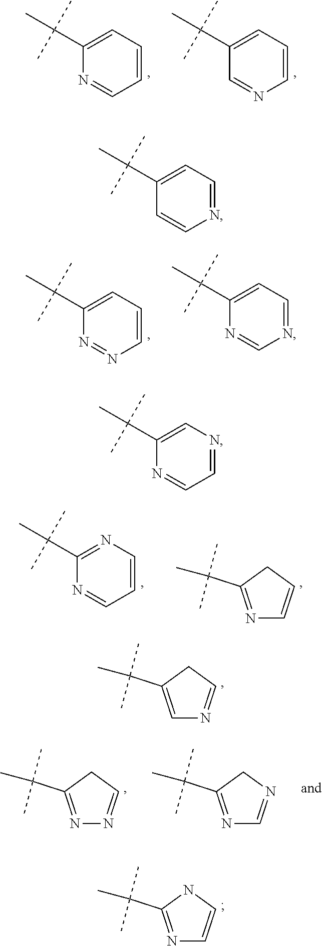

—Ar— is selected from the group consisting of

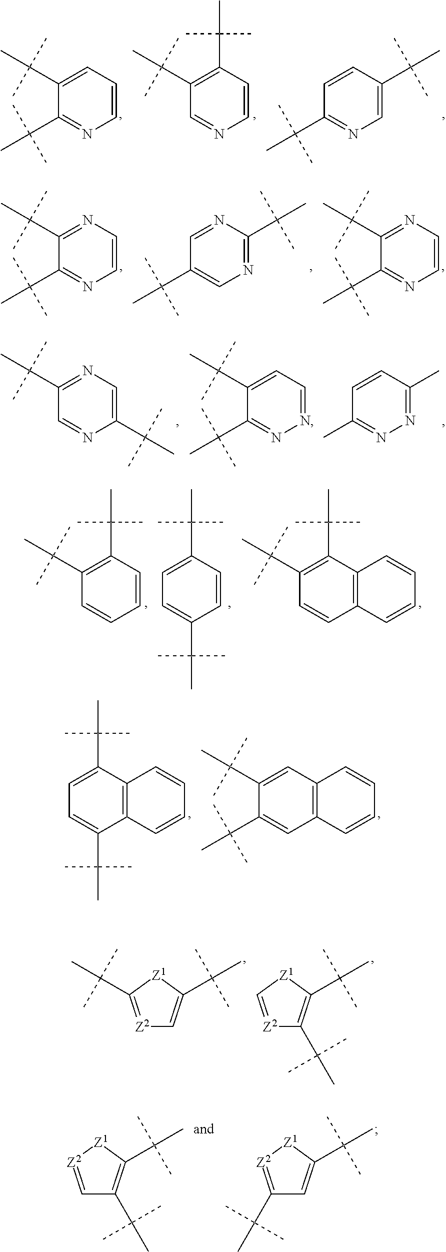

wherein
dashed lines indicate attachment to the remainder of -L¹-,
—Z¹— is selected from the group consisting of —O—, —S— and —N(R⁷)—, and
—Z²— is —N(R⁷)—; and
—R⁷, —R^{7a}, —R^{7b} are independently of each other selected from the group consisting of —H, C₁₋₆ alkyl, C₂₋₆ alkenyl and C₂₋₆ alkynyl;
wherein -L¹- is optionally further substituted.

In certain embodiments, -L¹- is of formula (IX-a), wherein the dashed line marked with the asterisk indicates attachment to a nitrogen of an amine of a lysine side chain of -D.

In certain embodiments, -L¹- is of formula (IX-a), wherein the dashed line marked with the asterisk indicates attachment to the nitrogen of the amine of the N-terminus of -D.

In certain embodiments, -L¹- of formula (IX-a) is not further substituted.

In certain embodiments, -L¹- is of formula (IX-b):

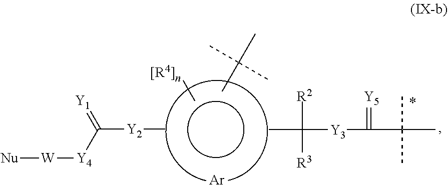

wherein
the dashed line marked with the asterisk indicates attachment to a nitrogen of -D and the unmarked dashed line indicates attachment to -L²-;
n is 0, 1, 2, 3, or 4;
═Y₁ is selected from the group consisting of ═O and ═S;
—Y₂— is selected from the group consisting of —O— and —S—;
—Y₃— is selected from the group consisting of —O— and —S—;
—Y₄— is selected from the group consisting of —O—, —NR⁵— and —C(R⁶R^{6a})—;
═Y₅ is selected from the group consisting of ═O and ═S;
—R², —R³, —R⁵, —R⁶, —R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—W— is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—;
-Nu is a nucleophile selected from the group consisting of —N(R⁷R^{7a}), —N(R⁷OH), —N(R⁷)—N(R^{7a}R^{7b}), —S(R⁷), —COOH,

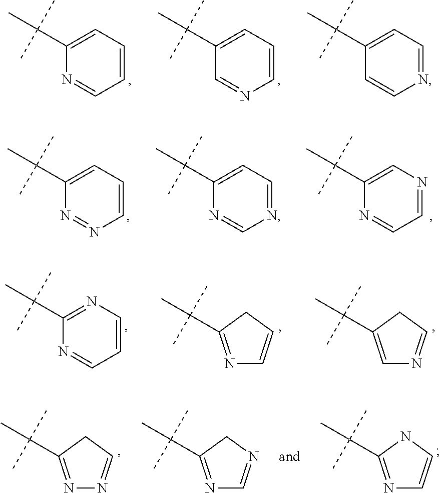

—Ar— is selected from the group consisting of

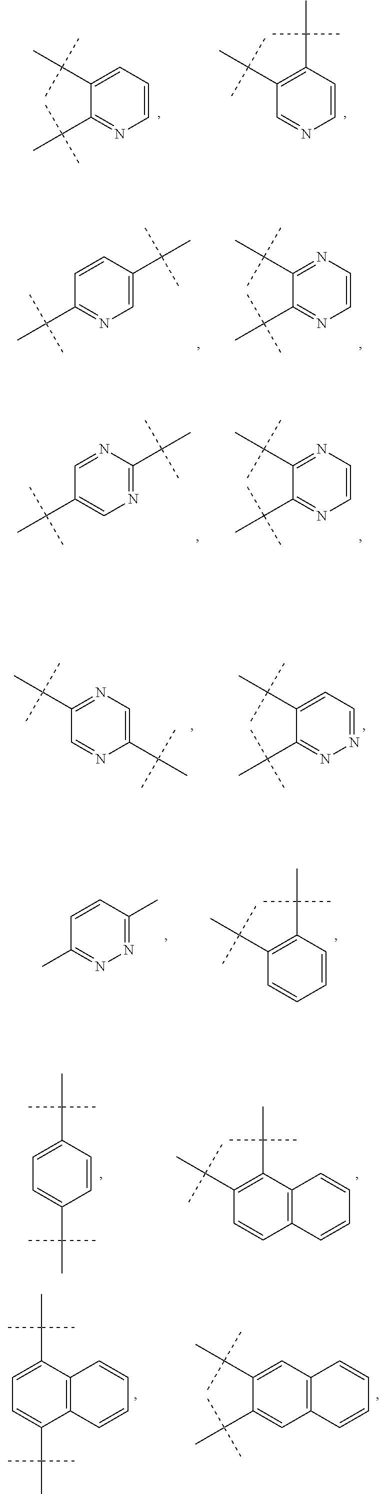

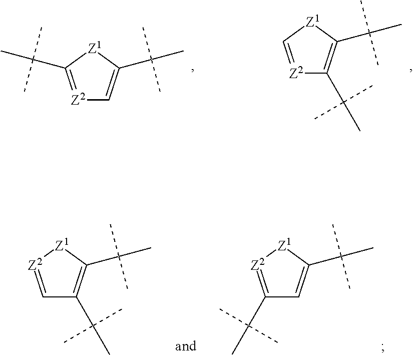

wherein
the dashed lines indicate attachment to the remainder of -L¹-,
—Z¹— is selected from the group consisting of —O—, —S— and —N(R⁷)—, and
—Z²— is —N(R⁷)—; and
—R⁷, —R^{7a}, —R^{7b} are independently of each other selected from the group consisting of —H, C₁₋₆ alkyl, C₂₋₆ alkenyl and C₂₋₆ alkynyl;
wherein -L¹- is optionally further substituted.

In certain embodiments, -L¹- is of formula (IX-b), wherein the dashed line marked with the asterisk indicates attachment to a nitrogen of an amine of a lysine side chain of -D.

In certain embodiments, -L¹- is of formula (IX-b), wherein the dashed line marked with the asterisk indicates attachment to the nitrogen of the amine of the N-terminus of -D.

In certain embodiments, -L¹- of formula (IX-b) is not further substituted.

In certain embodiments, ═Y¹ of formula (IX-a) and (IX-b) is ═O.

In certain embodiments, —Y²— of formula (IX-a) and (IX-b) is —O—.

In certain embodiments, —Y³— of formula (IX-a) and (IX-b) is —O—.

In certain embodiments, —Y⁴— of formula (IX-a) and (IX-b) is —NR⁵—.

In certain embodiments, ═Y⁵ of formula (IX-a) and (IX-b) is ═O.

In certain embodiments, n of formula (IX-a) and (IX-b) is 0 or 1. In certain embodiments, n of formula (IX-a) and (IX-b) is 0. In certain embodiments, n of formula (IX-a) and (IX-b) is 1.

In certain embodiments, —R² of formula (IX-b) is selected from the group consisting of —H, methyl, ethyl, n-propyl, isopropyl, n-butyl, isobutyl, sec-butyl and tert-butyl. In certain embodiments, —R² of formula (IX-b) is selected from the group consisting of —H, methyl, ethyl, n-propyl and isopropyl. In certain embodiments, —R² of formula (IX-b) is selected from —H, methyl and ethyl. In certain embodiments —R² of formula (IX-b) is —H.

In certain embodiments, —R³ of formula (IX-a) and (IX-b) is selected from the group consisting of —H, methyl, ethyl, n-propyl, isopropyl, n-butyl, isobutyl, sec-butyl and tert-butyl. In certain embodiments, —R³ of formula (IX-a) and (IX-b) is selected from the group consisting of —H, methyl, ethyl, n-propyl and isopropyl. In certain embodiments, —R³ of formula (IX-a) and (IX-b) is selected from —H, methyl and ethyl. In certain embodiments, —R³ of formula (IX-a) and (IX-b) is —H.

In certain embodiments, each —R⁴ of formula (IX-a) and (IX-b) is independently selected from methyl, ethyl, n-propyl, isopropyl, n-butyl, isobutyl, sec-butyl and tert-butyl. In certain embodiments, —R⁴ of formula (IX-a) and (IX-b) is selected from the group consisting of methyl, ethyl, n-propyl and isopropyl. In certain embodiments, —R⁴ of formula (IX-a) and (IX-b) is selected from methyl and ethyl.

In certain embodiments, —R⁵ of formula (IX-a) and (IX-b) is selected from the group consisting of —H, methyl, ethyl, n-propyl, isopropyl, n-butyl, isobutyl, sec-butyl and tert-butyl. In certain embodiments, —R⁵ of formula (IX-a) and (IX-b) is selected from the group consisting of —H, methyl, ethyl, n-propyl and isopropyl. In certain embodiments, —R⁵ of formula (IX-a) and (IX-b) is selected from methyl and ethyl. In certain embodiments, —R⁵ of formula (IX-a) and (IX-b) is methyl.

In certain embodiments, —R⁶ and —R^{6a} of formula (IX-a) and (IX-b) are independently selected from the group consisting of —H, methyl, ethyl, n-propyl, isopropyl, n-butyl, isobutyl, sec-butyl and tert-butyl. In certain embodiments, —R⁶ and —R^{6a} of formula (IX-a) and (IX-b) are independently selected from the group consisting of —H, methyl, ethyl, n-propyl and isopropyl. In certain embodiments, —R⁶ and —R^{6a} of formula (IX-a) and (IX-b) are independently selected from —H, methyl and ethyl. In certain embodiments, —R⁶ and —R^{6a} of formula (IX-a) and (IX-b) are both —H.

In certain embodiments, Ar of formula (IX-a) and (IX-b) is phenyl. In certain embodiments, Ar of formula (IX-a) and (IX-b) is

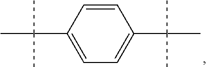

wherein the dashed lines indicate attachment to the remainder of the moiety of formula (IX-a) and (IX-b).

In certain embodiments, W of formula (IX-a) and (IX-b) is C₁₋₂₀ alkyl, optionally interrupted with C₃₋₁₀ cycloalkyl, —C(O)—, —C(O)N(R⁷)—, —O—, —S— and —N(R⁷)—. In certain embodiments, W of formula (IX-a) and (IX-b) is C₁₋₁₀ alkyl, optionally interrupted with C₃₋₁₀ cycloalkyl, —C(O)—, —C(O)N(R⁷)—, —O—, —S— and —N(R⁷)—. In certain embodiments, W of formula (IX-a) and (IX-b) is C₁₋₆ alkyl, optionally interrupted with C₃₋₁₀ cycloalkyl, —C(O)—, —C(O)N(R⁷)—, —O—, —S— and —N(R⁷)—. In certain embodiments, W of formula (IX-a) and (IX-b) is

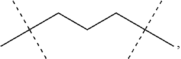

wherein
the dashed lines indicate attachment to the remainder of the moiety of formula (IX-a) or (IX-b), respectively.

In certain embodiments, -Nu is of formula (IX-a) and (IX-b) is —N(R⁷R^{7a}).

In certain embodiments, —R⁷, —R^{7a} and —R^{7b} of formula (IX-a) and (IX-b) are independently of each other selected from the group consisting of —H, methyl, ethyl, n-propyl, isopropyl, n-butyl, isobutyl, sec-butyl and tert-butyl. In certain embodiments, —R⁷, —R^{7a} and —R^{7b} of formula (IX-a) and (IX-b) are independently of each other selected from —H, methyl, ethyl, n-propyl and isopropyl. In certain embodiments, —R⁷, —R^{7a} and —R^{7b} of formula (IX-a) and (IX-b) are independently of each other selected from methyl or ethyl. In certain embodiments, —R⁷, —R^{7a} and —R^{7b} of formula (IX-a) and (IX-b) are both methyl.

In certain embodiments, -L¹- is of formula (IX-c):

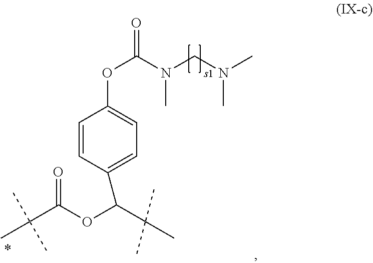

wherein
the dashed line marked with the asterisk indicates attachment to a nitrogen of -D;
the unmarked dashed line indicates attachment to -L²-; and
s1 is an integer selected from the group consisting of 1, 2, 3, 4, 5, 6, 7, 8, 9 and 10.

In certain embodiments, -L¹- is of formula (IX-c), wherein the dashed line marked with the asterisk indicates attachment to a nitrogen of an amine of a lysine side chain of -D.

In certain embodiments, -L¹- is of formula (IX-c), wherein the dashed line marked with the asterisk indicates attachment to the nitrogen of the amine of the N-terminus of -D.

In certain embodiments, s1 of formula (IX-c) is an integer selected from the group consisting of 1, 2, 3, 4 and 5. In certain embodiments, s1 of formula (IX-c) is 1. In certain embodiments, s1 of formula (IX-c) is 2. In certain embodiments, s1 of formula (IX-c) is 3. In certain embodiments, s1 of formula (IX-c) is 4. In certain embodiments, s1 of formula (IX-c) is 5.

In certain embodiments, -L¹- is of formula (IX-d):

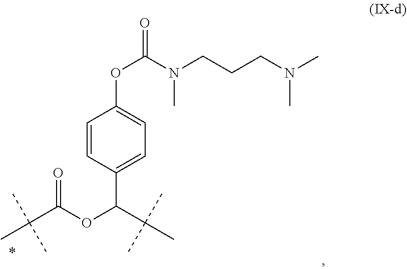

wherein
the dashed line marked with the asterisk indicates attachment to a nitrogen of -D; and
the unmarked dashed line indicates attachment to -L²-.

In certain embodiments, -L¹- is of formula (IX-d), wherein the dashed line marked with the asterisk indicates attachment to a nitrogen of an amine of a lysine side chain of -D.

In certain embodiments, -L¹- is of formula (IX-d), wherein the dashed line marked with the asterisk indicates attachment to the nitrogen of the amine of the N-terminus of -D.

In certain embodiments, -L¹- has a structure as disclosed in WO 2020/206358 A1, which is hereby incorporated by reference in its entirety. Accordingly, in certain embodiments the moiety -L¹- is of formula (X):

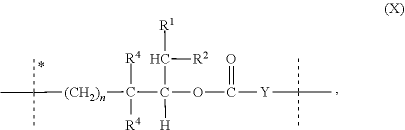

wherein
the unmarked dashed line indicates attachment to -D;
the dashed line marked with the asterisk indicates attachment to -L²-;
n is an integer selected from the group consisting of 0, 1, 2, 3, 4, 5 and 6;
—R¹ and —R² are independently an electron-withdrawing group, alkyl, or —H, and wherein at least one of —R¹ or —R² is an electron-withdrawing group;
each —R⁴ is independently C₁-C₃ alkyl or the two —R⁴ are taken together with the carbon atom to which they are attached to form a 3- to 6-membered ring; and
—Y— is absent when -D is a drug moiety connected through an amine, or —Y— is —N(R⁶)CH₂— when -D is a drug moiety connected through a phenol, alcohol, thiol, thiophenol, imidazole, or non-basic amine; wherein —R⁶ is optionally substituted C₁-C₆ alkyl, optionally substituted aryl, or optionally substituted heteroaryl.

In certain embodiments, n of formula (X) is an integer selected from 1, 2, 3, 4, 5 and 6. In certain embodiments, n of formula (X) is an integer selected from 1, 2 and 3. In certain embodiments, n of formula (X) is an integer from 0, 1, 2 and 3. In certain embodiments, n of formula (X) is 1. In certain embodiments, n of formula (X) is 2. In certain embodiments, n of formula (X) is 3.

In certain embodiments, the electron-withdrawing group of —R¹ and —R² of formula (X) is selected from the group consisting of —CN; —NO₂; optionally substituted aryl; optionally substituted heteroaryl; optionally substituted alkenyl; optionally substituted alkynyl; —COR³, —SOR³, or —SO₂R³, wherein —R³ is —H, optionally substituted alkyl, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl, or both —R⁸ groups are taken together with the nitrogen to which they are attached to form a heterocyclic ring; or —SR⁹, wherein —R⁹ is optionally substituted alkyl, optionally substituted aryl, optionally substituted arylalkyl, optionally substituted heteroaryl, or optionally substituted heteroarylalkyl.

In certain embodiments, the electron-withdrawing group of —R¹ and —R² of formula (X) is —CN. In certain embodiments, the electron-withdrawing group of —R¹ and —R² of formula (X) is —NO₂. In certain embodiments, the electron-withdrawing group of —R¹ and —R² of formula (X) is optionally substituted aryl comprising 6 to 10 carbons. In certain embodiments, the electron-withdrawing group of —R¹ and —R² of formula (X) is optionally substituted phenyl, naphthyl, or anthracenyl. In certain embodiments, the electron-withdrawing group of —R¹ and —R² of formula (X) is optionally substituted heteroaryl comprising 3 to 7 carbons and comprising at least one N, O, or S atom. In certain embodiments, the electron-withdrawing group of —R¹ and —R² of formula (X) is optionally substituted pyrrolyl, pyridyl, pyrimidinyl, imidazolyl, oxazolyl, isoxazolyl, thiazolyl, isothiazolyl, quinolyl, indolyl, or indenyl. In certain embodiments, the electron-withdrawing group of —R¹ and —R² of formula (X) is optionally substituted alkenyl containing 2 to 20 carbon atoms. In certain embodiments, the electron-withdrawing group of —R¹ and —R² of formula (X) is optionally substituted alkynyl comprising 2 to 20 carbon atoms. In certain embodiments, the electron-withdrawing group of —R¹ and —R² of formula (X) is —COR³, —SOR³, or —SO₂R³, wherein —R³ is —H, optionally substituted alkyl comprising 1 to 20 carbon atoms, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl comprising 1 to 20 carbon atoms, or both —R⁸ groups are taken together with the nitrogen to which they are attached to form a heterocyclic ring. In certain embodiments, the electron-withdrawing group of —R¹ and —R² of formula (X) is —SR⁹, wherein —R⁹ is an optionally substituted alkyl comprising 1 to 20 carbon atoms, optionally substituted aryl, optionally substituted arylalkyl, optionally substituted heteroaryl, or optionally substituted heteroarylalkyl.

In certain embodiments, at least one of —R¹ or —R² of formula (X) is —CN, —SOR³ or —SO₂R³. In certain embodiments, at least one of —R¹ and —R² of formula (X) is —CN or —SO₂R³. In certain embodiments, at least one of —R¹ and —R² of formula (X) is —CN or —SO₂R³, wherein —R³ is optionally substituted alkyl, optionally substituted aryl, or —NR⁸₂. In certain embodiments, at least one of —R¹ and —R² of formula (X) is —CN, —SO₂N(CH₃)₂, —SO₂CH₃, phenyl substituted with —SO₂, phenyl substituted with —SO₂ and —Cl, —SO₂N(CH₂CH₂)₂O, —SO₂CH(CH₃)₂, —SO₂N(CH₃)(CH₂CH₃), or —SO₂N(CH₂CH₂OCH₃)₂.

In certain embodiments, each —R⁴ of formula (X) is independently C₁-C₃ alkyl. In certain embodiments, both —R⁴ are methyl.

In certain embodiments, —Y— of formula (X) is absent. In certain embodiments, —Y— of formula (X) is —N(R⁶)CH₂—.

In certain embodiments, -L¹- is of formula (X), wherein n is 1, —R¹ is —CN, —R² is —H, and —R⁴ is —CH₃. In certain embodiments, -L¹- is of formula (X), wherein n is 1, —R¹ is —SO₂N(CH₃)₂, —R² is —H, and —R⁴ is —CH₃. In certain embodiments, -L¹- is of formula (X), wherein n is 1, —R¹ is SO₂CH₃, —R² is —H, and —R⁴ is —CH₃. In certain embodiments, -L¹- is of formula (X), wherein n is 1, —R¹ is —SO₂N(CH₂CH₂)₂CHCH₃, —R² is —H, and —R⁴ is —CH₃. In certain embodiments, -L¹- is of formula (X), wherein n is 1, —R¹ is phenyl substituted with —SO₂, —R² is —H, and —R⁴ is —CH₃. In certain embodiments, -L¹- is of formula (X), wherein n is 1, —R¹ is phenyl substituted with —SO₂ and —Cl, —R² is —H, and —R⁴ is —CH₃. In certain embodiments, -L¹- is of formula (X), wherein n is 1, —R¹ is —SO₂N(CH₂CH₂)₂O, —R² is —H, and —R⁴ is —CH₃. In certain embodiments, -L¹- is of formula (X), wherein n is 1, —R¹ is —SO₂CH(CH₃)₂, —R² is —H, and —R⁴ is —CH₃. In certain embodiments, -L¹- is of formula (X), wherein n is 1, —R¹ is —SO₂N(CH₃)(CH₂CH₃), —R² is —H, and —R⁴ is —CH₃. In certain embodiments, -L¹- is of formula (X), wherein n is 1, —R¹ is —SO₂N(CH₂CH₂OCH₃)₂, —R² is —H, and —R⁴ is —CH₃. In certain embodiments, -L¹- is of formula (X), wherein n is 1, —R¹ is phenyl substituted with —SO₂ and —CH₃, —R² is —H, and —R⁴ is —CH₃.

In certain embodiments, -L¹- is of formula (X), wherein n is 2, —R¹ is —CN, —R² is —H, and —R⁴ is —CH₃. In certain embodiments, -L¹- is of formula (X), wherein n is 2, —R¹ is —SO₂N(CH₃)₂, —R² is —H, and —R⁴ is —CH₃. In certain embodiments, -L¹- is of formula (X), wherein n is 2, —R¹ is SO₂CH₃, —R² is —H, and —R⁴ is —CH₃. In certain embodiments, -L¹- is of formula (X), wherein n is 2, —R¹ is —SO₂N(CH₂CH₂)₂CHCH₃, —R² is —H, and —R⁴ is —CH₃. In certain embodiments, -L¹- is of formula (X), wherein n is 2, —R¹ is phenyl substituted with —SO₂, —R² is —H, and —R⁴ is —CH₃. In certain embodiments, -L¹- is of formula (X), wherein n is 2, —R¹ is phenyl substituted with —SO₂ and —Cl, —R² is —H, and —R⁴ is —CH₃. In certain embodiments, -L¹- is of formula (X), wherein n is 2, —R¹ is —SO₂N(CH₂CH₂)₂O, —R² is —H, and —R⁴ is —CH₃. In certain embodiments, -L¹- is of formula (X), wherein n is 2, —R¹ is —SO₂CH(CH₃)₂, —R² is —H, and —R⁴ is —CH₃. In certain embodiments, -L¹- is of formula (X), wherein n is 2, —R¹ is —SO₂N(CH₃)(CH₂CH₃), —R² is —H, and —R⁴ is —CH₃. In certain embodiments, -L¹- is of formula (X), wherein n is 2, —R¹ is —SO₂N(CH₂CH₂OCH₃)₂, —R² is —H, and —R⁴ is —CH₃. In certain embodiments, -L¹- is of formula (X), wherein n is 2, —R¹ is phenyl substituted with —SO₂ and —CH₃, —R² is —H, and —R⁴ is —CH₃.

In certain embodiments, -L¹- is of formula (X), wherein n is 3, —R¹ is —CN, —R² is —H, and —R⁴ is —CH₃. In certain embodiments, -L¹- is of formula (X), wherein n is 3, —R¹ is —SO₂N(CH₃)₂, —R² is —H, and —R⁴ is —CH₃. In certain embodiments, -L¹- is of formula (X), wherein n is 3, —R¹ is SO₂CH₃, —R² is —H, and —R⁴ is —CH₃. In certain embodiments, -L¹- is of formula (X), wherein n is 3, —R¹ is —SO₂N(CH₂CH₂)₂CHCH₃, —R² is —H, and —R⁴ is —CH₃. In certain embodiments, -L¹- is of formula (X), wherein n is 3, —R¹ is phenyl substituted with —SO₂, —R² is —H, and —R⁴ is —CH₃. In certain embodiments, -L¹- is of formula (X), wherein n is 3, —R¹ is phenyl substituted with —SO₂ and —Cl, —R² is —H, and —R⁴ is —CH₃. In certain embodiments, -L¹- is of formula (X), wherein n is 3, —R¹ is —SO₂N(CH₂CH₂)₂O, —R² is —H, and —R⁴ is —CH₃. In certain embodiments, -L¹- is of formula (X), wherein n is 3, —R¹ is —SO₂CH(CH₃)₂, —R² is —H, and —R⁴ is —CH₃. In certain embodiments, -L¹- is of formula (X), wherein n is 3, —R¹ is —SO₂N(CH₃)(CH₂CH₃), —R² is —H, and —R⁴ is —CH₃. In certain embodiments, -L¹- is of formula (X), wherein n is 3, —R¹ is —SO₂N(CH₂CH₂OCH₃)₂, —R² is —H, and —R⁴ is —CH₃. In certain embodiments, -L¹- is of formula (X), wherein n is 3, —R¹ is phenyl substituted with —SO₂ and —CH₃, —R² is —H, and —R⁴ is —CH₃.

Only in the context of formula (X) the terms used have the following meaning: The term “alkyl” refers to linear, branched, or cyclic saturated hydrocarbon groups of 1 to 20, 1 to 12, 1 to 8, 1 to 6, or 1 to 4 carbon atoms. In certain embodiments an alkyl is linear or branched.

Examples of linear or branched alkyl groups include methyl, ethyl, n-propyl, isopropyl, n-butyl, t-butyl, isobutyl, sec-butyl, n-pentyl, n-hexyl, n-heptyl, n- octyl, n-nonyl, and n-decyl. In certain embodiments an alkyl is cyclic. Examples of cyclic alkyl groups include cyclopropyl, cyclobutyl, cyclopentyl, cyclopentadienyl, and cyclohexyl.

The term “alkoxy” refers to alkyl groups bonded to oxygen, including methoxy, ethoxy, isopropoxy, cyclopropoxy, and cyclobutoxy.

The term “alkenyl” refers to non-aromatic unsaturated hydrocarbons with carbon-carbon double bonds and 2 to 20, 2 to 12, 2 to 8, 2 to 6, or 2 to 4 carbon atoms.

The term “alkynyl” refers to non-aromatic unsaturated hydrocarbons with carbon-carbon triple bonds and 2 to 20, 2 to 12, 2 to 8, 2 to 6, or 2 to 4 carbon atoms.

The term “aryl” refers to aromatic hydrocarbon groups of 6 to 18 carbons, preferably 6 to 10 carbons, including groups such as phenyl, naphthyl, and anthracenyl. The term “heteroaryl” refers to aromatic rings comprising 3 to 15 carbons comprising at least one N, O or S atom, preferably 3 to 7 carbons comprising at least one N, O or S atom, including groups such as pyrrolyl, pyridyl, pyrimidinyl, imidazolyl, oxazolyl, isoxazolyl, thiazolyl, isothiazolyl, quinolyl, indolyl, and indenyl.

In certain embodiments, alkenyl, alkynyl, aryl or heteroaryl moieties may be coupled to the remainder of the molecule through an alkyl linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” or “halo” refers to bromo, fluoro, chloro and iodo.

The term “heterocyclic ring” or “heterocyclyl” refers to a 3- to 15-membered aromatic or non-aromatic ring comprising at least one N, O, or S atom. Examples include piperidinyl, piperazinyl, tetrahydropyranyl, pyrrolidine, and tetrahydrofuranyl, as well as the exemplary groups provided for the term “heteroaryl” above. In certain embodiments a heterocyclic ring or heterocyclyl is non-aromatic. In certain embodiments a heterocyclic ring or heterocyclyl is aromatic.

The term “optionally substituted” refers to a group may be unsubstituted or substituted by one or more (e.g., 1, 2, 3, 4 or 5) of the substituents which may be the same or different. Examples of substituents include alkyl, alkenyl, alkynyl, halogen, —CN, —OR^{aa}, —SR^{aa}, —NR^{aa}R^{bb}, —NO₂, —C═NH(OR^{aa}), —C(O)R^{aa}, —OC(O)R^{aa}, —C(O)OR^{aa}, —C(O)NR^{aa}R^{bb}, —OC(O)NR^{aa}R^{bb}, —NR^{aa}C(O)R^{bb}, —NR^{aa}C(O)OR^{bb}, —S(O)R^{aa}, —S(O)₂R^{aa}, —NR^{aa}(O)R^{bb}, —C(O)NR^{aa}S(O)R^{bb}, —NR^{aa}S(O)₂R^{bb}, —C(O)NR^{aa}S(O)₂R^{bb}, —S(O)NR^{aa}R^{bb}, —S(O)₂NR^{aa}R^{bb}, —P(O)(OR^{aa})(OR^{bb}), heterocyclyl, heteroaryl, or aryl, wherein the alkyl, alkenyl, alkynyl, cycloalkyl, heterocyclyl, heteroaryl, and aryl are each independently optionally substituted by —R^{cc}, wherein —R^{aa} and —R^{bb} are each independently —H, alkyl, alkenyl, alkynyl, heterocyclyl, heteroaryl, or aryl, or —R^{aa} and —R^{bb} are taken together with the nitrogen atom to which they attach to form a heterocyclyl, which is optionally substituted by alkyl, alkenyl, alkynyl, halogen, hydroxyl, alkoxy, or —CN, and wherein: each —R^{oo} is independently alkyl, alkenyl, alkynyl, halogen, heterocyclyl, heteroaryl, aryl, —CN, or —NO₂.

In certain embodiments, -L¹- has a structure as disclosed in WO 2021/136808 A1, which is hereby incorporated by reference in its entirety. Accordingly, in certain embodiments the moiety -L¹- is of formula (XI):

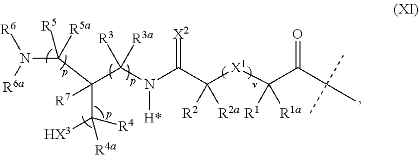

wherein
the dashed line indicates the attachment to the nitrogen of the primary or secondary amine of -D;
v is selected from the group consisting of 0 or 1;
—X¹— is selected from the group consisting of —C(R⁸)(R^{8a})—, —N(R⁹)— and —O—;
═X² is selected from the group consisting of ═O and ═N(R¹⁰);
—X³ is selected from the group consisting of —O, —S and —Se;
each p is independently selected from the group consisting of 0 or 1, provided that at most one p is 0;
—R⁶, —R^{6a}, —R¹⁰ are independently selected from the group consisting of —H, —C(R¹¹)(R^{11a})(R^{11b}) and -T;
—R⁹ is selected from the group consisting of —C(R¹¹)(R^{11a})(R^{11b}) and -T;
—R¹, —R^{1a}, —R², —R^{2a}, —R³, —R^{3a}, —R⁴, —R^{4a}, —R⁵, —R^{5a}, —R⁷, —R⁸ —R^{8a}, —R¹¹, —R^{11a} and —R^{11b} are independently selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12′}), —OC(O)N(R¹²)(R^{12a}), -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
   —R¹², —R^{12a}, —R^{12b} are independently selected from the group consisting of —H, -T, C₁₋₆ alkyl, C₂₋₆ alkenyl and C₂₋₆ alkynyl; wherein -T, C₁₋₆ alkyl, C₂₋₆ alkenyl and C₂₋₆ alkynyl are optionally substituted with one or more —R¹³, which are the same or different and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
      wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R¹³, which are the same or different;
—R¹³ is selected from the group consisting of halogen, —CN, oxo, —C(O)OR¹⁵, —OR¹⁵, —C(O)R¹⁵, —C(O)N(R¹⁵)(R^{15a}), —S(O)₂N(R¹⁵)(R^{15a}), —S(O)N(R¹⁵) (R^{15a}), —S(O)₂R¹⁵, —S(O)R¹⁵, —N(R¹⁵)S(O)₂N(R^{15a})(R^{15b}), —SR¹⁵, —N(R¹⁵)(R^{15a}), —NO₂, —OC(O)R¹⁵, —N(R¹⁵)C(O)R^{15a}, —N(R¹⁵)S(O)₂R^{15a}, —N(R¹⁵)S(O)R^{15a}, —N(R¹⁵)C(O)OR^{15a}, —N(R¹⁵)C(O)N(R^{15a})(R^{15b}), —OC(O)N(R¹⁵)(R^{15a}) and C₁₋₆ alkyl; wherein C₁₋₆ alkyl is optionally substituted with one or more halogen, which are the same or different;
   wherein —R¹⁴, —R^{14a}, —R¹⁵, —R¹⁵ and —R^{15b} are independently selected from the group consisting of —H and C₁₋₆ alkyl; wherein C₁₋₆ alkyl is optionally substituted with one or more halogen, which are the same or different;
optionally, one or more of the pairs —R¹/—R^{1a}, —R²/—R^{2a}, —R³/—R^{3a}, —R⁴/—R^{4a}, —R⁵/—R^{5a} or —R⁸/—R^{8a} are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁸, —R¹/—R⁹, —R²/—R⁹ or —R²/—R¹⁰ are joined together with the atoms to which they are attached to form a ring -A-;
   wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, one or more of the pairs —R³/—R⁶, —R⁴/—R⁶, —R⁵/—R⁶, —R⁶/—R^{6a} or —R⁶/—R⁷ form together with the atoms to which they are attached a ring -A′-;
   wherein -A′- is selected from the group consisting of 3- to 10-membered heterocyclyl and 8- to 11-membered heterobicyclyl; and
-L¹- is substituted with -L²- and optionally further substituted provided that the hydrogen marked with the asterisk in formula (XI) is not replaced by a substituent.

In certain embodiments, the dashed line in formula (XI) indicates attachment to a nitrogen of a primary amine of -D. In certain embodiments, the dashed line in formula (XI) indicates attachment to a nitrogen of a secondary amine of -D.

In certain embodiments, —X³ of formula (XI) is —O. In certain embodiments, —X³ of formula (XI) is —S. In certain embodiments, —X³ of formula (XI) is —Se.

In certain embodiments, —R⁶ of formula (XI) is —H. In certain embodiments, —R⁶ of formula (XI) is —C(R¹¹)(R^{11a})(R^{11b}). In certain embodiments, —R⁶ of formula (XI) is -T.

In certain embodiments, —R^{6a} of formula (XI) is —H. In certain embodiments, —R^{6a} of formula (XI) is —C(R¹¹)(R^{11a})(R^{11b}). In certain embodiments, —R^{6a} of formula (XI) is -T.

In certain embodiments, both —R⁶ and —R^{6a} of formula (XI) are —H.

In certain embodiments, v of formula (XI) is 0. In certain embodiments, v of formula (XI) is 1.

In certain embodiments, —X¹— of formula (XI) is —C(R⁸)(R^{8a})—. In certain embodiments, —X¹— of formula (XI) is —N(R⁹)—. In certain embodiments, —X¹— of formula (XI) is —O—.

In certain embodiments, ═X² of formula (XI) is ═O. In certain embodiments, ═X² of formula (XI) is ═N(R¹⁰).

In certain embodiments, —R⁹ of formula (XI) is —C(R¹¹)(R^{11a})(R^{11b}). In certain embodiments, —R⁹ of formula (XI) is -T.

In certain embodiments, —R¹⁰ of formula (XI) is —H. In certain embodiments, —R¹⁰ of formula (XI) is —C(R¹¹)(R^{11a})(R^{11b}). In certain embodiments, —R¹⁰ of formula (XI) is -T.

In certain embodiments, —R¹ of formula (XI) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R¹ of formula (XI) is —H. In certain embodiments, —R¹ of formula (XI) is halogen. In certain embodiments, —R¹ of formula (XI) is -T. In certain embodiments, —R¹ of formula (XI) is C₁₋₆ alkyl. In certain embodiments, —R¹ of formula (XI) is C₂₋₆ alkenyl. In certain embodiments, —R¹ of formula (XI) is C₂₋₆ alkynyl. In certain embodiments, —R¹ of formula (XI) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{1a} of formula (XI) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{1a} of formula (XI) is —H. In certain embodiments, —R^{1a} of formula (XI) is halogen. In certain embodiments, —R^{1a} of formula (XI) is -T. In certain embodiments, —R^{1a} of formula (XI) is C₁₋₆ alkyl. In certain embodiments, —R^{1a} of formula (XI) is C₂₋₆ alkenyl. In certain embodiments, —R^{1a} of formula (XI) is C₂₋₆ alkynyl. In certain embodiments, —R^{1a} of formula (XI) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R² of formula (XI) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R² of formula (XI) is —H. In certain embodiments, —R² of formula (XI) is halogen. In certain embodiments, —R² of formula (XI) is -T. In certain embodiments, —R² of formula (XI) is C₁₋₆ alkyl. In certain embodiments, —R² of formula (XI) is C₂₋₆ alkenyl. In certain embodiments, —R² of formula (XI) is C₂₋₆ alkynyl. In certain embodiments, —R² of formula (XI) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{2a} of formula (XI) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{2a} of formula (XI) is —H. In certain embodiments, —R^{2a} of formula (XI) is halogen. In certain embodiments, —R^{2a} of formula (XI) is -T. In certain embodiments, —R^{2a} of formula (XI) is C₁₋₆ alkyl. In certain embodiments, —R^{2a} of formula (XI) is C₂₋₆ alkenyl. In certain embodiments, —R^{2a} of formula (XI) is C₂₋₆ alkynyl. In certain embodiments, —R^{2a} of formula (XI) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R³ of formula (XI) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R³ of formula (XI) is —H. In certain embodiments, —R³ of formula (XI) is halogen. In certain embodiments, —R³ of formula (XI) is -T. In certain embodiments, —R³ of formula (XI) is C₁₋₆ alkyl. In certain embodiments, —R³ of formula (XI) is C₂₋₆ alkenyl. In certain embodiments, —R³ of formula (XI) is C₂₋₆ alkynyl. In certain embodiments, —R³ of formula (XI) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{3a} of formula (XI) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{3a} of formula (XI) is —H. In certain embodiments, —R^{3a} of formula (XI) is halogen. In certain embodiments, —R^{3a} of formula (XI) is -T. In certain embodiments, —R^{3a} of formula (XI) is C₁₋₆ alkyl. In certain embodiments, —R^{3a} of formula (XI) is C₂₋₆ alkenyl. In certain embodiments, —R^{3a} of formula (XI) is C₂₋₆ alkynyl. In certain embodiments, —R^{3a} of formula (XI) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R⁴ of formula (XI) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R⁴ of formula (XI) is —H. In certain embodiments, —R⁴ of formula (XI) is halogen. In certain embodiments, —R⁴ of formula (XI) is -T. In certain embodiments, —R⁴ of formula (XI) is C₁₋₆ alkyl. In certain embodiments, —R⁴ of formula (XI) is C₂₋₆ alkenyl. In certain embodiments, —R⁴ of formula (XI) is C₂₋₆ alkynyl. In certain embodiments, —R⁴ of formula (XI) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{4a} of formula (XI) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{4a} of formula (XI) is —H. In certain embodiments, —R^{4a} of formula (XI) is halogen. In certain embodiments, —R^{4a} of formula (XI) is -T. In certain embodiments, —R^{4a} of formula (XI) is C₁₋₆ alkyl. In certain embodiments, —R^{4a} of formula (XI) is C₂₋₆ alkenyl. In certain embodiments, —R^{4a} of formula (XI) is C₂₋₆ alkynyl. In certain embodiments, —R^{4a} of formula (XI) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R⁵ of formula (XI) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R⁵ of formula (XI) is —H. In certain embodiments, —R⁵ of formula (XI) is halogen. In certain embodiments, —R⁵ of formula (XI) is -T. In certain embodiments, —R⁵ of formula (XI) is C₁₋₆ alkyl. In certain embodiments, —R⁵ of formula (XI) is C₂₋₆ alkenyl. In certain embodiments, —R⁵ of formula (XI) is C₂₋₆ alkynyl. In certain embodiments, —R⁵ of formula (XI) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{5a} of formula (XI) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{5a} of formula (XI) is —H. In certain embodiments, —R^{5a} of formula (XI) is halogen. In certain embodiments, —R^{5a} of formula (XI) is -T. In certain embodiments, —R^{5a} of formula (XI) is C₁₋₆ alkyl. In certain embodiments, —R^{5a} of formula (XI) is C₂₋₆ alkenyl. In certain embodiments, —R^{5a} of formula (XI) is C₂₋₆ alkynyl. In certain embodiments, —R^{5a} of formula (XI) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R⁷ of formula (XI) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R⁷ of formula (XI) is —H. In certain embodiments, —R⁷ of formula (XI) is halogen. In certain embodiments, —R⁷ of formula (XI) is -T.

In certain embodiments, —R⁷ of formula (XI) is C₁₋₆ alkyl. In certain embodiments, —R⁷ of formula (XI) is C₂₋₆ alkenyl. In certain embodiments, —R⁷ of formula (XI) is C₂₋₆ alkynyl. In certain embodiments, —R⁷ of formula (XI) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R⁸ of formula (XI) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R⁸ of formula (XI) is —H. In certain embodiments, —R⁸ of formula (XI) is halogen. In certain embodiments, —R⁸ of formula (XI) is -T. In certain embodiments, —R⁸ of formula (XI) is C₁₋₆ alkyl. In certain embodiments, —R⁸ of formula (XI) is C₂₋₆ alkenyl. In certain embodiments, —R⁸ of formula (XI) is C₂₋₆ alkynyl. In certain embodiments, —R⁸ of formula (XI) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{8a} of formula (XI) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{8a} of formula (XI) is —H. In certain embodiments, —R^{8a} of formula (XI) is halogen. In certain embodiments, —R^{8a} of formula (XI) is -T. In certain embodiments, —R^{8a} of formula (XI) is C₁₋₆ alkyl. In certain embodiments, —R^{8a} of formula (XI) is C₂₋₆ alkenyl. In certain embodiments, —R^{8a} of formula (XI) is C₂₋₆ alkynyl. In certain embodiments, —R^{8a} of formula (XI) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R¹¹ of formula (XI) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R¹¹ of formula (XI) is —H. In certain embodiments, —R¹¹ of formula (XI) is halogen. In certain embodiments, —R¹¹ of formula (XI) is -T. In certain embodiments, —R¹¹ of formula (XI) is C₁₋₆ alkyl. In certain embodiments, —R¹¹ of formula (XI) is C₂₋₆ alkenyl. In certain embodiments, —R¹¹ of formula (XI) is C₂₋₆ alkynyl. In certain embodiments, —R¹¹ of formula (XI) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{11a} of formula (XI) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{11a} of formula (XI) is —H. In certain embodiments, —R^{11a} of formula (XI) is halogen. In certain embodiments, —R^{11a} of formula (XI) is -T. In certain embodiments, —R^{11a} of formula (XI) is C₁₋₆ alkyl. In certain embodiments, —R^{11a} of formula (XI) is C₂₋₆ alkenyl. In certain embodiments, - of formula (XI) is C₂₋₆ alkynyl. In certain embodiments, —R^{11a} of formula (XI) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R¹² of formula (XI) is selected from the group consisting of —H, -T, C₁₋₆ alkyl, C₂₋₆ alkenyl and C₂₋₆ alkynyl. In certain embodiments, —R¹² of formula (XI) is —H. In certain embodiments, —R¹² of formula (XI) is -T. In certain embodiments, —R¹² of formula (XI) is C₁₋₆ alkyl. In certain embodiments, —R¹² of formula (XI) is C₂₋₆ alkenyl. In certain embodiments, —R¹² of formula (XI) is C₂₋₆ alkynyl.

In certain embodiments, —R^{12a} of formula (XI) is selected from the group consisting of —H, -T, C₁₋₆ alkyl, C₂₋₆ alkenyl and C₂₋₆ alkynyl. In certain embodiments, —R^{12a} of formula (XI) is —H. In certain embodiments, —R^{12a} of formula (XI) is -T. In certain embodiments, —R^{12a} of formula (XI) is C₁₋₆ alkyl. In certain embodiments, —R^{12a} of formula (XI) is C₂₋₆ alkenyl. In certain embodiments, —R^{12a} of formula (XI) is C₂₋₆ alkynyl.

In certain embodiments, —R^{12b} of formula (XI) is selected from the group consisting of —H, -T, C₁₋₆ alkyl, C₂₋₆ alkenyl and C₂₋₆ alkynyl. In certain embodiments, —R^{12b} of formula (XI) is —H. In certain embodiments, —R^{12b} of formula (XI) is -T. In certain embodiments, —R^{12b} of formula (XI) is C₁₋₆ alkyl. In certain embodiments, —R^{12b} of formula (XI) is C₂₋₆ alkenyl. In certain embodiments, —R^{12b} of formula (XI) is C₂₋₆ alkynyl.

In certain embodiments, T of formula (XI) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, T of formula (XI) is phenyl. In certain embodiments, T of formula (XI) is naphthyl. In certain embodiments, T of formula (XI) is indenyl. In certain embodiments, T of formula (XI) is indanyl. In certain embodiments, T of formula (XI) is tetralinyl. In certain embodiments, T of formula (XI) is tetralinyl. In certain embodiments, T of formula (XI) is C₃₋₁₀ cycloalkyl. In certain embodiments, T of formula (XI) is 3- to 10-membered heterocyclyl. In certain embodiments, T of formula (XI) is 8- to 11-membered heterobicyclyl.

In certain embodiments, T of formula (XI) is substituted with one or more —R¹³ of formula (XI), which are the same of different.

In certain embodiments, T of formula (XI) is substituted with one —R¹³ of formula (XI). In certain embodiments, T of formula (XI) is not substituted with —R¹³.

In certain embodiments, —R¹³ of formula (XI) is selected from the group consisting of halogen, —CN, oxo, —C(O)OR¹⁵, —OR¹⁵, —C(O)R¹⁵, —C(O)N(R¹⁵)(R^{15a}), —S(O)₂N(R¹⁵)(R^{15a}), —S(O)N(R¹⁵)(R^{15a}), —S(O)₂R¹⁵, —S(O)R¹⁵, —N(R¹⁵)S(O)₂N(R^{15a})(R^{15b}), —SR¹⁵, —N(R¹⁵)(R^{5a}), —NO₂, —OC(O)R¹⁵, —N(R¹⁵)C(O)R^{15a}, —N(R⁵)S(O)₂R^{15a}, —N(R¹⁵)S(O)R^{15a}, —N(R¹⁵)C(O)OR^{15a}, —N(R¹⁵)C(O)N(R^{15a})(R^{15b}), —OC(O)N(R¹⁵)(R^{15a}) and C₁₋₆ alkyl. In certain embodiments, —R¹³ of formula (XI) is halogen. In certain embodiments, —R¹³ of formula (XI) is —CN. In certain embodiments, —R¹³ of formula (XI) is oxo. In certain embodiments, —R¹³ of formula (XI) is —C(O)OR¹⁵. In certain embodiments, —R¹³ of formula (XI) is —OR¹⁵. In certain embodiments, —R¹³ of formula (XI) is —C(O)R¹⁵. In certain embodiments, —R¹³ of formula (XI) is —C(O)N(R¹⁵)(R^{15a}). In certain embodiments, —R¹³ of formula (XI) is —S(O)₂N(R¹⁵)(R^{15a}). In certain embodiments, —R¹³ of formula (XI) is —S(O)N(R¹⁵)(R^{15a}). In certain embodiments, —R¹³ of formula (XI) is —S(O)₂R¹⁵. In certain embodiments, —R¹³ of formula (XI) is —S(O)R¹⁵. In certain embodiments, —R¹³ of formula (XI) is —N(R¹⁵)S(O)₂N(R^{15a})(R^{15b}). In certain embodiments, —R¹³ of formula (XI) is —SR¹⁵. In certain embodiments, —R¹³ of formula (XI) is —N(R¹⁵)(R^{15a}). In certain embodiments, —R¹³ of formula (XI) is —NO₂. In certain embodiments, —R¹³ of formula (XI) is —OC(O)R¹⁵. In certain embodiments, —R¹³ of formula (XI) is —N(R¹⁵)C(O)R^{15a}. In certain embodiments, —R¹³ of formula (XI) is —N(R¹⁵)S(O)₂R^{15a}. In certain embodiments, —R¹³ of formula (XI) is —N(R¹⁵)S(O)R^{15a}. In certain embodiments, —R¹³ of formula (XI) is —N(R¹⁵)C(O)OR^{5a}. In certain embodiments, —R¹³ of formula (XI) is —N(R¹⁵)C(O)N(R^{15a})(R^{15b}). In certain embodiments, —R¹³ of formula (XI) is —OC(O)N(R¹⁵)(R^{15a}). In certain embodiments, —R¹³ of formula (XI) is C₁₋₆ alkyl.

In certain embodiments, —R¹⁴ of formula (XI) is selected from the group consisting of —H and C₁₋₆ alkyl. In certain embodiments, —R¹⁴ of formula (XI) is —H. In certain embodiments, —R¹⁴ of formula (XI) is C₁₋₆ alkyl.

In certain embodiments, —R^{14a} of formula (XI) is selected from the group consisting of —H and C₁₋₆ alkyl. In certain embodiments, —R^{14a} of formula (XI) is —H. In certain embodiments, —R^{14a} of formula (XI) is C₁₋₆ alkyl.

In certain embodiments, —R¹⁵ of formula (XI) is selected from the group consisting of —H and C₁₋₆ alkyl. In certain embodiments, —R¹⁵ of formula (XI) is —H. In certain embodiments, —R¹⁵ of formula (XI) is C₁₋₆ alkyl.

In certain embodiments, —R^{a15} of formula (XI) is selected from the group consisting of —H and C₁₋₆ alkyl. In certain embodiments, —R^{a15} of formula (XI) is —H. In certain embodiments, —R^{a15} of formula (XI) is C₁₋₆ alkyl.

In certain embodiments, —R^{15b} of formula (XI) is selected from the group consisting of —H and C₁₋₆ alkyl. In certain embodiments, —R^{15b} of formula (XI) is —H. In certain embodiments, —R^{15b} of formula (XI) is C₁₋₆ alkyl.

In certain embodiments, —R¹ and —R^{1a} of formula (XI) are joined together with the atom to which they are attached to form C₃₋₁₀ cycloalkyl. In certain embodiments, —R¹ and —R^{1a} of formula (XI) are joined together with the atom to which they are attached to form 3- to 10-membered heterocyclyl. In certain embodiments, —R¹ and —R^{1a} of formula (XI) are joined together with the atom to which they are attached to form an 8- to 11-membered heterobicyclyl.

In certain embodiments, —R² and —R^{2a} of formula (XI) are joined together with the atom to which they are attached to form a C₃₋₁₀ cycloalkyl. In certain embodiments, —R² and —R^{2a} of formula (XI) are joined together with the atom to which they are attached to form a 3- to 10-membered heterocyclyl. In certain embodiments, —R² and —R^{2a} of formula (XI) are joined together with the atom to which they are attached to form an 8- to 11-membered heterobicyclyl.

In certain embodiments, —R³ and —R^{3a} of formula (XI) are joined together with the atom to which they are attached to form a C₃₋₁₀ cycloalkyl. In certain embodiments, —R³ and —R^{3a} of formula (XI) are joined together with the atom to which they are attached to form a 3- to 10-membered heterocyclyl. In certain embodiments, —R³ and —R^{3a} of formula (XI) are joined together with the atom to which they are attached to form an 8- to 11-membered heterobicyclyl.

In certain embodiments, —R⁴ and —R^{4a} of formula (XI) are joined together with the atom to which they are attached to form a C₃₋₁₀ cycloalkyl. In certain embodiments, —R⁴ and —R^{4a} of formula (XI) are joined together with the atom to which they are attached to form a 3- to 10-membered heterocyclyl. In certain embodiments, —R⁴ and —R^{4a} of formula (XI) are joined together with the atom to which they are attached to form an 8- to 11-membered heterobicyclyl.

In certain embodiments, —R⁵ and —R^{5a} of formula (XI) are joined together with the atom to which they are attached to form a C₃₋₁₀ cycloalkyl. In certain embodiments, —R⁵ and —R^{5a} of formula (XI) are joined together with the atom to which they are attached to form a 3- to 10-membered heterocyclyl. In certain embodiments, —R⁵ and —R^{5a} of formula (XI) are joined together with the atom to which they are attached to form an 8- to 11-membered heterobicyclyl.

In certain embodiments, —R⁸ and —R^{8a} of formula (XI) are joined together with the atom to which they are attached to form a C₃₋₁₀ cycloalkyl. In certain embodiments, —R⁸ and —R^{8a} of formula (XI) are joined together with the atom to which they are attached to form a 3- to 10-membered heterocyclyl. In certain embodiments, —R⁸ and —R^{8a} of formula (XI) are joined together with the atom to which they are attached to form an 8- to 11-membered heterobicyclyl.

In certain embodiments, —R¹ and —R² of formula (XI) are joined together with the atoms to which they are attached to form a ring -A- of formula (XI).

In certain embodiments, —R¹ and —R⁸ of formula (XI) are joined together with the atoms to which they are attached to form a ring -A- of formula (XI).

In certain embodiments, —R¹ and —R⁹ of formula (XI) are joined together with the atoms to which they are attached to form a ring -A- of formula (XI).

In certain embodiments, —R² and —R⁹ of formula (XI) are joined together with the atoms to which they are attached to form a ring -A- of formula (XI).

In certain embodiments, —R² and —R¹⁰ of formula (XI) are joined together with the atoms to which they are attached to form a ring -A- of formula (XI).

In certain embodiments, -A- of formula (XI) is phenyl. In certain embodiments, -A- of formula (XI) is naphthyl. In certain embodiments, -A- of formula (XI) is indenyl. In certain embodiments, -A-of formula (XI) is indanyl. In certain embodiments, -A- of formula (XI) is tetralinyl. In certain embodiments, -A- of formula (XI) is C₃₋₁₀ cycloalkyl. In certain embodiments, -A- of formula (XI) is 3- to 10-membered heterocyclyl. In certain embodiments, -A- of formula (XI) is 8- to 11-membered heterobicyclyl.

In certain embodiments, —R³ and —R⁶ of formula (XI) are joined together with the atoms to which they are attached to form a ring -A′- of formula (XI).

In certain embodiments, —R⁴ and —R⁶ of formula (XI) are joined together with the atoms to which they are attached to form a ring -A′- of formula (XI).

In certain embodiments, —R⁵ and —R⁶ of formula (XI) are joined together with the atoms to which they are attached to form a ring -A′- of formula (XI).

In certain embodiments, —R⁶ and —R^{6a} of formula (XI) are joined together with the atoms to which they are attached to form a ring -A′- of formula (XI).

In certain embodiments, —R⁶ and —R⁷ of formula (XI) are joined together with the atoms to which they are attached to form a ring -A′- of formula (XI).

In certain embodiments, -A′- of formula (XI) is 3- to 10-membered heterocyclyl. In certain embodiments, -A′- of formula (XI) is 8- to 11-membered heterobicyclyl.

In certain embodiments, -L¹- is of formula (XIa):

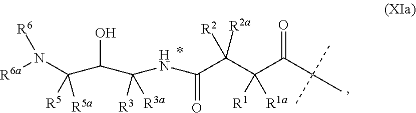

wherein
the dashed line indicates attachment to the nitrogen of the primary or secondary amine of -D;
—R¹, —R^{a} —R², R^{2a} —R³, R^{3a}, —R⁵, —R^{5a} —R⁶ and —R^{6a} are used as defined in formula (XI); and -L¹- is substituted with -L²- and is optionally further substituted, provided that the hydrogen marked with the asterisk in formula (XIa) is not replaced by a substituent.

In certain embodiments, the dashed line in formula (XIa) indicates attachment to a nitrogen of a primary amine of -D. In certain embodiments, the dashed line in formula (XIa) indicates attachment to a nitrogen of a secondary amine of -D.

In certain embodiments, —R¹ is —H. In certain embodiments, —R^{1a} is —H. In certain embodiments, —R² is —H. In certain embodiments, —R^{2a} is —H. In certain embodiments, —R³ is —H. In certain embodiments, —R^{3a} is —H. In certain embodiments, —R⁵ is —H. In certain embodiments, —R^{5a} is —H. In certain embodiments, —R⁶ is —H. In certain embodiments, —R^{6a} is —H.

In certain embodiments, -L¹- of formula (XIa) is not further substituted.

In certain embodiments, —R¹ is —H, which —H is substituted with -L²-. In certain embodiments, —Ria is —H, which —H is substituted with -L²-. In certain embodiments, —R² is —H, which —H is substituted with -L²-. In certain embodiments, —R^{2a} is —H, which —H is substituted with -L²-. In certain embodiments, —R³ is —H, which —H is substituted with -L²-. In certain embodiments, —R^{3a} is —H, which —H is substituted with -L²-. In certain embodiments, —R⁵ is —H, which —H is substituted with -L²-. In certain embodiments, —R^{5a} is —H, which —H is substituted with -L²-. In certain embodiments, —R⁶ is —H, which —H is substituted with -L²-. In certain embodiments, —R^{6a} is —H, which —H is substituted with -L²-.

In certain embodiments, -L¹- is of formula (XIb):

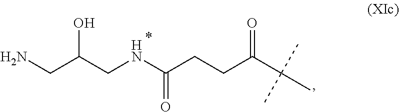

wherein
the dashed line indicates attachment to the nitrogen of the primary or secondary amine of -D; and
-L¹- is substituted with -L²- and is optionally further substituted, provided that the hydrogen marked with the asterisk in formula (XIb) is not replaced by a substituent.

In certain embodiments, the dashed line in formula (XIb) indicates attachment to a nitrogen of a primary amine of -D. In certain embodiments, the dashed line in formula (XIb) indicates attachment to a nitrogen of a secondary amine of -D.

In certain embodiments, -L¹- of formula (XIb) is not further substituted.

In certain embodiments, -L¹- is of formula (XIc):

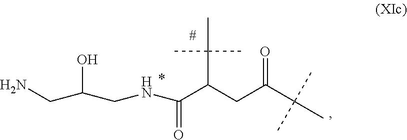

wherein
the unmarked dashed line indicates attachment to the nitrogen of the primary or secondary amine of -D, and
the dashed line marked with # indicates attachment to -L²-.

In certain embodiments, the unmarked dashed line in formula (XIc) indicates attachment to a nitrogen of a primary amine of -D. In certain embodiments, the unmarked dashed line in formula (XIc) indicates attachment to a nitrogen of a secondary amine of -D.

In certain embodiments, -L¹- has a structure as disclosed in WO 2020/254603 A1, which is hereby incorporated by reference in its entirety. Accordingly, in certain embodiments the moiety -L¹- is of formula (XII):

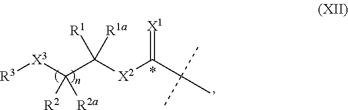

wherein
the dashed line indicates the attachment to the rc-electron-pair-donating heteroaromatic N of -D;
n is an integer selected from the group consisting of 0, 1, 2, 3 and 4;
═X¹ is selected from the group consisting of ═O, ═S and ═N(R⁴);
—X²- is selected from the group consisting of —O—, —S—, —N(R⁵)— and —C(R⁶)(R^{6a})—;
—X³- is selected from the group consisting of

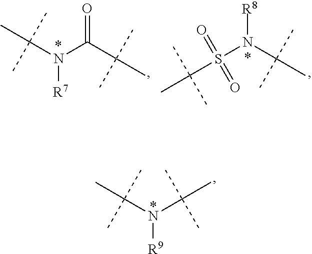

—C(R¹⁰)(R^{10a})—, —C(R¹¹)(R^{11a})—C(R¹²)(R^{12a})—, —O— and —C(O)—;
—R¹, —R^{1a}, —R⁶, —R^{6a}, —R¹⁰, —R^{10a}, —R¹¹, —R^{11a}, —R¹², —R^{12a} and each of —R² and —R^{2a} are independently selected from the group consisting of —H, —C(O)OH, halogen, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
—R³, —R⁴, —R⁵, —R⁷, —R⁸ and —R⁹ are independently selected from the group consisting of —H, -T, —CN, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —N(R¹⁴)—, —OC(OR¹⁴)(R^{14a})—, —N(R¹⁴)C(O)N(R^{14a})— and —OC(O)N(R¹⁴)—;
   each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R¹³, which are the same or different;
   wherein —R¹³ is selected from the group consisting of —H, —NO₂, —OCH₃, —CN, —N(R¹⁴)(R^{14a}), —OH, —C(O)OH and C₁₋₆ alkyl; wherein C₁₋₆ alkyl is optionally substituted with one or more halogen, which are the same or different;
   wherein —R¹⁴ and —R^{14a} are independently selected from the group consisting of —H and C₁₋₆ alkyl; wherein C₁₋₆ alkyl is optionally substituted with one or more halogen, which are the same or different;
optionally, one or more of the pairs —R¹/—R^{1a}, —R²/—R^{2a}, two adjacent —R², —R⁶/—R^{6a}, —R¹⁰/—R^{10a}, —R¹¹/—R^{11a} and —R¹²/—R^{12a} joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁵, —R¹/—R⁶, —R¹/—R⁹, —R¹/—R¹⁰, —R³/—R^{6a}, —R⁴/—R⁵, —R⁴/—R⁶, —R⁵/—R¹⁰ and —R⁶/—R¹⁰ are joined together with the atoms to which they are attached to form a ring -A-;
   wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, —R¹ and an adjacent —R² form a carbon-carbon double bond provided that n is selected from the group consisting of 1, 2, 3 and 4;
optionally, two adjacent —R² form a carbon-carbon double bond provided that n is selected from the group consisting of 2, 3 and 4;
provided that —X²— is —N(R⁵)—, —X³— is selected from the group consisting of

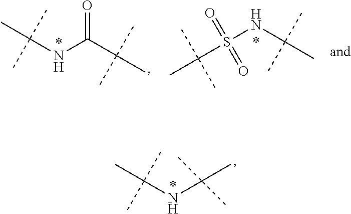

and the distance between the nitrogen atom marked with an asterisk and the carbon atom marked with an asterisk in formula (XII) is 5, 6 or 7 atoms and if present the carbon-carbon double bond formed between —R¹ and —R² or two adjacent —R² is in a cis configuration; and each -L¹- is substituted with -L²- and optionally further substituted.

It is understood that the “N” in the phrase “rc-electron-pair-donating heteroaromatic N” refers to nitrogen.

It is understood that two adjacent —R² in formula (XII) can only exist if n is at least 2.

It is understood that the expression “distance between the nitrogen atom marked with an asterisk and the carbon atom marked with an asterisk” refers to the total number of atoms in the shortest distance between the nitrogen and carbon atoms marked with the asterisk and also includes the nitrogen and carbon atoms marked with the asterisk. For example, in the structure below, n is 1 and the distance between the nitrogen marked with an asterisk and the carbon marked with an asterisk is 5:

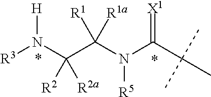

and in the structure below, n is 2, —R¹ and —R^{1a} form a cyclohexyl and the distance between the nitrogen marked with an asterisk and the carbon marked with an asterisk is 6:

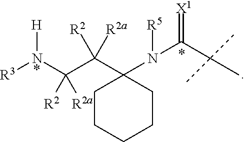

In certain embodiments, ═X¹ of formula (XII) is ═O. In certain embodiments, ═X¹ of formula (XII) is ═S. In certain embodiments, ═X¹ of formula (XII) is ═N(R⁴).

In certain embodiments, —X²— of formula (XII) is —O—. In certain embodiments, —X²— of formula (XII) is —S—. In certain embodiments, —X²— of formula (XII) is —N(R⁵)—. In certain embodiments, —X²— of formula (XII) is —C(R⁶)(R^{6a})—.

In certain embodiments, —X³— of formula (XII) is

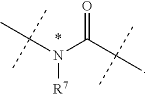

In certain embodiments, —X³— of formula (XII) is

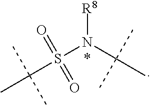

In certain embodiments, —X³— of formula (XII) is

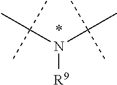

In certain embodiments, —X³— of formula (XII) is —C(R¹⁰)(R^{10a})—. In certain embodiments, —X³— of formula (XII) is —C(R¹¹)(R^{11a})—C(R¹²)(R^{12a})—. In certain embodiments, —X³— of formula (XII) is —O—. In certain embodiments, —X³— of formula (XII) is —C(O)—.

In certain embodiments, —X²— of formula (XII) is —N(R⁵)—, —X³— of formula (XII) is

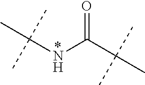

and the distance between the nitrogen atom marked with an asterisk and the carbon atom marked with an asterisk in formula (XII) is 5 atoms.

In certain embodiments, —X² of formula (XII) is —N(R⁵)—, —X³— of formula (XII) is

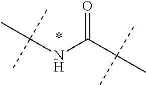

and the distance between the nitrogen atom marked with an asterisk and the carbon atom marked with an asterisk in formula (XII) is 6 atoms.

In certain embodiments, —X²— of formula (XII) is —N(R⁵)—, —X³— of formula (XII) is

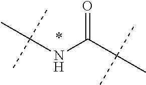

and the distance between the nitrogen atom marked with an asterisk and the carbon atom marked with an asterisk in formula (XII) is 7 atoms.

In certain embodiments, —X²— of formula (XII) is —N(R⁵)—, —X³— of formula (XII) is

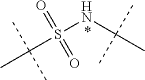

and the distance between the nitrogen atom marked with an asterisk and the carbon atom marked with an asterisk in formula (XII) is 5 atoms.

In certain embodiments, —X²— of formula (XII) is —N(R⁵)—, —X³— of formula (XII) is

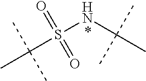

and the distance between the nitrogen atom marked with an asterisk and the carbon atom marked with an asterisk in formula (XII) is 6 atoms.

In certain embodiments, —X²— of formula (XII) is —N(R⁵)—, —X³— of formula (XII) is

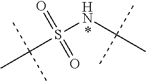

and the distance between the nitrogen atom marked with an asterisk and the carbon atom marked with an asterisk in formula (XII) is 7 atoms.

In certain embodiments, —X²— of formula (XII) is —N(R⁵)—, —X³— of formula (XII) is

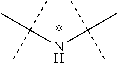

and the distance between the nitrogen atom marked with an asterisk and the carbon atom marked with an asterisk in formula (XII) is 5 atoms.

In certain embodiments, —X²— of formula (XII) is —N(R⁵)—, —X³— of formula (XII) is

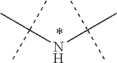

and the distance between the nitrogen atom marked with an asterisk and the carbon atom marked with an asterisk in formula (XII) is 6 atoms.

In certain embodiments, —X²— of formula (XII) is —N(R⁵)—, —X³— of formula (XII) is

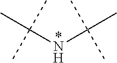

and the distance between the nitrogen atom marked with an asterisk and the carbon atom marked with an asterisk in formula (XII) is 7 atoms.

In certain embodiments, —X²— of formula (XII) is —N(R⁵)—, —X³— of formula (XII) is

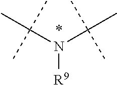

and the distance between the nitrogen atom marked with an asterisk and the carbon atom marked with an asterisk in formula (XII) is 5 atoms.

In certain embodiments, —X²— of formula (XII) is —N(R⁵)—, —X³— of formula (XII) is

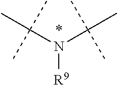

and the distance between the nitrogen atom marked with an asterisk and the carbon atom marked with an asterisk in formula (XII) is 6 atoms.

In certain embodiments, —X²— of formula (XII) is —N(R⁵)—, —X³— of formula (XII) is

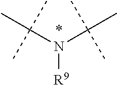

and the distance between the nitrogen atom marked with an asterisk and the carbon atom marked with an asterisk in formula (XII) is 7 atoms.

In certain embodiments, ═X¹ of formula (XII) is ═O, —X²— of formula (XII) is —C(R⁶)(R^{6a})—, —X³— of formula (XII) is

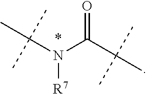

and —R³ does not comprise an amine.

In certain embodiments, —R¹, —R^{1a}, —R⁶, —R^{6a} —R¹⁰, —R^{10a}, —R¹¹, —R^{11a}, —R¹², —R^{12a} of formula (XII) and each of —R² and —R^{2a} of formula (XII) are independently selected from the group consisting of —H, —C(O)OH, halogen, —CN, —OH, C₁₋₆ alkyl, C₂₋₆ alkenyl and C₂₋₆ alkynyl.

In certain embodiments, -L¹- has a structure as disclosed in WO 2020/254602 A1, which is hereby incorporated by reference in its entirety. Said -L¹- is suitable for drugs D that when bound to -L¹- comprise an electron-donating heteroaromatic N⁺ moiety or a quaternary ammonium cation and becomes a moiety -D⁺ upon linkage. Accordingly, in certain embodiments -L¹- is of formula (XII):

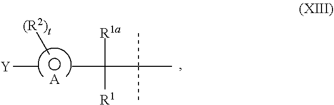

wherein
the dashed line indicates the attachment to the N⁺ of -D⁺;
t is selected from the group consisting of 0, 1, 2, 3, 4, 5 and 6;
-A- is a ring selected from the group consisting of monocyclic or bicyclic aryl and heteroaryl, provided that -A- is connected to —Y and —C(R¹)(R^{1a})—via carbon atoms; wherein said monocyclic or bicyclic aryl and heteroaryl are optionally substituted with one or more
—R², which are the same or different;
—R¹, —R^{1a} and each —R² are independently selected from the group consisting of —H, —C(O)OH, -halogen, —NO₂, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})— and —OC(O)N(R⁴)—;
   each -T- is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each -T- is independently optionally substituted with one or more —R³, which are the same or different;
   wherein —R³ is selected from the group consisting of —H, —NO₂, —OCH₃, —CN, —N(R⁴)(R^{4a}), —OH, —C(O)OH and C₁₋₆ alkyl; wherein C₁₋₆ alkyl is optionally substituted with one or more halogen, which are the same or different;
   wherein —R⁴ and —R^{4a} are independently selected from the group consisting of —H and C₁₋₆ alkyl; wherein C₁₋₆ alkyl is optionally substituted with one or more halogen, which are the same or different;
—Y is selected from the group consisting of:

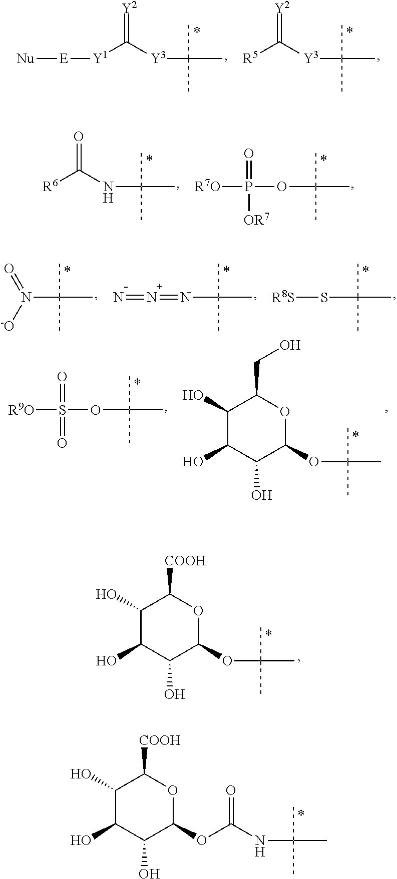

and a peptidyl moiety;
wherein
the dashed line marked with an asterisk indicates the attachment to -A-;
-Nu is a nucleophile;
—Y¹— is selected from the group consisting of —O—, —C(R¹⁰)(R^{10a})—, —N(R¹¹)— and —S—;
═Y² is selected from the group consisting of ═O, ═S and ═N(R¹²);
—Y³— is selected from the group consisting of —O—, —S— and —N(R¹³)—;
-E- is selected from the group consisting of C₁₋₆ alkyl, C₂₋₆ alkenyl, C₂₋₆ alkynyl and
-Q-; wherein C₁₋₆ alkyl, C₂₋₆ alkenyl, C₂₋₆ alkynyl are optionally substituted with one or more —R¹⁴, which are the same or different;
—R⁵, —R⁶, each —R⁷, —R⁸, —R⁹, —R¹⁰, —R^{10a}, —R¹¹, —R¹² and —R¹³ are independently selected from the group consisting of C₁₋₂₀ alkyl, C₂₋₂₀ alkenyl, C₂₋₂₀ alkynyl and -Q; wherein C₁₋₂₀ alkyl, C₂₋₂₀ alkenyl and C₂₋₂₀ alkynyl are optionally substituted with one or more —R¹⁴, which are the same or different; and wherein C₁₋₂₀ alkyl, C₂₋₂₀ alkenyl and C₂₋₂₀ alkynyl are optionally interrupted by one or more groups selected from the group consisting of -Q-, —C(O)O—, —O—, —C(O)—, —C(O)N(R¹⁵)—, —S(O)₂N(R¹⁵)—, —S(O)N(R¹⁵)—, —S(O)₂—, —S(O)—, —N(R¹⁵)S(O)₂N(R^{15a})—, —S—, —N(R¹⁵)—, —OC(OR¹⁵)R^{15a}—, —N(R¹⁵)C(O)N(R^{15a})— and —OC(O)N(R¹⁵)—;
   each Q is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q is independently optionally substituted with one or more —R¹⁴, which are the same or different;
   wherein —R¹⁴, —R¹⁵ and —R^{a15} are independently selected from the group consisting of —H and C₁₋₆ alkyl; wherein C₁₋₆ alkyl is optionally substituted with one or more halogen, which are the same or different; and each -L¹- is substituted with -L²- and optionally further substituted.

It is understood that in certain embodiments -D⁺ may comprise both an electron-donating heteroaromatic N⁺ and a quaternary ammonium cation and analogously the corresponding D may comprise both an electron-donating heteroaromatic N and a tertiary amine. It is also understood that if D is conjugated to -L¹-, then -D⁺ and -L¹- form a quaternary ammonium cation, for which there may be a counter anion. Examples of counter anions include, but are not limited to, chloride, bromide, acetate, bicarbonate, sulfate, bisulfate, nitrate, carbonate, alkyl sulfonate, aryl sulfonate and phosphate.

The optional further substituents of -L¹- of formula (XIII) are as described elsewhere herein.

In certain embodiments, -L¹- of formula (XIII) is not further substituted.

Such drug moiety -D⁺ comprises at least one, such as one, two, three, four, five, six, seven, eight, nine or ten electron-donating heteroaromatic N⁺ or quaternary ammonium cations and analogously the corresponding released drug D comprises at least one, such as one, two, three, four, five, six, seven, eight, nine or ten electron-donating heteroaromatic N or tertiary amines. Examples of chemical structures including heteroaromatic nitrogen atoms i.e. N⁺ or N, that donate an electron to the aromatic π-system include, but are not limited to, pyridine, pyridazine, pyrimidine, quinoline, quinazoline, quinoxaline, pyrazole, imidazole, isoindazole, indazole, purine, tetrazole, triazole and triazine. For example, in the imidazole ring below the heteroaromatic nitrogen which donates one electron to the aromatic π-system is marked with “§”:

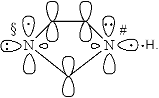

Such electron-donating heteroaromatic nitrogen atoms do not comprise heteroaromatic nitrogen atoms which donate one electron pair (i.e. not one electron) to the aromatic if-system, such as for example the nitrogen that is marked with “#” in the abovementioned imidazole ring structure. The drug D may exist in one or more tautomeric forms, such as with one hydrogen atom moving between at least two heteroaromatic nitrogen atoms. In all such cases, the linker moiety is covalently and reversibly attached at a heteroaromatic nitrogen that donates an electron to the aromatic π-system.

As used herein, the term “monocyclic or bicyclic aryl” means an aromatic hydrocarbon ring system which may be monocyclic or bicyclic, wherein the monocyclic aryl ring consists of at least 5 ring carbon atoms and may comprise up to 10 ring carbon atoms and wherein the bicylic aryl ring consists of at least 8 ring carbon atoms and may comprise up to 12 ring carbon atoms. Each hydrogen atom of a monocyclic or bicyclic aryl may be replaced by a substituent as defined below.

As used herein, the term “monocyclic or bicyclic heteroaryl” means a monocyclic aromatic ring system that may comprise 2 to 6 ring carbon atoms and 1 to 3 ring heteroatoms or a bicyclic aromatic ring system that may comprise 3 to 9 ring carbon atoms and 1 to 5 ring heteroatoms, such as nitrogen, oxygen and sulfur. Examples for monocyclic or bicyclic heteroaryl groups include, but are not limited to, benzofuranyl, benzothiophenyl, furanyl, imidazolyl, indolyl, azaindolyl, azabenzimidazolyl, benzoxazolyl, benzthiazolyl, benzthiadiazolyl, benzotriazolyl, tetrazinyl, tetrazolyl, isothiazolyl, oxazolyl, isoxazolyl, pyrazinyl, pyrazolyl, pyridazinyl, pyridinyl, pyrimidinyl, pyrrolyl, quinolinyl, quinazolinyl, quinoxalinyl, triazolyl, thiazolyl and thiophenyl.

Each hydrogen atom of a monocyclic or bicyclic heteroaryl may be replaced by a substituent as defined below.

As used herein, the term “nucleophile” refers to a reagent or functional group that forms a bond to its reaction partner, i.e. the electrophile by donating both bonding electrons.

In certain embodiments, t of formula (XIII) is 0. In certain embodiments, t of formula (XIII) is 1.

In certain embodiments, t of formula (XIII) is 2. In certain embodiments, t of formula (XIII) is 3.

In certain embodiments, t of formula (XIII) is 4. In certain embodiments, t of formula (XIII) is 5.

In certain embodiments, t of formula (XIII) is 6.

In certain embodiments, -A- of formula (XIII) is a ring selected from the group consisting of monocyclic or bicyclic aryl and heteroaryl, provided that -A- is connected to —Y and —C(R¹)(R^{1a})—via carbon atoms. In certain embodiments, -A- of formula (XIII) is substituted with one or more —R² of formula (XIII) which are the same or different. In certain embodiments, -A- of formula (XIII) is not substituted with —R² of formula (XIII). In certain embodiments, -A- of formula (XIII) is selected from the group consisting of.

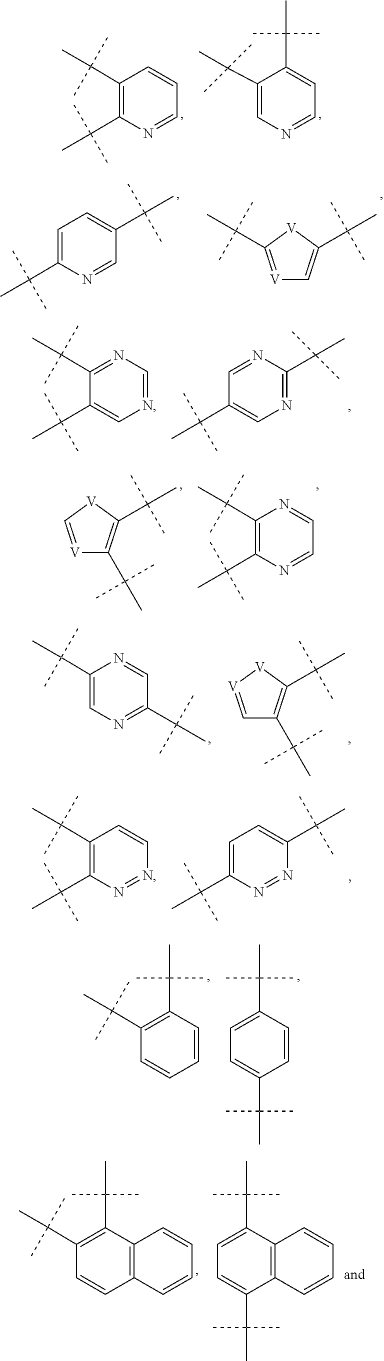

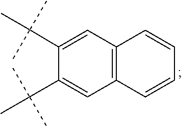

wherein each V is independently selected from the group consisting of O, S and N.

In certain embodiments, —R¹, —R^{1a} and each —R² of formula (XIII) are independently selected from the group consisting of —H, —C(O)OH, -halogen, —CN, —NO₂, —OH, C₁₋₆ alkyl, C₂₋₆ alkenyl and C₂₋₆ alkynyl. In certain embodiments, —R¹, —R^{1a} and each —R² of formula (XIII) are independently selected from the group consisting of —H, —C(O)OH, —CN, C₁₋₆ alkyl, C₂₋₆ alkenyl and C₂₋₆ alkynyl. In certain embodiments, —R¹ of formula (XIII) is —H. In certain embodiments, —R¹ of formula (XIII) is —C(O)OH. In certain embodiments, —R¹ of formula (XIII) is -halogen. In certain embodiments, —R¹ of formula (XIII) is —F. In certain embodiments, —R¹ of formula (XIII) is —CN. In certain embodiments, —R¹ of formula (XIII) is —NO₂. In certain embodiments, —R¹ of formula (XIII) is —OH. In certain embodiments, —R¹ of formula (XIII) is C₁₋₆ alkyl. In certain embodiments, —R¹ of formula (XIII) is C₂₋₆ alkenyl. In certain embodiments, —R¹ of formula (XIII) is C₂₋₆ alkynyl. In certain embodiments, —R^{1a} of formula (XIII) is —H. In certain embodiments, —R^{1a} of formula (XIII) is —C(O)OH. In certain embodiments, —R^{1a} of formula (XIII) is -halogen. In certain embodiments, —R^{1a} of formula (XIII) is —F. In certain embodiments, —R^{1a} of formula (XIII) is —CN. In certain embodiments, —R^{1a} of formula (XIII) is —NO₂. In certain embodiments, —R^{1a} of formula (XIII) is —OH. In certain embodiments, —R^{1a} of formula (XIII) is C₁₋₆ alkyl. In certain embodiments, —Ria of formula (XIII) is C₂₋₆ alkenyl. In certain embodiments, —R^{1a} of formula (XIII) is C₂₋₆ alkynyl.

In certain embodiments, each of —R² of formula (XIII) is independently selected from the group consisting of —H, —C(O)OH, -halogen, —CN, —NO₂, —OH, C₁₋₆ alkyl, C₂₋₆ alkenyl and C₂₋₆ alkynyl. In certain embodiments, each of —R² of formula (XIII) is —H. In certain embodiments, each of —R² of formula (XIII) is —C(O)OH. In certain embodiments, each of —R² of formula (XIII) is -halogen. In certain embodiments, each of —R² of formula (XIII) is —F. In certain embodiments, each of —R² of formula (XIII) is —CN. In certain embodiments, each of —R² of formula (XIII) is —NO₂. In certain embodiments, each of —R² of formula (XIII) is —OH. In certain embodiments, each of —R² of formula (XIII) is C₁₋₆ alkyl. In certain embodiments, each of —R² of formula (XIII) is C₂₋₆ alkenyl. In certain embodiments, each of —R² of formula (XIII) is C₂₋₆ alkynyl.

In certain embodiments, T of formula (XIII) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, T of formula (XIII) is phenyl. In certain embodiments, T of formula (XIII) is naphthyl. In certain embodiments, T of formula (XIII) is indenyl. In certain embodiments, T of formula (XIII) is indanyl. In certain embodiments, T of formula (XIII) is tetralinyl. In certain embodiments, T of formula (XIII) is C₃₋₁₀ cycloalkyl. In certain embodiments, T of formula (XIII) is 3- to 10-membered heterocyclyl. In certain embodiments, T of formula (XIII) is 8- to 11-membered heterobicyclyl.

In certain embodiments, T of formula (XIII) is substituted with one or more —R³ of formula (XIII), which are the same or different. In certain embodiments, T of formula (XIII) is substituted with one —R³ of formula (XIII). In certain embodiments, T of formula (XIII) is not substituted with —R³ of formula (XIII).

In certain embodiments, —R³ of formula (XIII) is selected from the group consisting of —H, —NO₂, —OCH₃, —CN, —N(R⁴)(R^{4a}), —OH, —C(O)OH and C₁₋₆ alkyl. In certain embodiments, —R³ of formula (XIII) is —H. In certain embodiments, —R³ of formula (XIII) is —NO₂. In certain embodiments, —R³ of formula (XIII) is —OCH₃. In certain embodiments, —R³ of formula (XIII) is —CN. In certain embodiments, —R³ of formula (XIII) is —N(R⁴)(R^{4a}). In certain embodiments, —R³ of formula (XIII) is —OH. In certain embodiments, —R³ of formula (XII) is —C(O)OH. In certain embodiments, —R³ of formula (XIII) is C₁₋₆ alkyl. In certain embodiments, —R⁴ and —R^{4a} of formula (XIII) are independently selected from the group consisting of —H and C₁₋₆ alkyl. In certain embodiments, —R⁴ of formula (XIII) is —H. In certain embodiments, —R⁴ is C₁₋₆ alkyl. In certain embodiments, —R^{4a} of formula (XIII) is —H. In certain embodiments, —R^{4a} of formula (XIII) is C₁₋₆ alkyl.

In certain embodiments, —Y of formula (XIII) is selected from the group consisting of

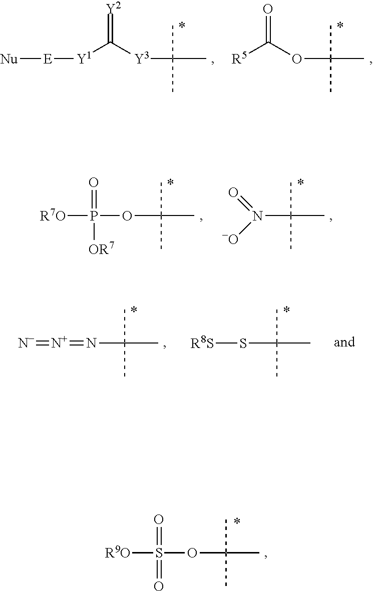

wherein -Nu, -E-, —Y¹—, ═Y², —Y³—, —R⁵, —R⁷, —R⁸ and —R⁹ are defined as above.

In certain embodiments, —Y of formula (XIII) is

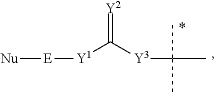

wherein -Nu, -E, —Y¹—, ═Y² and —Y³— are as defined elsewhere herein and the dashed line marked with an asterisk indicates the attachment to -A- of formula (XII). It is understood that in this instance the release of the drug D is not triggered by an enzyme, and that the drug is released in its unmodified, pharmacologically fully active form in the absence of an enzyme.

In certain embodiments, -Nu of formula (XIII) is a nucleophile selected from the group consisting of primary, secondary, or tertiary amine and amide. In certain embodiments, -Nu of formula (XIII) is a primary amine. In certain embodiments, -Nu of formula (XIII) is a secondary amine. In certain embodiments, -Nu of formula (XIII) is a tertiary amine. In certain embodiments, -Nu of formula (XIII) is an amide.

In certain embodiments, —Y¹— of formula (XIII) is selected from the group consisting of —O—, —C(R¹⁰)(R^{10a})—, —N(R¹¹)— and —S—. In certain embodiments, —Y¹— of formula (XIII) is —O—. In certain embodiments, —Y¹— of formula (XIII) is —C(R¹⁰)(R^{10a})—. In certain embodiments, —Y¹— of formula (XIII) is —N(R¹¹)—. In certain embodiments, —Y¹— of formula (XIII) is —S—.

In certain embodiments, ═Y² of formula (XIII) is selected from the group consisting of ═O, ═S and ═N(R¹²). In certain embodiments, ═Y² of formula (XIII) is ═O. In certain embodiments ═Y² of formula (XIII) is ═S. In certain embodiments, ═Y² of formula (XIII) is ═N(R¹²).

In certain embodiments, —Y³— of formula (XIII) is selected from the group consisting of —O—, —S— and —N(R¹³). In certain embodiments, —Y³— of formula (XIII) is —O—. In certain embodiments, —Y³— of formula (XIII) is —S—. In certain embodiments, —Y³— of formula (XIII) is —N(R¹³)—.

In certain embodiments, —Y¹— of formula (XIII) is —N(R¹¹)—, ═Y² of formula (XIII) is ═O and —Y³— is —O—.

In certain embodiments, —Y¹— of formula (XIII) is —N(R¹¹)—, ═Y² of formula (XIII) is ═O, —Y³— of formula (XIII) is —O— and -Nu of formula (XIII) is —N(CH₃)₂.

In certain embodiments, -E- of formula (XIII) is selected from the group consisting of C₁₋₆ alkyl, C₂₋₆ alkenyl, C₂₋₆ alkynyl and —Q-. In certain embodiments, -E- of formula (XIII) is C₁₋₆ alkyl. In certain embodiments, -E- of formula (XIII) is C₂₋₆ alkenyl. In certain embodiments, -E- of formula (XIII) is C₂₋₆ alkynyl. In certain embodiments, -E- of formula (XIII) is —Q-.

In certain embodiments, Q of formula (XIII) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, Q of formula (XIII) is phenyl. In certain embodiments, Q of formula (XIII) is naphthyl. In certain embodiments Q of formula (XIII) is indenyl. In certain embodiments, Q of formula (XIII) is indanyl. In certain embodiments, Q of formula (XIII) is tetralinyl. In certain embodiments, Q of formula (XIII) is C₃₋₁₀ cycloalkyl. In certain embodiments, Q of formula (XIII) is 3- to 10-membered heterocyclyl. In certain embodiments, Q of formula (XIII) is 8- to 11-membered heterobicyclyl. In certain embodiments, Q of formula (XIII) is substituted with one or more —R¹⁴. In certain embodiments, Q of formula (XIII) is not substituted with —R¹⁴.

In certain embodiments, —R⁵, —R⁶, each —R⁷, —R⁸, —R⁹, —R¹⁰, —R^{10a}, —R¹¹, —R¹² and —R¹³ of formula (XIII) are independently selected from the group consisting of C₁₋₂₀ alkyl, C₂₋₂₀ alkenyl, C₂₋₂₀ alkynyl and -Q.

In certain embodiments, —R⁵ of formula (XIII) is C₁₋₂₀ alkyl. In certain embodiments, —R⁵ of formula (XIII) is C₂₋₂₀ alkenyl. In certain embodiments, —R⁵ of formula (XIII) is C₂₋₂₀ alkynyl. In certain embodiments, —R⁵ of formula (XIII) is -Q.

In certain embodiments, —R⁶ of formula (XIII) is C₁₋₂₀ alkyl. In certain embodiments, —R⁶ of formula (XIII) is C₂₋₂₀ alkenyl. In certain embodiments, —R⁶ of formula (XIII) is C₂₋₂₀ alkynyl. In certain embodiments, —R⁶ is -Q.

In certain embodiments, each of —R⁷ of formula (XIII) is independently selected from the group consisting of C₁₋₂₀ alkyl, C₂₋₂₀ alkenyl, C₂₋₂₀ alkynyl and -Q. In certain embodiments, each of —R⁷ of formula (XIII) is C₁₋₂₀ alkyl. In certain embodiments, each of —R⁷ of formula (XIII) is C₂₋₂₀ alkenyl. In certain embodiments, each of —R⁷ of formula (XIII) is C₂₋₂₀ alkynyl. In certain embodiments, each of —R⁷ of formula (XIII) is -Q.

In certain embodiments, —R⁸ of formula (XIII) is C₁₋₂₀ alkyl. In certain embodiments, —R⁸ of formula (XIII) is C₂₋₂₀ alkenyl. In certain embodiments, —R⁸ of formula (XIII) is C₂₋₂₀ alkynyl. In certain embodiments, —R⁸ of formula (XIII) is -Q.

In certain embodiments, —R⁹ of formula (XIII) is C₁₋₂₀ alkyl. In certain embodiments, —R⁹ of formula (XII) is C₂₋₂₀ alkenyl. In certain embodiments, —R⁹ of formula (XIII) is C₂₋₂₀ alkynyl. In certain embodiments, —R⁹ of formula (XIII) is -Q.

In certain embodiments, —R¹⁰ of formula (XIII) is C₁₋₂₀ alkyl. In certain embodiments, —R¹⁰ of formula (XIII) is C₂₋₂₀ alkenyl. In certain embodiments, —R¹⁰ of formula (XIII) is C₂₋₂₀ alkynyl. In certain embodiments, —R¹⁰ of formula (XIII) is -Q.

In certain embodiments, —R^{10a} of formula (XIII) is C₁₋₂₀ alkyl. In certain embodiments, —R^{10a} of formula (XIII) is C₂₋₂₀ alkenyl. In certain embodiments, —R^{10a} of formula (XIII) is C₂₋₂₀ alkynyl. In certain embodiments, —R^{10a} of formula (XIII) is -Q.

In certain embodiments, —R¹¹ of formula (XIII) is C₁₋₂₀ alkyl. In certain embodiments, —R¹¹ of formula (XIII) is C₂₋₂₀ alkenyl. In certain embodiments, —R¹¹ of formula (XIII) is C₂₋₂₀ alkynyl. In certain embodiments, —R¹¹ of formula (XIII) is -Q.

In certain embodiments, —R¹² of formula (XIII) is C₁₋₂₀ alkyl. In certain embodiments, —R¹² of formula (XIII) is C₂₋₂₀ alkenyl. In certain embodiments, —R¹² of formula (XIII) is C₂₋₂₀ alkynyl. In certain embodiments, —R¹² of formula (XIII) is -Q.

In certain embodiments, —R¹³ of formula (XIII) is C₁₋₂₀ alkyl. In certain embodiments, —R¹³ of formula (XIII) is C₂₋₂₀ alkenyl. In certain embodiments, —R¹³ of formula (XIII) is C₂₋₂₀ alkynyl. In certain embodiments, —R¹³ of formula (XIII) is -Q.

In certain embodiments, —R¹⁴, —R¹⁵ and —R^{a15} of formula (XIII) are selected from the group consisting of —H and C₁₋₆ alkyl.

In certain embodiments, —R¹⁴ of formula (XIII) is —H. In certain embodiments, —R¹⁴ of formula (XIII) is C₁₋₆ alkyl.

In certain embodiments, —R¹⁵ of formula (XIII) is —H. In certain embodiments, —R¹⁵ of formula (XIII) is C₁₋₆ alkyl.

In certain embodiments, —R^{a15} of formula (XIII) is —H. In certain embodiments, —R^{a15} of formula (XIII) is C₁₋₆ alkyl.

In certain embodiments, -L¹- has a structure as disclosed in WO 2020/254606 A1, which is hereby incorporated by reference in its entirety. Accordingly, in certain embodiments the moiety -L¹- is of formula (XIV):

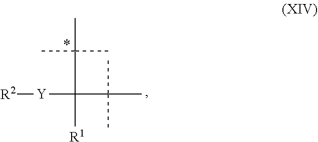

wherein
the dashed line marked with an asterisk indicates the attachment to -L²-;
the unmarked dashed line indicates the attachment to the π-electron-pair-donating heteroaromatic N of -D;
—Y— is selected from the group consisting of —N(R³)—, —O— and —S—;
—R¹, —R² and —R³ are independently selected from the group consisting of —H, -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R⁴, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
   each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each T is independently optionally substituted with one or more —R⁴, which are the same or different;
   wherein —R⁴, —R⁵ and —R^{5a} are independently selected from the group consisting of —H and C₁₋₆ alkyl; wherein C₁₋₆ alkyl is optionally substituted with one or more halogen, which are the same or different; and
each -L¹- is substituted with -L²- and optionally further substituted.

In certain embodiments, —Y— of formula (XIV) is —N(R³)—. In certain embodiments, —Y— of formula (XIV) is —O—. In certain embodiments, —Y— of formula (XIV) is —S—.

In certain embodiments, —R¹, —R² and —R³ of formula (XIV) are independently selected from the group consisting of —H, -T, C₁₋₆ alkyl, C₂₋₆ alkenyl and C₂₋₆ alkynyl.

In certain embodiments, —R¹ of formula (XIV) is independently selected from the group consisting of —H, -T, C₁₋₆ alkyl, C₂₋₆ alkenyl and C₂₋₆ alkynyl. In certain embodiments, —R¹ of formula (XIV) is —H. In certain embodiments, —R¹ of formula (XIV) is -T. In certain embodiments, —R¹ of formula (XIV) is C₁₋₆ alkyl. In certain embodiments, —R¹ of formula (XIV) is C₂₋₆ alkenyl. In certain embodiments, —R¹ of formula (XIV) is C₂₋₆ alkynyl.

In certain embodiments, —R² of formula (XIV) is independently selected from the group consisting of —H, -T, C₁₋₆ alkyl, C₂₋₆ alkenyl and C₂₋₆ alkynyl. In certain embodiments, —R² of formula (XIV) is —H. In certain embodiments, —R² of formula (XIV) is -T. In certain embodiments, —R² of formula (XIV) is C₁₋₆ alkyl. In certain embodiments, —R² of formula (XIV) is C₂₋₆ alkenyl. In certain embodiments, —R² of formula (XIV) is C₂₋₆ alkynyl.

In certain embodiments, —R³ of formula (XIV) is independently selected from the group consisting of —H, -T, C₁₋₆ alkyl, C₂₋₆ alkenyl and C₂₋₆ alkynyl. In certain embodiments, —R³ of formula (XIV) is —H. In certain embodiments, —R³ of formula (XIV) is -T. In certain embodiments, —R³ of formula (XIV) is C₁₋₆ alkyl. In certain embodiments, —R³ of formula (XIV) is C₂₋₆ alkenyl. In certain embodiments, —R³ of formula (XIV) is C₂₋₆ alkynyl.

In certain embodiments, T of formula (XIV) is selected from the group consisting of phenyl, naphthyl, indenyl, indanyl, tetralinyl, C₃₋₁₀ cycloalkyl, 3- to 10-membered heterocyclyl and 8- to 11-heterobicyclyl. In certain embodiments, T of formula (XIV) is phenyl. In certain embodiments, T of formula (XIV) is naphthyl. In certain embodiments, T of formula (XIV) is indenyl. In certain embodiments, T of formula (XIV) is indanyl. In certain embodiments, T of formula (XIV) is tetralinyl. In certain embodiments, T of formula (XIV) is C₃₋₁₀ cycloalkyl. In certain embodiments, T of formula (XIV) is 3- to 10-membered heterocyclyl. In certain embodiments, T of formula (XIV) is 8- to 11-heterobicyclyl.

In certain embodiments, T of formula (XIV) is substituted with one or more —R⁴. In certain embodiments, T of formula (XIV) is substituted with one —R⁴. In certain embodiments, T of formula (XIV) is not substituted with —R⁴.

In certain embodiments, —R⁴, —R⁵ and —R^{5a} of formula (XIV) are independently selected from the group consisting of —H and C₁₋₆ alkyl.

In certain embodiments, —R⁴ of formula (XIV) is selected from the group consisting of —H and C₁₋₆ alkyl. In certain embodiments, —R⁴ of formula (XIV) is —H. In certain embodiments, —R⁴ of formula (XIV) is C₁₋₆ alkyl.

In certain embodiments, —R⁵ of formula (XIV) is selected from the group consisting of —H and C₁₋₆ alkyl. In certain embodiments, —R⁵ of formula (XIV) is —H. In certain embodiments, —R⁵ of formula (XIV) is C₁₋₆ alkyl.

In certain embodiments, —R^{5a} of formula (XIV) is selected from the group consisting of —H and C₁₋₆ alkyl. In certain embodiments, —R^{5a} of formula (XIV) is —H. In certain embodiments, —R^{5a} of formula (XIV) is C₁₋₆ alkyl.

In certain embodiments, —Y— of formula (XIV) is —O— and —R² of formula (XIV) is C₁₋₆ alkyl. In certain embodiments, —Y— of formula (XIV) is —O— and —R² of formula (XIV) is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In certain embodiments, —Y— of formula (XIV) is —O— and —R² of formula (XIV) is methyl. In certain embodiments, —Y— of formula (XIV) is —O— and —R² of formula (XIV) is ethyl.

In certain embodiments, —Y— of formula (XIV) is —O— and —R² of formula (XIV) is C₁₋₆ alkyl, wherein C₁₋₆ alkyl is interrupted by —C(O)—.

In certain embodiments, —Y— of formula (XIV) is —N(R³)— and —R² of formula (XIV) is C₁₋₆ alkyl, wherein C₁₋₆ alkyl is interrupted by —C(O)O— and —R³ of formula (XIV) is as defined in formula (XIV).

In certain embodiments, —Y— of formula (XIV) is —N(R³)— and —R² of formula (XIV) is C₁₋₆ alkyl, wherein C₁₋₆ alkyl is interrupted by —C(O)O— and —R³ of formula (XIV) is selected from the group consisting of —H, methyl, ethyl and propyl.

In certain embodiments, -L²- is absent. In certain embodiments, -L²- is a spacer moiety.

In certain embodiments, -L²-, -L^{2′}- or -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl and C₂₋₅₀ alkynyl; wherein -T′-, C₁₋₅₀ alkyl, C₂₋₅₀ alkenyl and C₂₋₅₀ alkynyl are optionally substituted with one or more —R^{y2}, which are the same or different and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})— and —OC(O)N(R^{y3})—;
—R^{y1} and —R^{y1a} are independently selected from the group consisting of —H, -T′, C₁₋₅₀ alkyl, C₂₋₅₀ alkenyl and C₂₋₅₀ alkynyl; wherein -T′, C₁₋₅₀ alkyl, C₂₋₅₀ alkenyl and C₂₋₅₀ alkynyl are optionally substituted with one or more —R^{y2}, which are the same or different, and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each T′ is independently optionally substituted with one or more —R^{y2}, which are the same or different;
each —R^{y2} is independently selected from the group consisting of halogen, —CN, oxo (═O), —C(O)OR^{y5}, —OR^{y5}, —C(O)R^{y5}, —C(O)N(R^{y5})(R^{y5a}), —S(O)₂N(R^{y5})(R^{y5a}), —S(O)N(R⁵)(R^{y5a}), —S(O)₂R^{y5}, —S(O)R^{y5}, —N(R^{y5})S(O)₂N(R^{y5})(R^{y5a}), —SR^{y5}, —N(R^{y5})(R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R⁵)(R^{y5a}), —OC(O)N(R^{y5})(R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²-, -L^{2′}- or -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T′-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently selected from the group consisting of —H, -T′, C₁₋₁₀ alkyl, C₂₋₁₀ alkenyl, and C₂₋₁₀ alkynyl; wherein -T′, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T′ is independently optionally substituted with one or more —R^{y2}, which are the same or different; —R^{y2} is selected from the group consisting of halogen, —CN, oxo (═O), —C(O)OR^{y5}, —OR^{y5}, —C(O)R^{y5}, —C(O)N(R⁵)(R^{y5a}), —S(O)₂N(R^{y5})(R^{y5a}), —S(O)N(R⁵)(R^{y5a}), —S(O)₂R^{y5}, —S(O)R^{y5}, —N(R^{y5})S(O)₂N(R^{y5a})(R^{y5b}), —SR^{y5}, —N(R^{y5})(R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a})(R^{y5b}), —OC(O)N(R^{y5})(R^{y5a}) and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²-, -L^{2′}- or -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T′-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})— and —OC(O)N(R^{y3})—;
—R^{y1} and —R^{y1a} are independently selected from the group consisting of —H, -T′, C₁₋₁₀ alkyl, C₂₋₁₀ alkenyl and C₂₋₁₀ alkynyl;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl;
each —R^{y2} is independently selected from the group consisting of halogen, and C₁₋₆ alkyl; and
each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²-, -L^{2′}- or -L^{2″}- is a C₁₋₂₀ alkyl chain, which is optionally interrupted by one or more groups independently selected from the group consisting of —O—, -T′- and —C(O)N(R^{y1})—; and which C₁₋₂₀ alkyl chain is optionally substituted with one or more groups independently selected from the group consisting of —OH, -T′ and —C(O)N(R⁶R^{y6a}); wherein —R^{y1}, —R^{y6}, —R^{y6a} are independently selected from the group consisting of H and C₁₋₄ alkyl and wherein T′ is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl.

In certain embodiments, -L²-, -L^{2′}- or -L²- has a molecular weight ranging from 14 g/mol to 750 g/mol.

In certain embodiments, -L²-, -L^{2′}- or -L^{2″}- comprises a moiety selected from the group consisting of:

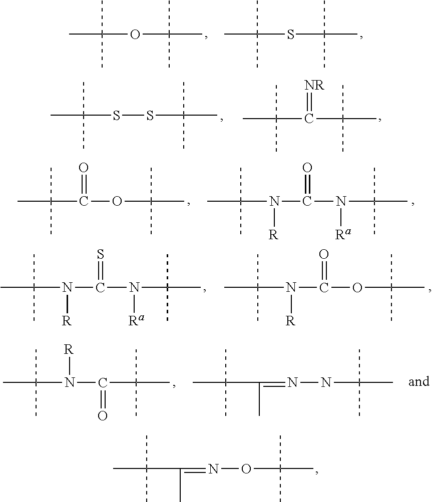

wherein for -L²- the dashed lines indicate the attachment to -L¹- and -L⁴-; and —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In certain embodiments, -L²- comprises the following moiety

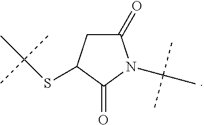

In certain embodiments, -L²-, -L^{2′}- or -L²- has a chain length of 1 to 20 atoms.

Suitably, -L²- is of formula (11):

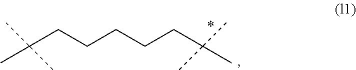

wherein the unmarked dashed indicates the attachment to -L⁴- while the dashed line marked with the asterisk indicates attachment to -L¹-.

Another aspect of the present invention is a method for preparing a functionalized HA, which comprises the following steps:
(i) introducing an amine group by converting at least one or more carboxyl groups of a HA into N-substituted amides having a spacer moiety, such as an -L^{2′}- spacer moiety, comprising an amine group at its end;
(ii) introducing a maleimide or thiol group into the resulting HA of step (i) by further converting the end amine groups into N-substituted amides having a spacer moiety, such as an -L^{2″}- spacer moiety comprising a thiol or maleimide group at its end.

Another aspect of the present invention is a method for preparing a functionalized HA, which comprises the following steps:
(i) introducing an amine group by deacetylating, preferably via enzyme catalysis, at least one or more amide groups of a HA into amine groups;
(ii) introducing a maleimide or thiol group into the resulting HA of step (i) by further converting the amine groups into N-substituted amide having a spacer moiety, such as an -L^{2″}-spacer moiety comprising a thiol or maleimide group at its end.

The invention is further described by the following non-limiting items.
1. A method for preparing hydrogel microspheres or pharmaceutically acceptable salts thereof comprising a crosslinked hyaluronic acid (HA), wherein the method comprises the steps of:
   (a) mixing a solution A with a solution B to form an emulsion, wherein solution A comprises a first functionalized HA that is modified with one or more -FG₁ and optionally further functional groups and a second functionalized HA that is modified with one or more -FG₂ and optionally further functional groups, wherein each -FG₁ and -FG₂ are functional group moieties that are different from each other, wherein -FG₁ on the first functionalized HA reacts with -FG₂ on the second functionalized HA to form a plurality of crosslinks which results in the formation of hydrogel HA microspheres;
   (b) optionally, adding a pH-adjusting agent to the emulsion of step (a); and
   (c) collecting the obtained hydrogel HA microspheres of step (a) or (b).
2. The method of item 1, wherein solution A of step (a) further comprises a buffering agent.
3. The method of item 1 or 2, wherein solution B of step (a) comprises an emulsifying agent and a solvent.
4. The method of any one of items 1 to 3, wherein the polymerization occurs via suspension polymerization.
5. The method of any one of items 1 to 4, wherein the method comprises the steps of:
   (a) mixing a solution A with a solution B to form an emulsion, wherein solution A comprises a first functionalized HA that is modified with one or more -FG₁ and a second functionalized HA that is modified with one or more -FG₂, wherein -FG₁ and -FG₂ are functional group moieties that are different from each other and wherein -FG₁ on the first functionalized HA reacts with -FG₂ on the second functionalized HA to form a plurality of crosslinks which results in the formation of hydrogel HA microspheres;
   (b) optionally, adding a pH-adjusting agent to the emulsion of step (a);
   (c) collecting the obtained hydrogel HA microspheres of step (b);
   (d) optionally, size fractionating the obtained hydrogel HA microspheres of step (c) to obtain microspheres with a particular particle size distribution;
   (e) optionally, washing the microspheres obtained in step (c) or (d);
   (f) optionally, incubating the hydrogel HA microspheres of step (c), (d) or (e) in a buffering agent of a pH ranging from about 8 to about 12;
   (g) optionally, incubating the hydrogel HA microspheres of step (c), (d), (e) or (f) with a reducing agent;
   (h) optionally, washing the microspheres obtained in step (f) or (g); and
   (i) optionally, collecting the hydrogel HA microspheres of step (d), (e), (f), (g) or (h).
6. The method of any one of items 1 to 5, wherein -FG₁ is independently selected from the group consisting of:

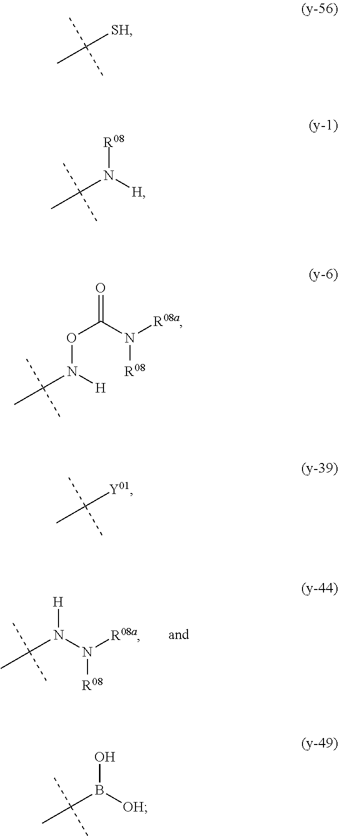

wherein the dashed line indicates the attachment to the first functionalized HA;
—Y⁰¹ is independently selected from the group consisting of —F, —Cl, —Br and —I;
each —R⁰⁸ and —R^{08a} is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰⁹, which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰¹⁰)—, —S(O)₂N(R⁰¹⁰)—, —S(O)N(R⁰¹⁰)—, —S(O)₂—, —S(O)—, —N(R⁰¹⁰)S(O)₂N(R^{010a})—, —S—, —N(R⁰¹⁰)—, —OC(OR⁰¹⁰)(R^{010a})—, —N(R⁰¹⁰)C(O)N(R^{010a})— and —OC(O)N(R⁰¹⁰)—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R⁰⁹, which are the same or different; and
each —R⁰⁹, —R⁰¹⁰ and —R^{010a} is independently selected from the group consisting of —H and C₁₋₆ alkyl; wherein C₁₋₆ alkyl is optionally substituted with one or more halogen, which are the same or different; and
-FG₂ is independently selected from the group consisting of

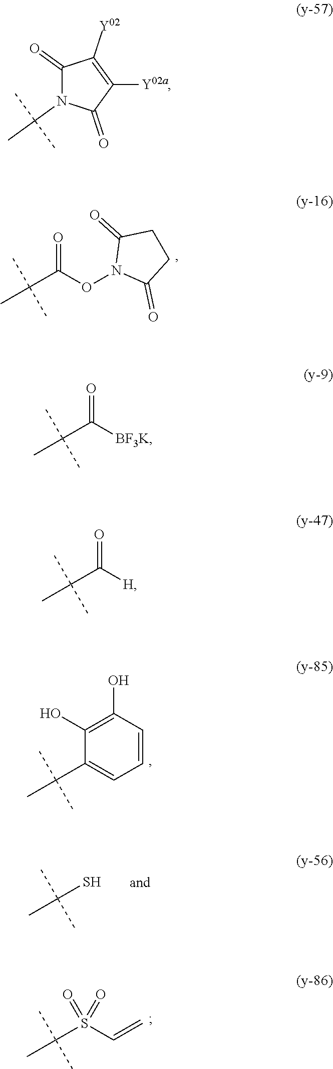

wherein the dashed line indicates the attachment to the second functionalized HA;
each —Y⁰² and —Y^{02a} is independently selected from the group consisting of —H and —Br; provided that -FG₁ is of formula (y-56) then -FG₂ is of formula (y-57) or (y-86); if -FG₁ is of formula (y-1) then -FG₂ is of formula (y-16) or (y-47); if -FG₁ is of formula (y-44) then -FG₂ is of formula (y-16) or (y-47); if -FG₁ is of formula (y-6) then -FG₂ is of formula (y-9); if -FG₁ is of formula (y-49) then -FG₂ is of formula (y-85); if -FG₁ is of formula (y-44) then -FG₂ is of formula (y-47); if -FG₁ is of formula (y-56) then -FG₂ is of formula (y-86) or if -FG₁ is of formula (y-39) then -FG₂ is of formula (y-56).
7. The method of any one of items 1 to 6, wherein the pH-adjusting agent increases the pH of the emulsion of step (a) and -FG₁ is independently selected from the group consisting of:

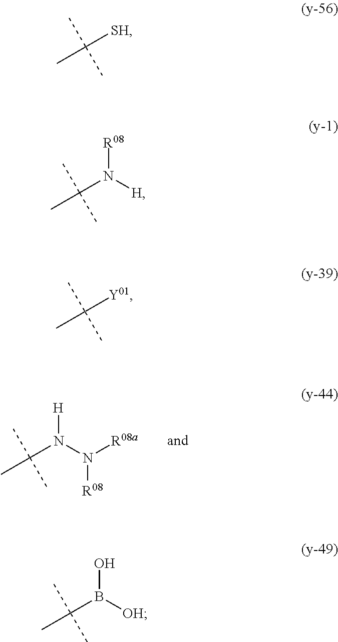

wherein the dashed line indicates the attachment to the first functionalized HA;
—Y⁰¹ is independently selected from the group consisting of —F, —Cl, —Br and —I;
each —R⁰⁸ and —R^{08a} is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰⁹, which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰¹⁰)—, —S(O)₂N(R⁰¹⁰)—, —S(O)N(R⁰¹⁰)—, —S(O)₂—, —S(O)—, —N(R⁰¹⁰)S(O)₂N(R^{010a})—, —S—, —N(R⁰¹⁰)—, —OC(OR⁰¹⁰)(R^{010a})—, —N(R⁰¹⁰)C(O)N(R^{010a})— and —OC(O)N(R⁰¹⁰)—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R⁰⁹, which are the same or different; and
each —R⁰⁹, —R⁰¹⁰ and —R^{010a} is independently selected from the group consisting of —H and C₁₋₆ alkyl; wherein C₁₋₆ alkyl is optionally substituted with one or more halogen, which are the same or different; and
FG₂ is independently selected from the group consisting of

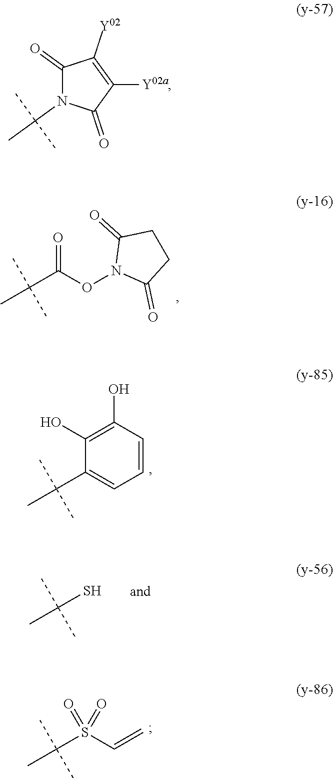

wherein the dashed line indicates the attachment to the second functionalized HA;
each —Y⁰² and —Y^{02a} is independently selected from the group consisting of —H and —Br; provided that -FG₁ is of formula (y-56) then -FG₂ is of formula (y-57) or (y-86); if -FG₁ is of formula (y-1) then -FG₂ is of formula (y-16); if -FG₁ is of formula (y-44) then -FG₂ is of formula (y-16); or if -FG₁ is of formula (y-39) then -FG₂ is of formula (y-56).

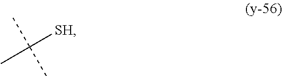

8. The method of any one of items 1 to 7, wherein -FG₁ is (y-56), wherein the dashed

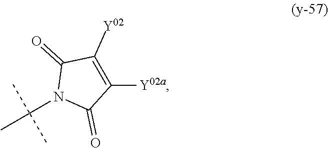

line indicates the attachment to the first functionalized HA, -FG₂ is (y-57), wherein the dashed line indicates the attachment to the second functionalized HA, the pH-adjusting agent increases the pH of the emulsion of step (a) from about 1 to about 9, preferably from about 1 to about 5.5, more preferably from about 2 to about 4 and wherein each —Y⁰² and —Y^{02a} is independently selected from the group consisting of —H and —Br.
9. The method of item 8, wherein both —Y⁰² and —Y^{02a} are —H.
10. The method of any one of items 1 to 9, wherein the method comprises the following steps:
   (a) mixing a solution A with a solution B to form an emulsion, wherein solution A comprises a first functionalized HA that is modified with one or more -FG₁ and a second functionalized HA that is modified with one or more -FG₂, wherein -FG₁ and -FG₂ are functional group moieties that are different from each other and wherein -FG₁ on the first functionalized HA reacts with -FG₂ on the second functionalized HA to form a plurality of crosslinks which results in the formation of hydrogel HA microspheres, wherein
      the first functionalized HA comprises a plurality of each of the following linearly connected Z¹ and Z⁵ units:

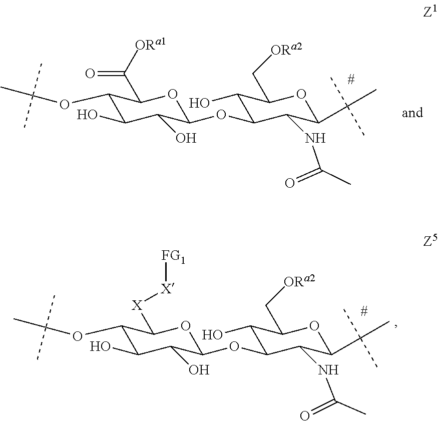

the second functionalized HA comprises a plurality of each of the following linearly connected Z¹ and Z⁶ units:

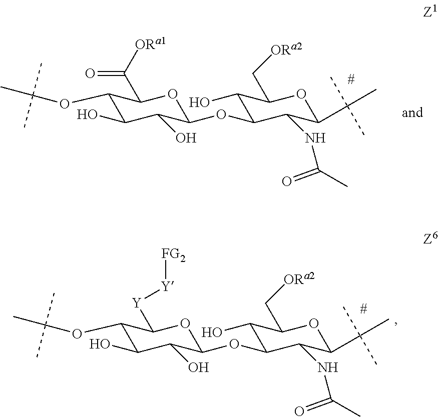

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is independently selected from the group consisting of —H, C₁₋₁₀ alkyl, an ammonium ion, a tetrabutylammonium ion, a cetyl trimethylammonium ion, an alkali metal ion and an alkaline earth metal ion;
each —R^{a2} is independently —H or C₁₋₁₀ alkyl;
each -FG₁, -FG₂ is independently the functional group moiety;
each —X—, —Y— is independently a carbonyl group or absent;
each —X′—, —Y′— is independently a spacer moiety or absent;
(b) adding a pH-adjusting agent to the emulsion of step (a);
(c) collecting the obtained hydrogel HA microspheres of step (a) or (b), wherein said hydrogel comprises a plurality of Z³ units:

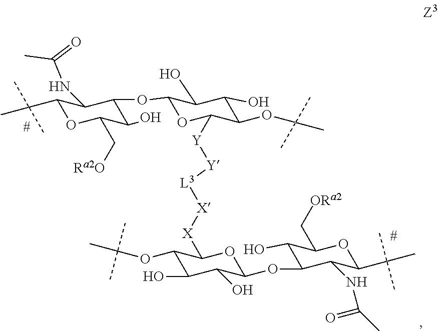

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each —R^{a2}, —X—, —Y—, —X′— and —Y′— are defined as in step (a);
each -L³- is independently a linkage moiety or absent;
(d) optionally, size fractionating the obtained hydrogel HA microspheres of step (a), (b) or (c) to obtain microspheres with a particular particle size distribution;
(e) optionally, washing the hydrogel HA microspheres obtained in step (a), (b), (c) or (d); (f) optionally, incubating the hydrogel HA microspheres of step (a), (b), (c), (d) or (e) in a buffering agent of a pH ranging from about 8 to about 12;
(g) optionally, incubating the hydrogel HA microspheres of step (a), (b), (c), (d), (e) or (f) with a reducing agent;
(h) optionally, washing the microspheres obtained in step (f) or (g); and
(i) optionally, collecting the hydrogel HA microspheres of step (d), (e), (f), (g) or (h).
11. The method of any one of items 1 to 10, wherein the method comprises the following steps:
   (a) mixing a solution A with a solution B to form an emulsion, wherein solution A comprises a first functionalized HA that is modified with one or more thiol functional groups and a second functionalized HA that is modified with one or more maleimide functional groups, wherein the thiol functional groups on the first functionalized HA react with the maleimide functional groups on the second functionalized HA to form a plurality of crosslinks which results in the formation of hydrogel HA microspheres, wherein
      the first functionalized HA comprises a plurality of each of the following linearly connected Z¹ and Z⁵-i units:

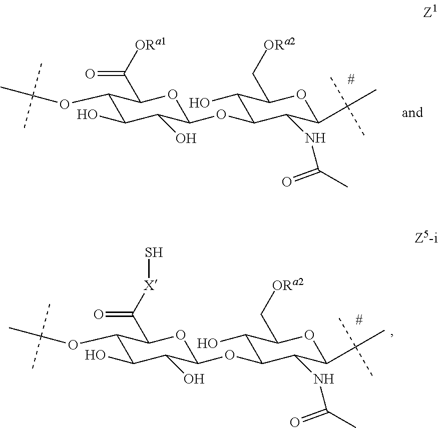

the second functionalized HA comprises a plurality of each of the following linearly connected Z¹ and Z⁶-i units:

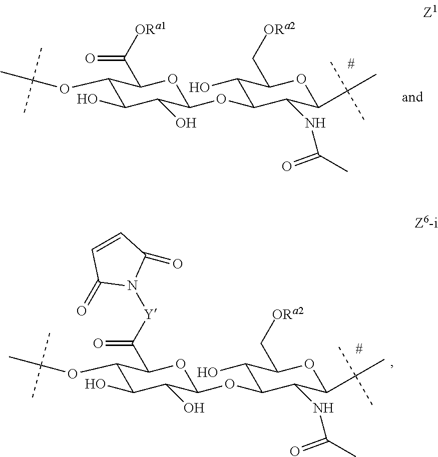

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is independently selected from the group consisting of —H, C₁₋₁₀ alkyl, an ammonium ion, a tetrabutylammonium ion, a cetyl trimethylammonium ion, an alkali metal ion and an alkaline earth metal ion;
each —R^{a2} is independently —H or C₁₋₁₀ alkyl;
each —X′—, —Y′— is independently a spacer moiety or absent;
(b) adding a pH-adjusting agent to the emulsion of step (a);
(c) collecting the obtained hydrogel HA microspheres of step (a) or (b), wherein said hydrogel comprises a plurality of Z³-i units:

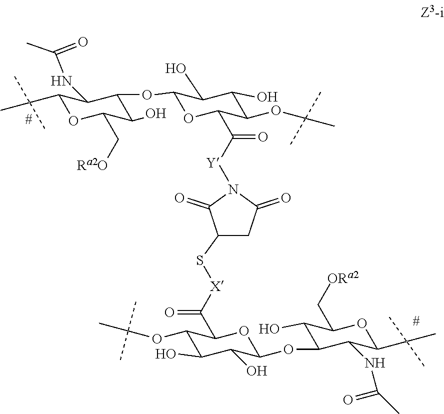

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each —R^{a2}, —X′— and —Y′— are defined as in step (a);
(d) optionally, size fractionating the obtained hydrogel HA microspheres of step (a), (b) or (c) to obtain microspheres with a particular particle size distribution;
(e) optionally, washing the hydrogel HA microspheres obtained in step (a), (b), (c) or (d);
(f) optionally, incubating the hydrogel HA microspheres of step (a), (b), (c), (d) or (e) in a buffering agent of a pH ranging from about 8 to about 12, to provide hydrogel HA microspheres comprising a plurality of Z³-i′ units:

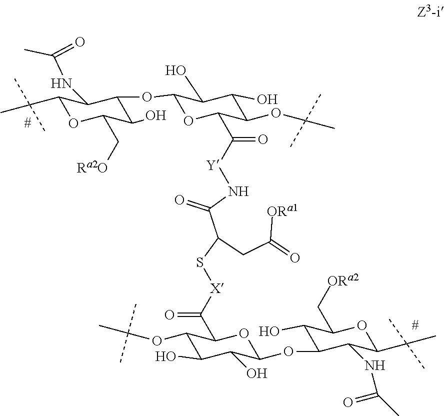

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1}, —R^{a2}, —X′— and —Y′— are defined as in step (a);
(g) optionally, incubating the hydrogel HA microspheres of step (a), (b), (c), (d), (e) or (f) with a reducing agent;
(h) optionally, washing the microspheres obtained in step (f) or (g); and
(i) optionally, collecting the hydrogel HA microspheres of step (d), (e), (f), (g) or (h).
12. The method of item 10 or 11, wherein steps (d) and (e) are not optional and steps (f) to (h) are not present.
13. The method of any one of items 1 to 12, wherein the pH-adjusting agent is selected from the group consisting of N,N,N′,N′-tetramethylethylene diamine (TMEDA), 1,4-dimethylpiperazine, 4-methylmorpholine, 4-ethylmorpholine, 1,4-diazabicyclo[2.2.2]octane, 1,1,4,7,10,10-hexamethyltriethylenetetramine, 1,4,7-trimethyl-1,4,7-triazacyclononane, tris[2-(dimethylamino)ethyl]amine, triethylamine, diisopropylethylamine (DIPEA), trimethylamine, N,N-dimethylethylamine, N,N,N′,N′-tetramethyl-1,6-hexanediamine, N,N,N′,N″,N″-pentamethyldiethylenetriamine, 1,8-diazabicyclo[5.4.0]undec-7-ene, 1,5-diazabicyclo[4.3.0]non-5-ene and hexamethylenetetramine.
14. The method of any one of items 1 to 13, wherein the pH-adjusting agent is N,N,N′,N′-tetramethylethylene diamine (TMEDA).
15. The method of any one of items 1 to 14, wherein each —X′— and —Y′— are independently a spacer moiety selected from the group consisting of -T′-, C₁₋₅₀ alkyl, C₂₋₅₀ alkenyl, and C₂₋₅₀ alkynyl; wherein C₁₋₅₀ alkyl, C₂₋₅₀ alkenyl and C₂₋₅₀ alkynyl are optionally substituted with one or more —R^{y1}, which are the same or different and wherein C₁₋₅₀ alkyl, C₂₋₅₀ alkenyl, and C₂₋₅₀ alkynyl are optionally interrupted by one or more groups selected from the group consisting of -T′- —C(O)O—, —O—, —C(O)—, —C(O)N(R^{y2})—, —S(O)₂N(R^{y2})—, —S(O)N(R^{y2})—, —S(O)₂—, —S(O)—, —N(R^{y2})S(O)₂N(R^{y2a})—, —N(R^{y2})—, —OC(OR^{y2})(R^{y2a})—, —N(R^{y2})C(O)N(R^{y2a})— and —OC(O)N(R^{y2})—;
   each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each T′ is independently optionally substituted with one or more —R^{y1}, which are the same or different;
   each —R^{y1} is independently selected from the group consisting of halogen, —CN, oxo (═O), —COOR^{y3}, —OR^{y3}, —C(O)R^{y3}, —C(O)N(R^{y3}R^{y3a}), —S(O)₂N(R³R^{y3a}), —S(O)N(R³R^{y3a}), —S(O)₂R^{y3}, —S(O)R^{y3}, —N(R^{y3})S(O)₂N(R^{y3a}R^{y3}b), —SR^{y3}, —N(R^{y3}R^{y3a}), —NO₂, —OC(O)R^{y3}, —N(R^{y3})C(O)R^{y3a}, —N(R^{y3})S(O)₂R^{y3a}, —N(R^{y3})S(O)R^{y3a}, —N(R^{y3})C(O)OR^{y3a}, —N(R^{y3})C(O)N(R^{y3a}R^{y3b}), —OC(O)N(R^{y3}R^{y3a}), and C₁₋₆ alkyl; wherein C₁₋₆ alkyl is optionally substituted with one or more halogen, which are the same or different; and each —R^{y2}, —R^{y2a}, —R^{y3}, —R^{y3a}, —R^{y3b} is independently selected from the group consisting of —H and C₁₋₆ alkyl; wherein C₁₋₆ alkyl is optionally substituted with one or more halogen, which are the same or different.
16. The method of any one of items 1 to 15, wherein each —X′— is of formula (x0):

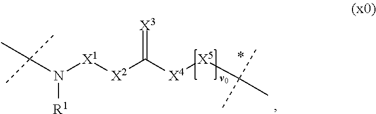

wherein
the unmarked dashed line indicates the attachment to —X— or to the carbonyl group and the dashed line marked with an asterisk indicates the attachment to -FG₁ or -L³-; v₀ is selected from the group consisting of 0 and 1;
—X¹—, —X⁴— are independently C₁₋₁₀ alkyl, which C₁₋₁₀ alkyl is optionally interrupted by one or more groups independently selected from —O—, -T-, —N(R^{y1})—, —C(O)O— and —C(O)N(R^{y1})—; and which C₁₋₁₀ alkyl chain is optionally substituted with one or more groups independently selected from —OH, -T, —NH(R^{y1}) and —C(O)N(R^{y2}R^{y2a});
wherein —R^{y1}, —R^{y2}, —R^{y2a} are independently selected from the group consisting of —H and C₁₋₄ alkyl;
—X²— is selected from the group consisting of —N(R¹)—, —O—, —S— and —Se—;
═X³ is selected from the group consisting of ═O, ═N(R¹) and ═S;
—X⁵— is C₁₋₂₀ alkyl, which C₁₋₂₀ alkyl is optionally interrupted by one or more groups independently selected from —O—, —C(O)O—, -T-, —N(R^{y1})— and —N(R^{y1})C(O)—; and which C₁₋₂₀ alkyl chain is optionally substituted with one or more groups independently selected from —OH, -T, —NH(R^{y1}) and —C(O)N(R^{y2}R^{y2a});
—R¹ is independently selected from the group consisting of —H, C₁₋₅ alkyl and -T;
wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R², which are the same or different;
   —R² is selected from the group consisting of halogen, —CN, oxo, —C(O)OR³, —OR³, —C(O)R³, —C(O)N(R³)(R^{3a}), —S(O)₂N(R³)(R^{3a}), —S(O)N(R³)(R^{3a}), —S(O)₂R³, —S(O)R³, —N(R³)S(O)₂N(R^{3a})(R^{3b}), —SR³, —N(R³)(R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a})(R^{3b}), —OC(O)N(R³)(R^{3a}) and C₁₋₆ alkyl; wherein C₁₋₆ alkyl is optionally substituted with one or more halogen, which are the same or different; and
   wherein —R³, —R^{3a} and —R^{3b} are independently selected from the group consisting of —H and C₁₋₆ alkyl; wherein C₁₋₆ alkyl is optionally substituted with one or more halogen, which are the same or different.
17. The method of any one of items 1 to 16, wherein each —X′— is of formula (x1):

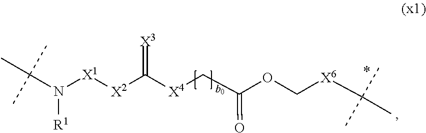

wherein
the unmarked dashed line indicates the attachment to —X— or to the carbonyl group and the dashed line marked with an asterisk indicates the attachment to -FG₁ or -L³-; b₀ is selected from the group consisting of 0, 1, 2, 3, 4, 5, 6, 7, 8, 9 and 10;
—X¹—, —X⁴— are independently C₁₋₅ alkyl, which C₁₋₅ alkyl is optionally interrupted by one or more groups independently selected from —O—, -T-, —N(R^{y1})— and —C(O)N(R^{y1})—; and which C₁₋₅ alkyl chain is optionally substituted with one or more groups independently selected from —OH, -T, —NH(R^{y1}) and —C(O)N(R^{y2}R^{y2a}); wherein —R^{y1}, —R^{y2}, —R^{y2a} are independently selected from the group consisting of —H and C₁₋₄ alkyl;
—X²— is selected from the group consisting of —N(R¹)—, —O—, —S— and —Se—;
═X³ is selected from the group consisting of ═O, ═N(R¹) and ═S;
—X⁶— is C₁₋₁₀ alkyl, which C₁₋₁₀ alkyl is optionally interrupted by one or more groups independently selected from —O—, —C(O)O—, -T-, —N(R^{y1})— and —N(R^{y1})C(O)—; and which C₁₋₁₀ alkyl chain is optionally substituted with one or more groups independently selected from —OH, -T, —NH(R^{y1}) and —C(O)N(R^{y2}R^{y2a});
—R¹ is independently selected from the group consisting of —H, C₁₋₅ alkyl and -T; wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R², which are the same or different;
—R² is selected from the group consisting of halogen, —CN, oxo, —C(O)OR³, —OR³, —C(O)R³, —C(O)N(R³)(R^{3a}), —S(O)₂N(R³)(R^{3a}), —S(O)N(R³)(R^{3a}), —S(O)₂R³, —S(O)R³, —N(R³)S(O)₂N(R^{3a})(R^{3b}), —SR³, —N(R³)(R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a})(R^{3b}), —OC(O)N(R³)(R^{3a}) and C₁₋₆ alkyl; wherein C₁₋₆ alkyl is optionally substituted with one or more halogen, which are the same or different; and
   wherein —R³, —R^{3a} and —R^{3b} are independently selected from the group consisting of —H and C₁₋₆ alkyl; wherein C₁₋₆ alkyl is optionally substituted with one or more halogen, which are the same or different.
18. The method of any one of items 1 to 17, wherein each —X′— is of formula (x2):

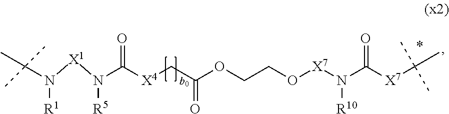

wherein
the unmarked dashed line indicates the attachment to —X— or to the carbonyl group and the dashed line marked with an asterisk indicates the attachment to -FG₁ or -L³-;
b₀ is selected from the group consisting of 0, 1, 2, 3, 4, 5, 6, 7, 8, 9 and 10;
—X¹—, —X⁴—, —X⁷—, —X′— are independently C₁₋₅ alkyl, which C₁₋₅ alkyl is optionally interrupted by one or more groups independently selected from —O—, -T-, —N(R^{y1})— and —C(O)N(R^{y1})—; and which C₁₋₅ alkyl chain is optionally substituted with one or more groups independently selected from —OH, -T, —NH(R^{y1}) and —C(O)N(R^{y2}R^{y2a}); wherein —R^{y1}, —R^{y2}, —R^{y2a} are independently selected from the group consisting of —H and C₁₋₄ alkyl;
—R¹, —R⁵, —R¹⁰ are independently selected from the group consisting of —H, C₁₋₅ alkyl and -T;
wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R⁶, which are the same or different;
   —R⁶ is selected from the group consisting of halogen, —CN, oxo, —C(O)OR⁷, —OR⁷, —C(O)R⁷, —C(O)N(R⁷)(R^{7a}), —S(O)₂N(R⁷)(R^{7a}), —S(O)N(R⁷)(R^{7a}), —S(O)₂R⁷, —S(O)R⁷, —N(R⁷)S(O)₂N(R^{7a})(R^{7b}), —SR⁷, —N(R⁷)(R^{7a}), —NO₂, —OC(O)R⁷, —N(R⁷)C(O)R^{7a}, —N(R⁷)S(O)₂R^{7a}, —N(R⁷)S(O)R^{7a}, —N(R⁷)C(O)OR^{7a}, —N(R⁷)C(O)N(R^{7a})(R^{7b}), —OC(O)N(R⁷)(R^{7a}) and C₁₋₆ alkyl; wherein C₁₋₆ alkyl is optionally substituted with one or more halogen, which are the same or different; and
      wherein —R⁷, —R^{7a} and —R^{7b} are independently selected from the group consisting of —H and C₁₋₆ alkyl; wherein C₁₋₆ alkyl is optionally substituted with one or more halogen, which are the same or different.
19. The method of any one of items 1 to 18, wherein each —X′— is of formula (x3):

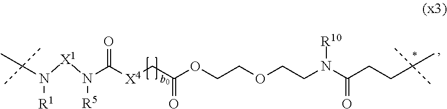

wherein
the unmarked dashed line indicates the attachment to —X— or to the carbonyl group and the dashed line marked with an asterisk indicates the attachment to -FG₁ or -L³-;
b₀ is selected from the group consisting of 0, 1, 2, 3, 4, 5, 6, 7, 8, 9 and 10;
—X¹—, —X⁴— are independently C₁₋₅ alkyl, which C₁₋₅ alkyl is optionally interrupted by one or more groups independently selected from —O—, -T-, —N(R^{y1})— and —C(O)N(R^{y1})—; and which C₁₋₅ alkyl chain is optionally substituted with one or more groups independently selected from —OH, -T, —NH(R^{y1}) and —C(O)N(R^{y2}R^{y2a}); wherein —R^{y1}, —R^{y2}, —R^{y2a} are independently selected from the group consisting of —H and C₁₋₄ alkyl;
—R¹, —R⁵, —R¹⁰ are independently selected from the group consisting of —H, C₁₋₅ alkyl and -T;
wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R⁶, which are the same or different;
   —R⁶ is selected from the group consisting of halogen, —CN, oxo, —C(O)OR⁷, —OR⁷, —C(O)R⁷, —C(O)N(R⁷)(R^{7a}), —S(O)₂N(R⁷)(R^{7a}), —S(O)N(R⁷)(R^{7a}), —S(O)₂R⁷, —S(O)R⁷, —N(R⁷)S(O)₂N(R^{7a})(R^{7b}), —SR⁷, —N(R⁷)(R^{7a}), —NO₂, —OC(O)R⁷, —N(R⁷)C(O)R^{7a}, —N(R⁷)S(O)₂R^{7a}, —N(R⁷)S(O)R^{7a}, —N(R⁷)C(O)OR^{7a}, —N(R⁷)C(O)N(R^{7a})(R^{7b}), —OC(O)N(R⁷)(R^{7a}) and C₁₋₆ alkyl; wherein C₁₋₆ alkyl is optionally substituted with one or more halogen, which are the same or different; and
      wherein —R⁷, —R^{7a} and —R^{7b} are independently selected from the group consisting of —H and C₁₋₆ alkyl; wherein C₁₋₆ alkyl is optionally substituted with one or more halogen, which are the same or different.
20. The method of any one of items 1 to 19, wherein each —X′— is of formula (x4):

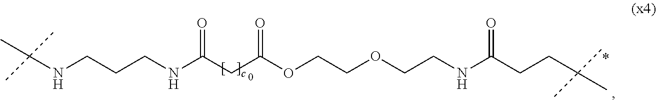

wherein
the unmarked dashed line indicates the attachment to —X— or carbonyl group and the dashed line marked with an asterisk indicates the attachment to -FG₁ or -L³-; and
c₀ is selected from the group consisting of 1, 2, 3, 4, 5, 6, 7, 8, 9 and 10, preferably c₀ is 7.
21. The method of any one of items 1 to 20, wherein each —Y′— is of formula (y0):

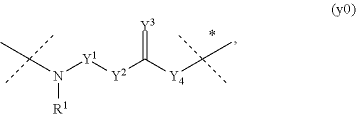

wherein
the unmarked dashed line indicates the attachment to —Y— or to the carbonyl group and the dashed line marked with an asterisk indicates the attachment to -FG₂ or -L³-;
—Y¹—, —Y⁴— are independently C₁₋₁₀ alkyl, which C₁₋₁₀ alkyl is optionally interrupted by one or more groups independently selected from —O—, -T-, —N(R^{y1})—, —C(O)O— and —C(O)N(R^{y1})—; and which C₁₋₁₀ alkyl chain is optionally substituted with one or more groups independently selected from —OH, -T, —NH(R^{y1}) and —C(O)N(R^{y2}R^{y2a}); wherein —R^{y1}, —R^{y2}, —R^{y2a} are independently selected from the group consisting of H and C₁₋₄ alkyl;
—Y²— is selected from the group consisting of —N(R²)—, —O—, —S— and —Se—;
═Y³ is selected from the group consisting of ═O, ═N(R²) and ═S;
—R¹, —R² are independently selected from the group consisting of —H, C₁₋₅ alkyl and -T; wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R³, which are the same or different;
   —R³ is selected from the group consisting of halogen, —CN, oxo, —C(O)OR⁴, —OR⁴, —C(O)R⁴, —C(O)N(R⁴)(R^{4a}), —S(O)₂N(R⁴)(R^{4a}), —S(O)N(R⁴)(R^{4a}), —S(O)₂R⁴, —S(O)R⁴, —N(R⁴)S(O)₂N(R^{4a})(R^{4b}), —SR⁴, —N(R⁴)(R^{4a}), —NO₂, —OC(O)R⁴, —N(R⁴)C(O)R^{4a}, —N(R⁴)S(O)₂R^{4a}, —N(R⁴)S(O)R^{4a}, —N(R⁴)C(O)OR^{4a}, —N(R⁴)C(O)N(R^{4a})(R^{4b}), —OC(O)N(R⁴)(R^{4a}) and C₁₋₆ alkyl; wherein C₁₋₆ alkyl is optionally substituted with one or more halogen, which are the same or different; and
      wherein —R⁴, —R^{4a} and —R^{4b} are independently selected from the group consisting of —H and C₁₋₆ alkyl; wherein C₁₋₆ alkyl is optionally substituted with one or more halogen, which are the same or different.
22. The method of any one of items 1 to 21, wherein each —Y′— is of formula (y1):

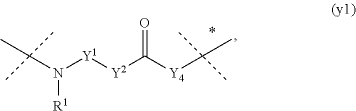

wherein
the unmarked dashed line indicates the attachment to —Y— or to the carbonyl group and the dashed line marked with an asterisk indicates the attachment to -FG₂ or -L³-;
—Y¹—, —Y⁴— are independently C₁₋₁₀ alkyl, which C₁₋₁₀ alkyl is optionally interrupted by one or more groups independently selected from —O—, -T-, —N(R^{y1})—, —C(O)O— and —C(O)N(R^{y1})—; and which C₁₋₁₀ alkyl chain is optionally substituted with one or more groups independently selected from —OH, -T, —NH(R^{y1}) and —C(O)N(R^{y2}R^{y2a}); wherein —R^{y1}, —R^{y2}, —R^{y2a} are independently selected from the group consisting of H and C₁₋₄ alkyl;
—Y²— is selected from the group consisting of —N(R²)—, —O—, —S— and —Se—;
—R¹, —R² are independently selected from the group consisting of —H, C₁₋₅ alkyl and -T; wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R³, which are the same or different;
   —R³ is selected from the group consisting of halogen, —CN, oxo, —C(O)OR⁴, —OR⁴, —C(O)R⁴, —C(O)N(R⁴)(R^{4a}), —S(O)₂N(R⁴)(R^{4a}), —S(O)N(R⁴)(R^{4a}), —S(O)₂R⁴, —S(O)R⁴, —N(R⁴)S(O)₂N(R^{4a})(R^{4b}), —SR⁴, —N(R⁴)(R^{4a}), —NO₂, —OC(O)R⁴, —N(R⁴)C(O)R^{4a}, —N(R⁴)S(O)₂R^{4a}, —N(R⁴)S(O)R^{4a}, —N(R⁴)C(O)OR^{4a}, —N(R⁴)C(O)N(R^{4a})(R^{4b}), —OC(O)N(R⁴)(R^{4a}) and C₁₋₆ alkyl; wherein C₁₋₆ alkyl is optionally substituted with one or more halogen, which are the same or different; and
      wherein —R⁴, —R^{4a} and —R^{4b} are independently selected from the group consisting of —H and C₁₋₆ alkyl; wherein C₁₋₆ alkyl is optionally substituted with one or more halogen, which are the same or different.
23. The method of any one of items 1 to 22, wherein each —Y′— is of formula (y2):

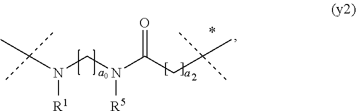

wherein
the unmarked dashed line indicates the attachment to —Y— or carbonyl group and the dashed line marked with an asterisk indicates the attachment to -FG₂ or -L³-;
—R¹, —R⁵ are independently selected from the group consisting of —H, methyl, ethyl, propyl and isopropyl; and
a₁, a₂ are independently selected from the group consisting of 1, 2, 3, 4, 5, 6, 7, 8, 9 and 10, preferably from the group consisting of 1, 2, 3, 4, 5, 6, 7 and 8, more preferably from the group consisting of 1, 2, 3, 4, 5 and 6 or even more preferably from the group consisting of 1,2 and 3.
24. The method of any one of items 1 to 23, wherein each —Y′— is of formula (y3):

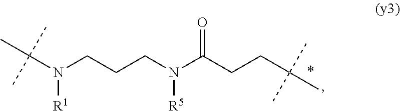

wherein
the unmarked dashed line indicates the attachment to —Y— or carbonyl group and the dashed line marked with an asterisk indicates the attachment to -FG₂ or -L³-; and
—R¹, —R⁵ are independently selected from the group consisting of —H, methyl, ethyl, propyl and isopropyl.
25. The method of any one of items 1 to 24, wherein each —Y′— is of formula (y4):

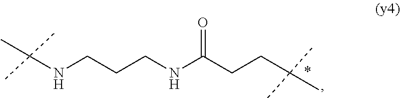

wherein
the unmarked dashed line indicates the attachment to —Y— or carbonyl group and the dashed line marked with an asterisk indicates the attachment to -FG₂ or -L³-.
26. The method of any one of items 1 to 25, wherein the method comprises the following steps:
   (a) mixing a solution A with a solution B to form an emulsion, wherein solution A comprises a first functionalized HA that is modified with one or more thiol functional groups and a second functionalized HA that is modified with one or more maleimide functional groups, wherein the thiol functional groups on the first functionalized HA react with the maleimide functional groups on the second functionalized HA to form a plurality of crosslinks which results in the formation of hydrogel HA microspheres, wherein,
      the first functionalized HA comprises a plurality of each of the following linearly connected Z¹ and Z⁵-i units:

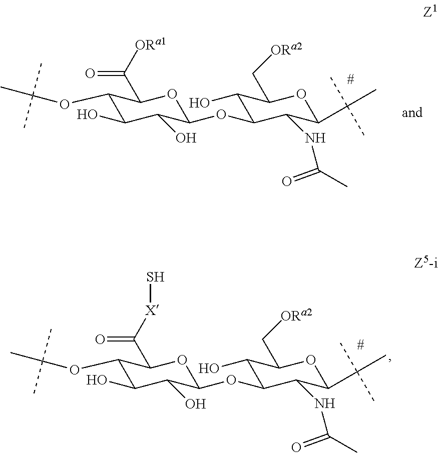

the second functionalized HA comprises a plurality of each of the following linearly connected Z¹ and Z⁶-i units:

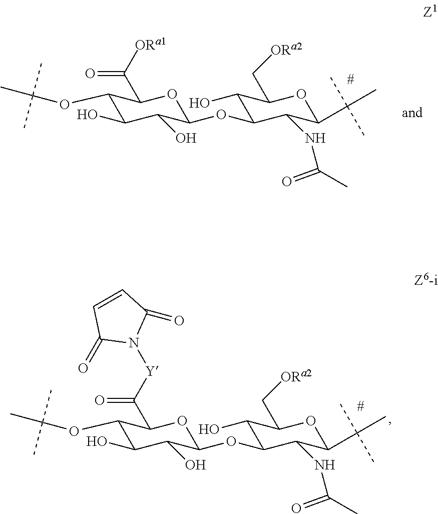

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is H or an alkali metal;
each —R^{a2} is —H;
each —X′— is of formula (x4):

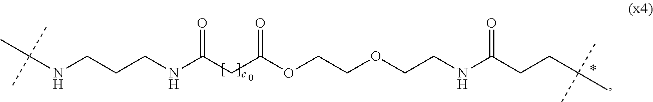

wherein the unmarked dashed line indicates the attachment to the carbonyl group, the dashed line marked with an asterisk indicates the attachment to the sulfur atom and c₀ is 7;
each —Y′— is of formula (y4):

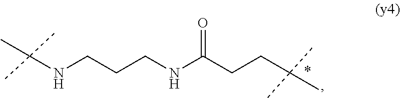

wherein the unmarked dashed line indicates the attachment to the carbonyl group and the dashed line marked with an asterisk indicates the attachment to the nitrogen atom of the maleimide ring;
(b) adding a pH-adjusting agent to the emulsion of step (a);
(c) collecting the obtained hydrogel HA microspheres of step (a) or (b), wherein said hydrogel comprises a plurality of Z³-i units:

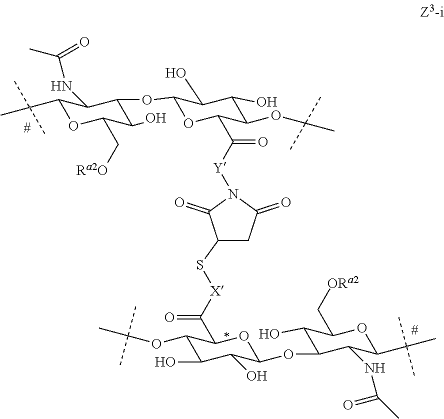

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each —R^{a2}, —X′— and —Y′— are defined as in step (a);
(d) size fractionating the obtained hydrogel HA microspheres of step (c) to obtain microspheres with a particular particle size distribution;
(e) washing the hydrogel HA microspheres obtained in step (d).
27. The method of any one of items 1 to 26, wherein the molecular weight of the first and second functionalized HA independently ranges from about 80 kDa to about 250 kDa, such as from about 90 kDa to about 200 kDa or such as from about 100 kDa to about 150 kDa.
28. The method of any one of items 1 to 27, wherein the molecular weight of the first and second functionalized HA ranges from about 100 kDa to about 150 kDa.
29. The method of any one of items 26 to 28, wherein the degree of thiol functionalization in Z⁵-i is about 5% and the degree of maleimide functionalization in Z⁶-i is about 10%,
30. The method of any one of items 1 to 29, wherein in step (a) solution A further comprises citrate and/or histidine, preferably at a pH of about 2.
31. The method of any one of items 1 to 30, wherein in step (a) solution B comprises heptane or tetradecane and sorbitan monooleate.
32. The method of any one of items 1 to 31, wherein in step (b) the pH-adjusting agent is N,N,N′,N′-tetramethylethylene diamine (TMEDA) and increases the pH of the emulsion of step (a), preferably to a pH of about 4.
33. The method of any one of items 1 to 32, wherein in step (d) size fractionating occurs via wet sieving.
34. Hydrogel HA microspheres or pharmaceutically acceptable salts thereof obtainable by any of the methods of items 1 to 33.
35. Use of the hydrogel HA microspheres or pharmaceutically acceptable salts thereof of item 34 as a carrier in a drug conjugate or pharmaceutically acceptable salt thereof.
36. A drug conjugate or pharmaceutically acceptable salt thereof comprising the hydrogel HA microspheres of item 34.
37. A drug conjugate or pharmaceutically acceptable salt thereof comprising a hyaluronic acid (HA) hydrogel microsphere comprising crosslinked HA chains or pharmaceutically acceptable salt thereof comprising crosslinked HA chains to which a plurality of drug moieties is covalently and reversibly conjugated, said drug conjugate comprising a plurality of each of the following units:

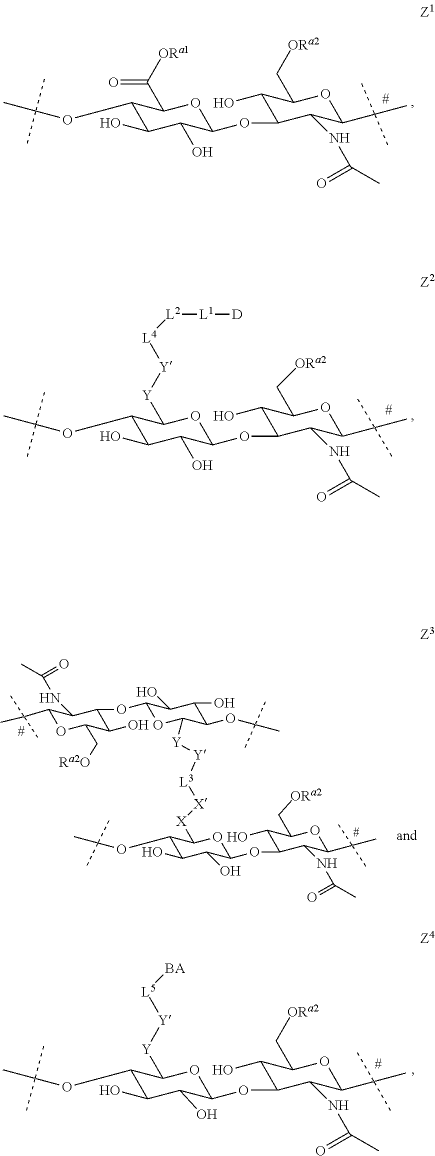

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is independently selected from the group consisting of —H, C₁₋₁₀ alkyl, an ammonium ion, a tetrabutylammonium ion, a cetyl trimethylammonium ion, an alkali metal ion and an alkaline earth metal ion;
each —R^{a2} is independently —H or C₁₋₁₀ alkyl;
each —X—, —Y— is independently a carbonyl group or absent;
each —X′—, —Y′— is independently a spacer moiety or absent;
wherein each -D is independently a drug moiety that is covalently and reversibly conjugated to -L¹-;
each -L¹- is independently a reversible linker moiety;
each -L²- is independently a spacer moiety or absent;
each -L³-, -L⁴-, -L⁵- is independently a linkage moiety or absent; and
each -BA is independently a blocking agent.
38. The drug conjugate or pharmaceutically acceptable salt thereof of item 37, wherein said drug conjugate comprises Z¹ in a range of about 50% to about 98%, Z² in a range of about 0.1% to about 20%, Z³ in a range of about 0.1% to about 20% and Z⁴ in a range of about 0.1% to about 10%.
39. The drug conjugate or pharmaceutically acceptable salt thereof of item 37 or 38, wherein said drug conjugate comprises Z¹ in a range of about 75% to about 98%, Z² in a range of about 0.1% to about 10%, Z³ in a range of about 0.1% to about 10% and Z⁴ in a range of about 0.1% to about 5%.
40. The drug conjugate or pharmaceutically acceptable salt thereof of any one of items 37 to 39, wherein said drug conjugate comprises Z¹ in a range of about 78% to about 96%, Z² in a range of about 2% to about 10%, Z³ in a range of about 1% to about 7% and Z⁴ in a range of about 0.5% to about 5%.
41. The drug conjugate or pharmaceutically acceptable salt thereof of any one of items 37 to 40, wherein -BA is selected from the group consisting of

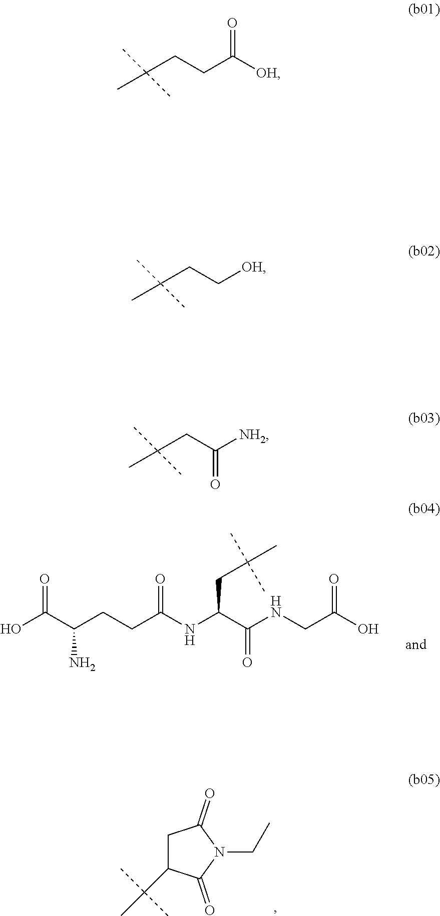

wherein the dashed line indicates the attachment to -L⁵-.
42. The drug conjugate or pharmaceutically acceptable salt thereof of item 37 to 41, wherein -BA is of formula (b01).
43. The drug conjugate or pharmaceutically acceptable salt thereof of any one of items 37 to 42, wherein said drug conjugate comprises a plurality of each of the following units:

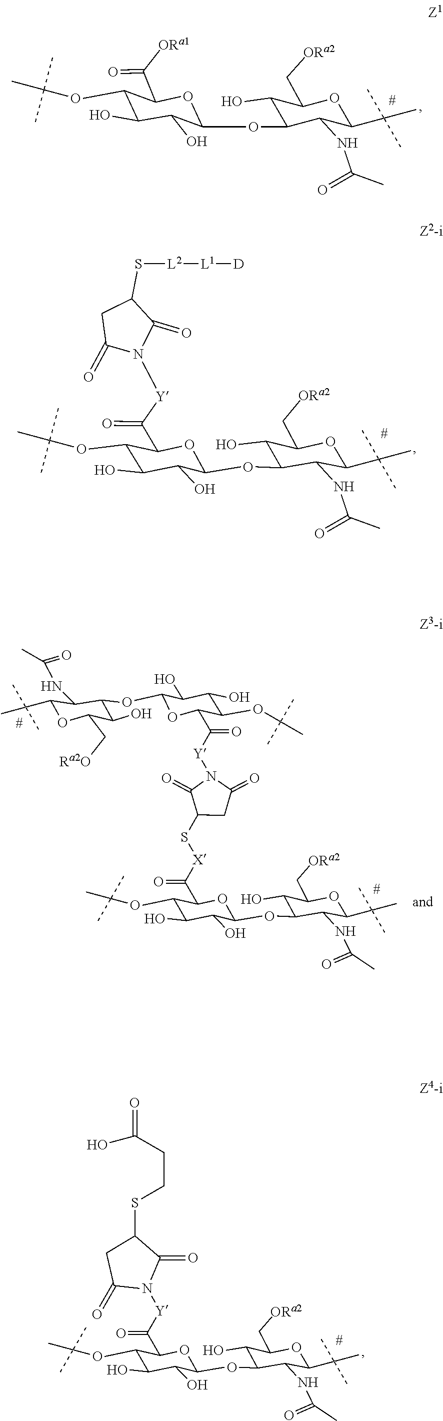

each R^{a1} is independently selected from the group consisting of —H, C₁₋₁₀ alkyl, an ammonium ion, a tetrabutylammonium ion, a cetyl trimethylammonium ion, an alkali metal ion and an alkaline earth metal ion;
each —R^{a2} is independently —H or C₁₋₁₀ alkyl;
each —X′—, —Y′— is independently a spacer moiety or absent;
wherein each -D is independently a drug moiety that is covalently and reversibly conjugated to -L¹-;
each -L¹- is independently a reversible linker moiety;
each -L²- is independently a spacer moiety or absent; and
wherein said drug conjugate comprises Z¹ in a range of about 50% to about 98%, Z²-i in a range of about 0.1% to about 20%, Z³-i in a range of about 0.1% to about 20% and Z⁴-i in a range of about 0.1% to about 10%.
44. The drug conjugate or pharmaceutically acceptable salt thereof of any one of items 37 to 43, wherein -D is selected from the group consisting of small molecule drug moieties, medium size drug moieties, peptide drug moieties and protein drug moieties.
45. The drug conjugate or pharmaceutically acceptable salt thereof of any one of items 37 to 44, wherein -D is a protein drug moiety.
46. The drug conjugate or pharmaceutically acceptable salt thereof of any one of items 37 to 45, wherein -D is a monoclonal or polyclonal antibody or fragment or fusion thereof.
47. The drug conjugate or pharmaceutically acceptable salt thereof of any one of items 37 to 46, wherein each R^{a1} is an alkali metal ion or H and —R^{a2} is —H.
48. The drug conjugate or pharmaceutically acceptable salt thereof of any one of items 37 to 47, wherein each of —X— and —Y— are a carbonyl group.
49. The drug conjugate or pharmaceutically acceptable salt thereof of any one of items 37 to 48, wherein each -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀alkenyl and C₂₋₅₀ alkynyl; wherein -T′-, C₁₋₅₀ alkyl, C₂₋₅₀ alkenyl and C₂₋₅₀ alkynyl are optionally substituted with one or more —R^{y2}, which are the same or different and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})— and —OC(O)N(R^{y3})—;
   —R^{y1} and —R^{y1a} are independently selected from the group consisting of —H, -T′, C₁₋₅₀ alkyl, C₂₋₅₀ alkenyl and C₂₋₅₀ alkynyl; wherein -T′, C₁₋₅₀ alkyl, C₂₋₅₀ alkenyl and C₂₋₅₀ alkynyl are optionally substituted with one or more —R^{y2}, which are the same or different, and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
   each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each T′ is independently optionally substituted with one or more —R^{y2}, which are the same or different;
   each —R^{y2} is independently selected from the group consisting of halogen, —CN, oxo (═O), —C(O)OR^{y5}, —OR^{y5}, —C(O)R^{y5}, —C(O)N(R⁵)(R^{y5a}), —S(O)₂N(R^{y5})(R^{y5a}), —S(O)N(R⁵)(R^{y5a}), —S(O)₂R^{y5}, —S(O)R^{y5}, —N(R^{y5})S(O)₂N(R⁵)(R^{y5a}), —SR^{y5}, —N(R^{y5})(R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5})(R^{y5a}), —OC(O)N(R^{y5})(R^{y5a}), and C₁₋₆ alkyl; wherein C₁₋₆ alkyl is optionally substituted with one or more halogen, which are the same or different; and
   each —R^{y3}, —R^{y3a}, —R^{y4}, —R^{y4a}, —R⁵, —R^{y5a} and —R^{y5b} is independently selected from the group consisting of —H and C₁₋₆ alkyl; wherein C₁₋₆ alkyl is optionally substituted with one or more halogen, which are the same or different.
50. The drug conjugate or pharmaceutically acceptable salt thereof of any one of items 37 to 49, wherein each -L¹- is of formula (I):

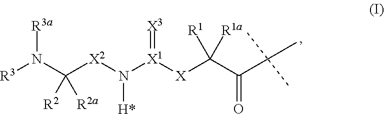

wherein
the dashed line indicates the attachment to a nitrogen of -D by forming an amide bond; —X— is —C(R⁴R^{4a})—; —N(R⁴)—; —O—; —C(R⁴R^{4a})—C(R⁵R^{5a})—; —C(R⁵R^{5a})—C(R⁴R^{4a})—; —C(R⁴R^{4a})—N(R⁶)—; —N(R⁶)—C(R⁴R^{4a})—; —C(R⁴R^{4a})—O—; —O—C(R⁴R^{4a})—; or —C(R⁷R^{7a})—;
X¹ is C; or S(O);
—X²— is —C(R⁸R^{8a})—; or —C(R⁸R^{8a})—C(R⁹R^{9a})—;
═X³ is ═O; ═S; or ═N—CN;
—R¹, —R^{1a}, —R², —R^{2a}, —R⁴, —R^{4a}, —R⁵, —R^{5a}, —R⁶, —R⁸, —R^{8a}, —R⁹, —R^{9a} are independently selected from the group consisting of —H; and C₁₋₆ alkyl;
—R³, —R^{3a} are independently selected from the group consisting of —H; and C₁₋₆ alkyl, provided that in case one of —R³, —R^{3a} or both are other than —H they are connected to N to which they are attached through a sp³-hybridized carbon atom;
—R⁷ is —N(R¹⁰R^{10a}); or —NR¹⁰—(C═O)—R¹¹;
—R^{7a}, —R¹⁰, —R^{10a}, —R¹¹ are independently of each other —H; or C₁₋₁₀ alkyl;
optionally, one or more of the pairs —R^{1a}/—R^{4a}, —R^{1a}/—R^{5a}, —R^{1a}/—R^{7a}, —R^{4a}/—R^{5a}, —R^{8a}/—R^{9a} form a chemical bond;
optionally, one or more of the pairs —R¹/—R^{1a}, —R²/—R^{2a}, —R⁴/—R^{4a}, —R⁵/—R^{5a}, —R¹/—R^{8a}, —R⁹/—R^{9a} are joined together with the atom to which they are attached to form a C₃₋₁₀ cycloalkyl; or 3- to 10-membered heterocyclyl;
optionally, one or more of the pairs —R¹/—R⁴, —R¹/—R⁵, —R¹/—R⁶, —R¹/—R^{7a}, —R⁴/—R⁵, —R⁴/—R⁶, —R¹/—R⁹, —R²/—R³ are joined together with the atoms to which they are attached to form a ring A;
optionally, —R³/—R^{3a} are joined together with the nitrogen atom to which they are attached to form a 3- to 10-membered heterocycle;
ring A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
each -L¹- is substituted with one -L²- and optionally further substituted provided that the hydrogen marked with the asterisk in formula (I) is not replaced by a substituent.
51. The drug conjugate or pharmaceutically acceptable salt thereof of item 50, wherein —X— is —C(R⁷R^{7a})—, —R⁷ is —NR¹⁰ —(C═O)—R¹¹ and —R^{7a} is —H.
52. The drug conjugate or pharmaceutically acceptable salt thereof of any one of items 37 to 49, wherein each -L¹- is of formula (XI):

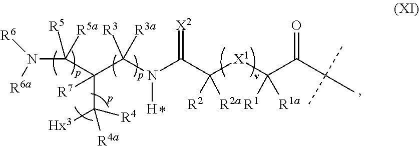

wherein
the dashed line indicates the attachment to the nitrogen of the primary or secondary amine of -D;
v is selected from the group consisting of 0 or 1;
—X¹— is selected from the group consisting of —C(R⁸)(R^{8a})—, —N(R⁹)— and —O—;
═X² is selected from the group consisting of ═O and ═N(R¹⁰);
—X³ is selected from the group consisting of —O, —S and —Se;
each p is independently selected from the group consisting of 0 or 1, provided that at most one p is 0;
—R⁶, —R^{6a}, —R¹⁰ are independently selected from the group consisting of —H, —C(R¹¹)(R^{11a})(R^{11b}) and -T;
—R⁹ is selected from the group consisting of —C(R¹¹)(R^{11a})(R^{11b}) and -T;
—R¹, —R^{1a}, —R², —R^{2a}, —R³, —R^{3a}, —R⁴, —R^{4a}, —R⁵, —R^{5a}, —R⁷, —R⁸ —R^{8a}, —R¹¹, —R^{11a} and
—R^{11b} are independently selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
—R¹², —R^{12a}, —R^{12b} are independently selected from the group consisting of —H, -T, C₁₋₆ alkyl, C₂₋₆ alkenyl and C₂₋₆ alkynyl; wherein -T, C₁₋₆ alkyl, C₂₋₆ alkenyl and C₂₋₆ alkynyl are optionally substituted with one or more —R¹³, which are the same or different and wherein C₁₋₆ alkyl, C₂₋₆ alkenyl and
   C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R¹³, which are the same or different;
—R¹³ is selected from the group consisting of halogen, —CN, oxo, —C(O)OR¹⁵, —OR¹⁵, —C(O)R¹⁵, —C(O)N(R¹⁵)(R^{15a}), —S(O)₂N(R¹⁵)(R^{15a}), —S(O)N(R¹⁵) (R^{15a}), —S(O)₂R¹⁵, —S(O)R¹⁵, —N(R¹⁵)S(O)₂N(R^{15a})(R^{15b}), —SR¹⁵, —N(R¹⁵)(R^{15a}), —NO₂, —OC(O)R¹⁵, —N(R¹⁵)C(O)R^{15a}, —N(R¹⁵)S(O)₂R^{15a}, —N(R¹⁵)S(O)R^{15a}, —N(R¹⁵)C(O)OR^{15a}, —N(R¹⁵)C(O)N(R^{15a})(R^{15b}), —OC(O)N(R¹⁵)(R^{15a}) and C₁₋₆ alkyl; wherein C₁₋₆ alkyl is optionally substituted with one or more halogen, which are the same or different;
wherein —R¹⁴, —R^{14a}, —R¹⁵, —R^{15a} and —R^{15b} are independently selected from the group consisting of —H and C₁₋₆ alkyl; wherein C₁₋₆ alkyl is optionally substituted with one or more halogen, which are the same or different;
optionally, one or more of the pairs —R¹/—R^{1a}, —R²/—R^{2a}, —R³/—R^{3a}, —R⁴/—R^{4a}, —R⁵/—R^{5a} or —R⁸/—R^{8a} are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁸, —R¹/—R⁹, —R²/—R⁹ or —R²/—R¹⁰ are joined together with the atoms to which they are attached to form a ring -A-;
wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, one or more of the pairs —R³/—R⁶, —R⁴/—R⁶, —R⁵/—R⁶, —R⁶/—R^{6a} or —R⁶/—R⁷ form together with the atoms to which they are attached a ring -A′-;
wherein -A′- is selected from the group consisting of 3- to 10-membered heterocyclyl and 8- to 11-membered heterobicyclyl; and
-L¹- is substituted with -L²- provided that the hydrogen marked with the asterisk in formula (XI) is not replaced by a substituent.
53. A pharmaceutical composition comprising the drug conjugate or pharmaceutically acceptable salt thereof of any one of items 36 to 52 and at least one pharmaceutically acceptable excipient.
54. The drug conjugate or pharmaceutically acceptable salt thereof of any one of items 36 to 52 or the pharmaceutical composition of item 53 for use as a medicament.
55. The drug conjugate or pharmaceutically acceptable salt thereof of any one of items 36 to 52 or the pharmaceutical composition of item 53 for use in a method of treating a disease that can be treated with D-H or its pharmaceutically acceptable salt thereof.
56. The drug conjugate or pharmaceutically acceptable salt thereof of any one of items 36 to 52 or the pharmaceutical composition of item 53 for use in treating an ocular disease.
57. The drug conjugate or pharmaceutically acceptable salt thereof of any one of items 36 to 52 or the pharmaceutical composition of item 53 for use in the manufacture of a medicament for the therapeutic treatment of a disease.
58. The drug conjugate or pharmaceutically acceptable salt thereof of any one of items 36 to 52 or the pharmaceutical composition of item 53 for use in the manufacture of a medicament for the therapeutic treatment of an ocular disease.
59. A method of preventing or treating a disease that can be prevented or treated with D-H, the method comprising the step of administering to a subject in need thereof a therapeutically effective amount of the drug conjugate or pharmaceutically acceptable salt thereof of any one of items 36 to 52 or the pharmaceutical composition of item 53.
60. The method of item 59, wherein the disease is an ocular disease.
61. A method of preparing a drug conjugate or pharmaceutically acceptable salt thereof, wherein the method comprises the following steps:
   (a) providing the hydrogel HA microspheres or pharmaceutically acceptable salts thereof obtained by any of the methods of items 1 to 33, wherein said hydrogel comprises one or more unreacted -FG₁ or -FG₂;
   (b) providing a monoconjugate reagent D-L¹-L²-FG₃, a bisconjugate reagent FG₃-L²-L¹-D-L¹-L²-FG₃- or a trisconjugate reagent of formula (t):

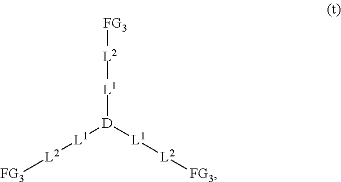

wherein each -D is independently a drug moiety that is covalently and reversibly conjugated to -L¹-;
each -L¹- is independently a reversible linker moiety;
each -L²- is independently a spacer moiety or absent;
each -FG₃ is independently a functional group that reacts with one -FG₁ or -FG₂;
(c) mixing the hydrogel HA microspheres of step (a) with the monoconjugate, bisconjugate or trisconjugate reagent of step (b);
(d) mixing the drug conjugate or pharmaceutically salt thereof of step (c) with a blocking reagent; and
(e) collecting the drug conjugate or pharmaceutically acceptable salt thereof of step (c) or (d).
62. The method of item 61, wherein said method comprises optional purification steps of the drug conjugate obtained in step (c) or (d).
63. The method of item 61 or 62, wherein in step (c) the hydrogel HA microspheres are mixed with a monoconjugate reagent D-L¹-L²-FG₃.
64. The method of any one of items 61 to 63, wherein the monoconjugate reagent D-L¹-L²-FG₃ is of formula (m1) or (m2):

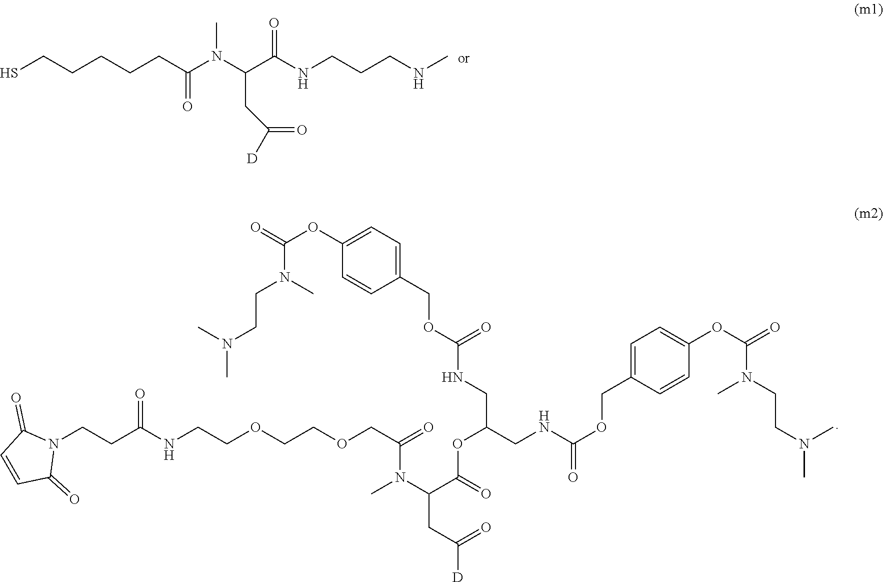 [SMILES: [2H]C(=O)CC(C(=O)NCCCNC)N(C)C(=O)CCCCCS ; [2H]C(=O)CC(C(=O)OC(CNC(=O)OCc1ccc(OC(=O)N(C)CCN(C)C)cc1)CNC(=O)OCc1ccc(OC(=O)N(C)CCN(C)C)cc1)N(C)C(=O)COCCOCCNC(=O)CCN1C(=O)C=CC1=O]

65. The method of any one of items 61 to 64, wherein the monoconjugate reagent D-L¹-L²-FG₃ is of formula (m′ 1) or (m′2):

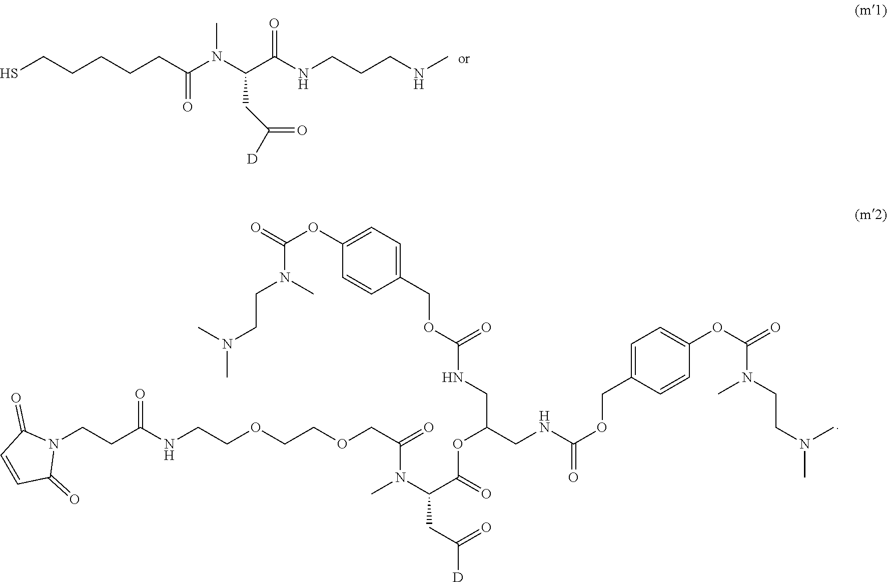 [SMILES: [2H]C(=O)C[C@@H](C(=O)NCCCNC)N(C)C(=O)CCCCCS ; [2H]C(=O)C[C@@H](C(=O)OC(CNC(=O)OCc1ccc(OC(=O)N(C)CCN(C)C)cc1)CNC(=O)OCc1ccc(OC(=O)N(C)CCN(C)C)cc1)N(C)C(=O)COCCOCCNC(=O)CCN1C(=O)C=CC1=O]

66. A drug conjugate or pharmaceutically acceptable salts thereof obtainable by any of the methods of items 61 to 65.

Another aspect of the present invention relates to a method for precipitating a polymer in a flow system.

The present invention also provides a method for isolating a polymer in a setup for precipitating and isolating a polymer.

The present invention also relates to polymers obtainable by the methods of the present invention.

Another aspect of the present invention relates to a flow system for precipitating a polymer.

The present invention also provides a setup for precipitating and isolating a polymer comprising a flow system and a collecting assembly.

The method for precipitating a polymer in a flow system may comprise the steps of:
(a′) flowing, optionally simultaneously, a first solution comprising the polymer through a first channel and a second solution comprising an anti-solvent through a second channel;
(b′) combining the first and second solutions of step (a′);
(c′) flowing the combined mixture of step (b′) into at least one precipitating unit; and
(d′) precipitating the polymer;
wherein if more than one precipitating unit is present, the mixture comprising the precipitated polymer that flows out from one precipitating unit is combined with the second solution comprising the anti-solvent or optionally with another solution comprising an anti-solvent before it is flowed into another precipitating unit.

The method for isolating a polymer in a setup for precipitating and isolating a polymer may comprise the steps of:
(a′) flowing, optionally simultaneously, a first solution comprising the polymer through a first channel and a second solution comprising an anti-solvent through a second channel;
(b′) combining the first and second solutions of step (a′);
(c′) flowing the combined mixture of step (b′) into at least one precipitating unit;
(d′) precipitating the polymer; and
(e′) isolating the precipitate of step (d′),
wherein if more than one precipitating unit is present, the mixture comprising the precipitated polymer that flows out from one precipitating unit is combined with the second solution comprising the anti-solvent or optionally with another solution comprising an anti-solvent before it is flowed into another precipitating unit.

The flow system for precipitating a polymer of the present invention may comprise:
a vessel comprising a first solution comprising the polymer;
at least one storage vessel comprising a second solution comprising an anti-solvent;
at least one combining unit for combining the first and second solutions or for combining the mixture that flows out from a precipitating unit with the second solution comprising an anti-solvent or optionally with another solution comprising an anti-solvent;
at least one precipitation unit for precipitating the polymer;
   wherein the vessel, the at least one storage vessel, the at least one combining unit, and the at least one precipitation unit are connected via connective channels to provide a continuous flow path, wherein
      the first solution flows through a first channel from the vessel to the combining unit;
      the second solution flows through a second channel from the at least one storage unit to the combining unit;
      the combined mixture flows through at least one of the precipitation units and wherein if more than one precipitating unit is present, the mixture comprising the precipitated polymer that flows out from one precipitating unit is combined with the second solution comprising the anti-solvent or optionally with another solution comprising an anti-solvent before it is flowed into another precipitating unit.

The setup for precipitating and isolating a polymer of the present invention may comprise:
I) a flow system for precipitating a polymer comprising:
   a vessel comprising a first solution comprising the polymer;
   at least one storage vessel comprising a second solution comprising an anti-solvent;
   at least one combining unit for combining the first and second solutions or for combining the mixture that flows out from a precipitating unit with the second solution comprising an anti-solvent or optionally with another solution comprising an anti-solvent;
   at least one precipitation unit for precipitating the polymer;
      wherein the vessel, the at least one storage vessel, the at least one combining unit, and the at least one precipitation unit are connected via connective channels to provide a continuous flow path, wherein
         the first solution flows through a first channel from the vessel to the combining unit;
         the second solution flows through a second channel from the at least one storage unit to the combining unit;
         the combined mixture flows through at least one of the precipitation units and wherein if more than one precipitating unit is present, the mixture comprising the precipitated polymer that flows out from one precipitating unit is combined with the second solution comprising the anti-solvent or optionally with another solution comprising an anti-solvent before it is flowed into another precipitating unit; and
II) a collecting assembly for isolating the precipitated polymer.

Exemplary polymers are selected from the group consisting of polysaccharides such as hyaluronic acid, hyaluronic acid and derivatives or functionalized hyaluronic acid, heparin, heparan sulfate, heparosan, chondroitin sulfate, dermatan sulfate, keratan sulfate, cellulose, carboxymethyl cellulose, hydroxypropyl methylcellulose, chitin, chitosan, dextran or dextrin; polyethers, such as poly(ethyleneglycol) or poly(propylene glycols); polyesters, such as polyhydroxybutyrate, poly(glycolic acid), polybutylene terephthalate, poly(caprolactone), poly(lactic acid) or poly(lactic-co-glycolic acid); proteins, such as gelatin or collagen; polyolefins, such as poly(2-methacryloyl-oxyethyl phosphorylcholine), poly(acrylic acid), poly(acrylate), poly(acrylamide), poly(cyanoacrylate), poly(dimethylacrylamide), polyethylene, poly(hydroxyethyl acrylate), poly(2-hydroxyethyl methacrylate), poly(N-(2-hydroxypropyl)methacrylamide), poly(hydroxypropyl methacrylate), poly(vinyl alcohol), poly(vinyl amine), poly(vinylmethylether) or poly(vinylpyrrolidone); poly(oxazolines), such as poly(methyloxazoline) or poly(ethyloxazoline); polyamides; poly(amidoamines); poly(amino acids); polyanhydrides; poly(aspartamides); polycarbonates; poly(alkylene phosphates) such as poly(ethylene phosphates); poly(iminocarbonates); poly(methacrylamides); poly(organophosphazenes); poly(ortho esters); poly(siloxanes) and poly(urethanes).

In certain embodiments, the polymer is a functionalized hyaluronic acid. In certain embodiments, the polymer is hyaluronic acid (i.e. native hyaluronic acid). In certain embodiments, the polymer is poly(ethyleneglycol). In certain embodiments, the polymer is collagen.

A functionalized HA may comprise a plurality of linearly connected Z¹ and Z⁵ units, Z¹ and Z⁶ units or Z¹ and Z⁷ units, wherein Z¹, Z⁵ and Z⁶ units are as described elsewhere herein.

In certain embodiments, a functionalized HA comprises a plurality of each of the following linearly connected Z¹ and Z⁵ units:

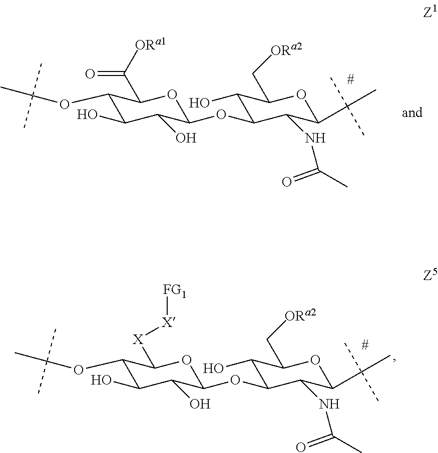

or a plurality of each of the following linearly connected Z¹ and Z⁶ units:

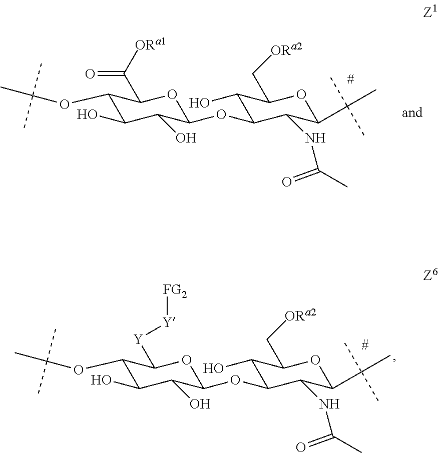

or a plurality of each of the following linearly connected Z¹ and Z⁷ units:

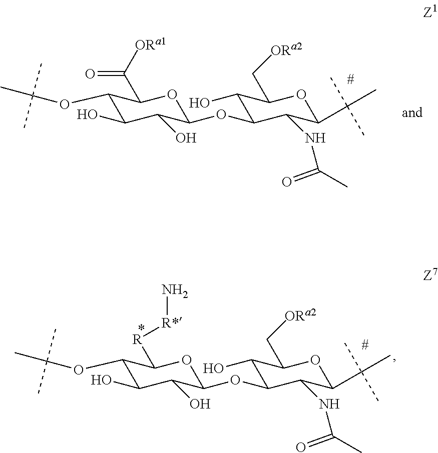

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is independently selected from the group consisting of —H, C₁₋₁₀ alkyl, an ammonium ion, a tetrabutylammonium ion, a cetyl trimethylammonium ion, an alkali metal ion and an alkaline earth metal ion;
each —R^{a2} is independently —H or C₁₋₁₀ alkyl;
each -FG₁, -FG₂ is defined as elsewhere herein;
each —X—, —Y—, —R*— is independently a carbonyl group or absent; and
each —X′—, —Y′—, —R*′— is independently a spacer moiety as defined elsewhere herein or absent.

In certain embodiments, a functionalized HA comprises a plurality of each of the following linearly connected Z¹ and Z⁵-i units:

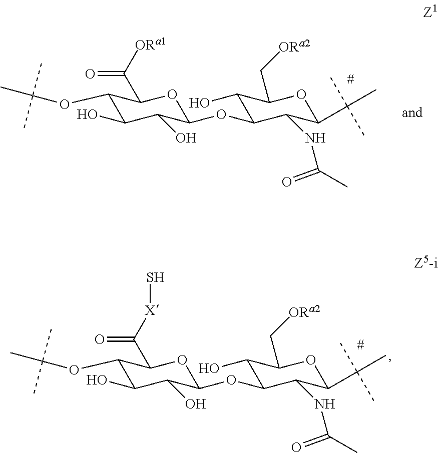

or a plurality of each of the following linearly connected Z¹ and Z⁶-i units:

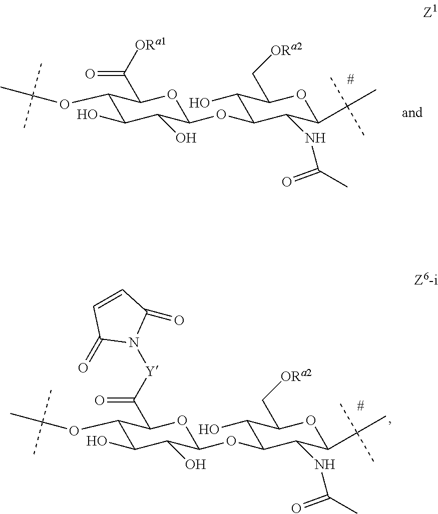

or a plurality of each of the following linearly connected Z¹ and Z⁷-i units:

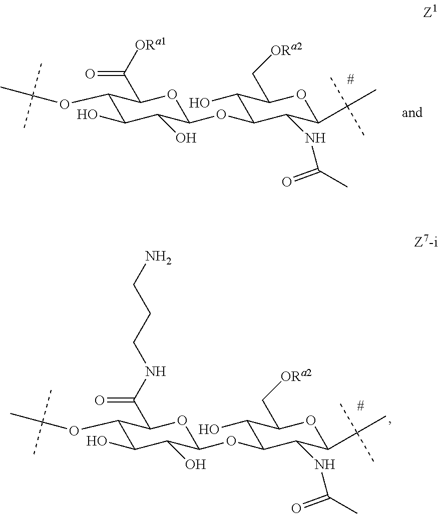

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
—X′— is of formula (x4):

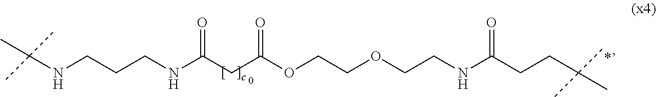

wherein the unmarked dashed line indicates the attachment to the carbonyl group, the dashed line marked with an asterisk indicates the attachment to the sulfur atom and c₀ is 7; —Y′— is of formula (y4):

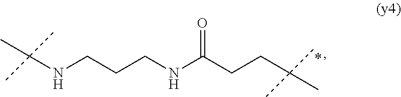

wherein the unmarked dashed line indicates the attachment to the carbonyl group and the dashed line marked with an asterisk indicates the attachment to the nitrogen atom of the maleimide ring;
each R^{a1} is H or an alkali metal;
each —R^{a2} is —H; and the second solution is ethanol.

The present invention also provides a method for precipitating a functionalized HA, wherein the method comprises continuous flow precipitation and wherein the functionalized HA comprises a plurality of each of the following linearly connected Z¹ and Z⁵-i units:

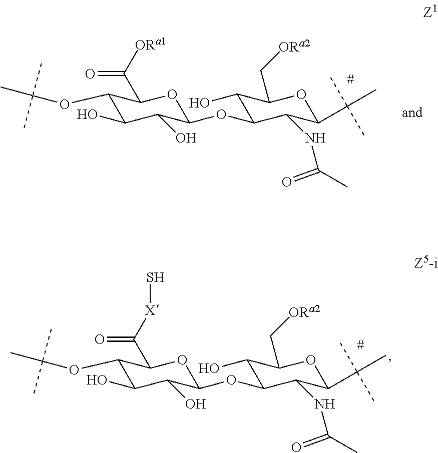

or a plurality of each of the following linearly connected Z¹ and Z⁶-i units:

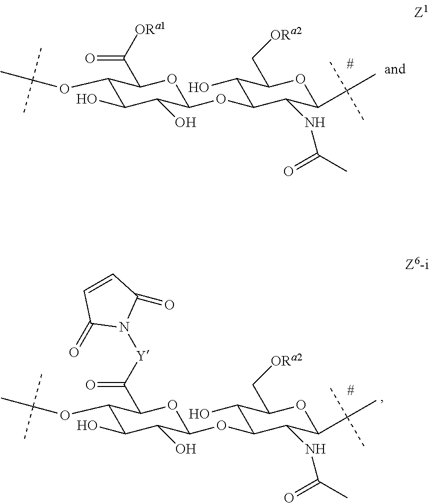

or a plurality of each of the following linearly connected Z¹ and Z⁷-i units:

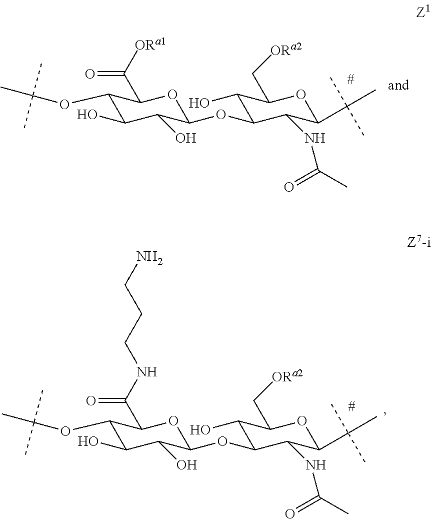

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
—X′— is of formula (x4):

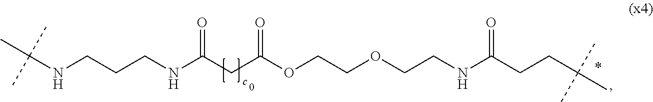

wherein the unmarked dashed line indicates the attachment to the carbonyl group, the dashed line marked with an asterisk indicates the attachment to the sulfur atom and c₀ is 7;
—Y′— is of formula (y4):

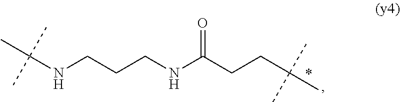

wherein the unmarked dashed line indicates the attachment to the carbonyl group and the dashed line marked with an asterisk indicates the attachment to the nitrogen atom of the maleimide ring;
each R^{a1} is H or an alkali metal; and
each —R^{a2} is —H.

One advantage of using the flow system, setup or method for precipitating or isolating a polymer described above is that said polymer, such as a functionalized HA as described elsewhere herein, may be obtained in higher yields and shorter processing time in comparison to corresponding batch processes. Another advantage lies in the ability to control and improve the morphology of an isolated polymer, such as of a functionalized HA as described elsewhere herein, that has a string, granular or non-sticky porous shape in contrast to a polymer obtained by a batch process that may have the appearance and properties of a sticky filter cake. A polymer, such as a functionalized HA, in the form of a granular shape provides a higher surface area that prevents the formation of large aggregates and has improved dissolution properties. In particular, in order to obtain polymers, such as a functionalized HA, with a suitable morphology, it is advantageous that at least two precipitating units are present and the mixture comprising the precipitated polymer that flows out from one precipitating unit is combined with the second solution comprising the anti-solvent or optionally with another solution comprising an anti-solvent before it is flowed into another precipitating unit.

An additional advantage of the flow system, setup or method for precipitating or isolating a polymer described above to corresponding batch processes is that it allows for the control of the residence time, temperature, requires reduced process volumes (e.g., a lower peak process volume of anti-solvent is required in the storage vessel at once), allows better control of the precipitation and isolation processes and improved scalability. Such a system is also less prone to contamination in comparison to its corresponding batch processes.

In general, within the flow system of the present invention, the solutions and mixtures may be flowed by gas overpressure or a pump. Said pump may be selected from the group consisting of gear pump; diaphragm pump (also known as a membrane pump); plunger pump such as a high-performance liquid chromatography pump; syringe pump; peristaltic pump and piston pump.

Typically, a pump is calibrated within the pressure and desired flow range, to ensure it is flowing or introducing the solution at a specific flow rate.

Throughout the invention, parameters such as flow rate, concentration, temperature, pressure and the like may be measured and optimized in order to obtain a high yield of precipitation and purity. Also, the flow system or setup for precipitating and isolating a polymer of the present invention may be sized and configured to allow for the purification and isolation of the polymer at a wide range and scale. Accordingly, the polymer, such as the functionalized HA may be purified and isolated in an amount greater than about 10 g, such as greater than 15 g, such as greater than 30 g, such as greater than 50 g, such as greater than 100 g, such as greater than 150 g, such as greater than 175 g, such as greater than 200 g, such as greater than 250 g, such as greater than 400 g, such as greater than 600 g, such as greater than 800 g, such as greater than 1 kg, such as greater than 10 kg, such as greater than 100 kg, such as greater than 200 kg, such as greater than 300 kg or such as greater than 500 kg.

In general, the dimensions of the channels incorporated in the flow system or setup described above may also be chosen, for example, to allow a certain volumetric or linear flow rate of fluid in the channel. The number of channels and the shape of the channels can be varied by any method known to those skilled in the art. In some cases, more than one channel or capillary may be used. For example, two or more channels may be used, where they are positioned inside each other, positioned adjacent to each other or positioned to intersect with each other.

The flow rate influences the residence time (assuming the same vessel volume). The effectiveness of mixing may be dependent on the flow rate, i.e. a low flow rate has low mixing efficiency, a high flow rate has high mixing efficiency. Mixing efficacy may also be influenced by the flow turbidity which is itself dependent on geometry and flow speed.

Typically, the flow rate for each solution will be held constant over the course of the flow process. Also, typically, in step (a′) the first solution is flowed through the first channel simultaneously with the second solution.

Within the method above, each solution may be independently flowed with a flow rate ranging from about 1 ml/min to about 100 l/min, such as from about 10 ml/min to about 90 l/min, such as from about 15 ml/min to about 60 l/min, such as from about 20 ml/min to about 40 l/min, such as from about 30 ml/min to about 30 l/min or such as from about 10 ml/min to about 10 l/min.

In certain embodiments, the first solution is flowed with a flow rate ranging from about 10 ml/min to about 10 l/min, such as from about 10 ml/min to about 5 l/min, such as from about 10 ml/min to about 200 ml/min, such as from about 15 ml/min to about 115 ml/min, such as from about 25 to about 105 ml/min, such as from about 35 to about 95 ml/min, such as from about 45 to about 85 ml/min or such as from about 55 to about 75 ml/min. Advantageously, the first solution is flowed with a flow rate of about 64 ml/min.

In certain embodiments, the second solution is flowed with a flow rate ranging from about 10 ml/min to about 10 l/min, such as from about 10 ml/min to about 5 l/min, such as from about 10 ml/min to about 200 ml/min, such as from about 55 ml/min to about 135 ml/min, such as from about 65 ml/min to about 125 ml/min, such as from about 75 ml/min to about 115 ml/min or such as from about 85 ml/min to about 105 ml/min. Advantageously, the second solution is flowed with a flow rate of about 96 ml/min.

In step (b′) the first solution is combined with the second solution comprising the anti-solvent in a combining unit, such as a mixer. Also, the mixture comprising the precipitated polymer, such as the precipitated functionalized HA, that flows from a precipitating unit is combined with the second solution comprising the anti-solvent or optionally with another solution comprising an anti-solvent in a combining unit, such as a mixer. Said mixer may be a Y-style, T-style, X-style, arrow-style mixer or static mixer. It is understood that some precipitation of the polymer may already occur upon combining the first solution with the second solution in the combining unit, i.e. upon first contact between the polymer and the anti-solvent.

In certain embodiments, the mixer is a Y-style mixer (a Y-style or Y-shaped mixer is mixing the solutions at 120°). In certain embodiments, the mixer is a T-style mixer (a T-style or T-shaped mixer is mixing two solutions in a head on fashion, or at 90°). In certain embodiments, the mixer is a X-style mixer. In certain embodiments, the mixer is an arrow-style mixer. Advantageously, in step (b′) the first solution is combined with the second solution comprising the anti-solvent in a Y-style mixer and the mixture comprising the precipitated polymer that flows from a precipitating unit is combined with the second solution comprising the anti-solvent in a T-style mixer, preferably in a Y-style mixer.

In step (c′) the combined solutions are flowed into at least one precipitating unit that may be a continuous flow reactor such as a coil reactor or tubular reactor. Said tubular reactor may be made of an inert plastic, an inert metal, an alloy or glass.

The length of the coil or tubular reactor determines or influences the residence time such as the precipitating or ripening time. Said coil or tubular reactor has a length ranging from about 1 m to about 1000 m, such as from about 1 m to about 900 m, such as from about 1 m to about 800 m, such as from about 1 m to about 700 m, such as from about 1 m to about 600 m, such as from about 1 m to about 500 m, such as from about 1 m to about 400 m, such as from about 1 m to about 300 m, such as from about 1 m to about 200 m, such as from about 1 m to about 100 m, such as from about 1 m to about 80 m, such as from about 1 m to about 60 m, such as from about 1 m to about 40 m, such as from about 1 m to about 20 m, such as from about 1 m to about 10 m, such as from about 2 m to about 8 m, such as from about 3 m to 7 m or such as from about 4 m to 6 m. Particularly, the length of the coil reactor is about 5 m. Said coil reactor may have an inner diameter ranging from about 1 mm to about 100 mm, such as from about 1 mm to about 80 mm, such as from about 1 mm to about 60 mm, such as from about 1 mm to about 30 mm, such as from about 1 mm to about 15 mm, such as from about 1 mm to about 8 mm, such as from about 2 mm to about 6 mm or such as from about 3 mm to 5 mm. Particularly, the diameter of said coil reactor is about 4 mm.

In step (e′) the precipitate may be isolated by any suitable means known in the art including standard purification or isolation methods. Said purification or isolation methods include, for example, sedimentation, filtration or centrifugation. Filtration can be achieved with a suction filter or an agitated filter dryer. Centrifugation can be achieved by using a centrifuge, such as a vertical centrifuge with manual discharge, a peeler centrifuge, a screen scroll centrifuge, a screen scroll centrifuge or a pusher centrifuge. For batch sizes below or equal to 1 kg, it is advantageous to use a centrifuge with manual discharge, while for batch sizes above 1 kg it is advantageous to use a peeler centrifuge.

As will be appreciated by those skilled in the art, the collecting unit, such as the centrifuge, may be equipped with an inertization control system that provides a gas supply. This is especially beneficial because the method of isolation and purification of the present invention uses anti-solvents, such as organic solvents, for the precipitation and washing of the polymer, such as of a functionalized HA. To be able to control the resulting vapour mixture, which poses an explosion risk, and in order to keep the obtained polymer inert, the centrifuge is purged with a gas, such as nitrogen or argon, prior to start. Once this has been done, the centrifuge remains in a gas atmosphere (nitrogen or argon atmosphere). Being able to also keep the isolated polymer, such as a functionalized HA, under an inert state is particularly useful for compounds that are less stable, such as for example a thiol-functionalized HA which may be prone to oxidation.

The second solution or optional solution may comprise or consist of a mixture of anti-solvents, it may comprise a mixture of water and an anti-solvent or it may consist of an anti-solvent. The anti-solvent may be any solvent known in the art that will precipitate a polymer, such as a functionalized HA, or in which said polymer is insoluble.

Examples of anti-solvents which may be employed in the second solution include ethanol, methanol, isopropanol, acetonitrile, tert-butanol, n-propanol, tetrahydrofuran, acetone, 1,4-dioxane, ethylene glycol, diethylene glycol, triethylene glycol, glycerol, dimethylformamide (DMF), N-methyl-2-pyrrolidone (NMP) or mixtures thereof.

In certain embodiments, the second solution is ethanol. In certain embodiments, the second solution is methanol. In certain embodiments, the second solution is isopropanol. In certain embodiments, the second solution is acetonitrile. In certain embodiments, the second solution is tert-butanol. In certain embodiments, the second solution is n-propanol. In certain embodiments, the second solution is tetrahydrofuran. In certain embodiments, the second solution is acetone. In certain embodiments, the second solution is 1,4-dioxane. In certain embodiments, the second solution is ethylene glycol. In certain embodiments, the second solution is diethylene glycol. In certain embodiments, the second solution is triethylene glycol.

In certain embodiments, the second solution is glycerol. In certain embodiments, the second solution is dimethylformamide (DMF). In certain embodiments, the second solution is N-methyl-2-pyrrolidone (NMP).

In certain embodiments, the second solution is a mixture of water and ethanol.

The first solution comprising the functionalized HA may be a mixture that results upon a method for preparing a functionalized HA. For example, it may be a mixture that results upon the synthesis (or method for preparing) of an amine-functionalized HA, i.e. by introducing an amine group by converting at least one more carboxyl groups of an HA into N-substituted amides having a spacer moiety, such as an -L^{2′}- spacer moiety as described elsewhere herein, comprising an amine group at its end. It may also be a mixture that results upon the synthesis of a maleimide-functionalized HA, i.e. by introducing a maleimide group into an amine-functionalized HA by converting the end amine group into N-substituted amides having a spacer moiety, such as an -L^{2z}- spacer moiety as described elsewhere herein, comprising a maleimide group at its end. Also, it may be a mixture that results upon the synthesis of a thiol-functionalized HA, i.e. by introducing a thiol group into an amine-functionalized HA by converting the end amine group into N-substituted amides having a spacer moiety, such as an -L^{2′}- spacer moiety as described elsewhere herein, comprising a thiol group at its end. Thus, it is understood that in addition to the functionalized HA, i.e. the product, the first solution may also comprise unreacted starting reagents or any byproducts.

Generally, if required, the first solution may be conditioned in the flow system prior to being combined with the second solution comprising an anti-solvent. Conditioning conditions include quenching, such as quenching with a quenching solution comprising an organic salt (e.g., sodium acetate).

Specifically, the first solution comprising an amine-functionalized HA, such as an amine-functionalized HA comprising a plurality of each of the following linearly connected Z¹ and Z⁷-i units:

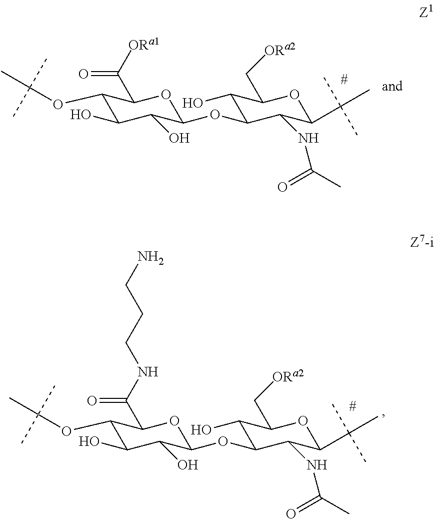

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is H or an alkali metal;
each —R^{a2} is —H;
   may also comprise water, acetate ions, chloride ions, alkali metal ions such as Na⁺ or K⁺, 2-(N-morpholino)ethanesulfonic acid (MES), unreacted 1,3-diaminopropane, EDC urea (N-[3-(dimethylamino)propyl]-N′-ethylurea) and/or hydroxybenzotriazole (HOBt).

Also, the first solution comprising a thiol functionalized HA, such as a thiol functionalized HA comprising a plurality of each of the following linearly connected Z¹ and Z⁵-i units:

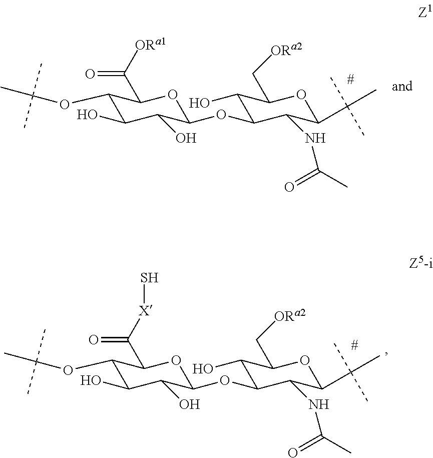

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is H or an alkali metal;
each —R^{a2} is —H;
—X′— is of formula (x4):

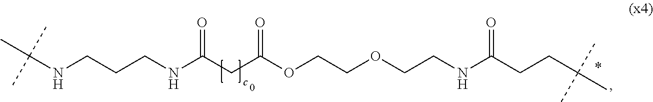

wherein the unmarked dashed line indicates the attachment to the carbonyl group, the
dashed line marked with an asterisk indicates the attachment to the sulfur atom and c₀ is 7; may also comprise N-2-hydroxyethylpiperazine-N-2-ethane sulfonic acid (HEPES), acetate ions, chloride ions, alkali metal ions such as Na⁺ or K⁺, acetonitrile, tris(2-carboxyethyl)phosphine hydrochloride (TCEP) and/or the following compounds:

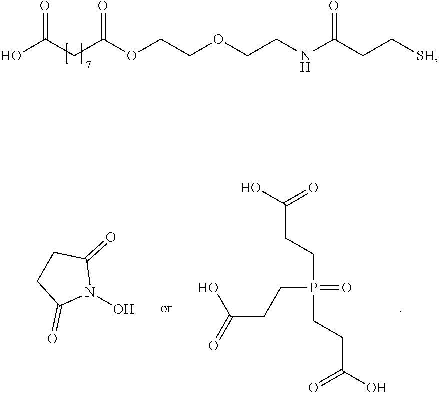

A first solution comprising a maleimide functionalized HA, such as a maleimide functionalized HA comprising a plurality of each of the following linearly connected Z¹ and Z⁶-i units:

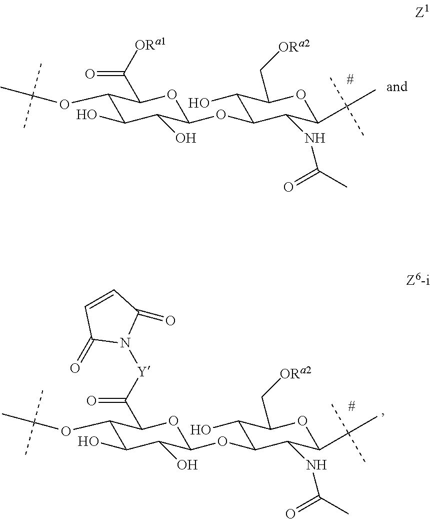

wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is H or an alkali metal;
each —R^{a2} is —H;
—Y′— is of formula (y4):

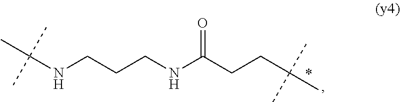

may also comprise N-2-hydroxyethylpiperazine-N-2-ethane sulfonic acid (HEPES), acetate ions, alkali metals ions such as Na⁺ or K⁺, acetonitrile and/or the following compounds:

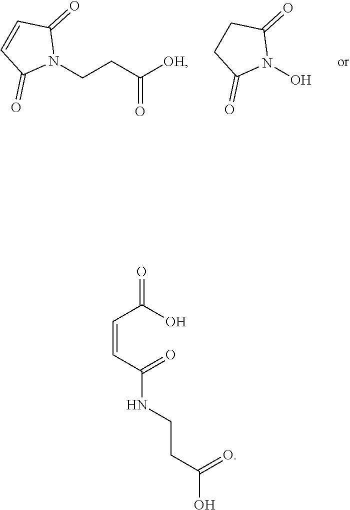

### BRIEF DESCRIPTION OF THE FIGURES

Various embodiments are described hereinafter with reference to the figures. Like reference numerals refer to the like elements throughout. Like elements will, thus, not be described in detail with respect to the description of each figure. It should also be noted that the figures are only intended to facilitate the description of the embodiments. They are not intended as an exhaustive description of the claimed invention or as a limitation on the scope of the claimed invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

FIG. 1 illustrates schematically an embodiment of a setup for precipitating and isolating a polymer.

FIG. 2 illustrates schematically another embodiment of a setup for precipitating and isolating a polymer.

FIG. 3 illustrates schematically yet another embodiment of a setup for precipitating and isolating a polymer.

### DETAILED DESCRIPTION OF THE FIGURES

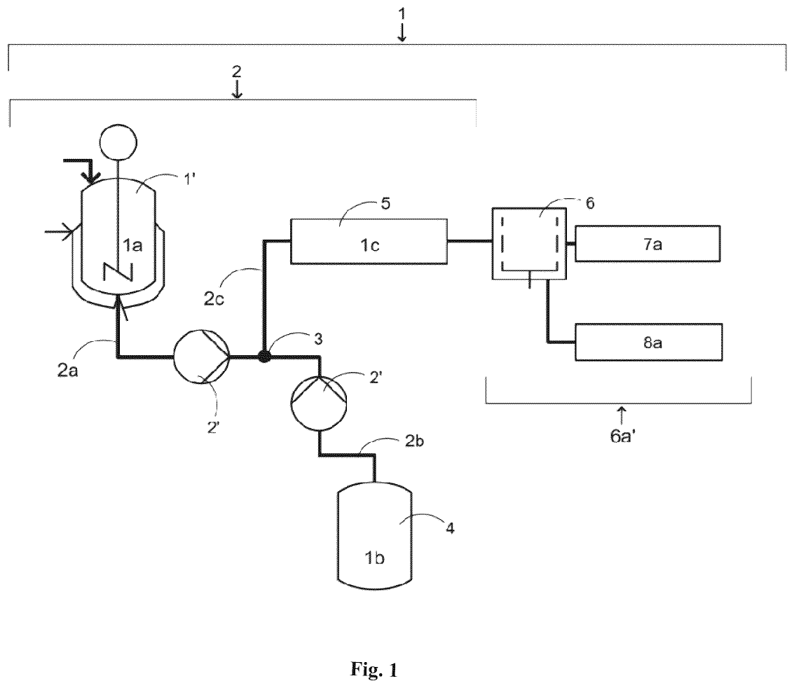

FIG. 1 illustrates schematically an embodiment of a setup 1 for precipitating and isolating a polymer. The setup 1 comprises a flow system 2 and a collecting assembly 6a′. The collecting assembly 6a′ comprises a collecting unit 6, a product container 7 and a waste container 8. In this embodiment, a first solution 1a comprising a polymer within a vessel 1′ is flowed through a first channel 2a. A second solution 1b comprising an anti-solvent within a storage vessel 4 is flowed through a second channel 2b. The first solution 1a and the second solution 1b are mixed in a combining unit 3. The combined mixture 1c is flowed from the combining unit 3 into a precipitating unit 5 through a third channel 2c. The mixture 1c comprising the precipitated polymer is flowed out from the precipitating unit 5 into the collecting unit 6 through channel 2c. All the beforementioned solutions and mixtures are flowed with the aid of pumps 2′. The precipitate 7a is collected into the product container 7, while the waste 8a is collected into the waste container 8.

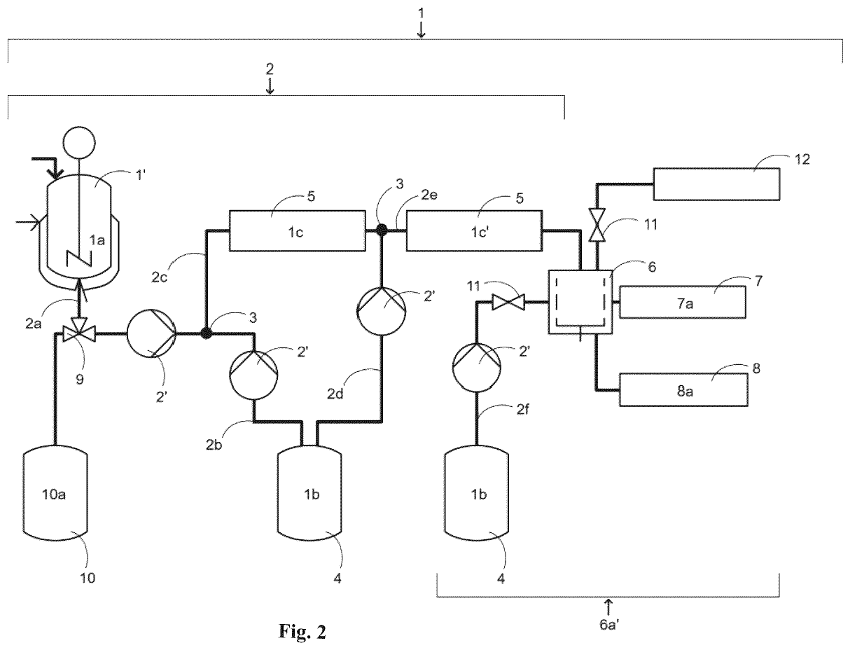

FIG. 2 illustrates schematically another embodiment of a setup 1 for precipitating and isolating a polymer comprising a flow system 2 and a collecting assembly 6a′. The collecting assembly 6a′ comprises a collecting unit 6, a product container 7, a waste container 8, an inertization control system 12 and a storage vessel 4. In this embodiment, a buffering agent 10a that is stored in a storage vessel 10 is flowed through the system 1 to equilibrate the pumps 2′. A first solution 1a comprising a polymer within a vessel 1′ is flowed through a first channel 2a. The first channel 2a is also connected via a three-way valve 9 to the storage vessel 10. A second solution 1b comprising an anti-solvent within a storage vessel 4 is flowed through a second channel 2b. The first solution 1a and second solution 1b are mixed in a combining unit 3. The combined mixture 1c is flowed from the combining unit 3 into a precipitating unit 5 through a third channel 2c. The mixture 1c comprising the precipitated polymer is flowed out from the precipitating unit 5 through channel 2c to a combining unit 3 where it is mixed with a solution 1b comprising an anti-solvent that is flowed through a fourth channel 2d from the storage vessel 4. The resulting mixture 1c′ is flowed from the combining unit 3 into a precipitating unit 5 through a fifth channel 2e. The mixture 1c′ comprising the precipitated polymer is flowed out from the second precipitating unit 5 into the collecting unit 6 through channel 2e. A solution 1b comprising the anti-solvent that is flowed from a storage container 4 through a sixth channel 2f, and which is connected to the collecting unit 6 via a two-way valve 11 is used to wash the precipitated polymer in the collecting unit 6. The inertization control system 12 is connected to the collecting unit 6 via a two-way valve 11. The precipitate 7a is collected into the product container 7, while the waste is collected into the waste container 8a. All the beforementioned solutions, buffering agents and mixtures are flowed with the aid of pumps 2′.

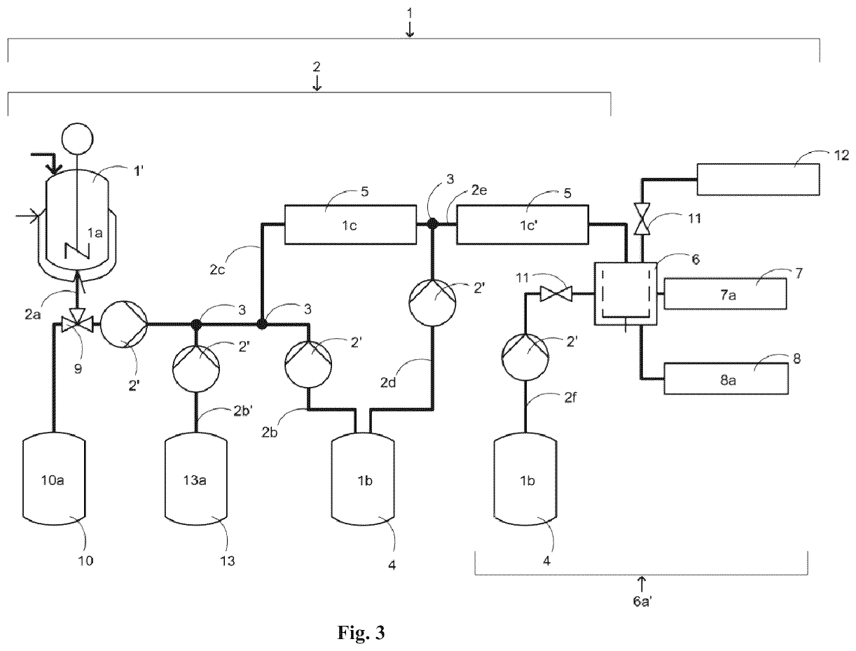

FIG. 3 illustrates schematically another embodiment of a setup 1 for precipitating and isolating a polymer comprising a flow system 2 and a collecting assembly 6a′. The collecting assembly 6a′ comprises a collecting unit 6, a product container 7, a waste container 8, an inertization control system 12 and a storage vessel 4. In this embodiment, a buffering agent 10a that is stored in a storage vessel 10 is flowed through the system to equilibrate the pumps 2′. A first solution 1a comprising a polymer within a vessel 1′ is flowed through a first channel 2a. The first channel 2a is also connected via a three-way valve 9 to the storage vessel 10. A quenching solution 13a stored within a vessel 13 for storing a quenching solution is flowed through a channel 2b′. The first solution 1a and the quenching solution 13a are mixed in a combining unit 3. A second solution 1b comprising an anti-solvent stored within a storage vessel 4 is flowed through a second channel 2b.

The second solution 1b and mixture 13a′ (not shown) comprising the quenched polymer are mixed in a combining unit 3. The combined mixture 1c is flowed from the combining unit 3 into a precipitating unit 5 through a third channel 2c. The mixture 1c comprising the precipitated polymer is flowed out from the precipitating unit 5 through channel 2c to a combining unit 3 where it is mixed with a solution 1b comprising an anti-solvent that is flowed through a fourth channel 2d from the storage vessel 4. The resulting mixture 1′ is flowed from the combining unit 3 into a precipitating unit 5 through a fifth channel 2e. The mixture 1′ comprising the precipitated polymer is flowed out from the second precipitating unit 5 into the collecting unit 6 through channel 2e. A solution 1b comprising the anti-solvent that is flowed from a storage container 4 through a sixth channel 2f, and which is connected to the collecting unit via a two-way valve 11 is used to wash the precipitated polymer in the collecting unit 6. The inertization control system 12 is connected to the collecting unit 6 via a two-way valve 11. The precipitate 7a is collected into the product container 7, while the waste 8a is collected into the waste container 8. All the beforementioned solutions and mixtures are flowed with the aid of pumps 2′.

Any of the aforementioned embodiments, including those described in FIG. 1, FIG. 2 and FIG. 3 may be modified in various ways. For example, the vessel 1′, the storage vessels 4, the precipitating units 5, the combining units 3 and the collecting unit 6 may each be independently temperature controlled. Also, for example, in the embodiments illustrated by FIG. 2 or FIG. 3 only one storage vessel 4 could be used and channel 2f which is connected to the collecting unit 6 could be connected to the same storage vessel 4. Furthermore, in the embodiments illustrated by FIG. 2 or FIG. 3 another solution comprising an anti-solvent that is different from the second solution could be comprised in a further storage vessel (not shown). Moreover, various flow rates, pressures, temperatures and concentrations may be employed depending on the polymer to be precipitated or the first solution comprising the polymer. In particular, it is desirable to cool the second solution comprising the anti-solvent prior to mixing it with the first solution or to the mixture that is flowed from a precipitating unit 5, such as to 0° C. or below.

The invention is also further described by the following non-limiting items.
1. A method for precipitating a polymer in a flow system, wherein the method comprises the steps of:
   (a′) flowing, optionally simultaneously, a first solution comprising the polymer through a first channel and a second solution comprising an anti-solvent through a second channel;
   (b′) combining the first and second solutions of step (a′);
   (c′) flowing the combined mixture of step (b′) into at least one precipitating unit; and
   (d′) precipitating the polymer;
   wherein if more than one precipitating unit is present, the mixture comprising the precipitated polymer that flows out from one precipitating unit is combined with the second solution comprising the anti-solvent or optionally with another solution comprising an anti-solvent before it is flowed into another precipitating unit.
2. A method for isolating a polymer in a setup for precipitating and isolating a polymer, wherein the method comprises the steps of
   (a′) flowing, optionally simultaneously, a first solution comprising the polymer through a first channel and a second solution comprising an anti-solvent through a second channel;
   (b′) combining the first and second solutions of step (a′);
   (c′) flowing the combined mixture of step (b′) into at least one precipitating unit;
   (d′) precipitating the polymer; and
   (e′) isolating the precipitate of step (d′),
   wherein if more than one precipitating unit is present, the mixture comprising the precipitated polymer that flows out from one precipitating unit is combined with the second solution comprising the anti-solvent or optionally with another solution comprising an anti-solvent before it is flowed into another precipitating unit.
3. The method of item 1 or 2, wherein one precipitating unit is present.
4. The method of item 1 or 2, wherein two precipitating units are present and the mixture comprising the precipitated polymer that flows out from the first precipitating unit is combined with the second solution comprising the anti-solvent before it is flowed into a second precipitating unit.
5. The method of any one of items 1 to 4, wherein the polymer is selected from the group consisting of polysaccharides such as hyaluronic acid, hyaluronic acid and derivatives or functionalized hyaluronic acid, heparin, heparan sulfate, heparosan, chondroitin sulfate, dermatan sulfate, keratan sulfate, cellulose, carboxymethyl cellulose, hydroxypropyl methylcellulose, chitin, chitosan, dextran or dextrin; polyethers, such as poly(ethyleneglycol) or poly(propylene glycols); polyesters, such as polyhydroxybutyrate, poly(glycolic acid), polybutylene terephthalate, poly(caprolactone), poly(lactic acid) or poly(lactic-co-glycolic acid); proteins, such as gelatin or collagen; polyolefins, such as poly(2-methacryloyl-oxyethyl phosphorylcholine), poly(acrylic acid), poly(acrylate), poly(acrylamide), poly(cyanoacrylate), poly(dimethylacrylamide), polyethylene, poly(hydroxyethyl acrylate), poly(2-hydroxyethyl methacrylate), poly(N-(2-hydroxypropyl)methacrylamide), poly(hydroxypropyl methacrylate), poly(vinyl alcohol), poly(vinyl amine), poly(vinylmethylether) or poly(vinylpyrrolidone); poly(oxazolines), such as poly(methyloxazoline) or poly(ethyloxazoline); polyamides; poly(amidoamines); poly(amino acids); polyanhydrides; poly(aspartamides); polycarbonates; poly(alkylene phosphates) such as poly(ethylene phosphates); poly(iminocarbonates); poly(methacrylamides); poly(organophosphazenes); poly(ortho esters); poly(siloxanes) and poly(urethanes).
6. The method of any one of items 1 to 5, wherein the polymer is selected from the group consisting of polysaccharides such as hyaluronic acid, hyaluronic acid and derivatives or functionalized hyaluronic acid, heparin, heparan sulfate, heparosan, chondroitin sulfate, dermatan sulfate, keratan sulfate, cellulose, carboxymethyl cellulose, hydroxypropyl methylcellulose, chitin, chitosan, dextran or dextrin; polyethers, such as poly(ethyleneglycol) and proteins, such as gelatin or collagen.
7. The method of any one of items 1 to 6, wherein the polymer is a functionalized HA.
8. The method of any one of items 1 to 7, wherein the first solution comprises a functionalized HA, wherein the functionalized HA comprises a plurality of each of the following linearly connected Z¹ and Z⁵-i units:

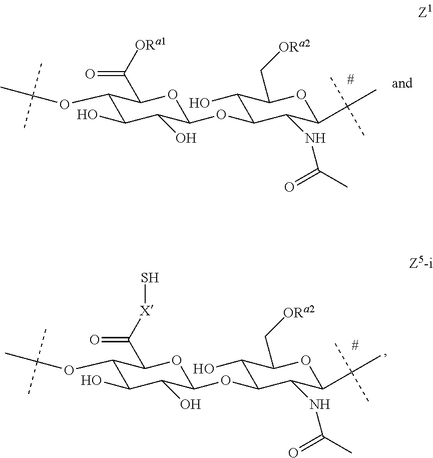

or a plurality of each of the following linearly connected Z¹ and Z⁶-i units.

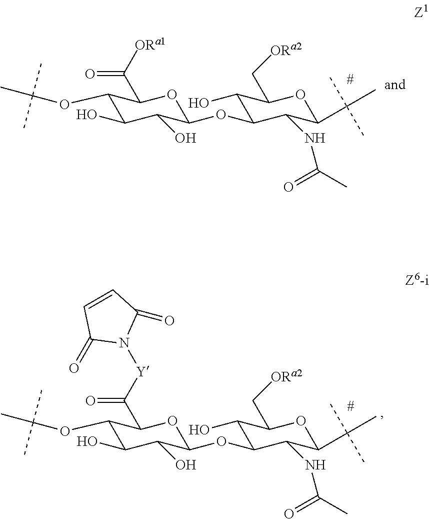

or a plurality of each of the following linearly connected Z¹ and Z⁷-i units:

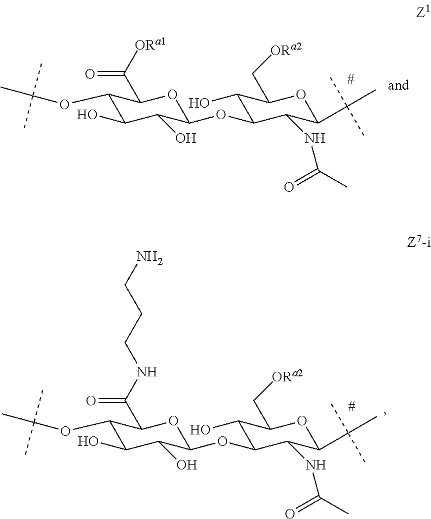

wherein an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
—X′— is of formula (x4):

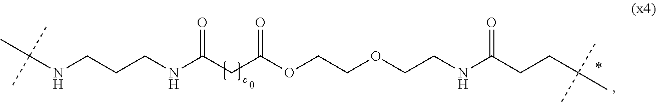

wherein the unmarked dashed line indicates the attachment to the carbonyl group, the dashed line marked with an asterisk indicates the attachment to the sulfur atom and c₀ is 7;
—Y′— is of formula (y4):

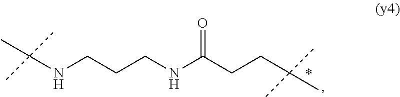

wherein the unmarked dashed line indicates the attachment to the carbonyl group and the dashed line marked with an asterisk indicates the attachment to the nitrogen atom of the maleimide ring;
each R^{a1} is H or an alkali metal;
each —R^{a2} is —H; and the second solution is ethanol.
9. A flow system for precipitating a polymer comprising:
   a vessel comprising a first solution comprising the polymer;
   at least one storage vessel comprising a second solution comprising an anti-solvent;
   at least one combining unit for combining the first and second solutions or for combining the mixture that flows out from a precipitating unit with the second solution comprising an anti-solvent or optionally with another solution comprising an anti-solvent;
   at least one precipitation unit for precipitating the polymer;
      wherein the vessel, at least one storage vessel, at least one combining unit and the at least one precipitation unit are connected via connective channels to provide a continuous flow path, wherein
         the first solution flows through a first channel from the vessel to the combining unit;
         the second solution flows through a second channel from the at least one storage unit to the combining unit;
         the combined mixture flows through at least one of the precipitation units and wherein if more than one precipitating unit is present, the mixture comprising the precipitated polymer that flows out from one precipitating unit is combined with the second solution comprising the anti-solvent or optionally with another solution comprising an anti-solvent before it is flowed into another precipitating unit.
10. The flow system of item 9, wherein the vessel and the at least one storage vessel are further connected to a valve and/or pump to control the flow rate of the mixture comprising the polymer and the anti-solvent.
11. The flow system of item 9 or 10, further comprising storage vessel for storing a buffering agent, wherein said storage vessel is connected via channels and a valve to an outflow of the vessel.
12. A setup for precipitating and isolating a polymer, wherein the setup comprises:
   I) a flow system for precipitating a polymer comprising:
      a vessel comprising a first solution comprising the polymer;
      at least one storage vessel comprising a second solution comprising an anti-solvent;
      at least one combining unit for combining the first and second solutions or for combining the mixture that flows out from a precipitating unit with the second solution comprising an anti-solvent or optionally with another solution comprising an anti-solvent;
      at least one precipitation unit for precipitating the polymer;
      wherein the vessel, at least one storage vessel, at least one combining unit and the at least one precipitation unit are connected via connective channels to provide a continuous flow path, wherein
         the first solution flows through a first channel from the vessel to the combining unit;
         the second solution flows through a second channel from the at least one storage unit to the combining unit;
         the combined mixture flows through at least one of the precipitation units and wherein if more than one precipitating unit is present, the mixture comprising the precipitated polymer that flows out from one precipitating unit is combined with the second solution comprising the anti-solvent or optionally with another solution comprising an anti-solvent before it is flowed into another precipitating unit; and
   II) a collecting assembly for isolating the precipitated polymer.
13. The flow system of any one of items 9 to 11 or the setup of item 12, wherein the combining unit is a Y-style, T-style, X-style, arrow-style mixer or static mixer.
14. The flow system of any one of items 9 to 11 or the setup of item 12, wherein the combining unit is a Y-style or T-style mixer.
15. The flow system of any one of items 9 to 11 or the setup of item 12, wherein the precipitating unit is a coil reactor or a tubular reactor.
16. The setup of item 12, wherein the collecting assembly comprises a centrifuge.

### Materials and Methods

All materials are commercially available except where stated otherwise.

### Amine Content Determination of Amine-HA:

Amine content of an amine-HA is determined by reacting the free amino groups with o-phthalaldehyde (OPA) and N-acetylcysteine under alkaline conditions and photometric quantification of the formed chromophores, as methodically described by Molnár-Perl (Ed.) (2015), Journal of Chromatography Library 70: 405-444.

### Thiol Content Determination of Thiolated Compounds:

The thiol content of a compound, which can either be soluble or insoluble in aqueous systems is determined by Ellman's assay by reacting the free compound thiol groups with DTNB reagent at neutral pH and subsequent photometric determination of the released 5-thio-2-nitrobenzoic acid (TNB) as methodically described in G. L. Ellman (1959), Archives of Biochemistry and Biophysics 82: 70-77.

### Maleimide Content Determination of Maleimide Compounds:

The maleimide content of a compound which can either be soluble or insoluble in aqueous systems is determined by reaction of the free compound maleimide groups with a known excess of 2-mercaptoethanol at neutral pH and determining the leftover thiols by Ellman's assay.

### Polymer Content Determination of Hydrogel Suspensions:

The polymer content of a hydrogel suspension is determined by successive washing of representative aliquots of the suspension in syringe reactors with PE frits with water and absolute ethanol and subsequent drying of the solid hydrogel portions under vacuum. The hydrogel content is calculated from the mass of the hydrogel residue per syringe and the respective aliquot volume of the hydrogel suspension.

### Example 1: Synthesis of NHS-Activated Disulfide Handle Reagent 1e

The NHS-activated azelaic acid disulfide building block 1e was synthesized according to the following scheme:

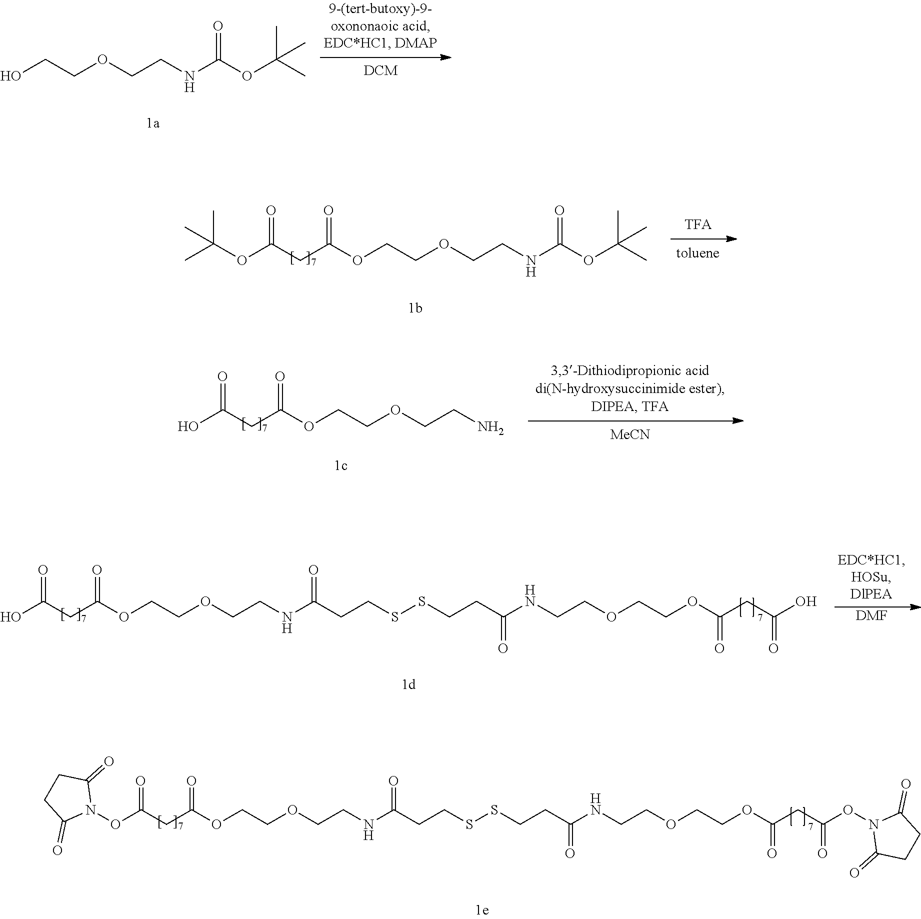

A mixture of 9-(tert-butoxy)-9-oxononaoic acid (18.4 g; 75.4 mmol; 1.00 eq.), 1a (15.5 g; 75.4 mmol; 1.00 eq.), EDC*HCl (15.9 g; 83.0 mmol; 1.10 eq.) and DMAP (2.29 g; 18.7 mmol; 0.25 eq.) in dichloromethane (300 mL) was stirred at r.t. for 2.5 h. All volatiles were removed under vacuo. The residue was taken up in EtOAc (500 mL) and the organic phase was successively washed with a 5% NaHSO₄ solution (3×500 mL), a mixture of saturated NaHCO₃ solution and water (1:1 v/v; 3×500 mL) and brine (500 mL). The organic phase was dried over NaSO₄, filtered and all volatiles were removed under reduced pressure. After drying under high vacuum, compound 1b was obtained as a yellowish oil.
Yield: 29.84 g (69 mmol, 91%)
MS: m/z 454.27=[M+Na]⁺, (calculated monoisotopic mass: [M]=431.56)

A solution of 1b (29.8 g; 69 mmol, 1.00 eq.) in TFA (50.0 mL; 0.65 mol, 9.39 eq.) was prepared and a stream of argon was passed through the reaction solution while stirring at r.t. for 160 minutes. Toluene (100 mL) was added, and the solution was concentrated to dryness under reduced pressure. This procedure was repeated four times. The product was dried under high vacuum overnight. After further drying under reduced pressure at 45° C. for three hours, compound 1c was obtained as TFA salt as yellow oil.
Yield: 28.11 g (max. 69 mmol)
MS: m/z 276.18=[M+H]⁺, (calculated monoisotopic mass: [M]=275.17)

3,3′-Dithiodipropionic acid di(N-hydroxysuccinimide ester) (9.13 g; 22.6 mmol; 1.00 eq.) and 1c (26.4 g; 67.7 mmol; 3.00 eq.) were dissolved in anhydrous acetonitrile (106 mL) and stirred at r.t. for five minutes before DIPEA (98.3 mL; 564 mmol; 25.00 eq.) was added and stirred at r.t. for additional 1.5 hours. The solution was cooled to 0° C. in an ice-bath and TFA (43.2 mL, 564 mmol, 25 eq.) was added. The solution was slowly warmed to r.t. whilst stirring for an additional hour. The reaction solution was diluted with dichloromethane (600 mL) and washed with 10% NaHSO₄ (3×600 mL). The combined aqueous phases were re-extracted with dichloromethane (2×100 mL). All organic phases were combined and dried over Na₂SO₄, filtered, and concentrated under reduced pressure. The residue was dried under high vacuum overnight. The crude material was recrystallized from acetonitrile to yield compound 1d as a yellow solid.
Yield: 14.95 g (20.6 mmol, 91% over two steps)
MS: m/z 725.34=[M+H]⁺, (calculated monoisotopic mass: [M]=724.32.)

1d (14.9 g; 20.5 mmol; 1.00 eq.) and N-hydroxysuccinimide (11.8 g; 102 mmol; 5.00 eq.) were dissolved in DMF (200 mL). DIPEA (35.7 mL; 204 mmol; 10.0 eq.) was added and the mixture was stirred at 60° C. for 15 min. To the clear solution, EDC*HCl (19.6 g; 102 mmol; 5.00 eq.) and additional DMF (100 mL) were added, and it was stirred at 60° C. for 1.5 hours. After cooling the mixture to 0° C. in an ice-bath, TFA (17.2 mL, 225 mmol, 11 eq.) was added. The solution was concentrated under reduced pressure and the resulting yellow oil was diluted with ethyl acetate (700 mL) and successively washed with 0.1 M HCl (5×200 mL), a mixture of saturated NaHCO₃ solution and water (1:1 v/v, 5×200 mL) and brine (100 mL). The organic phase was dried over MgSO₄, filtered, and all volatiles were removed under reduced pressure before the residue was dried under high vacuum for 12 hours. The crude material was purified by flash column chromatography to yield product 1e as a yellow solid.
Yield: 11.4 g (12.4 mmol, 60.4%)
MS: m/z 919.37=[M+H]⁺, (calculated monoisotopic mass: [M]=918.36.)

### Example 1a: Flow Precipitation of Native HA 2

A solution of native HA 2 was prepared by dissolving hyaluronic acid sodium salt (100-150 kDa, 60.0 g, 150 mmol COOH functionality, 1.00 eq.) in 2.5 L 100 mM MES, 0.4 M 1,3-diaminopropane buffer of pH 6.5 for 16 h at r.t. After dissolution, sodium acetate (339 g; 2.49 mol, 16.6 eq.) was added and the mixture was stirred for 15 min and then kept at r.t.

The flow precipitation system was conditioned as follows. At ambient temperature, 100 mM MES, 0.4 M 1,3-diaminopropane buffer of pH 6.5 was pumped with a flow rate of 64 mL/min, and ethanol stream 1 was pumped with a flow rate of 96 mL/min. Both streams were combined in a Y-piece and flowed into a tube reactor (5 m long, inner diameter 4 mm, 63 mL volume, residence time 24 sec). Subsequently, the stream coming from the tube reactor and ethanol stream 2 were added through another Y-piece with a flow rate of 160 mL/min. The combined flow entered a PTFE tube reactor (30 m long, inner diameter 4 mm, 377 mL volume, residence time 71 sec). Afterwards, the stream was directed into a screen centrifuge spinning at 800 rpm equipped with a filter bag of 20 μm pore size. Conditioning was complete when the stream reached the centrifuge, and then all the pumps were stopped.

HA 2 was precipitated as follows. The buffer feed from the conditioning was replaced by the native HA feed, the native HA solution was pumped with a flow rate of 64 mL/min, and all other parameters were set equal to those in the conditioning process. Precipitation could be observed starting from the first Y-piece. After the HA solution was consumed, the feed was changed back to the buffer reservoir to flush the lines. The flow of all pumps was kept constant until no more precipitate could be visually detected when entering the centrifuge, and then all pumps were stopped. For washing the cake in the centrifuge, 2.5 L of 80% ethanol in water and subsequently 2.5 L of pure ethanol were pumped with a flow rate of 500 mL/min through the spray nozzle of the centrifuge. Spinning at 800 rpm was continued for 1 min, then the centrifuge was stopped. The product was collected and dried under fine vacuum overnight to give flow-precipitated HA 2 as a white solid.
Recovery: 54.71 g (91.2%)

### Example 2: Synthesis of Amine-HA Building Blocks 3, 3a, 3b, 3c, 3d, 3e, 3f, 3g and 3h

Exemplary reaction scheme for the synthesis of amine-functionalized HA derivatives:

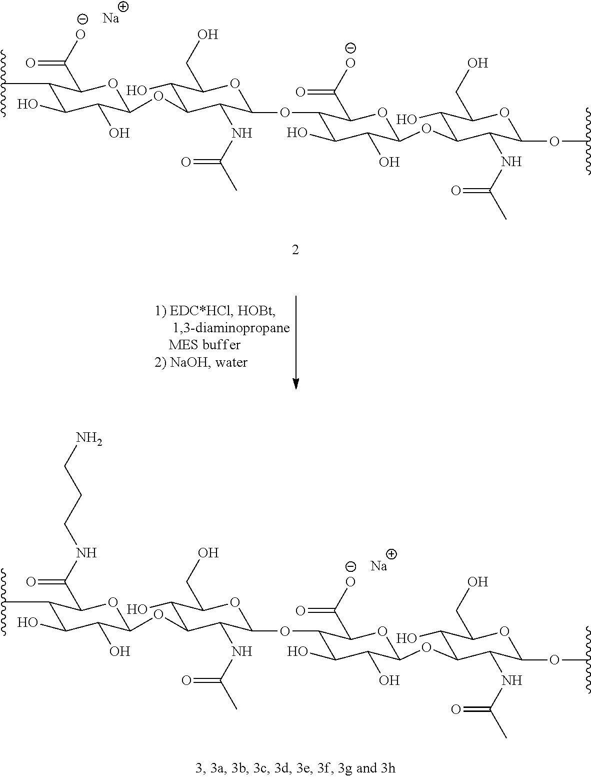 [SMILES: [Na+] ; *OC1OC(CO)C(O)C(OC2OC(C(=O)[O-])C(OC3OC(CO)C(O)C(OC4OC(C(=O)[O-])C(*)C(O)C4O)C3NC(C)=O)C(O)C2O)C1NC(C)=O ; *OC1OC(CO)C(O)C(OC2OC(C(=O)[O-])C(OC3OC(CO)C(O)C(OC4OC(C(=O)NCCCN)C(*)C(O)C4O)C3NC(C)=O)C(O)C2O)C1NC(C)=O ; [Na+]]

2 (average molecular weight: 100-150 kDa, 5 g, 12.5 mmol COOH functionality, 1.00 eq.) was dissolved in 100 mM MES, 0.4 M 1,3-diaminopropane buffer of pH 6.5 to form an 8 mg/mL solution. HOBt*H₂O (5.72 g, 37.4 mmol, 3.00 eq) and EDC*HCl (2.68 g, 0.46 mmol, 1.12 eq.) were successively added and the solution was stirred at 37° C. for 24 h. To the reaction mixture, sodium acetate (84.8 g; 24.9 mmol, 50.0 eq.) was added. After complete dissolution, the intermediate was precipitated by addition of absolute ethanol, washed with ethanol, and dried under high vacuum overnight. The crude material was dissolved in water (400 mL), NaOH (4 M, 133 mL) was added, and the solution was stirred at ambient temperature for two hours before glacial acetic acid (30.5 mL) was added. The derivatized HA was precipitated by addition of absolute ethanol, washed with ethanol, and dried under high vacuum to give amine-HA 3 as a white solid. The amine content was determined using the OPA assay.
Yield: 4.78 g (amine content: 0.266 mmol/g, 11.0% DS)

Another amine-functionalized HA 3a was prepared analogously to the procedure described above using hyaluronic acid sodium salt (100-150 kDa, 5 g, 12.5 mmol COOH functionality, 1.00 eq.), HOBt*H₂O (5.73 g, 37.4 mmol, 3.00 eq), EDC*HCl (1.24 g, 6.47 mmol, 0.52 eq.) and 100 mM MES, 0.4 M 1,3-diaminopropane buffer of pH 5.5 and reacting at 22° C. for 18.5 hours. The amine content was determined using the OPA assay.
Yield: 4.53 g (amine content: 0.126 mmol/g, 5.1% DS)

Another amine-functionalized HA 3b was prepared analogously to the procedure described above using hyaluronic acid sodium salt (100-150 kDa, 5 g, 12.5 mmol COOH functionality, 1.00 eq.), HOBt*H₂O (5.73 g, 37.4 mmol, 3.00 eq), EDC*HCl (1.55 g, 8.11 mmol, 0.65 eq.) and 100 mM MES, 0.4 M 1,3-diaminopropane buffer of pH 5.5 and reacting at 22° C. for 18.5 hours. The amine content was determined using the OPA assay.
Yield: 4.39 g (amine content: 0.162 mmol/g, 6.6% DS)

Another amine-functionalized HA 3c was prepared analogously to the procedure described above using hyaluronic acid sodium salt (100-150 kDa, 10 g, 24.9 mmol COOH functionality, 1.00 eq.), HOBt*H₂O (11.5 g, 74.8 mmol, 3.00 eq), EDC*HCl (3.11 g, 16.2 mmol, 0.65 eq.) and 100 mM MES, 0.4 M 1,3-diaminopropane buffer of pH 5.5 and reacting at 22° C. for 18.5 hours. The amine content was determined using the OPA assay.
Yield: 9.15 g (amine content: 0.151 mmol/g, 6.2% DS)

Another amine-functionalized HA 3d was prepared analogously to the procedure described above using hyaluronic acid sodium salt (100-150 kDa, 7 g, 17.4 mmol COOH functionality, 1.00 eq.), HOBt*H₂O (8.01 g, 52.3 mmol, 3.00 eq) and EDC*HCl (583 mg, 3.04 mmol, 0.17 eq.). The amine content was determined using the OPA assay.
Yield: 6.16 g (amine content: 0.127 mmol/g, 5.2% DS)

Another amine-functionalized HA 3e was prepared analogously to the procedure described above using hyaluronic acid sodium salt (100-150 kDa, 2.50 g, 6.24 mmol COOH functionality, 1.00 eq.), HOBt*H₂O (2.86 g, 18.7 mmol, 3.00 eq) and EDC*HCl (473 mg, 2.47 mmol, 0.40 eq.). The amine content was determined using the OPA assay.
Yield: 2.13 g (amine content: 0.269 mmol/g, 11.1% DS)

Another amine-functionalized HA 3f was prepared analogously to the procedure described above using hyaluronic acid sodium salt (100-150 kDa, 5.01 g, 12.5 mmol COOH functionality, 1.00 eq.), HOBt*H₂O (5.73 g, 37.4 mmol, 2.99 eq), EDC*HCl (2.54 g, 13.3 mmol, 1.06 eq.) and 100 mM MES, 0.4 M 1,3-diaminopropane buffer of pH 5.5 and reacting at 22° C. for 18.5 hours. The amine content was determined using the OPA assay.
Yield: 4.67 g (amine content: 0.232 mmol/g, 9.5% DS)

Another amine-functionalized HA 3g was prepared analogously to the procedure described above using hyaluronic acid sodium salt (150-300 kDa, 90.0 g, 224 mmol COOH functionality, 1.00 eq.), HOBt*H₂O (34.3 g, 224 mmol, 1.00 eq.), EDC*HCl (3.96 g, 20.7 mmol, 0.092 eq.) and 3.75 L 100 mM MES, 0.4 M 1,3-diaminopropane buffer of pH 6.5 and reacting these components at 37° C. for 24 hours. The amine content was determined using an OPA assay.
Yield: 83.0 g (amine content: 0.137 mmol/g, 4.2% DS)

Amine-HA 3h was prepared following the procedure described above using hyaluronic acid sodium salt (100-150 kDa, 120.0 g, 299 mmol COOH functionality, 1.00 eq.), HOBt*H₂O (45.7 g, 299 mmol, 1.00 eq.), EDC*HCl (14.9 g, 77.5 mmol, 0.26 eq.) and 5.0 L 100 mM MES, 0.4 M 1,3-diaminopropane buffer of pH 6.5 and reacting these components at 37° C. for 24 hours. To the reaction mixture, sodium acetate (678.2 g; 4.98 mol, 16.7 eq.) was added and the mixture was stirred at 20° C. for 4 h. The crude amine-HA was collected by precipitation and the washing was performed as described for the HA 2 in Example 1a, except for pumping the product stream directly out of the reactor. The crude material was dissolved in water (3.2 L), then NaOH (4 M, 1.08 L) was added, and the solution was stirred at 20° C. for 2 h. Glacial acetic acid (248 mL) was added. This mixture was used for the second precipitation following the procedure as described in Example 1a. Amine-HA 3h was isolated from the centrifuge filter, dried in fine vacuum for 5 days and was obtained as a white solid. The amine content was determined using an OPA assay.
Yield: 109.23 g (amine content: 0.307 mmol/g, 12.2% DS)

### Example 3: Synthesis of Maleimide-HA Building Blocks 5, 5a, 5b, 5c, 5d-i, 5d-ii and 5e

Exemplary reaction scheme for the synthesis of maleimide-functionalized HA derivatives:

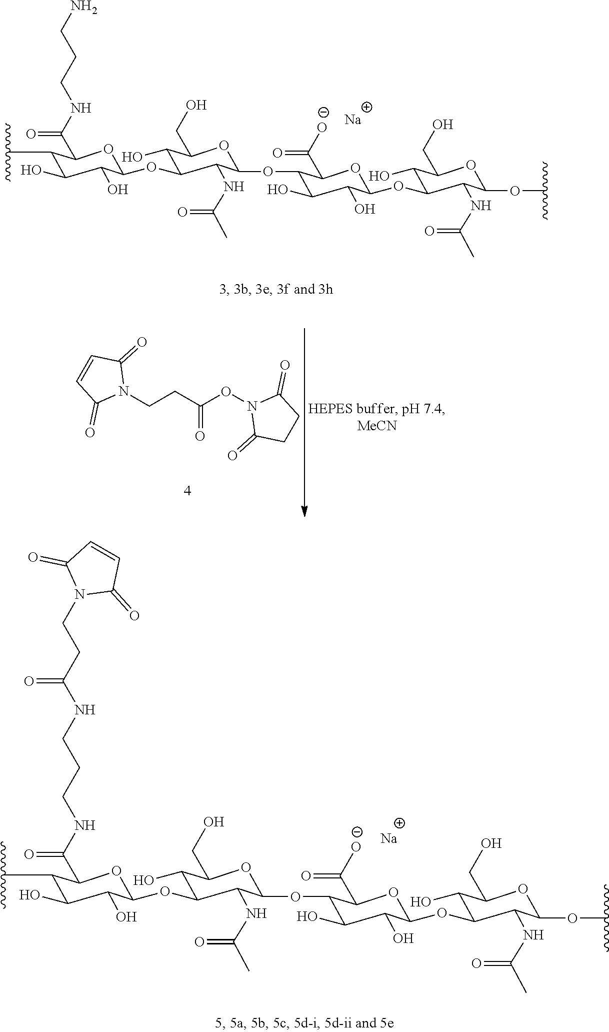 [SMILES: *OC1OC(CO)C(O)C(OC2OC(C(=O)[O-])C(OC3OC(CO)C(O)C(OC4OC(C(=O)NCCCN)C(*)C(O)C4O)C3NC(C)=O)C(O)C2O)C1NC(C)=O ; [Na+] ; O=C(CCN1C(=O)C=CC1=O)ON1C(=O)CCC1=O ; *OC1OC(CO)C(O)C(OC2OC(C(=O)[O-])C(OC3OC(CO)C(O)C(OC4OC(C(=O)NCCCNC(=O)CCN5C(=O)C=CC5=O)C(*)C(O)C4O)C3NC(C)=O)C(O)C2O)C1NC(C)=O ; [Na+]]

3 (4.28 g, amine functionalities: 0.266 mmol/g, 1.14 mmol, 1.00 eq.) was dissolved in 100 mM HEPES buffer pH 7.4 to form a 10 mg/mL solution. A solution of 4 (3.03 g, 11.4 mmol; 10.0 eq.) in MeCN (75.6 mL) was added and the reaction mixture was stirred at r.t. for 60 minutes. 1 M sodium acetate solution pH 5.5 (504 mL) was added to the solution under stirring before absolute ethanol was added to precipitate the polymer. The precipitate was washed with ethanol and dried under high vacuum. The crude material was dissolved in 1% acetic acid (428 mL). To the solution, 1 M sodium acetate solution pH 5.5 (504 mL) was added. The polymer was precipitated by addition of absolute ethanol, washed with ethanol, and dried under high vacuum to give maleimide-HA 5 as a white solid. The maleimide content was determined using an inverse Ellman assay.
Yield: 3.75 g (maleimide content: 0.258 mmol/g)

Another maleimide-functionalized HA 5a was prepared analogously to the procedure described above using 3b (2.00 g, 0.162 mmol/g amines, 0.32 mmol, 1.00 eq.) and a solution of 4 (0.86 g, 3.24 mmol; 10.0 eq.) in acetonitrile (35 mL). The maleimide content was determined using an inverse Ellman assay.
Yield: 1.67 g (maleimide content: 0.153 mmol/g)

Another maleimide-functionalized HA 5b was prepared analogously to the procedure described above using 3e (2.04 g, 0.269 mmol/g amines, 0.55 mmol, 1.00 eq.) and a solution of 4 (1.53 g, 5.75 mmol; 10.5 eq.) in acetonitrile (110 mL). The maleimide content was determined using an inverse Ellman assay.
Yield: 1.59 g (maleimide content: 0.231 mmol/g)

Another maleimide-functionalized HA 5c was prepared analogously to the procedure described above using 3f (2.00 g, 0.232 mmol/g amines, 0.46 mmol, 1.00 eq.) and a solution of 4 (1.24 g, 4.64 mmol; 10.0 eq.) in acetonitrile (35 mL). The maleimide content was determined using an inverse Ellman assay.
Yield: 1.78 g (maleimide content: 0.212 mmol/g)

Another two batches of maleimide-HA 5d-i and 5d-ii were prepared analogously to the procedure described above using 3h (50.15 g, 0.307 mmol/g amines, 15.4 mmol, 1.00 eq.) in 2.00 L 100 mM HEPES buffer pH 7.4 to form a 25 g/L solution and a solution of 4 (8.17 g, 30.7 mmol; 2.0 eq.) in acetonitrile (500 mL). After 1 h, 2 M sodium acetate solution pH 4.0 (833 mL) was added to form solution A.

The first half of solution A was precipitated using tubing with a 6 mm inner diameter and an 8 mm outer diameter. For conditioning of the flow precipitation system, a mixture of 80 vol % 100 mM HEPES buffer pH 7.4 and 20 vol % acetonitrile was pumped with a flow rate of 144 mL/min, and ethanol stream 1 was pumped with a flow rate of 216 mL/min. Both streams were combined in a Y-piece and flowed into a tube reactor (5 m long, inner diameter 6 mm, 141 mL volume, residence time 23 sec). Subsequently, ethanol stream 2 was added through a Y-piece with a flow rate of 360 mL/min. The combined flow entered a PTFE tube reactor (30 m long, inner diameter 6 mm, 848 mL volume, residence time 70 sec). Afterwards, the stream was directed into a screen centrifuge spinning at 800 rpm equipped with a filter bag of 20 μm pore size. Conditioning was complete when the stream reached the centrifuge, and then all the pumps were stopped.

The buffer feed from the conditioning was changed to reactor feed and solution A was pumped with a flow rate of 144 mL/min. All other parameters were set equal to those of the conditioning process. Washing, isolation and drying was done in the same manner as described in Example 1a to give compound 5d-i as a white solid.

The second half of solution A was precipitated using tubing with a 4 mm inner diameter and a 6 mm outer diameter. Conditioning, precipitation and washing steps were carried out in the same manner as described in Example 1a for the native HA 2, except for the buffer, which was a mixture of 80 vol % 100 mM HEPES buffer pH 7.4 and 20 vol % acetonitrile. Material 5d-ii was isolated after drying in fine vacuum as white solid.

The maleimide contents were determined using an inverse Ellman assay.
Yield (compound 5d-i): 26.26 g (maleimide content: 0.290 mmol/g)
Yield (compound 5d-ii): 23.71 g (maleimide content: 0.290 mmol/g)

Another maleimide-HA 5e is prepared analogously to the procedure described above using 3h (50.15 g, 0.307 mmol/g amines, 121 mmol, 1.00 eq.) in 2.00 L, 100 mM HEPES buffer, pH 7.4 to form a 25 g/L solution and a solution of 4 (32.3 g, 121 mmol; 1.0 eq.) in acetonitrile (500 mL). Quenching of the reaction is done in flow as well as precipitation of the product.

For conditioning the system, a mixture of 80 vol % 100 mM HEPES buffer pH 7.4 and 20 vol % acetonitrile is pumped with a flow rate of 108 mL/min, and 2 M sodium acetate pH 4.0 is pumped with a flow rate of 36 mL/min. Both streams are combined in a Y-piece with attached static mixer entering a tube reactor (5 m long, inner diameter 6 mm, 141 mL volume, residence time 59 sec). Subsequently, the ethanol stream is added through a Y piece with a flow rate of 576 mL/min. The combined flow enters a PTFE tube reactor (30 m long, inner diameter 6 mm, 848 mL volume, residence time 71 sec). Afterwards the stream is directed into a screen centrifuge spinning at 800 rpm equipped with a filter bag of 20 μm pore size. Conditioning is complete when the stream reaches the centrifuge, and then all pumps are stopped.

The buffer feed from the conditioning step is changed to reactor feed and the maleimide-HA solution is pumped with a flow rate of 108 mL/min. All other parameters are set equal to the conditioning process. The washing is done in the same manner as described in Example 1. Material 5e is isolated and dried in fine vacuum to give a compound with comparable properties to compounds 5d-i and 5d-ii.

### Example 4: Synthesis of Degradable Thiol-HA Building Blocks 6, 6a, 6b and 6c

Exemplary reaction scheme for the synthesis of degradable thiol-functionalized HA:

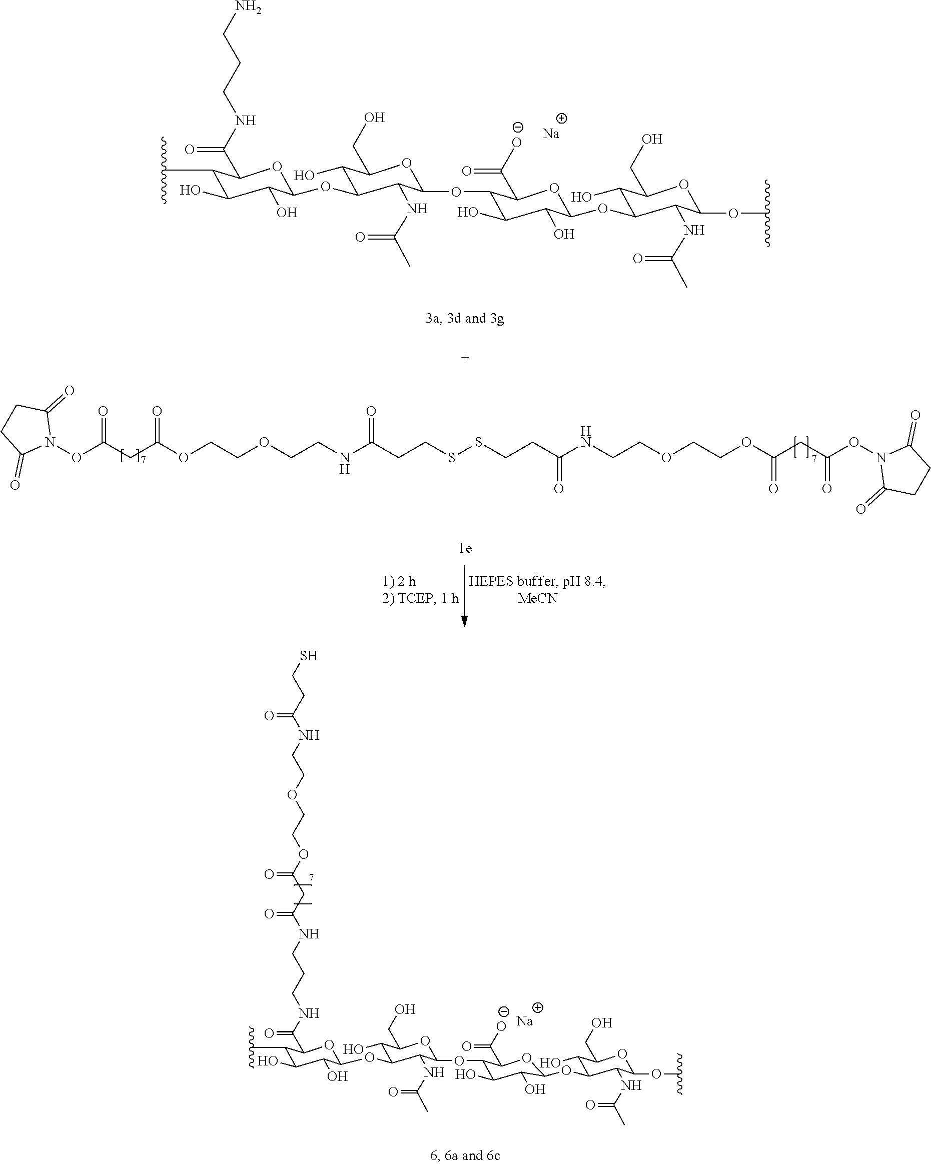

3a (1.50 g, amine-functionalities: 0.126 mmol/g, 0.19 mmol, 1.00 eq.) was dissolved in 100 mM HEPES buffer pH 8.4 to form a 20 mg/mL solution. A freshly prepared solution of 1e (0.52 g, 0.57 mmol; 3.00 eq.) in MeCN (40 mL) was added to the mixture and it was stirred at r.t. for three hours. A freshly prepared solution of TCEP*HCl (0.33 g, 1.14 mmol; 6.00 eq.) in water (11.5 mL) was added to the reaction mixture and it was stirred at r.t. for one hour. 1 M sodium acetate solution pH 5.5 (127 mL) was added to the solution under stirring before absolute ethanol was added to precipitate the polymer. The precipitate was washed with ethanol and dried under high vacuum. The crude material was dissolved in 1% acetic acid (150 mL) by vigorous stirring under an argon atmosphere. 1 M sodium acetate solution pH 5.5 (150 mL) was added to the solution. The polymer was precipitated by addition of absolute ethanol, washed with ethanol, and dried under high vacuum to give degradable thiol-HA 6 as a white solid. The thiol content was determined using an Ellman assay.
Yield: 1.07 g (thiol content: 0.103 mmol/g)

Another degradable thiol HA 6a was prepared analogously to the procedure described above using 3d (5 g, 0.127 mmol/g amines, 0.63 mmol, 1.00 eq.), bis-NHS ester 1e (1.75 g, 1.9. mmol; 3.0 eq.) and TCEP*HCl (1.10 g, 3.83 mmol; 6.03 eq.). After isolation, a third re-precipitation was carried out by dissolving the material (2.5 g) in a solution of TCEP*HCl (302 mg, 1.06 mmol) in 1% acetic acid (250 mL). After dilution with 1 M acetate, 1 mM histidine solution pH 5.5 (250 mL), the material was precipitated by addition of absolute EtOH, washed with absolute EtOH and dried under high vacuum. The thiol content was determined using an Ellman assay.
Yield: 2.23 g (thiol content: 0.102 mmol/g)

Another degradable thiol HA 6c was prepared analogously to the procedure described for 6 using 3g (40.0 g, 0.137 mmol/g amines, 5.48 mmol, 1.00 eq.), bis-NHS ester 1e (15.1 g, 16.5 mmol; 3.0 eq.), but stirring for 2 h at 40° C. TCEP*HCl (9.43 g, 32.9 mmol; 6.00 eq.) in 288 mL was added and the mixture was stirred for 1 h, followed by the addition of 3 M acetate, 1 mM histidine buffer, pH 5.5 (524 mL).

Conditioning and precipitation of the thiol-HA were carried out in the same manner as described in Example 1a, except for the following deviations. The buffer was a mixture of 80 vol % 100 mM HEPES, pH 8.0 and 20 vol % acetonitrile. T-pieces were used as connectors instead of Y-pieces. Washing of the filter cake was done using 5 L of each, 80% ethanol and absolute ethanol. The material was dried in fine vacuum for 5 days to give crude thiol-HA as a white material.

The crude compound was dissolved in 0.25 M acetate, 1 mM histidine buffer pH 4.0 (4.0 L) and the second precipitation was carried out in the same manner as the first one. The material was dried in fine vacuum for 2 days to give compound 6c as white material.
Yield: 35.0 g (thiol content: 0.099 mmol/g)

Exemplary reaction scheme for an alternative synthesis of degradable thiol-functionalized HA:

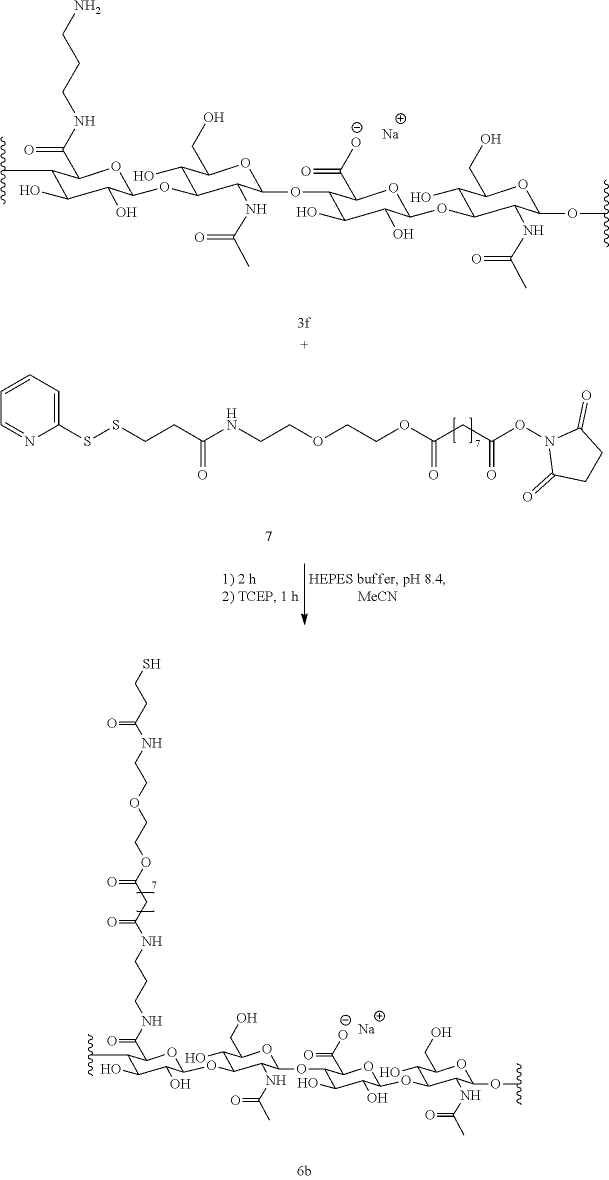

Another degradable thiol HA 6b was prepared analogously to the procedure described above using 3f (1.50 g, 0.126 mmol/g amines, 0.189 mmol, 1.00 eq.), 7 (described in patent WO2018175788A1 as compound d9; 562 mg, 0.95 mmol; 5.0 eq.) and TCEP*HCl (543 mg, 1.90 mmol; 10.0 eq.). The thiol content was determined using an Ellman assay.
Yield: 1.19 g (thiol content: 0.088 mmol/g)

### Example 5: Synthesis of Permanent Thiol-HA Building Block 9

Exemplary reaction scheme for the synthesis of non-degradable thiol-functionalized HA:

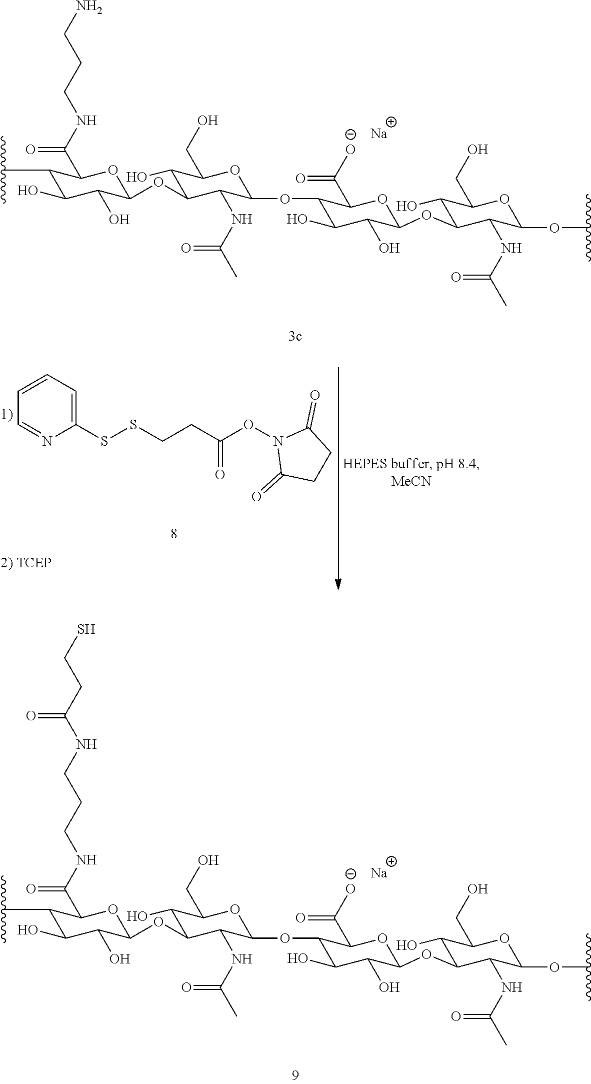 [SMILES: *OC1OC(CO)C(O)C(OC2OC(C(=O)[O-])C(OC3OC(CO)C(O)C(OC4OC(C(=O)NCCCN)C(*)C(O)C4O)C3NC(C)=O)C(O)C2O)C1NC(C)=O ; [Na+] ; O=C(CCSSc1ccccn1)ON1C(=O)CCC1=O ; *OC1OC(CO)C(O)C(OC2OC(C(=O)[O-])C(OC3OC(CO)C(O)C(OC4OC(C(=O)NCCCNC(=O)CCS)C(*)C(O)C4O)C3NC(C)=O)C(O)C2O)C1NC(C)=O ; [Na+]]

A non-degradable thiol HA 9 was prepared analogously to the procedure described in Example 4 using 3c (7 g, 0.151 mmol/g amines, 1.06 mmol, 1.00 eq.), 3-(2-pyridyldithio)-propionic acid N-succinimidylester (3.32 g, 10.6 mmol; 10.0 eq.) and TCEP*HCl (6.08 g, 21.2 mmol; 20.0 eq.). The thiol content was determined using an Ellman assay.
Yield: 5.6 g (thiol content: 0.120 mmol/g)

### Example 6: Synthesis of Degradable Crosslinked HA Microspheres 10, 10a, 10b, 10c, 10d and 10e with Free Maleimide Functionalities

Exemplary reaction scheme for the synthesis of degradable crosslinked HA microspheres with free maleimides:

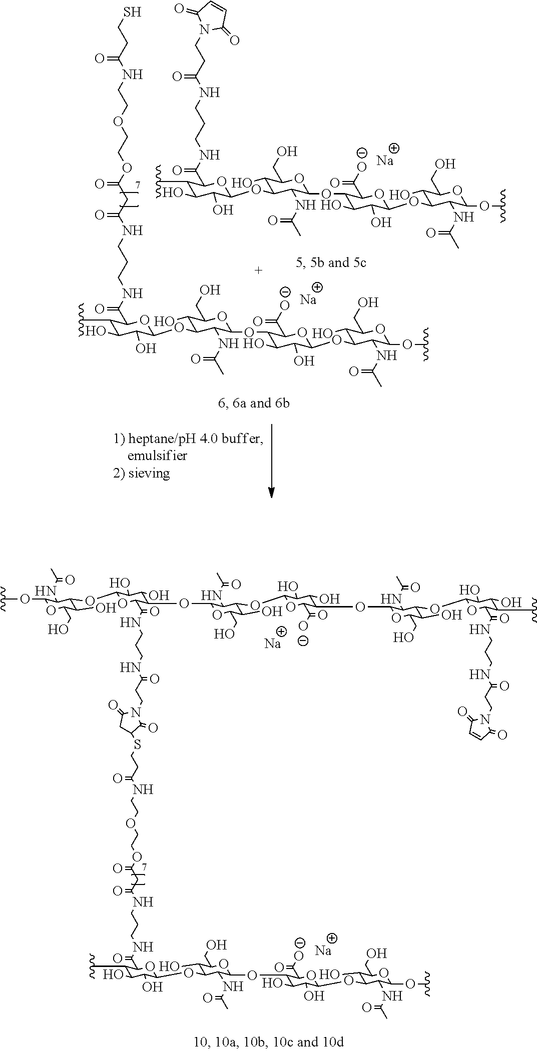

In a 250 mL reactor, a solution of Span® 80 in heptane (0.5%, 75 mL) was stirred at r.t. A 30 mg/mL solution of 6 (0.103 mmol/g thiols) in 100 mM citrate, 150 mM NaCl buffer pH 2 was prepared as solution A. A 30 mg/mL solution of 5 (0.258 mmol/g maleimides) in 100 mM citrate, 150 mM NaCl buffer pH 2 was prepared as solution B. An aliquot of 4.82 mL of solution A was mixed with 9.33 mL of solution B and 0.75 mL 100 mM citrate, 150 mM NaCl buffer pH 2 and the resulting mixture was added to the reactor, and it was stirred for emulsification. A solution of TMEDA (10.6 μL) in a solution of Span® 80 in heptane (0.5%, 5 mL) was added to the reactor and the mixture was stirred at r.t. overnight before 10 mM succinate, 200 mM sodium sulfate, 1 mM EDTA buffer pH 4 (50 mL) was added under stirring. The aqueous phase containing the microparticles was harvested after phase separation. For size fractionation, the water-hydrogel suspension was mixed 4:1 v/v with isopropanol and wet-sieved using 315, 200, 160, 100 and 50 μm sieves with a 4:1 v/v mixture of 10 mM succinate, 1 mM EDTA, 200 mM sodium sulfate buffer pH 4 and isopropanol as sieving buffer. Particle fractions that were retained on the 200, 160 and 100 μm sieves were separately collected and washed with 20 mM succinate, 150 mM NaCl, 3 mM EDTA, 0.1% Tween® 20 buffer pH 4 and then formulated in the same buffer to give suspensions of hydrogel 10. The hydrogel contents of the suspensions were determined by drying representative samples under high vacuum after washing the particles with water and ethanol. An inverse Ellman assay of the 100 μm sieve fraction was conducted, and the resulting maleimide content was used as representative value for all sieve fractions.
Yield: sieve fraction 100 μm: 22.5 mL with 4.3 mg/mL particle content
   sieve fraction 160 μm: 25.0 mL with 4.7 mg/mL particle content
   sieve fraction 200 μm: 7.5 mL with 5.9 mg/mL particle content
Maleimide content: 129 μmol/g (calculated for dry material)

Another degradable crosslinked HA with free maleimides was prepared as described for compound 10, using 5 (0.258 mmol/g maleimides, 6.96 mL of a 30 mg/mL solution in 100 mM citrate, 100 mM NaCl buffer pH 2), 6b (0.088 mmol/g thiols, 4.08 mL of a 30 mg/mL solution in 100 mM citrate, 100 mM NaCl buffer pH 2), 100 mM citrate, 100 mM NaCl buffer pH 2 (153 μL) and TMEDA (119 μL). Wet sieving was performed using 300, 212, 150, 100 and 50 μm sieves to give degradable crosslinked HA 10a as microparticle suspensions. The hydrogel contents of the different sieve fractions were determined by drying representative samples under high vacuum after washing the particles with water and ethanol. An inverse Ellman assay of the 150 μm sieve fraction was conducted, and the resulting maleimide content was used as representative value for all sieve fractions.
Yield: sieve fraction 100 μm: 25.0 mL with 2.3 mg/mL particle content
   sieve fraction 150 μm: 25.0 mL with 4.2 mg/mL particle content
   sieve fraction 212 μm: 25.0 mL with 2.2 mg/mL particle content
Maleimide content: 117 μmol/g (calculated for dry material)

Another degradable crosslinked HA 10b with free maleimide functionalities was prepared using 6a and 5b. In a 250 mL reactor, a solution of Span® 80 in heptane (0.5%, 75 mL) was stirred at r.t. A 32.5 mg/mL solution of 6a (0.102 mmol/g thiols) in 100 mM citrate, 5 mM histidine, 150 mM NaCl buffer pH 4 was prepared as solution A. A 32.5 mg/mL solution of 5b (0.231 mmol/g maleimides) in 100 mM citrate, 5 mM histidine, 150 mM NaCl buffer pH 4 was prepared as solution B. An aliquot of 13.1 mL of solution B was mixed with water (7.1 μL) and 6 M HCl (620 μL) before an aliquot of 6.32 mL of solution A was added and it was mixed again. The viscous solution was added to the reactor, and it was stirred for emulsification. A solution of TMEDA (271 μL) in a solution of Span® 80 in heptane (0.5%, 5 mL) was added to the reactor and the mixture was stirred at r.t. overnight before 10 mM succinate, 200 mM sodium sulfate, 1 mM EDTA buffer pH 4 (80 mL) was added under stirring. The aqueous phase containing the microparticles was harvested after phase separation. For size fractionation, the water-hydrogel suspension was mixed 4:1 v/v with isopropanol and wet-sieved using 315, 200, 160, 100 and 50 μm sieves with a 4:1 v/v mixture of 10 mM succinate, 1 mM EDTA, 200 mM sodium sulfate buffer pH 4 and isopropanol as sieving buffer. Particle fractions that were retained on the 200, 160 and 100 μm sieves were separately collected and washed with 20 mM succinate, 150 mM NaCl, 3 mM EDTA, 0.1% Tween® 20 buffer pH 4 and then formulated in the same buffer to give suspensions of hydrogel 10b. The hydrogel contents of the suspensions were determined by drying representative samples under high vacuum after washing the particles with water and ethanol. Inverse Ellman assays of the 100 μm, the 160 μm and the 200 μm sieve fractions were conducted, and the average maleimide content of the three fractions was used as representative value for all sieve fractions.
Yield: sieve fraction 100 μm: 10.0 mL with 4.6 mg/mL particle content
   sieve fraction 160 μm: 20.0 mL with 5.2 mg/mL particle content
   sieve fraction 200 μm: 55.0 mL with 5.3 mg/mL particle content
Maleimide content: 119 μmol/g (calculated for dry material)

Another degradable crosslinked HA 10c with free maleimides was prepared using 6b, 5c and Cithrol™ DPHS as emulsifier. A 50 mL Falcon tube was charged with a solution of Cithrol™ DPHS in heptane (0.25%, 6 mL) and the mixture was incubated end-over-end. A 30 mg/mL solution of 6b (0.088 mmol/g thiols) in 100 mM histidine, 100 mM NaCl buffer pH 2 was prepared as solution A. A 30 mg/mL solution of 5c (0.212 mmol/g maleimides) in 100 mM histidine, 100 mM NaCl buffer pH 2 was prepared as solution B. An aliquot of 372 μL of solution A was mixed with 618 μL of solution B and 91 μL of 100 mM histidine, 100 mM NaCl buffer pH 2 and the resulting mixture was added to the Falcon tube. The emulsion was agitated end over end, TMEDA (31.8 μL) was added, and the mixture was further agitated at r.t. After addition of a 4:1 v/v mixture of 10 mM succinate, 1 mM EDTA buffer pH 4 and iPrOH (6 mL), the tube was agitated shortly and the microparticles were harvested as aqueous suspension after centrifugation. The material was successively washed with a 4:1 v/v mixture of 10 mM succinate, 1 mM EDTA buffer pH 4 and iPrOH, iPrOH and 20 mM succinate, 150 mM NaCl, 3 mM EDTA, 0.1% Tween® 20 buffer pH 4 and adjusted to a volume of 10 mL in 20 mM succinate, 150 mM NaCl, 3 mM EDTA, 0.1% Tween® 20 buffer pH 4 to give degradable crosslinked HA 10c as a microparticle suspension.
Yield: 10.0 mL with 2.4 mg/mL particle content
Maleimide content: 60 μmol/g (calculated for dry material)

Another degradable crosslinked HA with free maleimides was prepared as described for compound 10c, using Hypermer 1083 as emulsifier to give degradable crosslinked HA 10d as a microparticle suspension.
Yield: 10.0 mL with 2.3 mg/mL particle content
Maleimide content: 67 μmol/g (calculated for dry material)

Another degradable crosslinked HA with free maleimides was prepared as described for compound 10c, using Lameform® TGI as emulsifier to give degradable crosslinked HA 10e as a microparticle suspension.
Yield: 10.0 mL with 2.5 mg/mL particle content
Maleimide content: 64 μmol/g (calculated for dry material)

### Example 7: Synthesis of Permanently Crosslinked HA Microspheres 11a with Free Thiol Functionalities

Exemplary reaction scheme for the synthesis of permanently crosslinked HA microspheres with free thiols:

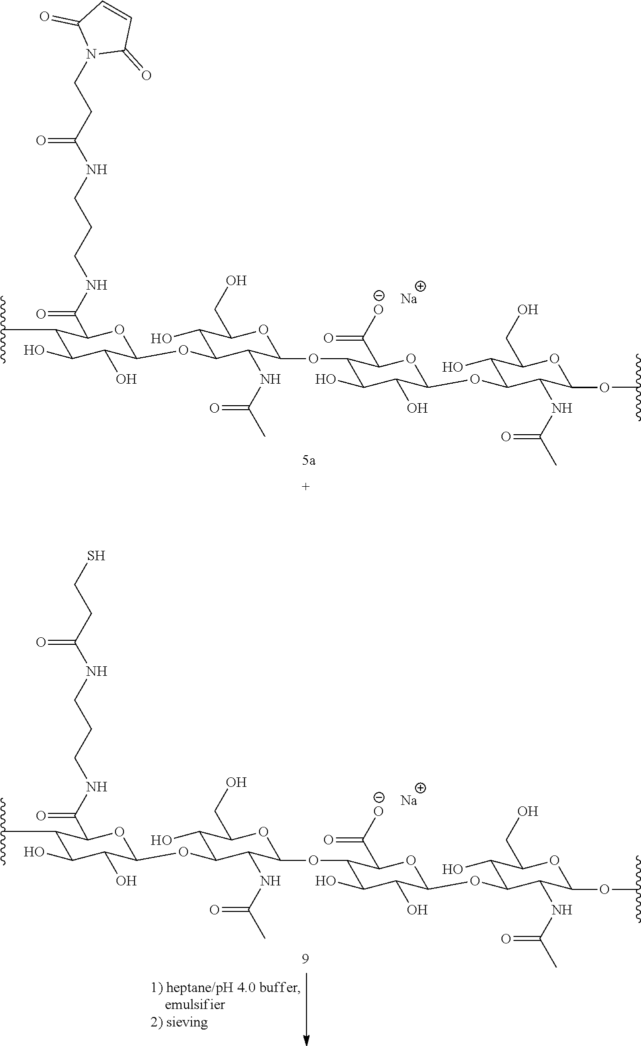 [SMILES: *OC1OC(CO)C(O)C(OC2OC(C(=O)[O-])C(OC3OC(CO)C(O)C(OC4OC(C(=O)NCCCNC(=O)CCN5C(=O)C=CC5=O)C(*)C(O)C4O)C3NC(C)=O)C(O)C2O)C1NC(C)=O ; [Na+] ; *OC1OC(CO)C(O)C(OC2OC(C(=O)[O-])C(OC3OC(CO)C(O)C(OC4OC(C(=O)NCCCNC(=O)CCS)C(*)C(O)C4O)C3NC(C)=O)C(O)C2O)C1NC(C)=O ; [Na+]]

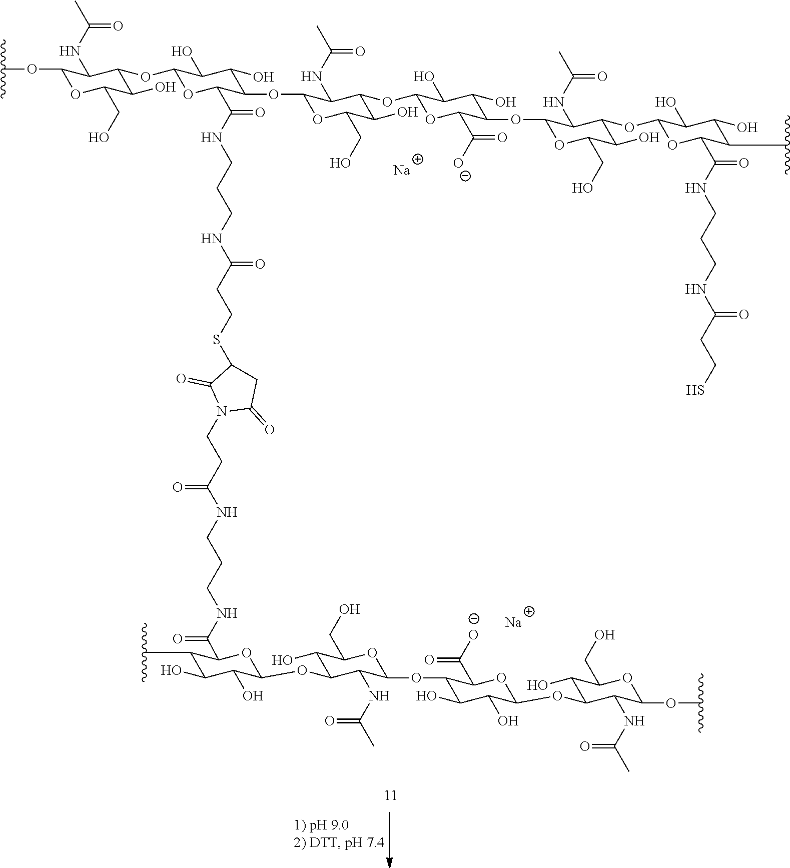 [SMILES: *OC1OC(CO)C(O)C(OC2OC(C(=O)[O-])C(OC3OC(CO)C(O)C(OC4OC(C(=O)NCCCNC(=O)CCN5C(=O)CC(SCCC(=O)NCCCNC(=O)C6OC(OC7C(O)C(CO)OC(O*)C7NC(C)=O)C(O)C(O)C6OC6OC(CO)C(O)C(OC7OC(C(=O)[O-])C(OC8OC(CO)C(O)C(OC9OC(C(=O)NCCCNC(=O)CCS)C(*)C(O)C9O)C8NC(C)=O)C(O)C7O)C6NC(C)=O)C5=O)C(*)C(O)C4O)C3NC(C)=O)C(O)C2O)C1NC(C)=O ; [Na+] ; [Na+]]

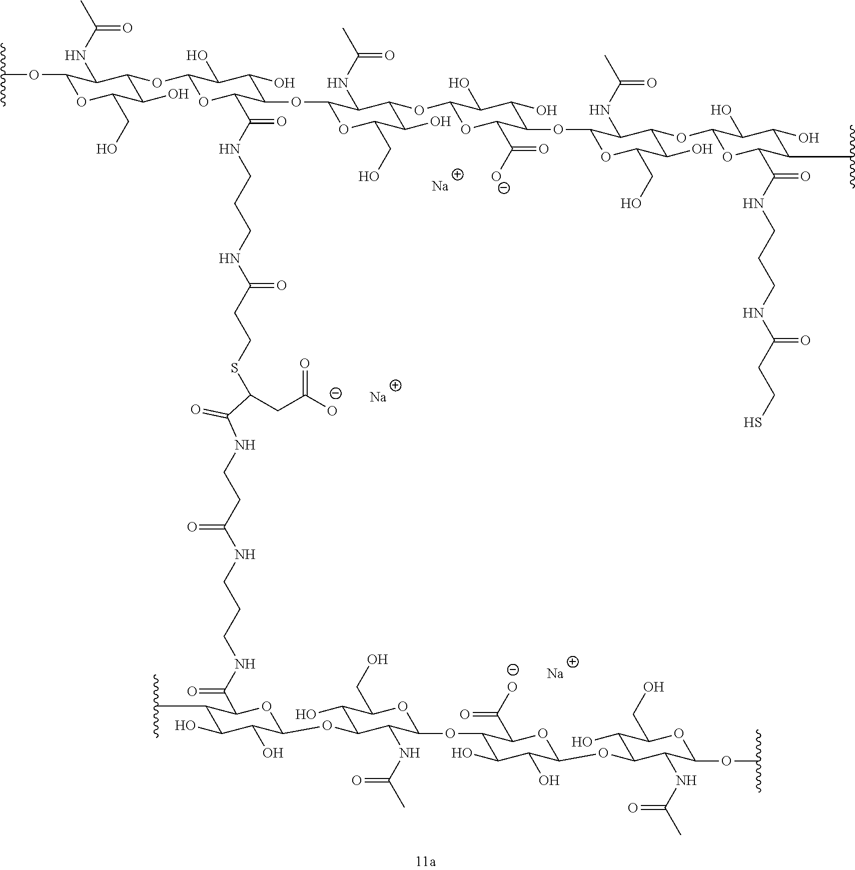 [SMILES: *OC1OC(CO)C(O)C(OC2OC(C(=O)[O-])C(OC3OC(CO)C(O)C(OC4OC(C(=O)NCCCNC(=O)CCNC(=O)C(CC(=O)[O-])SCCC(=O)NCCCNC(=O)C5OC(OC6C(O)C(CO)OC(O*)C6NC(C)=O)C(O)C(O)C5OC5OC(CO)C(O)C(OC6OC(C(=O)[O-])C(OC7OC(CO)C(O)C(OC8OC(C(=O)NCCCNC(=O)CCS)C(*)C(O)C8O)C7NC(C)=O)C(O)C6O)C5NC(C)=O)C(*)C(O)C4O)C3NC(C)=O)C(O)C2O)C1NC(C)=O ; [Na+] ; [Na+] ; [Na+]]

In a 1000 mL reactor, a mixture of Cithrol™ DPHS in heptane (15%, 3.36 mL) and heptane (240 mL) was stirred at r.t. A 30 mg/mL solution of 9 (0.120 mmol/g thiols) in 100 mM histidine, 150 mM NaCl buffer pH 2 was prepared as solution A. A 30 mg/mL solution of 5a (0.153 mmol/g maleimides) in 100 mM histidine, 100 mM NaCl buffer pH 2 was prepared as solution B. An aliquot of 24.6 mL of solution A was mixed with 8.69 mL of solution B and 0.27 mL 100 mM histidine, 100 mM NaCl buffer pH 2 and the resulting mixture was added to the reactor at once and it was stirred at r.t. A solution of TMEDA (186 μL) in heptane (5 mL) was added to the mixture and it was stirred at r.t. overnight before a 15% NaCl-solution (150 mL) was added under stirring. The aqueous phase containing the microparticles was harvested after phase separation. For size fractionation, the water-hydrogel suspension was mixed 4:1 v/v with ethanol and wet-sieved using 300, 212, 150, 100 and 50 μm sieves with a 4:1 v/v mixture of 10 mM succinate, 1 mM EDTA, pH 4 and ethanol as sieving buffer. Particle fractions that were retained on the 150 μm and 100 μm sieves were washed with 20 mM succinate, 200 mM NaCl, 5 mM Histidine, 0.1% Pluronic buffer pH 5.5 and formulated in the same buffer to yield 11. The hydrogel contents of the suspensions were determined by drying representative samples under high vacuum after washing the particles with water and ethanol. Compound 11a only shows that the ring-opening hydrolysis took place at one of the carbonyl groups of the thiosuccinimide ring, but it is understood that said reaction can also take place at the other carbonyl group of the thiosuccinimide ring.
Yield: sieve fraction 100 μm: 45.0 mL with 7.5 mg/mL particle content
   sieve fraction 150 μm: 45.0 mL with 6.1 mg/mL particle content

Aliquots of 11 (44 mL of fraction 100 μm 25 mL of fraction 150 μm) were mixed and the microparticles were washed with 500 mM borate, 0.01% Pluronic F-68 buffer pH 9.0 and then incubated in the same buffer at r.t. for 24 hours. The hydrogel was washed with 0.5 M phosphate buffer pH 7.4 and then incubated in a 50 mM solution of DTT in 0.5 M phosphate buffer pH 7.4 at r.t. for 40 minutes. The DTT incubation was repeated once before the microparticles were washed with 20 mM succinate, 200 mM NaCl, 5 mM histidine, 0.1% Pluronic buffer pH 5.5 and formulated in the same buffer to give 11a as microparticle suspension. The hydrogel content of the suspension was determined by drying a representative sample under high vacuum after washing the particles with water and ethanol. An Ellman assay of the product suspension was conducted, and the resulting thiol content of the particle suspension was used to calculate the thiol content of the dry material.
Yield: 75.0 mL with 6.6 mg/mL particle content
Thiol content: 59.8 μmol/g (calculated for dry material)

### Example 8: Synthesis of Protected Ranibizumab Linker Conjugates 14

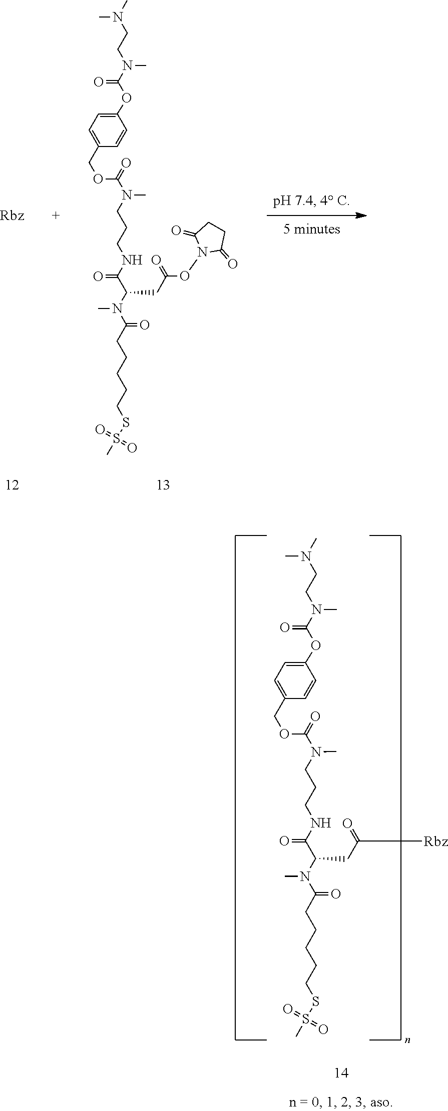

The buffer of a solution of ranibizumab (Rbz) in 10 mM histidine, 10 wt % α,α-D-trehalose, 0.01% Tween® 20 buffer pH 5.5 (62 mL, 2.48 g protein) was exchanged to 30 mM phosphate buffer pH 7.4. The solution was concentrated, sterile filtered and diluted to about 40 mg/mL by addition of 30 mM phosphate buffer pH 7.4 to give solution 12.
Yield: 53.2 g solution with 2.12 g Rbz (85%)
Rbz content: 39.8 mg/mL

To ice-cold 12 (50 mL, 39.8 mg/mL, 1.99 g protein, 41.1 μmol, 1.00 eq.) was added a solution of 13 in DMSO (as described in WO2018175788A1 as compound b8, 100 mM, 8.00 mL, 0.62 mmol, 15.0 eq. active NHS ester) and the mixture was agitated carefully at 0-4° C. for 5 minutes. The pH of the solution was lowered to about pH 4.0 by addition of 0.5 M succinate buffer pH 3.0 (6.0 mL) and it was shaken carefully. After buffer exchange to 5 mM succinate buffer pH 4.0, conjugate mixture 14 was obtained. The different components of the mixture were characterized via MS and deconvolution of the spectra.
Yield: 134 mL solution with 1.94 g Rbz (calculated as native protein, 97%)
Rbz content: 14.5 mg/mL (calculated as native Rbz)
MS: 48382 (native Rbz, calculated average mass: 48380)
   49066 (Rbz linker monoconjugate, calculated average mass: 49066)
   49754 (Rbz linker bisconjugate, calculated average mass: 49752)
   50437 (Rbz linker trisconjugate, calculated average mass: 50438)

### Example 9: Synthesis of Tagylated, Deprotected Ranibizumab Linker Monoconjugate 16

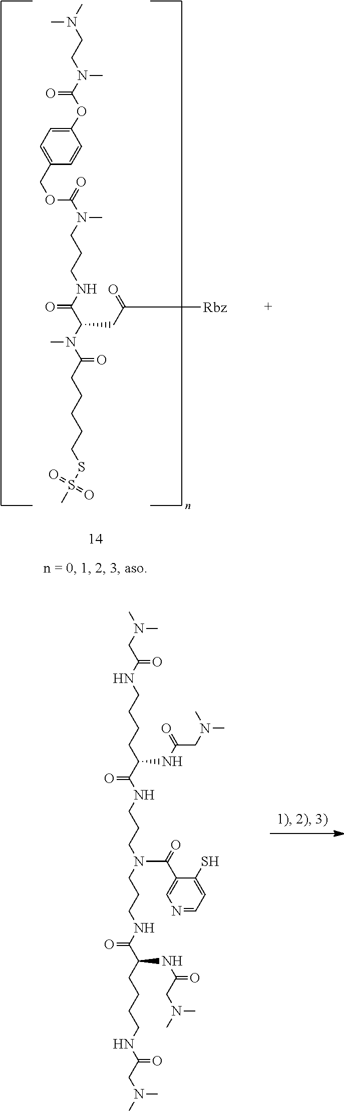

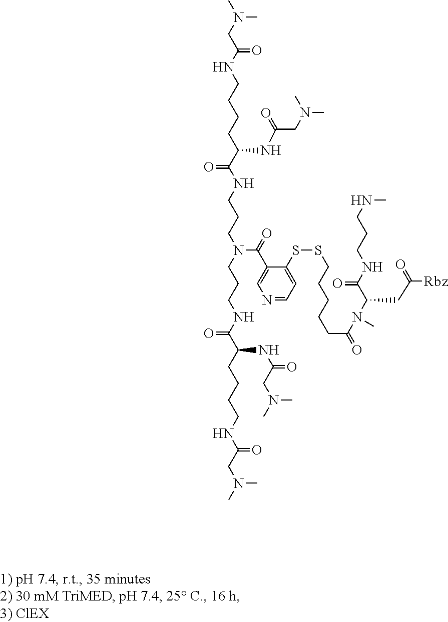

To 14 (14.5 mg/mL, 125 mL, 1.82 g protein, 1.00 eq.) was added an aqueous solution of 15 (as described in WO2018175788A1 as compound e8, 50 mM, 1.89 mL, 2.50 eq.) and the mixture was agitated for 35 minutes. The pH was shifted to pH 7.4 by addition of 0.5 M phosphate, 200 mM TriMED buffer pH 7.8 (21.4 mL, 0.17 vol. eq. with respect to 14) and it was incubated at 25° C. overnight. An aliquot of 148 mL of the incubated mixture was further processed by addition of 0.5 M succinate buffer pH 3.0 (52.7 mL, 0.42 vol. eq. with respect to 14) to shift the pH to about pH 4.0. The tagylated ranibizumab linker monoconjugate 16 was purified and isolated from the mixture by CIEX using succinate buffer pH 4 with a gradient of NaCl. The material was characterized via MS and deconvolution of the spectra.
Yield: 450 mL solution with 383 mg Rbz (calculated as native protein, 21%)
Rbz content: 0.85 mg/mL (calculated as native Rbz)
MS: 49573 (tagylated Rbz monoconjugate, calculated average mass: 49572)

### Example 10: Synthesis of Ranibizumab Linker Monoconjugate 17

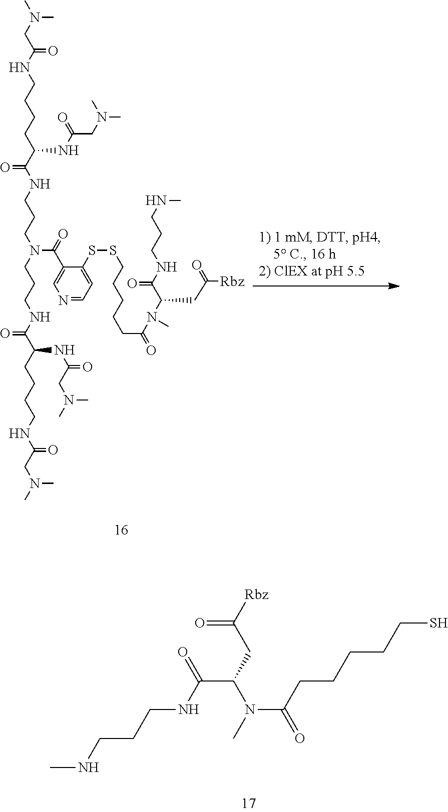

16 (0.85 mg/mL, 450 mL, 383 mg protein, 1.0 eq.) was concentrated to about 4.8 mg/mL using a TFF system. To the concentrated solution (68 mL), a solution of DTT in 20 mM succinate buffer pH 4.0 (25 mM, 2.84 mL, 9.2 eq.) was added, it was shaken carefully and incubated at 2-8° C. overnight. The mixture was purified by CIEX using 10 mM histidine buffer pH 5.5 and a gradient of NaCl. After adjusting the concentration of NaCl in the solution to 150 mM by addition of 10 mM histidine, 500 mM NaCl buffer pH 5.5, the solution was concentrated by ultrafiltration to yield 17. The material was characterized via MS and deconvolution of the spectra.
Yield: 4.54 g solution with 293 mg Rbz (calculated as native protein, 77%)
Rbz content: 64.6 mg/mL (calculated as native Rbz)
MS: 48710 (Rbz monoconjugate, calculated average mass: 48709)

### Example 11: Synthesis of Degradable Microspheres with Transiently Bound Ranibizumab 18a

Exemplary reaction scheme for the synthesis of degradable microspheres with transiently bound ranibizumab:

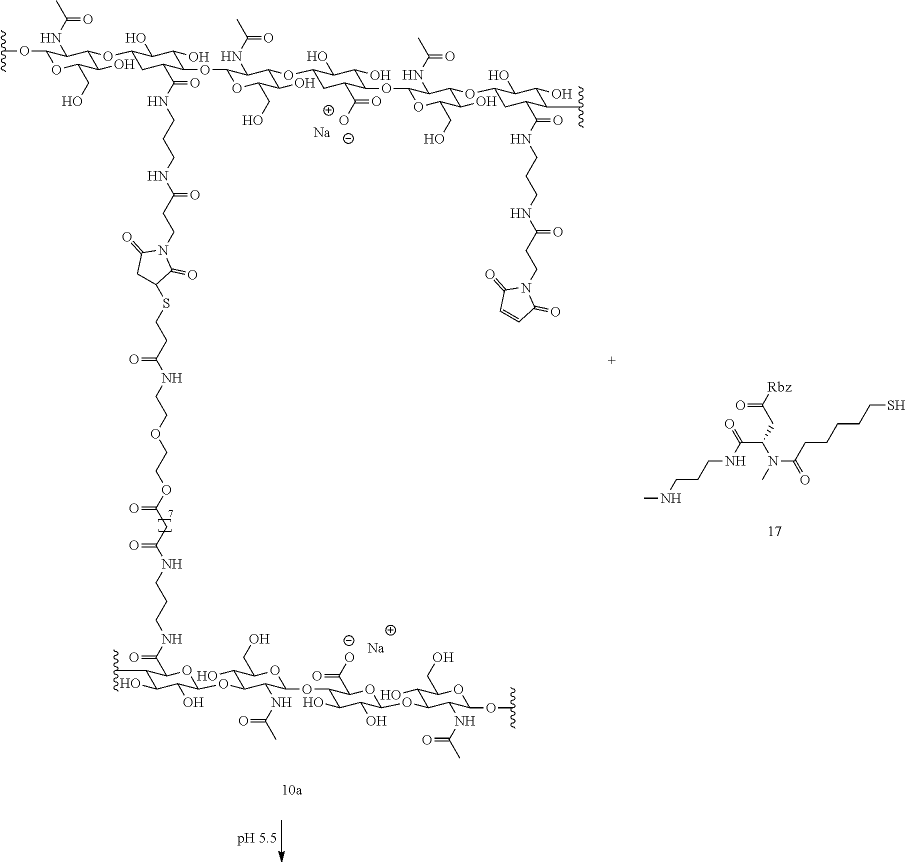

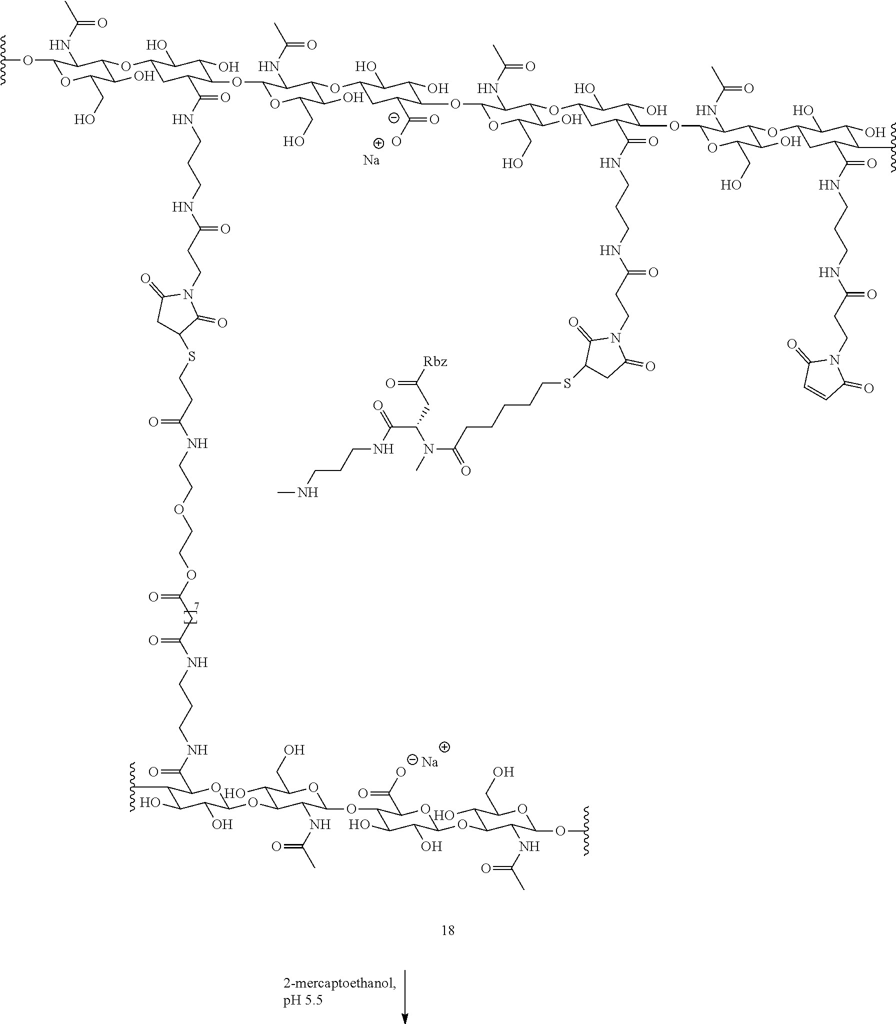

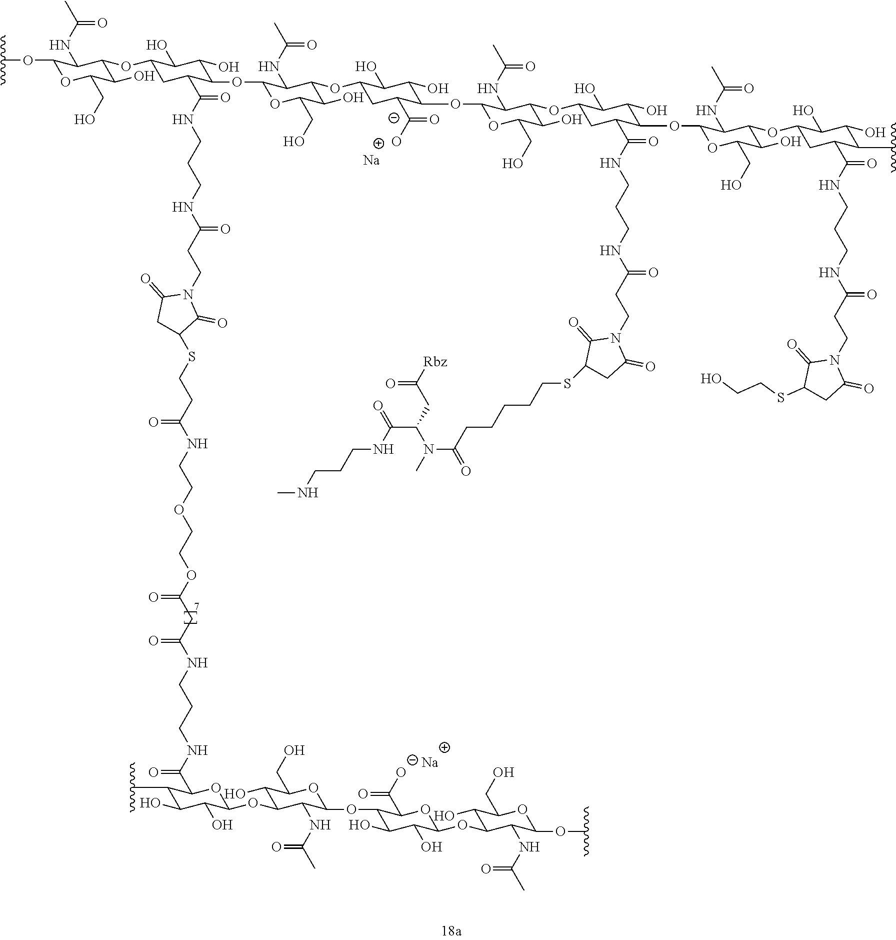

An aliquot of sieve fraction 150 μm of 10a (737 μL, 363 nmol maleimides, 4.2 mg/mL, 117 μmol/g maleimides, 1.26 eq.) was washed with 10 mM histidine, 150 mM NaCl, 0.01% Tween® 20 buffer pH 5.5 and the supernatant was removed. To 17 (721 μL, 302 nmol, 20.4 mg/mL protein), 10 mM histidine, 150 mM NaCl, 0.2% Tween® 20 buffer pH 5.5 (38 μL) and 10 mM histidine, 150 mM NaCl, 0.01% Tween® 20 buffer pH 5.5 (223 μL) were added. An aliquot of this solution (936 μL, 288 nmol, 15.3 mg/mL protein, 1.00 eq.) was added to the hydrogel and the mixture was agitated at r.t. overnight to give a suspension of protein-loaded microspheres 18. The supernatant was removed, and the hydrogel was washed with a 1 mM solution of 2-mercaptoethanol in 10 mM histidine, 150 mM NaCl, 0.01% Tween® 20 buffer pH 5.5 and left standing in the same solution at r.t. for two hours. The microparticles were washed with 10 mM histidine, 150 mM NaCl, 0.01% Tween® 20 buffer pH 5.5 and formulated in the same buffer to yield product 18a.
Yield: 375 μL suspension with 9.7 mg hydrogel-bound ranibizumab (68%)
Rbz content: 26.0 mg/mL (calculated as native Rbz)

### Example 12: Synthesis of Degradable Microspheres with Transiently Bound Ranibizumab 20a

Exemplary reaction scheme for the synthesis of degradable microspheres with transiently bound ranibizumab:

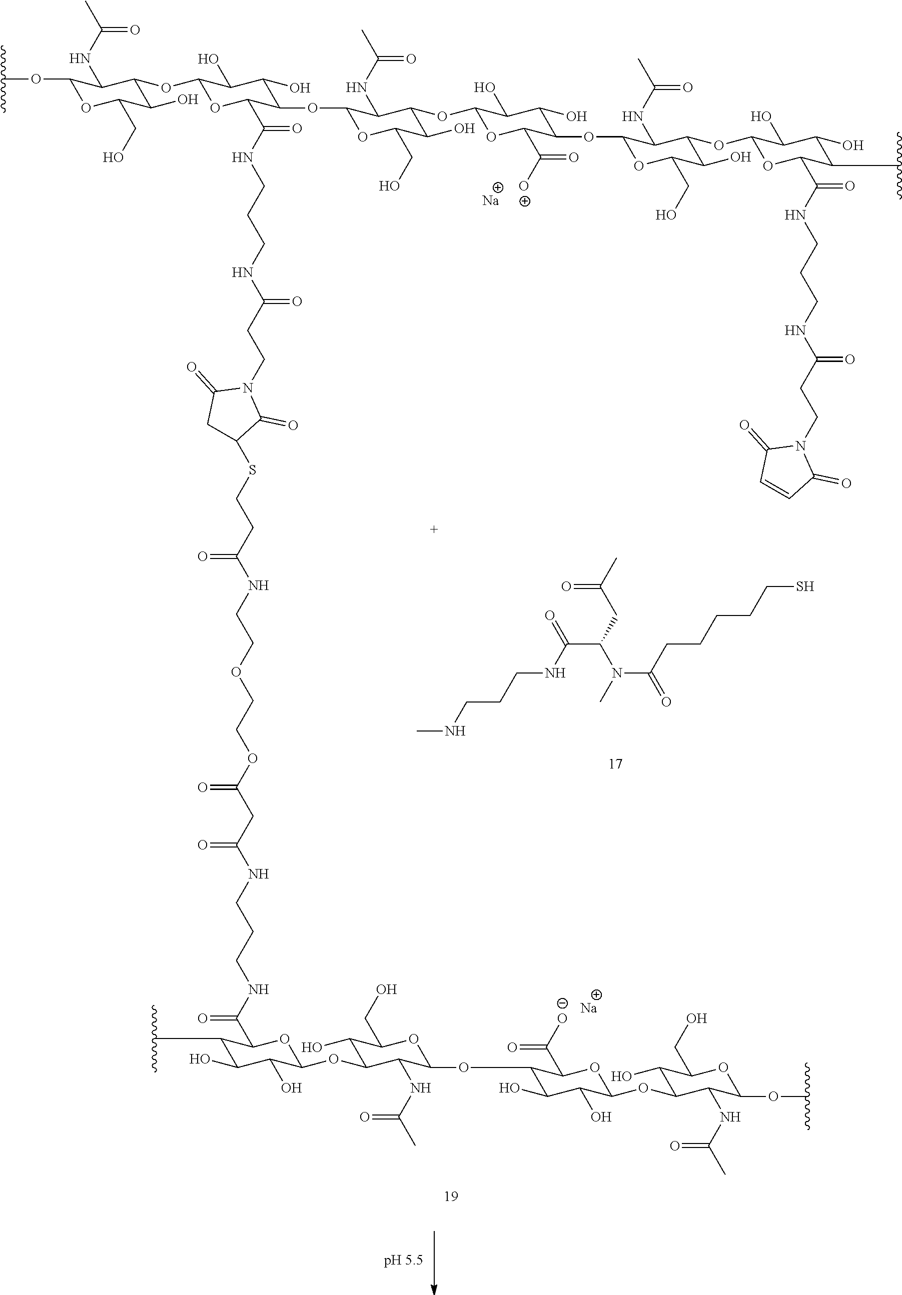 [SMILES: *CC1C(C(=O)NCCCNC(=O)CC(=O)OCCOCCNC(=O)CCSC2CC(=O)N(CCC(=O)NCCCNC(=O)C3OC(OC4C(O)C(CO)OC(O*)C4NC(C)=O)C(O)C(O)C3OC3OC(CO)C(O)C(OC4OC(C(=O)[OH2+])C(OC5OC(CO)C(O)C(OC6OC(C(=O)NCCCNC(=O)CCN7C(=O)C=CC7=O)C(C*)C(O)C6O)C5NC(C)=O)C(O)C4O)C3NC(C)=O)C2=O)OC(OC2C(O)C(CO)OC(OCC3C(C(=O)[O-])OC(OC4C(O)C(CO)OC(O*)C4NC(C)=O)C(O)C3O)C2NC(C)=O)C(O)C1O ; CNCCCNC(=O)[C@H](CC(C)=O)N(C)C(=O)CCCCCS ; [Na+] ; [Na+]]

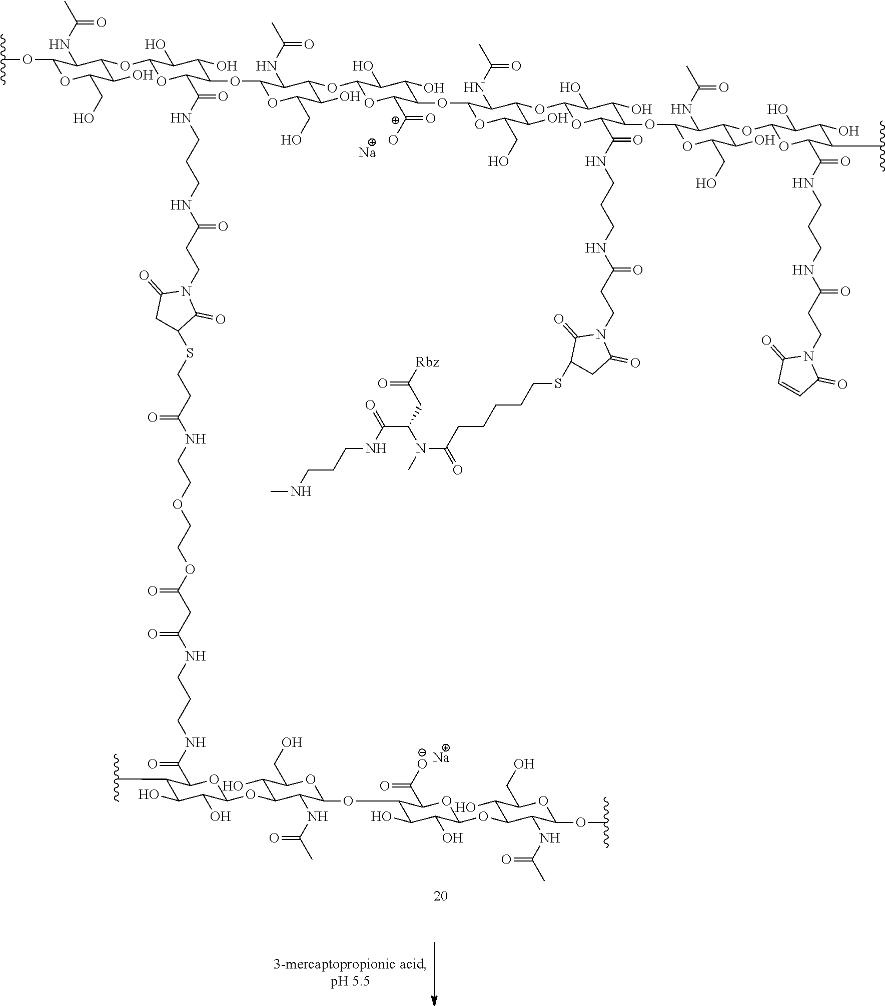

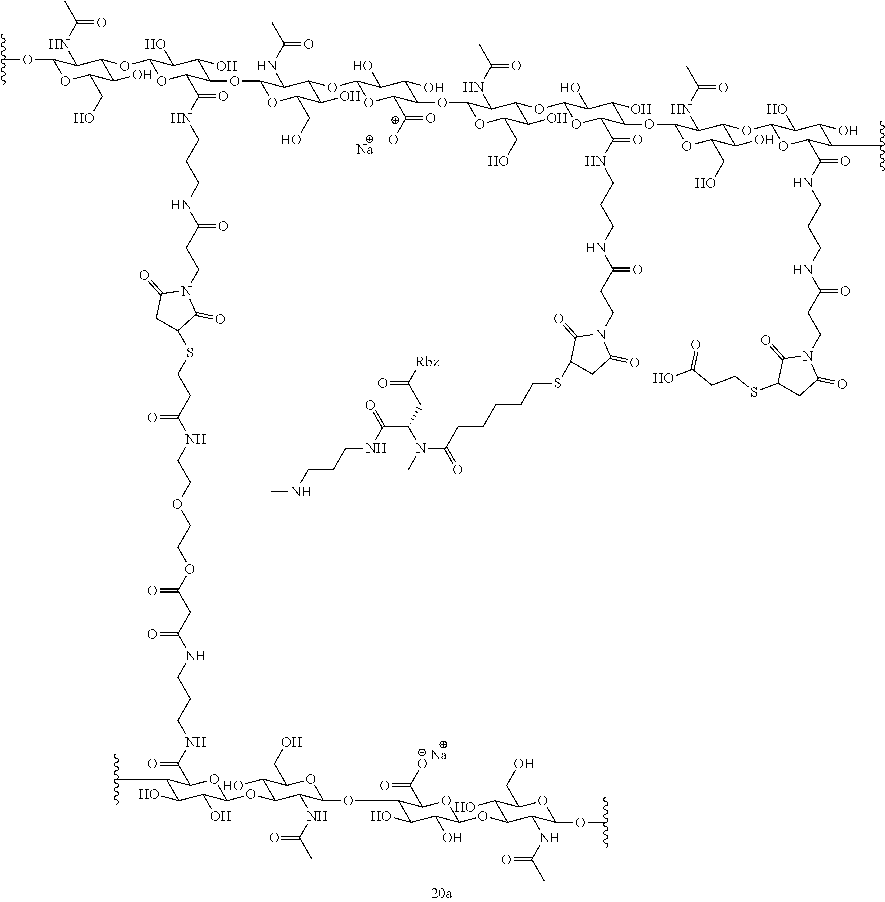

An aliquot of maleimide functionalized microspheres 19, prepared as described for compound 10a, (22.783 mL, 13.5 μmol maleimides, 4.7 mg/mL, 126 μmol/g maleimides) was washed with 10 mM histidine, 150 mM NaCl, 0.01% Tween® 20 buffer pH 5.5 and the supernatant was removed. To 17 (94.3 g, 10 μmol, 5.1 mg/mL protein), 10 mM histidine, 150 mM NaCl, 0.2% Tween® 20 buffer pH 5.5 (4.909 mL) was added. The obtained solution was added to the hydrogel and the mixture was agitated at r.t. overnight to give a suspension of protein-loaded microspheres 20. The supernatant was removed, and the hydrogel was washed with a 1 mM solution of 3-mercaptopropionic acid in 10 mM histidine, 150 mM NaCl, 0.01% Tween® 20 buffer pH 5.5 and was agitated in the same solution at r.t. for two hours. The microparticles were washed and formulated in pH 4 acetate buffer to yield product 20a.
Yield: 7.58 g suspension with 463 mg hydrogel-bound ranibizumab (96.3%)
Rbz content: 61.1 mg/mL (calculated as native Rbz)

### Example 13: Synthesis of Protected CTLA-4 mAB Linker Conjugates 23

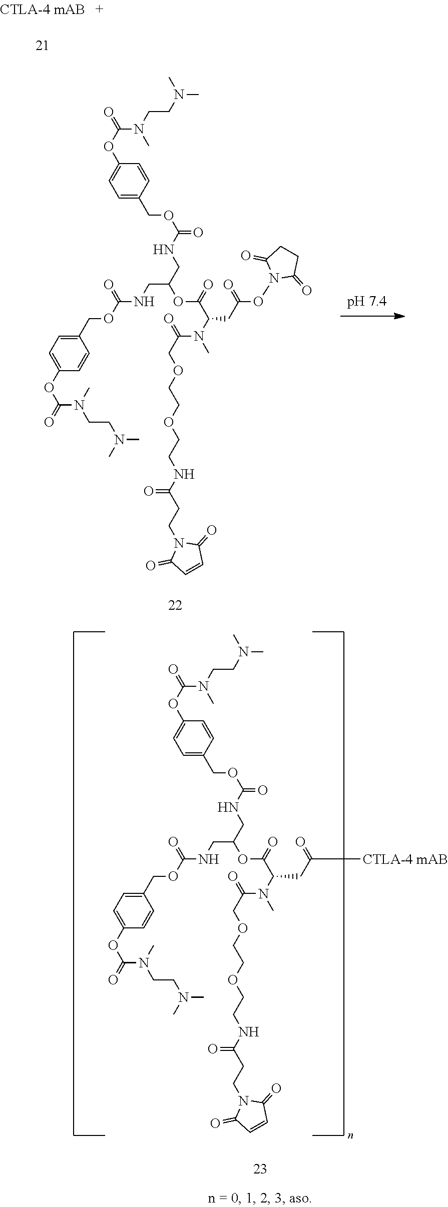

The buffer of a solution of CTLA-4 mAB in 20 mM Tris-HCl, 100 mM NaCl, 1% mannitol, 0.1 mM pentetic acid, 0.01% Tween® 80 buffer pH 7.0 (307 mL, 1.71 g protein) was exchanged to 30 mM sodium phosphate buffer pH 7.4 and concentrated using a TFF system and the protein concentration was afterwards adjusted to 10 mg/mL using additional 30 mM sodium phosphate buffer pH 7.4 to give solution 21, which was stored at 4° C.
Yield: 156 g solution with 1.56 g CTLA-4 mAB (91%)
CTLA-4 mAB content: 10.0 mg/mL

To 21 (100.5 mL, 9.88 mg/mL, 993 mg protein, 6.68 μmol, 1.00 eq.) was added a solution of 22 in DMSO (as described in WO2021136808A1 as compound 9g, 95 mM, 212 μL, 20.1 μmol, 3.0 eq. active NHS ester) and the mixture was agitated at r.t. for 5 minutes. After addition of 500 mM succinate buffer pH 3.0 (12.1 mL), the protein content of the mixture was isolated by CIEX using succinate buffer pH 5.5 with a gradient of NaCl. An aliquot of the conjugate mixture was characterized regarding its composition of the different conjugate types by the reaction of a sample aliquot with excess PEG-20 kDa-SH and subsequent SE-HPLC analysis. 20 mM succinate, 100 mM EDTA, 0.2% Tween® 20 buffer pH 5.5 (6 mL) was added and the mixture was sterile filtered to yield 23.
Yield: 118 mL solution with 991 mg CTLA-4 mAB
   (calculated as native protein, quant.)
CTLA-4 mAB content: 8.4 mg/mL, 56.8 μM (M_{CTLA-4mAB}=148000 kDa)
   (calculated as native protein)
CTLA-4 mAB conjugates: 54% native CTLA-4 mAB
   34% CTLA-4 mAB monoconjugates
   10% CTLA-4 mAB bisconjugates
   2% CTLA-4 mAB trisconjugates
Mal content: 1×34%+2×10%+3×2%=60% of molar protein concentration

### Example 14: Synthesis of Microspheres with Transiently Bound CTLA-4 mAB 24a

An aliquot of 11a (30.5 mL, 12.0 μmol thiols, 6.6 mg/mL, 59.8 μmol/g thiols, 4.0 eq.) was washed with 20 mM succinic acid, 200 mM NaCl, 5 mM EDTA, 0.01% Tween® 20 buffer pH 5.5 and the supernatant was removed. An aliquot of 23 (88.2 mL, 8.4 mg/mL, 741 mg, 5 μmol CTLA-4 mAB, calculated as native protein, 3 μmol maleimides, 1.0 eq.) was added and the mixture was agitated at r.t. overnight to give protein loaded microspheres 24. The material was washed with 30 mM sodium phosphate, 10 mM iodoacetamide, 50 mM TriMED, 0.01% Tween® 20 buffer pH 7.4 and agitated in the same buffer at r.t. for one hour. The microspheres were washed with 30 mM sodium phosphate, 200 mM TriMED, 0.01% Tween® 20 buffer, pH 7.4 and incubated in the same buffer at 25° C. for 24 h. The microspheres were washed with and formulated in a buffer at pH 4, to yield 24a. The protein content was determined by QAAA.
Yield: 35.0 mL with 304 mg polymer-bound CTLA-4 mAB
   (calculated as native protein, 41%)
CTLA-4 mAB content: 8.7 mg/mL (calculated as native protein)

### Abbreviations

CIEX Cation exchange chromatography
CTLA-4 Cytotoxic T-lymphocyte-associated protein 4
DCM Dichloromethane
DIPEA N,N-Diisopropylethylamine
DMAP 4-(Dimethylamino)pyridine
DMF N,N-Dimethylformamide
DMSO Dimethylsulfoxide
DPHS Dipolyhydroxystearate
DS Degree of substitution
DTNB 5,5′-Dithiobis(2-nitrobenzoic acid)
DTT Dithiothreitol
EDC N-(3-Dimethylaminopropyl)-N′-ethylcarbodiimide Hydrochloride
EDTA Ethylenediaminetetraacetic acid
eq. Equivalents
EtOAc Ethyl acetate
EtOH Ethanol
HA Hyaluronic acid
HEPES (4-(2-Hydroxyethyl)-1-piperazineethanesulfonic acid)
HOBt 1-Hydroxybenzotriazole
HOSu N-hydroxysuccinimide
iPrOH Isopropanol
mAB Monoclonal antibody
MeCN Acetonitrile
MES 2-(N-Morpholino)ethanesulfonic acid
MS Mass spectrometry
NHS N-Hydroxysuccinimide
OPA o-Phthalaldehyde
PE Polyethylene
PEG Poly(ethylene glycol)
PES Polyethersulfone
QAAA Quantitative amino acid analysis
r.t. Room temperature
Rbz Ranibizumab
SE-HPLC Size exclusion high performance liquid chromatography
TCEP Tris(2-carboxyethyl)phosphine
TFA Trifluoroacetic acid
TFF Tangential flow filtration
TGI Triglyceryl diisostearate
TMEDA N,N,N′,N′-Tetramethylethylenediamine
TNB 5-Thio-2-nitrobenzoic acid
TriMED N,N,N′-Trimethylethylenediamine

1. A method for preparing hydrogel microspheres or pharmaceutically acceptable salts thereof comprising a crosslinked hyaluronic acid (HA), wherein the method comprises the steps of:
(a) mixing a solution A with a solution B to form an emulsion, wherein solution A comprises a first functionalized HA that is modified with one or more -FG₁ and optionally further functional groups and a second functionalized HA that is modified with one or more -FG₂ and optionally further functional groups, wherein each -FG₁ and -FG₂ are functional group moieties that are different from each other, wherein -FG₁ on the first functionalized HA reacts with -FG₂ on the second functionalized HA to form a plurality of crosslinks which results in the formation of hydrogel HA microspheres;
(b) optionally, adding a pH-adjusting agent to the emulsion of step (a); and
(c) collecting the obtained hydrogel HA microspheres of step (a) or (b).

2. The method of claim 1, wherein solution A of step (a) further comprises a buffering agent.

3. The method of claim 1, wherein solution B of step (a) comprises an emulsifying agent and a solvent

4. The method of claim 1, wherein the method comprises the steps of:
(a) mixing a solution A with a solution B to form an emulsion, wherein solution A comprises a first functionalized HA that is modified with one or more -FG₁ and a second functionalized HA that is modified with one or more -FG₂, wherein -FG₁ and -FG₂ are functional group moieties that are different from each other and wherein -FG₁ on the first functionalized HA reacts with -FG₂ on the second functionalized HA to form a plurality of crosslinks which results in the formation of hydrogel HA microspheres;
(b) optionally, adding a pH-adjusting agent to the emulsion of step (a);
(c) collecting the obtained hydrogel HA microspheres of step (b);
(d) optionally, size fractionating the obtained hydrogel HA microspheres of step (c) to obtain microspheres with a particular particle size distribution;
(e) optionally, washing the microspheres obtained in step (c) or (d);
(f) optionally, incubating the hydrogel HA microspheres of step (c), (d) or (e) in a buffering agent of a pH ranging from about 8 to above 12;
(g) optionally, incubating the hydrogel HA microspheres of step (c), (d), (e) or (f) with a reducing agent;
(h) optionally, washing the microspheres obtained in step (f) or (g); and
(i) optionally, collecting the hydrogel HA microspheres of step (d), (e), (f), (g) or (h).

5. The method claim 1, wherein -FG₁ is independently selected from the group consisting of: 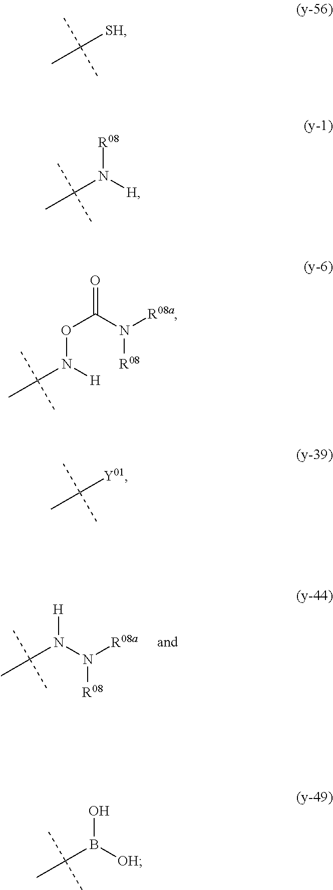
wherein the dashed line indicates the attachment to the first functionalized HA;
—Y⁰¹ is independently selected from the group consisting of —F, —Cl, —Br and —I;
each —R⁰⁸ and —R^{08a} is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰⁹, which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰¹⁰)—, —S(O)₂N(R⁰¹⁰)—, —S(O)N(R⁰¹⁰)—, —S(O)₂—, —S(O)—, —N(R⁰¹⁰)S(O)₂N(R^{010a})—, —S—, —N(R⁰¹⁰)—, —OC(OR⁰¹⁰)(R^{010a})—, —N(R⁰¹⁰)C(O)N(R^{010a})— and —OC(O)N(R⁰¹⁰)—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R⁰⁹ which are the same or different; and
each —R⁰⁹, —R⁰¹⁰ and —R^{010a} is independently selected from the group consisting of —H and C₁₋₆ alkyl; wherein C₁₋₆ alkyl is optionally substituted with one or more halogen, which are the same or different; and
-FG₂ is independently selected from the group consisting of: 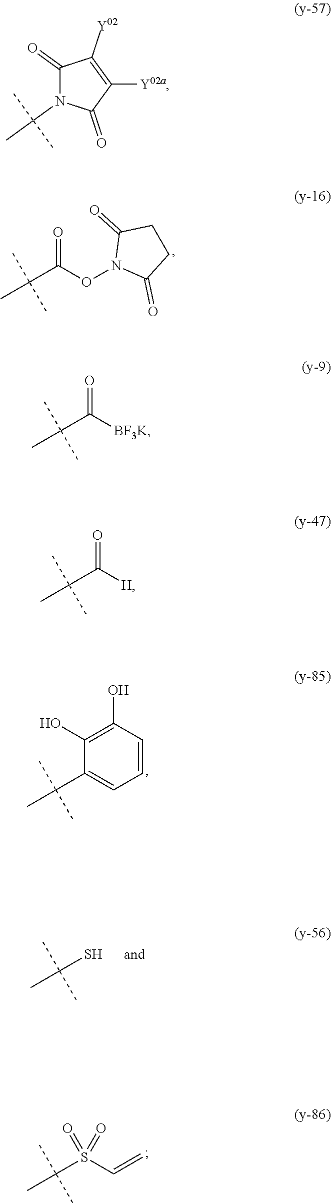
wherein the dashed line indicates the attachment to the second functionalized HA;
each —Y⁰² and —Y^{02a} is independently selected from the group consisting of —H and —Br;
provided that -FG₁ is of formula (y-56) then -FG₂ is of formula (y-57) or (y-86); if -FG₁ is of formula (y-1) then -FG₂ is of formula (y-16) or (y-47); if -FG₁ is of formula (y-44) then -FG₂ is of formula (y-16) or (y-47); if -FG₁ is of formula (y-6) then -FG₂ is of formula (y-9); if -FG₁ is of formula (y-49) then
-FG₂ is of formula (y-85); if -FG₁ is of formula (y-44) then -FG₂ is of formula (y-47); or if
-FG₁ is of formula (y-39) then -FG₂ is of formula (y-56).

6. The method of claim 1, wherein the pH-adjusting agent increases the pH of the emulsion of step (a) and -FG₁ is independently selected from the group consisting of: 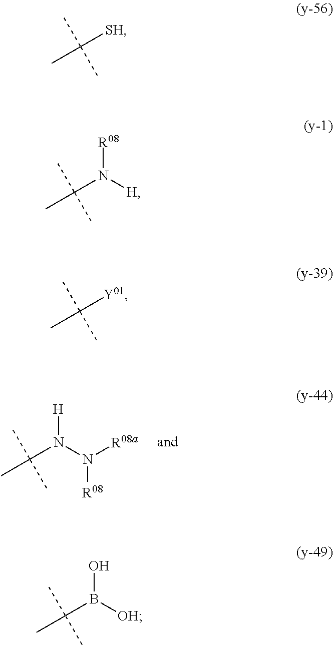
wherein the dashed line indicates the attachment to the first functionalized HA;
—Y⁰¹ is independently selected from the group consisting of —F, —Cl, —Br and —I;
each —R⁰⁸ and —R^{08a} is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰⁹, which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰¹⁰)—, —S(O)₂N(R⁰¹⁰)—, —S(O)N(R⁰¹⁰)—, —S(O)₂—, —S(O)—, —N(R⁰¹⁰)S(O)₂N(R^{010a})—, —S—, —N(R⁰¹⁰)—, —OC(OR⁰¹⁰)(R^{010a})—, —N(R⁰¹⁰)C(O)N(R^{010a})— and —OC(O)N(R⁰¹⁰)—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R⁰⁹ which are the same or different; and
each —R⁰⁹, —R⁰¹⁰ and —R^{010a} is independently selected from the group consisting of —H and C₁₋₆ alkyl; wherein C₁₋₆ alkyl is optionally substituted with one or more halogen, which are the same or different; and
-FG₂ is independently selected from the group consisting of: 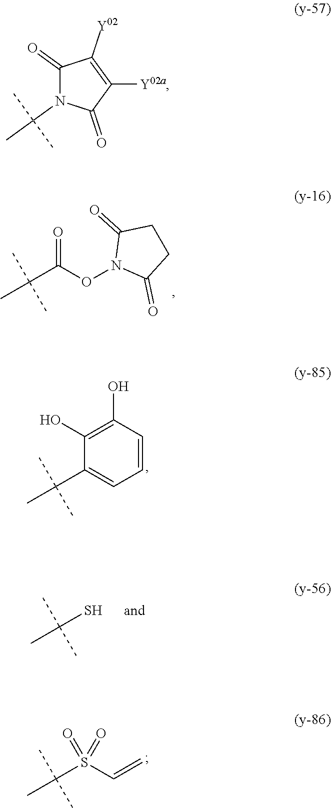
wherein the dashed line indicates the attachment to the second functionalized HA;
each —Y⁰² and —Y^{02a} is independently selected from the group consisting of —H and —Br;
provided that -FG₁ is of formula (y-56) then -FG₂ is of formula (y-57) or (y-86); if -FG₁ is of formula (y-1) then -FG₂ is of formula (y-16); if -FG₁ is of formula (y-44) then -FG₂ is of formula (y-16); or if -FG₁ is of formula (y-39) then -FG₂ is of formula (y-56).

7. The method of claim 1, wherein -FG₁ is 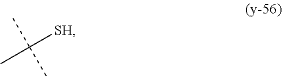
wherein the dashed line indicates the attachment to the first functionalized HA, -FG₂ is 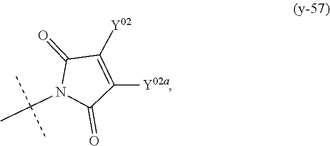
wherein the dashed line indicates the attachment to the second functionalized HA, the
pH-adjusting agent increases the pH of the emulsion of step (a) from about 1 to about 9, preferably from about 1 to about 5.5, more preferably from about 2 to about 4 and wherein each —Y⁰² and —Y^{02a} is independently selected from the group consisting of —H and —Br.

8. The method of claim 1, wherein the method comprises the following steps:
(a) mixing a solution A with a solution B to form an emulsion, wherein solution A comprises a first functionalized HA that is modified with one or more -FG₁ and a second functionalized HA that is modified with one or more -FG₂, wherein -FG₁ and -FG₂ are functional group moieties that are different from each other and wherein -FG₁ on the first functionalized HA reacts with -FG₂ on the second functionalized HA to form a plurality of crosslinks which results in the formation of hydrogel HA microspheres, wherein
the first functionalized HA comprises a plurality of each of the following linearly connected Z¹ and Z⁵ units: 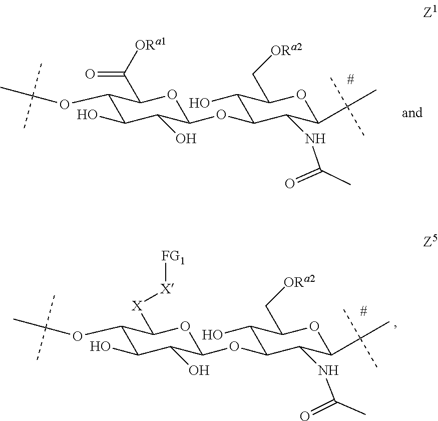
the second functionalized HA comprises a plurality of each of the following linearly connected Z¹ and Z⁶ units: 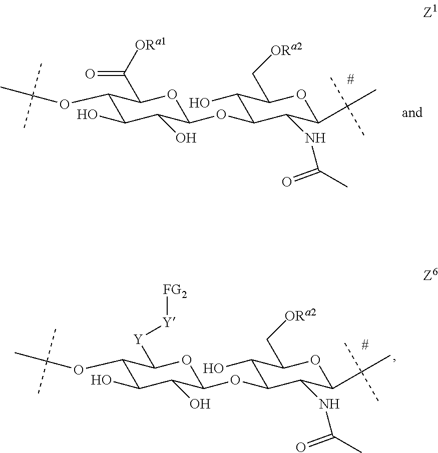
wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is independently selected from the group consisting of —H, C₁₋₁₀ alkyl,
an ammonium ion, a tetrabutylammonium ion, a cetyl trimethylammonium ion,
an alkali metal ion and an alkaline earth metal ion;
each —R^{a2} is independently —H or C₁₋₁₀ alkyl;
each -FG₁, -FG₂ is independently the functional group moiety;
each —X—, —Y— is independently a carbonyl group or absent;
each —X′—, —Y′— is independently a spacer moiety or absent;
(b) adding a pH-adjusting agent to the emulsion of step (a);
(c) collecting the obtained hydrogel HA microspheres of step (a) or (b), wherein said hydrogel comprises a plurality of Z³ units: 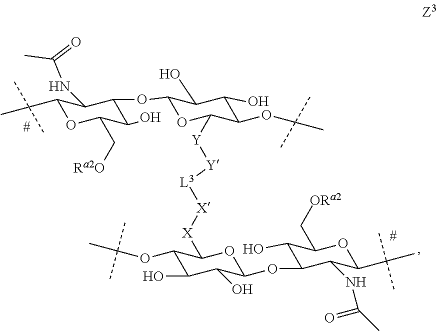
wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each —R a₂, —X—, —Y—, —X′— and —Y′— are defined as in step (a);
each -L³- is independently a linkage moiety or absent;
(d) optionally, size fractionating the obtained hydrogel HA microparticles of step (a), (b) or (c) to obtain microspheres with a particular particle size distribution;
(e) optionally, washing the hydrogel HA microspheres obtained in step (a), (b), (c) or (d);
(f) optionally, incubating the hydrogel HA microspheres of step (a), (b), (c), (d) or (e) in a buffering agent of a pH ranging from about 8 to about 12;
(g) optionally, incubating the hydrogel HA microspheres of step (a), (b), (c), (d), (e) or (f) with a reducing agent;
(h) optionally, washing the microspheres obtained in step (f) or (g); and
(i) optionally, collecting the hydrogel HA microspheres of step (d), (e), (f), (g) or (h).

9. The method of claim 1, wherein the method comprises the following steps:
(a) mixing a solution A with a solution B to form an emulsion, wherein solution A comprises a first functionalized HA that is modified with one or more thiol functional groups and a second functionalized HA that is modified with one or more maleimide functional groups, wherein the thiol functional groups on the first functionalized HA react with the maleimide functional groups on the second functionalized HA to form a plurality of crosslinks which results in the formation of hydrogel HA microspheres, wherein
the first functionalized HA comprises a plurality of each of the following linearly connected Z¹ and Z⁵-i units: 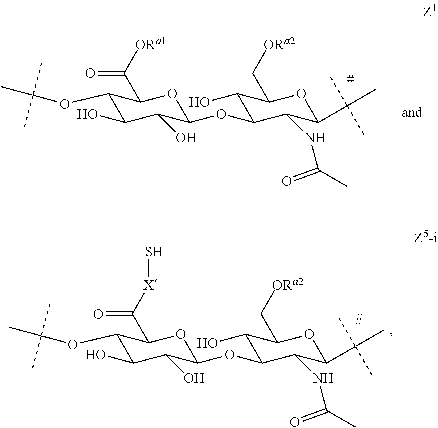
the second functionalized HA comprises a plurality of each of the following linearly connected Z¹ and Z⁶-i units: 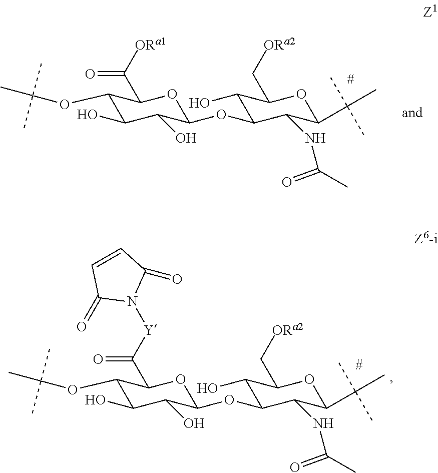
wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is independently selected from the group consisting of —H, C₁₋₁₀ alkyl,
an ammonium ion, a tetrabutylammonium ion, a cetyl trimethylammonium ion,
an alkali metal ion and an alkaline earth metal ion;
each —R^{a2} is independently —H or C₁₋₁₀ alkyl;
each —X′—, —Y′— is independently a spacer moiety or absent;
(b) adding a pH-adjusting agent to the emulsion of step (a);
(c) collecting the obtained hydrogel HA microspheres of step (a) or (b), wherein said hydrogel comprises a plurality of Z³-i units: 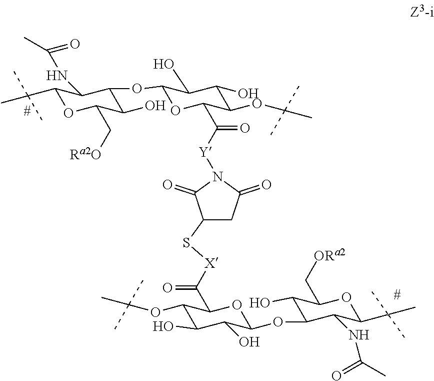
wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each —R^{a2}, —X′— and —Y′— are defined as in step (a);
(d) optionally, size fractionating the obtained hydrogel HA microparticles of step (a), (b) or (c) to obtain microspheres with a particular particle size distribution;
(e) optionally, washing the hydrogel HA microspheres obtained in step (a), (b), (c) or (d);
(f) optionally, incubating the hydrogel HA microspheres of step (a), (b), (c), (d) or (e) in a buffering agent of a pH ranging from about 8 to about 12, to provide hydrogel HA microspheres comprising a plurality of Z³-i′ units: 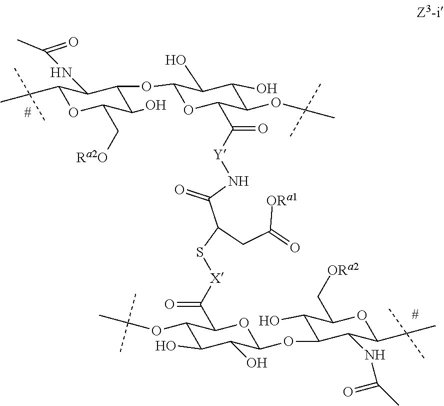
wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1}, —R^{a2}, —X′— and —Y′— are defined as in step (a);
(g) optionally, incubating the hydrogel HA microspheres of step (a), (b), (c), (d), (e) or (f) with a reducing agent;
(h) optionally, washing the microspheres obtained in step (f) or (g); and
(i) optionally, collecting the hydrogel HA microspheres of step (d), (e), (f), (g) or (h).

10. The method of claim 8, wherein steps (d) and (e) are not optional and steps (f) to (h) are not present.

11. The method of claim 1, wherein the pH-adjusting agent is selected from the group consisting of N,N,N′,N′-tetramethylethylene diamine (TMEDA), 1,4-dimethylpiperazine, 4-methylmorpholine, 4-ethylmorpholine, 1,4-diazabicyclo[2.2.2]octane, 1,1,4,7,10,10-hexamethyltriethylenetetramine, 1,4,7-trimethyl-1,4,7-triazacyclononane, tris[2-(dimethylamino)ethyl]amine, triethylamine, diisopropylethylamine (DIPEA), trimethylamine, N,N-dimethylethylamine, N,N,N′,N′-tetramethyl-1,6-hexanediamine, N,N,N′,N′″,N″-pentamethyldiethylenetriamine, 1,8-diazabicyclo[5.4.0]undec-7-ene, 1,5-diazabicyclo[4.3.0]non-5-ene and hexamethylenetetramine.

12. The method of claim 1, wherein each —X′— and —Y′— are independently a spacer moiety selected from the group consisting of -T′-, C₁₋₅₀ alkyl, C₂₋₅₀ alkenyl, and C₂₋₅₀ alkynyl; wherein C₁₋₅₀ alkyl, C₂₋₅₀ alkenyl and C₂₋₅₀ alkynyl are optionally substituted with one or more —R^{y1}, which are the same or different and wherein C₁₋₅₀ alkyl, C₂₋₅₀ alkenyl, and C₂₋₅₀ alkynyl are optionally interrupted by one or more groups selected from the group consisting of -T′-, —C(O)O—, —O—, —C(O)—, —C(O)N(R^{y2})—, —S(O)₂N(R^{y2})—, —S(O)N(R^{y2})—, —S(O)₂—, —S(O)—, —N(R^{y2})S(O)₂N(R^{y2a})—, —N(R^{y2})—, —OC(OR^{y2})(R^{y2a})—, —N(R^{y2})C(O)N(R^{y2a})— and —OC(O)N(R^{y2})—;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each T′ is independently optionally substituted with one or more —R^{y1}, which are the same or different;
each —R^{y1} is independently selected from the group consisting of halogen, —CN, oxo (═O), —COOR^{y3}, —OR^{y3}, —C(O)R^{y3}, —C(O)N(R^{y3}R^{y3a}), —S(O)₂N(R^{y3}R^{y3a}),
—S(O)N(R^{y3}R^{y3a}), —S(O)₂R^{y3}, —S(O)R^{y3}, —N(R^{y3})S(O)₂N(R^{y3a}R^{y3}b), —SR^{y3}, —N(R^{y3}R^{y3a}),
—NO₂, —OC(O)R^{y3}, —N(R^{y3})C(O)R^{y3a}, —N(R^{y3})S(O)₂R^{y3a}, —N(R^{y3})S(O)R^{y3a},
—N(R^{y3})C(O)OR^{y3a}, —N(R^{y3})C(O)N(R^{y3a}R^{y3b}), —OC(O)N(R^{y3}R^{y3a}), and C₁₋₆ alkyl; wherein
C₁₋₆ alkyl is optionally substituted with one or more halogen, which are the same or different; and
each —R^{y2}, —R^{y2a}, —R^{y3}, —R^{y3a}, —R^{y3b} is independently selected from the group consisting of —H and C₁₋₆ alkyl; wherein C₁₋₆ alkyl is optionally substituted with one or more halogen, which are the same or different.

13. The method of claim 1, wherein the method comprises the following steps:
(a) mixing a solution A with a solution B to form an emulsion, wherein solution A comprises a first functionalized HA that is modified with one or more thiol functional groups and a second functionalized HA that is modified with one or more maleimide functional groups, wherein the thiol functional groups on the first functionalized HA react with the maleimide functional groups on the second functionalized HA to form a plurality of crosslinks which results in the formation of hydrogel HA microspheres, wherein,
the first functionalized HA comprises a plurality of each of the following linearly connected Z¹ and Z⁵-i units: 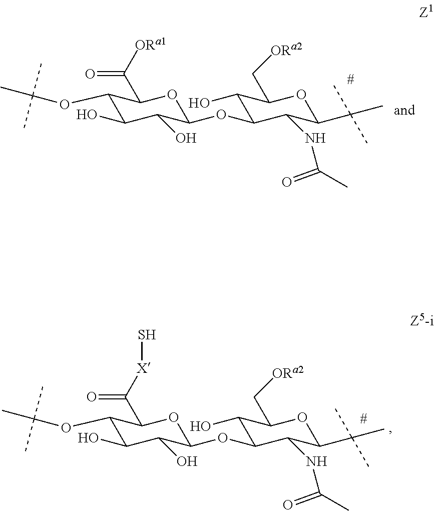
the second functionalized HA comprises a plurality of each of the following linearly connected Z¹ and Z⁶-i units: 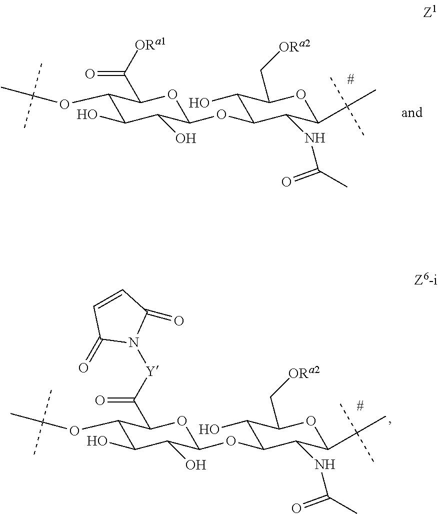
wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is H or an alkali metal ion;
each —R^{a2} is —H;
each —X′— is of formula (x4): 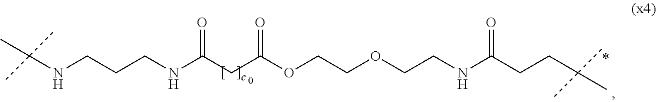
wherein the unmarked dashed line indicates the attachment to the carbonyl group, the dashed line marked with an asterisk indicates the attachment to the sulfur atom and c₀ is 7;
each —Y′— is of formula (y4): 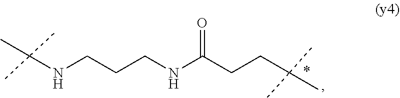
wherein the unmarked dashed line indicates the attachment to the carbonyl group and the dashed line marked with an asterisk indicates the attachment to the nitrogen atom of the maleimide ring;
(b) adding TMEDA to the emulsion of step (a);
(c) collecting the obtained hydrogel HA microspheres of step (a) or (b), wherein said hydrogel comprises a plurality of Z³-i units: 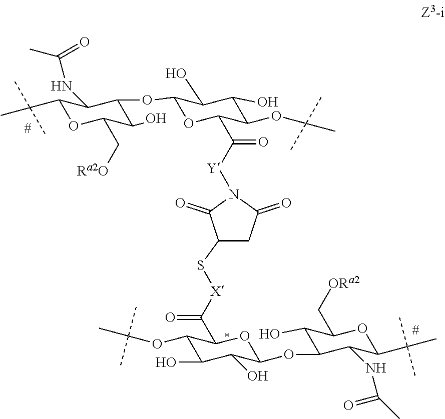
wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each —R^{a2}, —X′— and —Y′— are defined as in step (a);
(d) size fractionating the obtained hydrogel HA microparticles of step (c) to obtain microspheres with a particular particle size distribution;
(e) washing the hydrogel HA microspheres obtained in step (d); and
(f) collecting the hydrogel HA microspheres of step (e).

14. The method of claim 1, wherein the molecular weight of the first and second functionalized HA independently ranges from about 80 kDa to about 250 kDa, such as from about 90 kDa to about 200 kDa or such as from about 100 kDa to about 150 kDa.

15. (canceled)

16. The method of claim 1, wherein in step (a) solution A further comprises citrate and/or histidine, preferably at a pH of about 2.

17. The method of claim 1, wherein in step (a) solution B comprises heptane and sorbitan monooleate or tetradecane and sorbitan monooleate.

18. (canceled)

19. (canceled)

20. Hydrogel HA microspheres or pharmaceutically acceptable salts thereof obtainable by the method of claim 1.

21. (canceled)

22. A drug conjugate or pharmaceutically acceptable salt thereof comprising the hydrogel HA microspheres of claim 20.

23. A drug conjugate or pharmaceutically acceptable salt thereof comprising a hyaluronic acid (HA) hydrogel microsphere comprising crosslinked HA chains or pharmaceutically acceptable salt thereof comprising crosslinked HA chains to which a plurality of drug moieties is covalently and reversibly conjugated, said drug conjugate comprising a plurality of each of the following units: 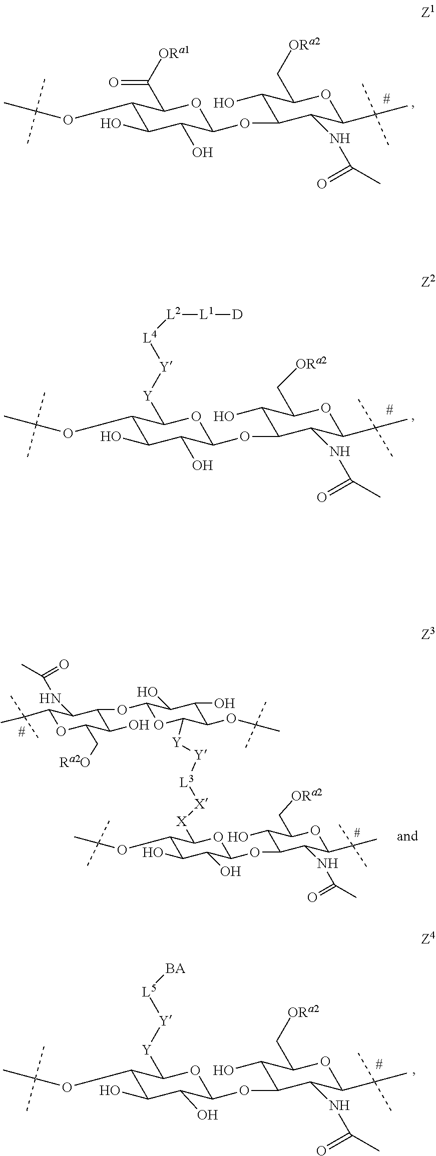
wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
each R^{a1} is independently selected from the group consisting of —H, C₁₋₁₀ alkyl, an ammonium ion, a tetrabutylammonium ion, a cetyl trimethylammonium ion, an alkali metal ion and an alkaline earth metal ion;
each —R^{a2} is independently —H or C₁₋₁₀ alkyl;
each —X—, —Y— is independently a carbonyl group or absent;
each —X′—, —Y′— is independently a spacer moiety or absent;
wherein each -D is independently a drug moiety that is covalently and reversibly conjugated to -L¹-;
each -L¹- is independently a reversible linker moiety;
each -L²- is independently a spacer moiety or absent;
each -L³-, -L⁴-, -L⁵- is independently a linkage moiety or absent; and
each -BA is independently a blocking agent.

24. The drug conjugate or pharmaceutically acceptable salt thereof of claim 23, wherein said drug conjugate comprises Z¹ in a range of about 50% to about 98%, Z² in a range of about 0.1% to about 20%, Z³ in a range of about 0.1% to about 20% and Z⁴ in a range of about 0.1% to about 10%.

25. The drug conjugate or pharmaceutically acceptable salt thereof of claim 23, wherein -BA is selected from the group consisting of 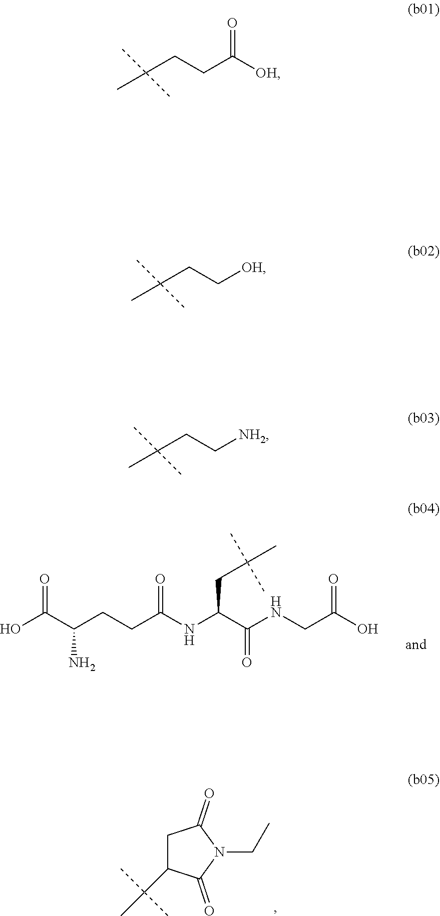
wherein the dashed line indicates the attachment to -L⁵-.

26. The drug conjugate or pharmaceutically acceptable salt thereof of claim 23, wherein said drug conjugate comprises a plurality of each of the following units: 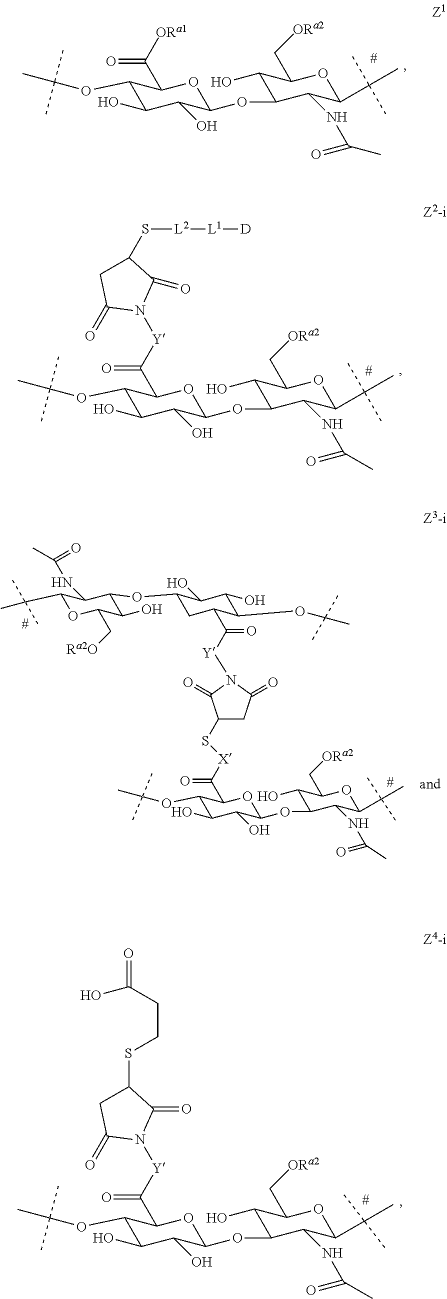
each R^{a1} is independently selected from the group consisting of —H, C₁₋₁₀ alkyl, an ammonium ion, a tetrabutyl ammonium ion, a cetyl tri methyl ammonium ion, an alkali metal ion and an alkaline earth metal ion;
each —R^{a2} is independently —H or C₁₋₁₀ alkyl;
each —X′—, —Y′— is independently a spacer moiety or absent;
wherein each -D is independently a drug moiety that is covalently and reversibly conjugated to -L¹-;
each -L¹- is independently a reversible linker moiety;
each -L²- is independently a spacer moiety or absent; and
wherein said drug conjugate comprises Z¹ in a range of about 50% to about 98%, Z²-i in a range of about 0.1% to about 20%, Z³-i in a range of about 0.1% to about 20% and Z⁴-i in a range of about 0.1% to about 10%.

27. The drug conjugate or pharmaceutically acceptable salt thereof of claim 23, wherein -D is selected from the group consisting of small molecule, medium size, peptide and protein drug moieties.

28. The drug conjugate or pharmaceutically acceptable salt thereof of claim 23, wherein -D is a protein drug moiety.

29. The drug conjugate or pharmaceutically acceptable salt thereof of claim 23, wherein -D is a monoclonal or polyclonal antibody or fragment or fusion thereof.

30. The drug conjugate or pharmaceutically acceptable salt thereof of claim 23, wherein each R^{a1} is H or an alkali metal ion and —R^{a2} is —H.

31. The drug conjugate or pharmaceutically acceptable salt thereof of claim 23, wherein each -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl and C₂₋₅₀ alkynyl; wherein -T′-, C₁₋₅₀ alkyl, C₂₋₅₀ alkenyl and C₂₋₅₀ alkynyl are optionally substituted with one or more —R^{y2}, which are the same or different and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})— and —OC(O)N(R^{y3})—;
—R^{y1} and —R^{y1a} are independently selected from the group consisting of —H, -T′, C₁₋₅₀ alkyl, C₂₋₅₀ alkenyl and C₂₋₅₀ alkynyl; wherein -T′, C₁₋₅₀ alkyl, C₂₋₅₀ alkenyl and C₂₋₅₀ alkynyl are optionally substituted with one or more —R^{y2}, which are the same or different, and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each T′ is independently optionally substituted with one or more —R^{y2}, which are the same or different;
each —R^{y2} is independently selected from the group consisting of halogen, —CN, oxo (═O), —C(O)OR^{y5}, —OR^{y5}, —C(O)R^{y5}, —C(O)N(R^{y5})(R^{y5a}), —S(O)₂N(R^{y5})(R^{y5a}), —S(O)N(R⁵)(R^{y5a}), —S(O)₂R^{y5}, —S(O)R^{y5}, —N(R^{y5})S(O)₂N(R⁵)(R^{y5a}), —SR^{y5}, —N(R^{y5})(R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5})(R^{y5a}), —OC(O)N(R⁵)(R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

32. The drug conjugate or pharmaceutically acceptable salt thereof of claim 23, wherein each -L¹- is of formula (I): 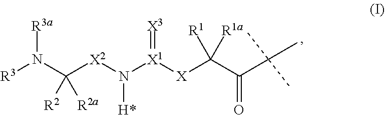
wherein
the dashed line indicates the attachment to a nitrogen of -D by forming an amide bond;
—X— is —C(R⁴R^{4a})—; —N(R⁴)—; —O—; —C(R⁴R^{4a})—C(R⁵R^{5a})—; —C(R⁵R^{5a})—C(R⁴R^{4a})—; —C(R⁴R^{4a})—N(R⁶)—; —N(R⁶)—C(R⁴R^{4a})—; —C(R⁴R^{4a})—O—; —O—C(R⁴R^{4a})—; or —C(R⁷R⁷a)—;
X¹ is C; or S(O);
—X²— is —C(R⁸R^{8a})—; or —C(R⁸R^{8a})—C(R⁹R^{9a})—;
═X³ is ═O; ═S; or ═N—CN;
—R¹, —R^{1a}, —R², —R^{2a}, —R⁴, —R^{4a}, —R⁵, —R^{5a}, —R⁶, —R⁸, —R^{8a}, —R⁹, —R^{9a} are independently selected from the group consisting of —H; and C₁₋₆ alkyl;
—R³, —R^{3a} are independently selected from the group consisting of —H; and C₁₋₆ alkyl, provided that in case one of —R³, —R^{3a} or both are other than —H they are connected to N to which they are attached through a sp³-hybridized carbon atom;
—R⁷ is —N(R¹⁰R^{10a}); or —NR¹⁰—(C═O)—R¹¹;
—R^{7a}, —R¹⁰, —R^{10a}, —R¹¹ are independently of each other —H; or C₁₋₁₀ alkyl;
optionally, one or more of the pairs —R^{1a}/—R^{4a}, —R^{1a}/—R^{5a}, —R^{1a}/—R^{7a}, —R^{4a}/—R^{5a}, —R^{8a}/—R^{9a} form a chemical bond;
optionally, one or more of the pairs —R¹/—R^{1a}, —R²/—R^{2a}, —R⁴/—R^{4a}, —R⁵/—R^{5a}, —R¹/—R^{8a}, —R⁹/—R^{9a} are joined together with the atom to which they are attached to form a C₃₋₁₀ cycloalkyl; or 3- to 10-membered heterocyclyl;
optionally, one or more of the pairs —R¹/—R⁴, —R¹/—R⁵, —R¹/—R⁶, —R¹/—R^{7a}, —R⁴/—R⁵, —R⁴/—R⁶, —R¹/—R⁹, —R²/—R³ are joined together with the atoms to which they are attached to form a ring A;
optionally, —R³/—R^{3a} are joined together with the nitrogen atom to which they are attached to form a 3- to 10-membered heterocycle;
ring A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
each -L¹- is substituted with -L²- provided that the hydrogen marked with the asterisk in formula (I) is not replaced by a substituent.

33. A pharmaceutical composition comprising the drug conjugate or pharmaceutically acceptable salt thereof of claim 23 and at least one pharmaceutically acceptable excipient.

34. (canceled)

35. A method of treating a patient in need thereof, the method comprising the step of administering a pharmaceutically effective amount of the drug conjugate or pharmaceutically acceptable salt thereof of claim 23 or a pharmaceutical composition comprising said drug conjugate or pharmaceutically acceptable salt thereof to said patient.

36. A method of preparing a drug conjugate or pharmaceutically acceptable salt thereof, wherein the method comprises the following steps:
(a) providing the hydrogel HA microspheres or pharmaceutically acceptable salts thereof obtained by the method of claim 1, wherein said hydrogel comprises one or more unreacted -FG₁ or -FG₂;
(b) providing a monoconjugate reagent D-L¹-L²-FG₃, a bisconjugate reagent FG₃-L²-L¹-D-L¹-L²-FG₃ or a trisconjugate reagent of formula (t): 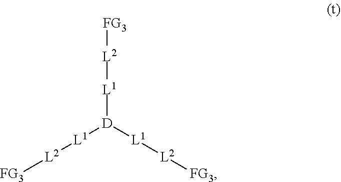
wherein each -D is independently a drug moiety that is covalently and reversibly conjugated to -L¹-;
each -L¹- is independently a reversible linker moiety;
each -L²- is independently a spacer moiety or absent;
each -FG₃ is independently a functional group that reacts with one -FG₁ or -FG₂;
(c) mixing the hydrogel HA microspheres of step (a) with the monoconjugate, bisconjugate or trisconjugate reagent of step (b);
(d) mixing the drug conjugate or pharmaceutically salt thereof of step (c) with a blocking reagent; and
(e) collecting the drug conjugate or pharmaceutically acceptable salt thereof of step (c) or (d).

37. A drug conjugate or pharmaceutically acceptable salts thereof obtainable by the method of claim 36.

38. A method for precipitating a polymer in a flow system, wherein the method comprises the steps of:
(a′) flowing, optionally simultaneously, a first solution comprising the polymer through a first channel and a second solution comprising an anti-solvent through a second channel;
(b′) combining the first and second solutions of step (a′);
(c′) flowing the combined mixture of step (b′) into at least one precipitating unit; and
(d′) precipitating the polymer;
wherein if more than one precipitating unit is present, the mixture comprising the precipitated polymer that flows out from one precipitating unit is combined with the second solution comprising the anti-solvent or optionally with another solution comprising an anti-solvent before it is flowed into another precipitating unit.

39. A method for isolating a polymer in a setup for precipitating and isolating a polymer, wherein the method comprises the steps of:
(a′) flowing, optionally simultaneously, a first solution comprising the polymer through a first channel and a second solution comprising an anti-solvent through a second channel;
(b′) combining the first and second solutions of step (a′);
(c′) flowing the combined mixture of step (b′) into at least one precipitating unit;
(d′) precipitating the polymer; and
(e′) isolating the precipitate of step (d′),
wherein if more than one precipitating unit is present, the mixture comprising the precipitated polymer that flows out from one precipitating unit is combined with the second solution comprising the anti-solvent or optionally with another solution comprising an anti-solvent before it is flowed into another precipitating unit.

40. The method of claim 38, wherein one precipitating unit is present.

41. The method of claim 38, wherein two precipitating units are present and the mixture comprising the precipitated polymer that flows out from the first precipitating unit is combined with the second solution comprising the anti-solvent before it is flowed into a second precipitating unit.

42. The method of claim 38, wherein the polymer is selected from the group consisting of polysaccharides such as hyaluronic acid, hyaluronic acid and derivatives or functionalized hyaluronic acid, heparin, heparan sulfate, heparosan, chondroitin sulfate, dermatan sulfate, keratan sulfate, cellulose, carboxymethyl cellulose, hydroxypropyl methylcellulose, chitin, chitosan, dextran or dextrin; polyethers, such as poly(ethyleneglycol) or poly(propylene glycols); polyesters, such as polyhydroxybutyrate, poly(glycolic acid), polybutylene terephthalate, poly(caprolactone), poly(lactic acid) or poly(lactic-co-glycolic acid); proteins, such as gelatin or collagen; polyolefins, such as poly(2-methacryloyl-oxyethyl phosphorylcholine), poly(acrylic acid), poly(acrylate), poly(acrylamide), poly(cyanoacrylate), poly(dimethylacrylamide), polyethylene, poly(hydroxyethyl acrylate), poly(2-hydroxyethyl methacrylate), poly(N-(2-hydroxypropyl)methacrylamide), poly(hydroxypropyl methacrylate), poly(vinyl alcohol), poly(vinyl amine), poly(vinylmethylether) or poly(vinylpyrrolidone); poly(oxazolines), such as poly(methyloxazoline) or poly(ethyloxazoline); polyamides; poly(amidoamines); poly(amino acids); polyanhydrides; poly(aspartamides); polycarbonates; poly(alkylene phosphates) such as poly(ethylene phosphates); poly(iminocarbonates); poly(methacrylamides); poly(organophosphazenes); poly(ortho esters); poly(siloxanes) and poly(urethanes).

43. (canceled)

44. The method of claim 38, wherein the polymer is a functionalized HA.

45. The method of claim 38, wherein the first solution comprises a functionalized HA, wherein the functionalized HA comprises a plurality of each of the following linearly connected Z¹ and Z⁵-i unit: 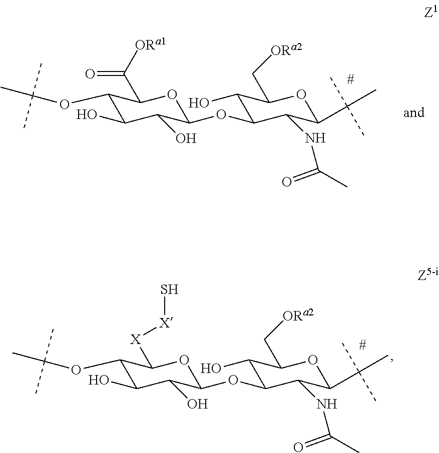
or a plurality of each of the following linearly connected Z¹ and Z⁶-i units: 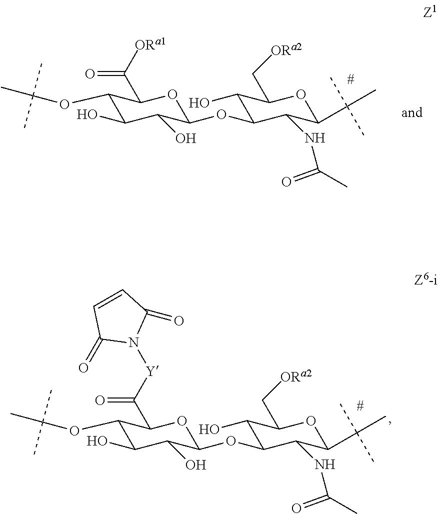
or a plurality of each of the following linearly connected Z¹ and Z⁷-i units: 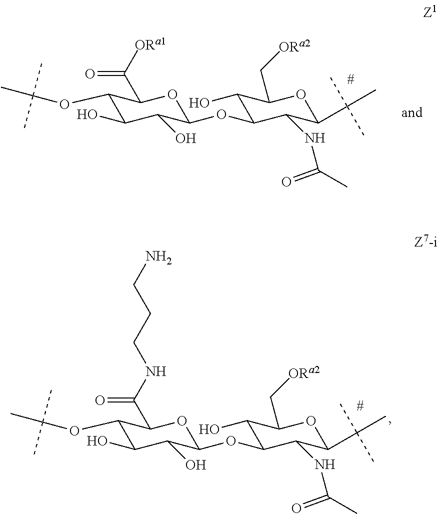
wherein
an unmarked dashed line indicates a point of attachment to an adjacent unit at a dashed line marked with # or to a hydrogen atom;
a dashed line marked with # indicates a point of attachment to an adjacent unit at an unmarked dashed line or to a hydroxyl group;
—X′— is of formula (x4): 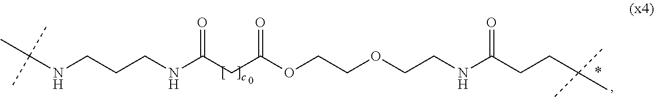
wherein the unmarked dashed line indicates the attachment to the carbonyl group, the dashed line marked with an asterisk indicates the attachment to the sulfur atom and c₀ is 7;
—Y′— is of formula (y4): 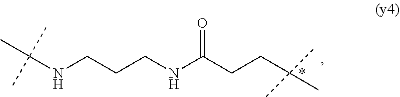
wherein the unmarked dashed line indicates the attachment to the carbonyl group and the dashed line marked with an asterisk indicates the attachment to the nitrogen atom of the maleimide ring;
each R^{a1} is H or an alkali metal;
each —R^{a2} is —H; and the second solution is ethanol.

46. A flow system for precipitating a polymer comprising:
a vessel comprising a first solution comprising the polymer;
at least one storage vessel comprising a second solution comprising an anti-solvent;
at least one combining unit for combining the first and second solutions or for combining the mixture that flows out from a precipitating unit with the second solution comprising an anti-solvent or optionally with another solution comprising an anti-solvent;
at least one precipitation unit for precipitating the polymer;
wherein the vessel, at least one storage vessel, at least one combining unit and the at least one precipitation unit are connected via connective channels to provide a continuous flow path, wherein
the first solution flows through a first channel from the vessel to the combining unit;
the second solution flows through a second channel from the at least one storage unit to the combining unit;
the combined mixture flows through at least one of the precipitation units and wherein if more than one precipitating unit is present, the mixture comprising the precipitated polymer that flows out from one precipitating unit is combined with the second solution comprising the anti-solvent or optionally with another solution comprising an anti-solvent before it is flowed into another precipitating unit.

47. The flow system of claim 46, wherein the vessel and the at least one storage vessel are further connected to a valve and/or pump to control the flow rate of the mixture comprising the polymer and the anti-solvent.

48. The flow system of claim 46, further comprising storage vessel for storing a buffering agent, wherein said storage vessel is connected via channels and a valve to an outflow of the vessel.

49.-53. (canceled)
